(19) United States
(12) Patent Application Publication
(10) Pub. No.: US 2021/0255170 A1
(43) Pub. Date: Aug. 19, 2021

(54) INDUCTIVE REGENERATION OF THE AIRWAY BY TRANSCRIPTIONAL FACTOR MODULATION OF GLANDULAR MYOEPITHELIAL STEM CELLS

(71) Applicant: University of Iowa Research Foundation, Iowa City, IA (US)

(72) Inventors: John F. Engelhardt, Iowa City, IA (US); Thomas J. Lynch, Iowa City, IA (US); Preston J. Anderson, Iowa City, IA (US); Weam Shahin, Iowa City, IA (US)

(21) Appl. No.: 16/980,268

(22) PCT Filed: Mar. 13, 2019
(86) PCT No.: PCT/US2019/022106

§ 371 Date: Sep. 11, 2020

Related U.S. Application Data
(60) Provisional application No. 62/642,320, filed on Mar. 13, 2018.

(51) Int. Cl. G01N 33/50; C07K 14/475; C07K 14/47

(52) U.S. Cl. CPC G01N 33/5061; C07K 14/4753; G01N 33/5073; G01N 33/5041; C07K 14/4705

(57) ABSTRACT

Compositions and methods to modulate Lef-1/TCF/Wnt signaling ex vivo or in vivo, and assays to detect those modulators, are described.

### CROSS-REFERENCE TO RELATED APPLICATIONS

This application claims the benefit of the filing date of U.S. application No. 62/642,320, filed on Mar. 13, 2018, the disclosure of which is incorporated by reference herein.

### STATEMENT OF GOVERNMENT RIGHTS

This invention was made with government support under DK047967, HL051670, and DK054759 awarded by National Institutes of Health. The Government has certain rights in the invention.

### SUMMARY

The disclosure provides a composition comprising an isolated transcription factor (Lymphoid enhancer factor 1 or Lef-1) that when introduced to or induced in a specific airway stem cell leads to self-limiting regenerative expansion of the airway and submucosal glands. Using genetically engineered mice, Lef-1 expression in glandular myoepithelial cells (MECs) was shown to either enhance airway repair following injury (with monoallelic Lef-1 expression) or spontaneously induce MEC-mediated airway regeneration (with biallelic Lef-1 expression). Thus, modulation of the level of Lef-1 expression in MECs controls lineage commitment of this progenitor toward 8 daughter cell lineages involved in airway regeneration. Lef-1 expression enhances the self-renewal of MECs in vitro and thus may be useful in their expansion and therapeutic applications. These findings open the door to enhancing the regenerative capacity of MECs ex vivo using cell therapy approaches and in vivo using small molecules that influence Lef-1 function and expression. Since surface airway basal cells are descendents of MECs, Lef1 or other TCF modulation in surface airway basal cells allows for phenotypes described herein. There are four TCFs in humans: Lef1, TCF1, TCF3 and TCF4.

Applications of the findings include but are not limited to: 1) modulating Lef-1 in order to treat degenerative lung diseases and/or conditions such asthma, COPD, cystic fibrosis, and other forms of airway epithelial damage; 2) the use of genetically or chemically modified airway stem cells using Wnt/Lef-1 pathways for applications in cell therapy for lung transplants in which glandular stem cell niches are exhausted and destroyed as obliterans bronchiolitis develops; 3) delivery of Wnt/Lef-1 analogs (chemical, RNA, miRNA, protein or DNA-based) that modulate stem cells in vivo or ex vivo followed by transplantation back into patients; and 4) assays disclosed herein to screen for small molecules that produce the same therapeutic effect as enhancing Lef-1 expression.

The disclosure provides for an in vitro method to identify modulators of LEF-1 or other related transcription factors (TFs) such as the T-cell factor (TCF) family of TFs, e.g., TCF-1, TCF3 or TCF-4, or Wnt signaling. The method includes contacting one or more test compounds with isolated mammalian myoepithelial stem cells (MECs) or basal cells derived therefrom, mammalian cells that exogenously express Lef-1 or TCF, or mammalian cells, the genome of which is altered with a reporter gene so as to detect LEF-1 or TCF expression or Wnt signaling; and detecting or determining whether the one or more compounds alter the expression of Lef-1 or TCF, or alter Wnt signaling. In one embodiment, at least one of the compounds is a Lef-1, TCF or Wnt activator. In one embodiment, at least one of the compounds is a Lef-1, TCF or Wnt inhibitor. In one embodiment, the compound is RNA, e.g., miRNA, DNA or protein. In one embodiment, the cells are human cells. In one embodiment, the genome of the cells is genetically altered with a vector having a reporter gene inserted into the 3′UTR of a Lef-1 or TCF, e.g., TCF-1, gene. In one embodiment, the marker gene is a fluorescent gene, e.g., a GFP gene.

Also provided is a pharmaceutical composition comprising an amount of LEF-1 or TCF having at least 80% amino acid sequence identity to one of SEQ ID Nos 1-2 or 5-9 or comprising an agent that induces the expression of LEF-1 or TCF in a mammalian cell. In one embodiment, the composition further comprises a pharmaceutically acceptable carrier. In one embodiment, the TCF or Lef-1 has at least 90% or 95% amino acid sequence identity to SEQ ID NO:5 (human TCF-1) or SEQ ID NO:9 (human Lef-1). In one embodiment, in the amount is effective to enhance airway repair following injury or induce MEC regeneration in an airway of a mammal.

Further provided is an in vitro method to culture and/or expand mammalian stem cells, comprising: culturing myoepithelial stem cells (MECs) or basal cells derived therefrom with a composition comprising an effective amount of one or more modulators of LEF-1 or TCF, or Wnt signaling. In one embodiment, the composition comprises LEF-1 or TCF having at least 80% amino acid sequence identity to SEQ ID Nos 1-2 or 5-9, or an agent that induces the expression of LEF-1 or TCF in a mammalian cell.

An in vitro method to prepare mammalian ionocytes is provided. The method includes culturing mammalian myoepithelial stem cells (MECs) or basal cells derived therefrom with a composition comprising an effective amount of an activator of LEF-1 or TCF, or a modulator of Wnt signaling.

A method to expand mammalian glandular myoepithelial stem cells (MECs) and optionally differentiate the MECs, or to induce ionocytes, in a mammal is provided. The method includes administering to the mammal an effective amount of a composition comprising one or more modulators of LEF-1, TCF or Wnt signaling or comprising cells exposed ex vivo to one or more modulators of LEF-1, TCF or Wnt signaling. In one embodiment, the composition comprises a LEF-1 having at least 80% amino acid sequence identity to SEQ ID NO:1 or SEQ ID NO:9, cells transduced with an expression cassette comprising a nucleic acid encoding the LEF-1, cells exposed ex vivo to isolated LEF-1 having at least 80% amino acid sequence identity to SEQ ID NO:1 or SEQ ID NO:9, or an agent that induces the expression of LEF-1 in a mammalian cell, or the composition comprises a TCF-1 having at least 80% amino acid sequence identity to SEQ ID NO:5, cells transduced with an expression cassette comprising a nucleic acid encoding the TCF-1, cells exposed ex vivo to isolated TCF-1 having at least 80% amino acid sequence identity to SEQ ID NO:5, or an agent that induces the expression of TCF-1 in a mammalian cell. In one embodiment, the composition comprises cells transduced with an expression cassette comprising a nucleic acid encoding the LEF-1 or TCF, or cells exposed ex vivo to an activator of LEF-1, TCF or Wnt signaling. In one embodiment, the mammal is a human. In one embodiment, the amount is administered before or after, or both before and after, a lung transplant. In one embodiment, the amount is administered during a lung transplant. In one embodiment, the composition is intratracheally, systemically or intranasally administered. In one embodiment, the composition is bronchoscopically administered. In one embodiment, the mammal has cystic fibrosis. In one embodiment, the amount increases the number of ionocytes and/or enhances airway regeneration. In one embodiment, the composition comprises lithium, CHIR 99021, BIO, SB-216763, CAS 853220-52-7, WAY 262611, R-spondin, norri, ICG-001, PNU-74654 or windorphen. In one embodiment, the composition comprises an activator of GSK-3 including but not limited to valproic acid, iodotubercidin, naproxen, cromolyn, famotidine, curcurmin, olanzapine, or a pyrimidine derivative.

A method to prevent, inhibit or treat a degenerative lung disease or disorder, or enhance airway repair, in a mammal is provided comprising: administering to the mammal an effective amount of a composition comprising one or more modulators of LEF-1, TCF or Wnt signaling. In one embodiment, the composition comprises LEF-1 having at least 80% amino acid sequence identity to SEQ ID NO:1 or SEQ ID NO:9, cells transduced with an expression cassette comprising a nucleic acid encoding the LEF-1, cells exposed ex vivo to isolated LEF-1 having at least 80% amino acid sequence identity to SEQ ID NO:1 or SEQ ID NO:9, or an agent that induces the expression of LEF-1 in a mammalian cell, or the composition comprises a TCF-1 having at least 80% amino acid sequence identity to SEQ ID NO:5, cells transduced with an expression cassette comprising a nucleic acid encoding the TCF-1, cells exposed ex vivo to isolated TCF-1 having at least 80% amino acid sequence identity to SEQ ID NO:5, or an agent that induces the expression of TCF-1 in a mammalian cell. In one embodiment, the disease is COPD, emphysema, cystic fibrosis, related to allograft rejection such as chronic lung allograft dysfunction (CLAD), including bronchiolitis obliterative syndrome and/or restrictive allograft syndrome, primary lung graft dysfunction or the result of graft versus host disease (GvHD). In one embodiment, the mammal is a human. In one embodiment, the amount is administered before or after, or both before and after, a lung transplant. In one embodiment, the amount is administered during a lung transplant. In one embodiment, the composition is intratracheally, systemically or intranasally administered. In one embodiment, the composition is bronchoscopically administered. In one embodiment, the mammal has cystic fibrosis. In one embodiment, the amount increases the number of ionocytes and/or enhances airway regeneration. In one embodiment, the composition comprises lithium, CHIR 99021, BIO, SB-216763, CAS 853220-52-7, WAY 262611, R-spondin, norri, ICG-001, PNU-74654 or windorphen. In one embodiment, the composition comprises an activator of GSK-3 including but not limited to valproic acid, iodotubercidin, naproxen, cromolyn, famotidine, curcurmin, olanzapine, or a pyrimidine derivative.

### BRIEF DESCRIPTION OF THE FIGURES

FIGS. 1A-L. MEC-derived cells emerge from SMGs and adopt a basal cell-like phenotype in the SAE following injury. (A) Timeline of lineage-tracing of myoepithelial cells with tamoxifen (Tmx) and airway injury with naphthalene (Naph). ACTA2-Cre^{ERT2}:ROSA-TG mice were treated with tamoxifen daily for 5 days, rested for five days, and then treated with either vehicle or naphthalene (300 mg/kg) and euthanized 14 or 21 days post-injury (DPI). (B-H) Tracheal sections at the indicated time points are oriented with the proximal region to the left and were stained for nuclei, Tomato, GFP, and the indicated phenotypic markers: (B) αSMA expression at 14 DPI (b: enlarged image of the boxed region shown in B); (C) Krt5 expression at 14 DPI (c: enlarged image of the boxed region in C); (D) Trop2 expression in a gland duct at 21 DPI; (E,F) NGFR expression at 21 DPI (fi and fii: enlarged images of the boxed regions shown in F); (G) Krt8 expression at 21 DPI; (H) Krt14 expression at 21 DPI; and (I) Control trachea at 21 days post-induction in the absence of epithelial injury. (J-K) Quantitation of lineage-traced cells in the SAE at 21 DPI as (J) % of total cells that are GFP⁺ cells in the C0-C4 region of the SAE, and (K) distribution of total GFP⁺ cells at various cartilage ring segments. Dotted line in J marks background level of signal close to the basal lamina in uninjured controls. (L) Distribution of total GFP⁺αSMA+/− and Krt8+/− cells ate various cartilage ring segments in the SAE at the % total C0-C4 GFP⁺ expressing cells at 21 DPI. Graphs show means+/−SEM for N=6 mice. P-values indicate significance of (J) unpaired one-tailed Student's t-test, and (K,L) one-way ANOVA followed by posttest for linear trend.

FIGS. 2A-L. MEC-derived cells produce ciliated cells on the SAE following injury. (A) Experimental design and summary of results: ACTA2-Cre^{ERT2}:ROSA-TG mice were induced with tamoxifen as in FIG. 1A, treated with vehicle or naphthalene to induce injury, and harvested at the indicated time points. Circles indicate the presence (closed) or absence (open) of lineage-traced ciliated cells on the SAE. Tracheal sections were stained for nuclei, Tomato, GFP, and acetylated αtubulin. (B) Vehicle treated mice harvested at 60 days post mock (DPM). (C-E) Naphthalene treated mice harvested at 14, 21, or 60 days post injury (DPI). (F) Color key for lineage detection of ciliated cells in mice shown in panels G-I. The cilia of MEC-derived (GFP⁺) cells appear either white (left or top panel) or cyan (right or bottom panel); those of cells lacking the lineage-marker GFP (Tomato+) are either magenta (left or top panel) or white (right or bottom panel). (G-I) Enlarged, two-channel images of boxed regions in C-E show GFP (green)/αtubulin (magenta) and Tomato (red)/αtubulin (cyan). Traced (GFP⁺) ciliated cells are marked by closed yellow arrowheads, whereas their non-traced (Tomato+) counterparts are marked by open yellow arrowheads. (J,K) Quantitation of (J) % of total SAE cells that are GFP⁺ various DPI and (K) % of total GFP⁺ cells that also express αtubulin at various DPI. Graphs show means+/−SEM for (N) mice as depicted on graphs. (L) Uninjured ACTA2-Cre^{ERT2}:ROSA-TG mouse at 1.5 years following tamoxifen-induction. P-values indicate significance of one-way ANOVA followed by posttest for linear trend.

FIGS. 3A-O. MEC-derived progenitors reestablish niches in the SAE that respond to repetitive injury and are multipotent for both SAE and SMG cell types. (A) Diagram of design for lineage tracing experiment comparing single injury (SI) and double injury (DI). Two other groups of control mice also included: uninduced/uninjured (UIND) and induced/uninjured (UI). (B-K) Tracheal sections of DI mice were stained for nuclei, Tomato, GFP, and the indicated phenotypic markers: (B) no marker, showing boundary of a lineage-traced group of cells (b: enlarged inset from B); (C) αtubulin (c: enlarged inset from C); (D) Scgb1a1; (E) Dolichos biflorus agglutinin (DBA) lectin; (F) Lysozyme (Lyz) in SMGs; (G, H) Ulex europaeus agglutinin I (UEA-1) lectin in (G) SMG and (H) SAE; (I,J) Muc5B in (I) SAE and (J) SMG; and (K) Scgb3a2. Arrows in c indicate ciliated cells that are GFP⁺ (yellow) or GFP⁻ (red). (L,M) Quantification of the % total SAE cells that are GFP⁺ in the (L) SAE and (M) SMGs for all four groups of mice evaluated. (N-P) Quantification of (N,O) the (N) SAE and (O) SMG compartments showing the % of the total GFP⁺ (cyna) or % of the total Tomato⁺ cells (magenta) that also express the indicated phenotype markers after single injury (solid bars) or double injury (checkered bars). Graphs show means+/−SEM for N=4-6 mice per group. P-values indicate significance of (L,M) one-way ANOVA and (N,O) two-way ANOVA followed by Sidak's multiple comparisons test (ns=not significant, *P<0.05, **P<0.01, ***P<0.001,****P<0.0001).

FIGS. 4A-L. Wnt/13-Catenin signaling is similarly activated in primordial gland stem cells during development and MECs following airway injury. (A,B) Glandular placodes (arrows) from newborn trachea localizing (A) TCF7 and (B) Lef-1 with Sox2. (C-J) Localization of αSMA with Sox2, Lef-1, TCF7, or β-Catenin in SMGs of (C-F) uninjured and (G-J) 24 hr post naphthalene (300 mg/kg) injury. Panels to the right of F and J show (fi, ji) nuclear β-catenin (NRC) staining overlapping with DAPI (intensity of β-catenin staining is retained) and superimposed over at outline of αSMA staining (red lines) and (fii, jii) representative segmented images after multiwavelength cell scoring each nuclei showing αSMA⁻NβC⁻ (blue) and αSMA⁺NβC⁺ (yellow) cells (K,L) Quantification of nuclear Sox2, Lef-1, TCF7, or nuclear β-Catenin staining as (K) the % of total SMG cells and (L) the % of αSMA⁺ MECs. Graphs show means+/−SEM for N=3-6 mice per group. All micron bars=25 μm. Single daggers indicate significance of one-way ANOVA, and double daggers indicate posttest for linear trend.

FIGS. 5A-U. Lef-1 expression activates lineage commitment of MECs and migration to the SAE. (A) Transgenic ROSA26 knock-in construct (Lef-1KI) used to conditionally activate Lef-1 expression in MECs. (B) Experimental design for evaluating how Lef-1 expression influences MEC fate in ACTA2-Cre^{ERT2}:Lef-1K1^{+/+} vs. ACTA2-Cre^{ERT2}:ROSA-TG mice in (C-F). (C) Uninduced and uninjured ACTA2-Cre^{ERT2}:Lef-1K1^{+/+} demonstrating GFP expression in the majority of cells. (D,E) Tamoxifen induced/uninjured mice labeled with EdU as in (B) and stained for the indicated markers (d and ei-eiii: enlarged insets from D,E). (F) Quantification of EdU⁺αSMA⁺ MECs (N=5 mice per group). (G) Experimental design for evaluating how Lef-1 dosage impacts MEC fate with and without naphthalene (300 mg/kg) injury in (H-U). (H-S) ACTA2-Cre^{ERT2}:Lef-K1^{+/-} and ACTA2-Cre^{ERT2}:Lef-1K1+/+ mice were treated under the various conditions as marked and sections stained for (H-L) GFP and αSMA or (M-S) GFP and Lef-1. Insets of the SAE in (N,O) are from different animals at C6. (T,U) Quantification of lineage-traced cells (GFP⁻) as a % of the total cells in the (T) SMGs and (U) SAE of the C0-C4 tracheal region (N=3-4 mice per group). Graphs show means+/−SEM. Micron bars: (C-E, H-O) 100 μm; (P-S and insets N, O, i-iv) 50 μm. Asterisks indicate significance of (F) unpaired two-tailed Student's t-test and (T,U) Newman-Keuls multiple comparisons testing (*P<0.05, **P<0.01, ***P<0.001).

FIGS. 6A-O. Lef-1 overexpression in MEC SCs promotes terminal differentiation toward multipotent progenitors in the absence of self-renewal. (A-J) ACTA2-Cre^{ERT2}:Lef-K1^{+/-} mice were subjected to the injury protocol in FIG. 5G and tracheal sections immunostained for the indicated antigens (B&W inset of boxed regions in G and I show a Trop2⁺ SMG duct and αtubulin⁺ ciliated ducts, respectively). Arrows mark duct openings at the SAE. All images are from the C0-C4 region, except for (B) which is at C6. (K) Quantification of lineage-traced (GFP) and untraced (GFP⁺) club and ciliated cells as a % of total cells in the SAE (C0-C4) from experiments in (A-J). (L) Experimental design for evaluating how Lef-1 expression in ACTA2-Cre^{ERT2}:Lef-K1+/+ mice impacts self-renewal of MECs following sequential SO₂ (600 ppm) injury in (M,N,O). (M,N) GFP and Lef-1 immunostaining of tracheas from two (M) induced/uninjured and (N) induced/2×SO₂ injured ACTA2-Cre^{ERT2}:Lef-K1+/+ mice. (O) Quantification of GFP⁻ and Lef-1+ cells as a % of total cells in the SAE and SMGs from experiments outlined in (L). Graphs show means+/−SEM for N=5 mice in (K) and N=3 mice per group in (O). Micron bars: 50 μm. Asterisks indicate significance of (K) paired two-tailed Student's t-tests and (O) two-way ANOVA followed by Sidak's multiple comparisons test (*P<0.05 and ****P<0.0001).

FIGS. 7A-M. Lef-1 expression in MECs induces regenerative and basal cell transcriptional programs. MECs were isolated from tamoxifen-induced ACTA2-Cre^{ERT2}:ROSA-TG (N=4) and ACTA2-Cre^{ERT2}:Lef-K1^{+/+} (N=5) mice and purified by FACS at P1 in culture for RNAseq. (A) Heat map of 360 differentially expressed genes following unsupervised hierarchical clustering (Benjamini-Hochberg adjusted P<0.05). (B) Lef-1 expression levels in the two genotypes. (C) Principle component analysis (PCA) of the 13,337 genes expressed in the two groups. (D) IPA biological processes and functions defined by gene expression patterns showing p-values and z-scores. (E-I) Heat maps of the indicated IPA gene sets following unsupervised hierarchical clustering. (J,K) Motility assays on purified MEC^{WT} and MEC^{Lef-1KI+/+} in culture showing (J) migration plot and (K) distance traveled with time. Measurements were taken of N=16 randomly selected cells traced from N=3 cultures for each genotype from a single experiment; mean+/−SEM is shown in K with the P-value indicating the comparison between nonlinear models fitting MEC^{WT} and MEC^{Lef-1KI} cells. (L,M) Heat maps of (L) differentially expressed transcription factors and (M) basal cell specific genes determined in FIG. 12. In all heat maps, red indicates positive enrichment while blue indicates negative enrichment.

FIGS. 8A-J. Severe injury to the tracheal SAE leads to expansion of αSMA⁺ cell populations. Mice were injected with vehicle, 200 mg/kg naphthalene, or 300 mg/kg naphthalene and tracheas were harvest on day 1, 3, 5, and 7 following vehicle or naphthalene injection. (A-C) Immunofluorescent staining for αSMA expression at 3 days following (A) vehicle, (B) 200 mg/kg naphthalene, and (C) 300 mg/kg naphthalene injection. Arrowheads (C) mark a gland duct (white) and αSMA⁺ cells in the SAE (red). Tracheal cartilage rings are marked as cricoid cartilage (C0) and cartilage ring 1 (C1). (D) The percentage of total SAE cells that are αSMA⁺ cells in the SAE at C0-C2 under the various injury conditions. (E) Fold change, relative to uninjured animals, in the percentage of αSMA⁺ cells in the SMGs. Data are shown as mean±SEM of N=3-6 mice from multiple sections >60 μm apart. Diamonds denote significance levels for Two-way ANOVA test: ⋄ P<0.05 and 0000 P<0.0001. Asterisks denote significance levels for Holm-Sidak's multiple comparisons test: * P<0.05, ** P<0.01, ***P<0.001, and **** P<0.0001. (F-H) Lineage-tracing using (F) ACTA2-CreERT2:ROSA-TG and (G,H) MYH11-CreERT2:ROSA-TG. Mice were given 5 daily IP injections of tamoxifen, rested for 5 days, and then sacrificed for tracheal harvest and analysis by immunofluorescence. Tracheal sections were stained for nuclei, Tomato, GFP, and the indicated phenotypic markers: (F and G) αSMA and (H) SMMHC. (I, J) Multi-wavelength cell scoring was used to quantify the lineage-tracing efficiency for the two lines as the percentage of αSMA or SMMHC positive cells that also express the lineage marker GFP. Values represent the mean+/−SEM of N=7-10 mice per group.

FIGS. 9A-F. MEC-derived cells emerge from SMGs and adopt a basal cell-like phenotype on the SAE of injured MYH11-Cre^{ERT2}:ROSA-TG mice. MYH11-Cre^{ERT2}:ROSA-TG mice were given 5 daily IP injections of tamoxifen, rested for 5 days, and then injured with naphthalene (300 mg/kg). (A-E) Tracheal sections at 21 days post-injury are oriented with the proximal region to the left and were stained for nuclei, Tomato, GFP, and the indicated phenotypic markers: (A) αSMA (ai and aii: enlarged images of the boxed regions shown in A); (B) Krt5 (bi: enlarged image of the boxed region shown in B); (C) Krt14; (D) DBA (di and dii: enlarged images of the boxed regions shown in D); and (E) αTubulin (arrows denote lineage-traced ciliated cells). (F) Quantification of the percentage of total SAE cells that are GFP⁺ cells in the SAE (dotted line denotes background level of signal close to the basal lamina in uninjured controls). Values represent the mean+/−SEM of N=3 uninjured mice and N=6 injured mice. P-values indicate significance of (F) unpaired one-tailed Student's t-test, **P<0.01.

FIGS. 10A-M. MEC-derived progenitors contribute to basal and luminal cells in the SAE following SO₂ injury. (A) Timeline of lineage-tracing of myoepithelial cells in ACTA2-Cre^{ERT2}:ROSA-TG mice induced with tamoxifen (Tmx) and injured with SO₂ (600 ppm). (B-J) Images of the GFP lineage trace with co-stained antigens as indicated for the (B-G) SAE and (I-J) SMGs. (K,L) Quantification of the percentage of total SAE cells that are GFP-positive cells in the (K) SAE and (L) SMGs. P-values indicate significance of one-way ANOVA followed by posttest for linear trend. (M) Quantification of the percentage of total GFP-positive cells that express each of the indicated markers. Values represent the mean+/−SEM of N=4-7 mice per group. Krt8⁺ lineage-traced cells significantly increased over time (one-way ANOVA P=0.0064 with a posttest for linear trend P=0.0016). Micron bars: (B-G) 50 vim; (H-J) 25 vim.

FIGS. 11A-I. Lef-1 expression in MECs using the MYH11-Cre^{ERT2} driver enhances lineage contribution to SAE and SMGs following airway injury. MYH11-Cre^{ERT2}:Lef-K1^{+/-} mice were induced tamoxifen daily for 5 days, rested for 5 days, and then injured with naphthalene (300 mg/kg). Uninduced and induced/uninjured animals were used as controls. Animals were harvested at 21 days post-mock or naphthalene injury. (A-H) Tracheal images localizing the lineage trace (GFP) and αSMA for (A-D) uninjured and (E-H) injured animals. (I) Quantification of the percentage of total cells that are GFP-negative cells in the SAE and SMGs. Values represent the mean+/−SEM for the (N) mice. P-values indicate significance of Kruskal-Wallis and Dunn's post-test, *P<0.05. Micron bars: 50 μm.

FIGS. 12A-D. Basal cell transcriptional profile. (A) Surface airway epithelial cells were harvested and isolated by FACS into basal, club, and ciliated cell populations. Microarray analysis was performed on RNA collected from each cell population. (B) Principal component analysis of each sample indicates good separation of each cell type. (C) Unsupervised hierarchical clustering of genes showing distinct expression profiles for each cell type with at least 4 major groups of genes indicated by K-means++ gene clustering. (D) Examples of several canonical phenotypic markers indicated as being enriched (z-score>1.75) in each cell type.

FIGS. 13A-O. MEC-derived progenitor cells are highly proliferative in primary cultures and Lef-1 expression enhances this phenotype. (A-F) ACTA2-Cre^{ERT2}:ROSA-TG mice were induced by five daily injections with tamoxifen and cells were isolated from the (A-C) SAE and (D-F) SMGs five days after the last tamoxifen injection. (B,C) SAE and (E,F) SMG cells were expanded from passage 0-10 (P0-P10) and the proportion of SAE cells expressing Tomato or GFP at each passage was quantified by FACS. (G-L) Mixing experiments of P3 (G-I) untraced (red/Tomato⁺) or (J-L) lineage-traced (green/GFP⁺) glandular progenitors isolated from induced ACTA2-Cre^{ERT2}:ROSA-TG mice and mixed with non-transgenic SAE progenitors at a ratio of 10% SMG:90% SAE. Mixed cultures were expanded from P3-P10 and the proportion of each phenotype was quantified by FACS at each passage. (M-O) ACTA2-Cre^{ERT2}:Lef-1K1^{+/+} and ACTA2-Cre^{ERT2}:ROSA-TG mice were induced by five daily injections with tamoxifen and SMG cells were isolated five days later. FACS purified populations of MEC^{WT} (GFP⁺) and MEC^{Lef-1KI} (GFP⁻) at P3 were mixed at a ratio of 15% MEC^{Lef-1KI}:85% MEC^{WT} and cultured to the 8^{th} passage. The proportion of each phenotype was quantified by FACS at each passage. Data represents the mean+/−SEM for N=6 cultures.

FIGS. 14A-Q. Differentiation of WT and Lef-1K1^{+/+} MECs in air-liquid interface cultures and tracheal xenografts. (A) Schematic of experimental procedure for isolation of GFP⁺ MEC^{WT} and GFP-MEC^{Lef-1KI} from glands of tamoxifen-induced ACTA2-Cre^{ERT2}: ROSA-TG and ACTA2-Cre^{ERT2}: Lef-K1^{+/+} mice. Mice were induced by 5 sequential tamoxifen injections and rested for 5 days prior to harvest. (B-I) Phenotypes of cells in air-liquid interface (ALI) cultures established from a 50:50 mixture of MEC^{WT} and MEC^{Lef-1KI} cells showing (B-E) orthogonal views and (F-I) maximum intensity projections of the ALI culture. Cultures were immunostained for the indicated markers of (B,F) club (Scgb1a1), (C,G) ciliated (odubulin), and (D,H) Muc5AC and (E,I) Muc5B mucin secreting cells. (J-L) Denuded tracheal xenografts reconstituted with 90% non-transgenic SAE and 10% P2 ACTA2-Cre^{ERT2}:ROSA-TG labeled SMG cells (˜4% GFP⁺ and 6% Tomato⁺). Phenotypic markers assessed by immunofluorescence were: (J) Krt14, (K) αtubulin, and (L) UEA-1. For panels J, boxed regions are enlarged and displayed to the right. (M-Q) Denuded tracheal xenografts reconstituted with FACS purified SMG cells isolated from tamoxifen-induced ACTA2-Cre^{ERT2}:ROSA-TG (GFP⁺) and ACTA2-Cre^{ERT2}:Lef-K1^{+/+} (GFP⁻) mice and seeded at a ratio of 50:50. Sections are stained for the GFP and/or αtubulin.

FIGS. 15A-I. Glandular MECs give rise to basal cells in the mouse tracheal SAE and serous cells of SMGs following injury. αSMA-CreERT2:ROSA-TG mice were treated with tamoxifen daily for 5 days, rested for five days, and then injured with naphthalene (300 mg/kg) and euthanized at 14 days post-injury. Immunofluorescence was used to evaluate tracheal sections for the antigens indicated in each panel. All panels are oriented with the proximal portion of the trachea to the left. (A) Uninjured controls. The majority of GFP+ cells are MECs that express αSMA. No cells in the SAE express the lineage marker. (B) Following injury, GFP+Trop2+ duct cells are apparent. (C) MEC-derived BCs on the airway surface adopt a CK5+ phenotype. (D) More proximal MEC-derived BCs express αSMA, whereas those in an adjacent distal clone lack αSMA expression. (E) More proximal MEC-derived BCs lack NGFR expression (iv), whereas more distal clones express NGFR (iii). (F) MEC-derived cells differentiate into lysozyme-expressing serous tubules. (G,H) Marked MECs in the SAE form columnar clones. (I) Model: Glandular MECs represent a facultative stem cell for the SAE. Once on the airway surface, MECs adopt a BC program as they move distally within its new niche. ST: Serous tubule; C1-C5: Cartilage rings.

FIGS. 16A-P. MEC-derived progenitors establish SAE niches that respond to repetitive injury and are multipotent for both SAE and SMG cell types. αSMA-CreERT2:ROSA-TG mice were treated with tamoxifen daily for 5 days, rested for five days, and then injured with naphthalene according to the following protocol. (A) Design for lineage tracing experiment. Double injured (DI) mice were severely injured with 300 mg/kg naphthalene, allowed to recover for 21 days, were moderately injured with 200 mg/kg naphthalene and harvested after an additional 39 days (total 60 days). Single-injured mice (SI) were injured with 300 mg/kg, were mock injured (oil) 21 days later, and were euthanized after an additional 39 days. Uninjured mice (UI) were induced, received a single mock injury (oil), and recovered for 60 days. Uninduced mice (UIND) were not induced with tamoxifen, not injured, and age matched to other conditions. (B-K) Tracheal sections from DI mice stained for nuclei, tdTomato, GFP, and the indicated phenotypic markers: (B) tdTomato and GFP only (enlarged images of boxed regions to right are single channels); (C) αtubulin (enlarged image of boxed region to right show dual and single channels); (D) Scgb1a1; (E) Dolichos biflorus agglutinin (DBA) lectin marking mucous glandular tubule; (F) Lysozyme (Lyz) marking serous tubule; (G and H) Ulex europaeus agglutinin I (UEA-1) lectin marking serous cells in SMGs and mucus-secreting cells in the SAE, (I-J) Muc5B in the SAE (I) and SMGs (J), and (K) Scgb3a2 in the SAE. (L, M) Quantitation of results from morphometric analysis of (L) GFP+ cells on the SAE from cartridge region C0-C4, and (M) GFP+ cells in the SMGs, under various injury conditions. (N-O) Phenotypic quantification, in SI (white bars) and DI (black bars) mice, of the percentage of GFP+ cells that also express markers for various cell types in the (N) SAE or (O) SMGs. (P) Quantitation of percentage of tdTomato+ (lineage-negative) cells on the SAE that also express the specified phenotypic markers (control for changes in the distribution following DI). Data represent the mean±SEM for N=4-10 mice from multiple sections >50 μm apart. Asterisks in L-P denote significance, as determined using two-tailed Student's t test; *P<0.05; **P<0.01; ***P<0.001; and ****P<0.0001.

FIGS. 17A-H. MECs induce Lef-1 and suppress Sox2 following airway injury. (A,B) Immunolocalization of (A) Lef-1/αSMA and (B) Sox2/αSMA in SMGs of uninjured C57BL/6J mice. (C,D) Immunolocalization of (C) Lef-1/αSMA and (D) Sox2/αSMA in SMGs of naphthalene (300 mg/kg) injured mice at 24 hrs post-injury. Insets to the right of A and C show single-channel images of Lef-1 and nuclear (HO342) stain. (E-H) Morphometric quantification of % SMG cells that are (E) Lef-1+ or (F) Sox2+ in the uninjured (UI) state and 12 hr and 24 hr post-injury, and % of αSMA+ MECs that are also (G) Lef-1+ or (H) Sox2+. Data represent the mean±SEM for N=4 mice, from multiple sections >50 μm apart. Note that following injury, glandular MECs adopt the same phenotype (Lef-1+/Sox2-) as primordial glandular stem cells during SMG development.

FIGS. 18A-C. Glandular MECs require the Lef-1 transcription factor to contribute to SAE and SMG cell types following airway injury. αSMA-CreERT2:ROSA-TG:Lef-1(Flx/Flx) mice were treated with tamoxifen daily for 5 days, rested for five days, and then injected with either (A) vehicle or (B, C) 300 mg/kg naphthalene. Mice were sacrificed at 21 days post-injury. (A-C) Immunolocalization of αSMA together with GFP and tdTomato. Single- and dual-channel images are shown to right of the main three-color panels in (A, B). Note that the MEC lineage trace (GFP) persists in the absence of Lef-1 only in the uninjured (A) animals. In the context of injury and in the absence of Lef-1, glands are repopulated with untraced MECs (i.e., GFP− αSMA+) (B), and the lineage-traced MECs do not contribute to the SAE (C). The efficiency of lineage tracing with the αSMA-CreERT2:ROSA-TG line is typically about 85% (i.e., 85% of αSMA+ cells are GFP+ after 5 days of induction and 5 days recovery). These data suggest that the 15% of untraced (Lef-1+) MECs account for the majority of glandular and SAE repair following injury.

FIGS. 19A-D. Lef-1 is required for the survival of glandular progenitor cells and MECs following airway injury. Lef-1(Flx/Flx):ROSA-TG:ROSA-CreERT2 mice were induced with tamoxifen 5×, rested for 5 days, and then naphthalene injured (day 0). (A) Expression of the GFP and tdTomato reporters on day 0 prior to injury. The majority of the glands and the SAE express not only GFP but also Tomato, due to lack of cellular turnover. Inset: αTubulin staining for cilia in the SAE co-localizes with membrane-bound GFP (white) in 50% of ciliated cells. (B,C) At 21 days post-injury, clonal areas of SMG tubules lose GFP expression, suggesting turnover of Lef-1 KO cells. The SAE also loses the majority of GFP+ Lef-1− cells, most notably BCs, and surviving GFP+ cells in the SAE are mostly columnar, with and without cilia (Inset in B,C). Those glandular tubules that retain GFP+ lumenal cells have primarily GFP− MECs (right panels in B). (D) Day 21 induced but uninjured controls demonstrate retention of Lef-1 KO (GFP+) cells in the SMGs and SAE

FIGS. 20A-J. Lef-1 expression drives the commitment of MECs toward more differentiated SMG and SAE cell types and increases the regenerative capacity of this stem cell compartment following injury. (A) Transgene expression cassette for inductive expression of human Lef-1 and protocol for tamoxifen induction of αSMA-CreERT2:Lef-1KI mice, naphthalene injury, and harvest. Mice are either heterozygous (Lef-1K1+/−) or homozygous (Lef-1K1+/+) for the human Lef-1 transgene as marked. (B) Tracheal sections from injured/uninduced mice, localizing GFP and αSMA. Nearly all cells in the SAE and SMGs are green. (C-F) Tracheal sections from tamoxifen-induced αSMA-CreERT2 mice of the (C) uninjured Lef-1K1+/−, (D) injured Lef-1K1+/−, (E) uninjured Lef-1K1+/+, and (F) injured Lef-1K1+/+ groups. (G, H) Quantification of the percentage of cells in the (G) SMG and (H) SAE that are GFP− (i.e., lineage-traced Lef-1KI+ cells). (I, J) Lef-1 and K5 immunostaining in (I) induced and (J) uninduced Lef-1K1+/+ mice.

FIG. 21. Lef-1 induced differentially expressed (DE) genes and IPA gene sets for various cellular functions. Primary glandular cells were isolated from αSMA-CreERT2:ROSA-TG and αSMA-CreERT2:Lef-1K1+/+ mice (N=4 each group) following 5 daily tamoxifen treatments and placed into culture. At confluence, cells were FACS isolated into RNA harvest buffer (GFP+ cells for WT, and GFP-cells for Lef-1KI). Benjamini-Hochberg (BH) adjusted P value of <0.05 identified 320 DE genes. 1436 genes with a relaxed BH P<0.10 were used for IPA analysis using Log 2FC between groups, IPA P values and Z-scores for various gene sets are also given.

FIGS. 22A-E. MEC growth rate and composition of glandular cell cultures. Tamoxifen induced αSMA-CreERT2:ROSA-TG mice were used to selectively isolate total SMG cells. (A,B) MECs constitute (A) 15% of cells in culture at P0 and (B) 75% at P10. (C) Proportion of SMG cells expressing tdTomato or GFP over serial passages. Note that the efficiency of lineage tracing is 85% in vivo (i.e., 85% of αSMA+ cells are GFP+). Thus, cultures of crude SMG cells are 90% MEC-derived by P5. (D,E) Organoid cultures with (D) SMG cells and (E) SAE BCs. Note: the organoids produced by SMG cells are tubular whereas those produced by SAE cells are spherical.

FIGS. 23A-G. Cas9-mediated gene editing in CRCs of primary airway BCs. (A) ROSA-TG construct expressed at homozygosity in primary BCs transduced and selected for Cas9 expression following lentivirus transduction. (B,C) Cells were (B) mock transfected or (C) transfected with a LoxP sgRNA. Deletion of LsL-tdTomato leads to expression of EGFP. (D-F) FACS analysis following transfection with (D) the indicated sgRNAs, and quantification of various phenotypes generated using (E) LoxP sgRNA or (F) tdTomato sgRNA. (G) Transcriptional activation of the Lef-1 gene in primary BCs using a dCas9 (i.e., nuclease-dead mutant) variant fused to VP64 with or without the MS2-p65 co-activator domain. Primary cultures of BCs were transduced with lentiviruses expressing these variants, and mixed populations were selected. Cells were then transfected with three sgRNAs (individually or in combination) that target the Lef-1 promoter. Levels of Lef-1 mRNA normalized to actin mRNA are shown. Graphs show means+/−SEM or range of N=2-3 transfections for each experimental point. Note: dCas9-VP64/p65 provides a higher level of transactivation than dCas9-VP64 alone, and more than one sgRNA increases expression in both cases.

FIGS. 24A-G. Creation of a ROSA-TG Cre reporter ferret and proposed approach for the creation of an αSMA-IRES-CreERT2 ferret. (A) Schematic for creation of ROSA-TG ferret by CRISPR/Cas9-mediated insertion of CAG-Loxp-tdTomato-stop-LoxP-EGFP transgene into intron 1 of the ferret ROSA-26 locus. (B) X-ray and tdTomato fluorescent images of a ROSA-TG founder ferret. (C,D) Fibroblasts generated from a ROSA-TG ferret were either (C) mock infected or (D) infected with a recombinant adenovirus expressing Cre. (E-F) Shown in (E) is the αSMA ferret gene with the proposed gRNA target site for insertion of the (F) IRES-CreERT2 cassette with 800 bp flanking homology at the target site. (G) The single-stranded DNA oligonucleotide will be generated in vitro by exonuclease degradation of one strand, and will be used for targeting in ferret zygotes.

FIGS. 25A-B. Primary ferret glandular myoepithelial cells reconstitute tracheal airway epithelium. A primary airway epithelial xenograft mode was used to assess if culture-expanded ferret myoepithelial stem cells can reconstitute airway epithelium. Briefly, tracheal scaffolds were decellularized by repeated freeze-thawing before being seeded with primary ferret submucosal gland (SMG) myoepithelial cells. Xenografts were maintained for five weeks prior to harvesting, and sections were stained for Krt14, Lef-1 and DAPI. (A) Ferret MECs are capable of reconstituting both SMGs and surface airway epithelium (SAE). (B) Lef-1 is expressed in glandular stem cells during de novo gland bud formation, similar to what is observed during tracheal development (Xie et al., 2014).

FIG. 26. Differentially expressed genes with Lef-1 overexpression in MECs. A total of 359 out of 13,336 genes were differentially expressed (BH-adjusted P-value <0.05) between WT and Lef-1KI sample groups. Of these, 338 genes were up-regulated (magenta-highlighted in column W) and 21 genes were down-regulated (green-highlighted in column W) with Lef-1 overexpression. Columns N through V are expression enrichment z-scores for each sample, and these values are plotted in FIG. 7A.

FIG. 27A. Biological processes and functions analysis. Relevant biological processes and functional pathways were identified in IPA software. Shown are the top 100 differentially regulated processes including several pathways highlighted in yellow that are referenced to in FIG. 7D-I and were selected based on their relevance to airway stem cell biology.

FIG. 27B. Gene set for IPA pathway Cell Movement. Shown are genes involved in cell movement that are expressed in WT and Lef-1KI cells. Expression enrichment values (columns K through S) are graphed in FIG. 7E.

FIG. 27C. Gene set for IPA pathway Migration of Cells. Shown are genes involved in migration of cells that are expressed in WT and Lef-1KI cells. Expression enrichment values (columns K through S) are graphed in FIG. 7F.

FIG. 27D. Gene set for IPA pathway Organismal Death. Shown are genes involved in organismal death that are expressed in WT and Lef-1KI cells. Expression enrichment values (columns K through S) are graphed in FIG. 7G.

FIG. 27E. Gene set for IPA pathway Formation of the Lung. Shown are genes involved in formation of the lung that are expressed in WT and Lef-1KI cells. Expression enrichment values (columns K through S) are graphed in FIG. 7H.

FIG. 27F. Gene set for IPA pathway Branching of Epithelial Tissue. Shown are genes involved in branching of epithelial tissue that are expressed in WT and Lef-1KI cells. Expression enrichment values (columns K through S) are graphed in FIG. 7I.

FIG. 27G. Transcriptional Regulators. Shown are transcriptional regulator genes that are expressed in WT and Lef-1KI cells. Expression enrichment values (columns K through S) are graphed in FIG. 7L.

FIG. 28. Bulk RNA-seq expression data for basal cell enriched genes expressed in MEC^{WT} and MEC^{Lef-}1KI cells. Shown are a subset of genes that were enriched in basal cells (Z-score >1.5). Columns D through L are Log 2 TPM values for each MEC sample. Columns M through U are expression enrichment Z-scores for each sample, and these values are plotted in FIG. 7M.

FIG. 29A-E. Lef1 Expression in Myoepithelial Cells Promotes Ionocyte Differentiation: Primary Myoepithelial Cells (MECs) were prepared from 4-week-old ROSA-^{LsL}nTomato ROSA-^{L}EGFP^{sL}-Lef-1KI mice. At P3, cells were treated with TatCre enzyme or vehicle to induce Cre recombination. Cells were FACS sorted at P5 then plated in Matrigel in transwells in SAGM media with supplement for 9 days. Media were then switched to pneumacult ALI for another 24 days. Wells were either fixed in 4% paraformaldehyde and imbedded in OCT for sectioning and immunofluorescence labeling or used for RNA preparation. (AC) Sections of Matrigel imbedded organoids were immunolabeled for FoxI1, GFP and tdTomato (A). (B) A higher magnification of the boxed areas in (A). (C) Quantification of FoxI1 positive cells. (D and E) qPCR for FoxH (D) and Cftr (E). n=3, **P<0.01 and ****P<0.0001, t-test. Error bars indicate SEM.

FIG. 30. Genes that interact with Lef-1.

FIG. 31. Probes that silence Lef-1.

### DETAILED DESCRIPTION

Currently, there are no therapies that modulate Wnt signaling in stem cells of the airway to enhance regeneration. Lef-1 is a key transcription factor in the Wnt signaling pathway. Lef-1 was found to enhance the multipotency of reserve stem cells in the airway found within SMGs. Lef-1 induction in MECs led to regeneration of the SAE and SMGs. SMGs are severely affected in cystic fibrosis, and there are currently no therapies that can target this region of the airway. Furthermore, SMGs are found throughout all cartilaginous airways and thus are important targets for therapy from the standpoint of stem cells and SMG disease pathology in human cystic fibrosis.

Airway submucosal glands (SMGs) orchestrate many vital processes that protect the lung from infections, and these glands are distinct epithelial units from the surface airway epithelium, by location, structure, function, and cellular composition. The current paradigm in the stem cell biology field is that the proximal surface airway epithelium is primarily repaired by surface basal cells following injury. SMGs also give rise to multipotent stem cells that are able to repair both glandular epithelium as well as surface airway epithelium; however, the pathways that control SMG stem cells and influence their ability to regenerate damaged epithelia have prior to this disclosure been largely unknown. Below, a specific transcription factor called lymphoid enhancer binding factor (Lef-1) was shown to control the cell fate decision of glandular myoepithelial cells (MECs) to regenerate and differentiate into 8 different cell types.

In particular, Lef-1 transcription factor controls proliferative expansion of glandular MECs and differentiation toward multipotent basal cells in the surface airway epithelium. As disclosed herein below, ectopic induction of Lef-1, specifically in MECs, enhances the regenerative capacity of this stem cell in a dose-dependent fashion for regeneration of both the airway surface and SMGs. Thus, the process of inducing Lef-1 in MECs to enhance airway epithelial regeneration is envisioned. Therefore, 1) sufficient induction of Lef-1 specifically in MECs leads to proliferative expansion and regeneration of the airway in the absence of injury, 2) Lef-1 expression enhances myoepithelial stem cell lineage commitment to normal multipotent SAE basal cell phenotypes as judged by RNAseq and lineage tracing, and 3) the induction of Lef-1 enhances self-renewal, capacity for differentiation, and engraftment in xenograft airways of myoepithelial stem cells. The present studies support applications in stem cell therapy and regenerative medicine in the lung.

In one embodiment, applications of this biology include in vitro growth and expansion of multipotent stem cells for use in cell therapy, the in vivo modulation of Lef-1-dependent pathways to enhance regeneration, the identification of therapeutic molecules that elicit the same processes that are induced by Lef-1 expression that may be more amenable to in vivo use as therapies, and the combined use of Lef-1 modulation (or its downstream targets) with gene editing tools in vivo that require active cell division for efficacy.

Applications also include: 1) modulating Lef-1 in order to treat degenerative lung diseases and/or conditions such asthma, COPD, cystic fibrosis, and other forms of airway epithelial damage; 2) the use of genetically or chemically modified airway stem cells using Wnt/Lef-1 pathways for applications in cell therapy for lung transplants in which glandular stem cell niches are exhausted and destroyed as obliterans bronchiolitis develops; 3) delivery of Wnt/Lef-1 analogs (chemical, protein or DNA-based) that modulate stem cells in vivo or ex vivo followed by transplantation back into patients; and 4) assays described herein to screen for small molecules that produce the same therapeutic effect as enhancing Lef-1 expression.

### Definitions

A “vector” or “delivery” vehicle refers to a macromolecule or association of macromolecules that comprises or associates with a polynucleotide or polypeptide, and which can be used to mediate delivery of the polynucleotide or polypeptide to a cell or intercellular space, either in vitro or in vivo. Illustrative vectors include, for example, plasmids, viral vectors, liposomes, nanoparticles, or microparticles and other delivery vehicles. In one embodiment, a polynucleotide to be delivered, sometimes referred to as a “target polynucleotide” or “transgene,” may comprise a coding sequence of interest in gene therapy (such as a gene encoding a protein of therapeutic interest), a coding sequence of interest and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. For example, a 2-fold enrichment, 10-fold enrichment, 100-fold enrichment, or a 1000-fold enrichment.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 82%, 85%, 87%, 90%, 92%, 95%, 97% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As used herein, “substantially pure” or “purified” means an object species is the predominant species present (i.e., on a molar basis it is more abundant than any other individual species in the composition), for instance, a substantially purified fraction is a composition wherein the object species comprises at least about 50 percent (on a molar basis) of all macromolecular species present. Generally, a substantially pure composition will comprise more than about 80 percent of all macromolecular species present in the composition, or more than about 85%, about 90%, about 95%, and about 99%. The object species may be purified to essential homogeneity (contaminant species cannot be detected in the composition by conventional detection methods) wherein the composition consists essentially of a single macromolecular species.

### Preparation of Expression Cassettes

To prepare expression cassettes encoding a polypeptide, a peptide thereof, or a fusion thereof, e.g., encoding one of

| AF288571_1 | |
|---|---|
| (SEQ ID NO: 1) | |
| 1 | mpqlsggggg gggdpelcat demipfkdeg dpqkekifae ishpeeegdl adiksslvne |
| 61 | seiipasngh evarqaqtsq epyhdkareh pddgkhpdgg lynkgpsyss ysgyimmpnm |
| 121 | nndpymsngs lsppiprtsn kvpvvqpsha vhpltplity sdehfspgsh pshipsdvns |
| 181 | kqgmsrhppa pdiptfypls pggvgqitpp lgwqgqpvyp itggfrqpyp sslsvdtsms |
| 241 | rfshhmipgp pgphttgiph paivtpqvkq ehphtdsdlm hvkpqheqrk eqepkrphik |
| 301 | kpinafmlym kemranvvae ctlkesaain qilgrrwhal sreeqakyye larkerqlhm |
| 361 | qlypgwsard nygkkkkrkr eklqesasgt gprmtaayi |
| AAF13268 | |
| (SEQ ID NO: 2) | |
| 1 | mpqlsggggg gggdpelcat demipfkdeg dpqkekifae ishpeeegdl adiksslvne |
| 61 | seiipasngh evarqaqtsq epyhdkareh pddgkhpdgg lynkgpsyss ysgyimmpnm |
| 121 | nndpymsngs lsppiprtsn kvpvvqpsha vhpltplity sdehfspgsh pshipsdvns |
| 181 | kqgmsrhppa pdiptfypls pggvgqitpp lgwqgqpvyp itggfrqpyp sslsvdtsms |
| 241 | rfshhmipgp pgphttgiph paivtpqvkq ehphtdsdlm hvkpqheqrk eqepkrphik |
| 301 | kpinafmlym kemranvvae ctlkesaain qilgrrwhal sreeqakyye larkerqlhm |
| 361 | qlypgwsard nygkkkkrkr eklqesasgt gprmtaayi |
| (SEQ ID NO: 5) | |
| 1 | mvsklsqlqt ellaallesg lskealiqal gepgpyllag egpldkgesc gggrgelael |
| 61 | pnglgetrgs edetdddged ftppilkele nlspeeaahq kavvetllqe dpwrvakmvk |
| 121 | sylqqhnipq revvdttgln qshlsqhlnk gtpmktqkra alytwyvrkq revaqqftha |
| 181 | gqgglieept gdelptkkgr rnrfkwgpas qqilfqayer qknpskeere tiveecnrae |
| 241 | ciqrgvspsq aqglgsnlvt evrvynwfan rrkeeafrhk lamdtysgpp pgpgpgpalp |
| 301 | ahsspg1ppp aispskvhgv ryguatset aevpsssggp lvtvstp1hq vsptglepsh |
| 361 | sllsteaklv saaggplppv stltalhsle qtspglnqqp qnlimaslpg vmtigpgepa |
| 421 | slgptftntg astiviglas tqaqsvpvin smgsslttlq pvqfsqplhp syqqp1mppv |
| 481 | qshvtqspfm atmaqlqsph alyshkpeva qythtgllpq tmlitdttnl salasltptk |
| 541 | qvftsdteas sesglhtpas qattlhvpsq dpagiqh1qp ahrlsasptv sssslvlyqs |
| 601 | sdssngqshl 1psnhsviet fistqmasss q |
| (SEQ ID NO: 6) | |
| 1 | mvskltslqq ellsallssg vtkevlvgal eellpspnfg vkletlplsp gsgaepdtkp |
| 61 | vfhtitngha kgrlsgdegs edgddydtpp ilkelqaint eeaaeqraev drm1sedpwr |
| 121 | aakmikgymq qhnipqrevv dvtglnqshl sqhlnkgtpm ktqkraalyt wyvrkqreil |
| 181 | rqfnqtvqss gnmtdkssqd ql1f1fpefs qqshgpgqsd dacseptnkk mrrnrfkwgp |
| 241 | asqqllyqay drqknpskee realveecnr aeclqrgvsp skahglgsnl vtevrvynwf |
| 301 | anrrkeeafr qklamdayss nqthslnpll shgsphhqps ssppnklsgv rysqqgnnel |
| 361 | tssstishhg nsamvtsqsv lqqvspasld pghnllspdg kmisysgggl ppvstltnih |
| 421 | slshhnpqqs qnlimtplsg vmaiaqslnt sqaqsvpvin svagslaalq pvqfsqqlhs |
| 481 | phqqplmqqs pgshmaqqpf maavtqlqns hmyahkqepp qyshtsrfps amvvtdtssi |
| 541 | stltnmsssk qcplqaw |
| (SEQ ID NO: 7) | |
| 1 | mnqpqrmapv gtdkelsdll dfsmmfplpv tngkgrpasl agaqfggsgk sgergayasf |
| 61 | grdagvgglt qagflsgela lnspgplsps gmkgtsqyyp sysgssrrra adgsldtqpk |
| 121 | kvrkvppglp ssvyppssge dygrdatayp saktpsstyp apfyvadgsl hpsaelwspp |
| 181 | gqagfgpmlg ggssplplpp gsgpvgssgs sstfgglhqh ermgyqlhga evngglpsas |
| 241 | sfssapgaty ggvsshtppv sgadsllgsr gttagssgda lgkalasiys pdhssnnfss |
| 301 | spstpvgspq glagtsqwpr agapgalsps ydgglhglqs kiedhldeai hvlrshavgt |
| 361 | agdmhtllpg hgalasgftg pmslggrhag lvggshpedg lagstslmhn haalpsqpgt |
| 421 | 1pdlsrppds ysglgragat aaaseikree kedeentsaa dhseeekkel kaprartrcq |
| 481 | ptprhsppsp hqdahvhrph ahrthtgrps agptlfpqph clplapsrrp phspdededd |
| 541 | llppeqkaer ekerrvanna rerlrvrdin eafkelgrmc qlhlnsekpq tkllilhqav |
| 601 | svilnleqqv rernlnpkaa clkrreeekv sgvvgdpqmv lsaphpglse ahnpaghm |
| (SEQ ID NO: 8) | |
| 1 | mhhqqrmaal gtdkelsdll dfsamfsppv ssgkngptsl asghftgsnv edrsssgswg |
| 61 | ngghpspsrn ygdgtpydhm tsrdlgshdn lsppfvnsri qsktergsys sygresnlqg |
| 121 | chqqsllggd mdmgnpgtls ptkpgsqyyq yssnnprrrp lhssamevqt kkvrkvppgl |
| 181 | pssvyapsas tadynrdspg ypsskpatst fpssffmqdg hhssdpwsss sgmnqpgyag |
| 241 | mlgnsshipq sssycslhph erlsypshss adinsslppm stfhrsgtnh ystssctppa |
| 301 | ngtdsimanr gsgaagssqt gdalgkalas iyspdhtnns fssnpstpvg sppslsagta |
| 361 | vwsrnggqas sspnyegplh slqsriedrl erlddaihvl rnhavgpsta mpgghgdmhg |
| 421 | ligpshngam gglgsgygtg llsanrhslm vgthredgva lrgshsllpn qvpvpqlpvq |
| 481 | satspdlnpp qdpyrgmppg lqgqsyssgs seiksddegd enlqdtksse dkkldddkkd |
| 541 | iksitrsrss nnddedltpe qkaerekerr mannarerlr vrdineafke lgrmvqlhlk |
| 601 | sdkpqtklli lhqavavils leqqvrernl npkaaclkrr eeekvssepp plslagphpg |
| 661 | mgdasnhmgq m |
| (SEQ ID NO: 9) | |
| 1 | mpqlsggggg gggdpelcat demipfkdeg dpqkekifae ishpeeegdl adiksslvne |
| 61 | seiipasngh evarqaqtsq epyhdkareh pddgkhpdgg lynkgpsyss ysgyimmpnm |
| 121 | nndpymsngs lsppiprtsn kvpvvqpsha vhpltplity sdehfspgsh pshipsdvns |
| 181 | kqgmsrhppa pdiptfypls pggvgqitpp lgwqgqpvyp itggfrqpyp sslsvdtsms |
| 241 | rfshhmipgp pgphttgiph paivtpqvkq ehphtdsdlm hvkpqheqrk eqepkrphik |
| 301 | kpinafmlym kemranvvae ctlkesaain qilgrrwhal sreeqakyye larkerqlhm |
| 361 | qlypgwsard nygkkkkrkr eklqesasgt gprmtaayi |

or an isolated protein having at least 80%, 85%, 87%, 90%, 92%, 93%, 94%, 95%, 98%, 99% or more amino acid identity to any one of SEQ ID Nos 1-2 or 5-9, for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotrophic virus. Such promoters include the CMV promoter, as well as the SV40 late promoter and retroviral LTRs (long terminal repeat elements), although many other promoter elements well known to the art may be employed, e.g., the MMTV, RSV, MLV or HIV LTR in the practice of the invention. In one embodiment, expression is inducible. In one embodiment, a tissue-specific promoter (or enhancer) is employed.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian, bacterial, yeast or insect cells, or prokaryotic cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, the recombinant DNA is stably integrated into the genome of the cell.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic cells, such as mammalian, e.g., human cells. Other viral vectors can be derived from poxviruses, e.g., vaccinia viruses, herpes viruses, adenoviruses, adeno-associated viruses, baculoviruses, and the like.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

### Vectors for Delivery

Delivery vectors include, for example, viral vectors, microparticles, nanoparticles, liposomes and other lipid-containing complexes, and other macromolecular complexes capable of mediating delivery of a gene or protein to a host cell, e.g., to provide for recombinant expression of a polypeptide encoded by the gene. Vectors can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Vectors for gene within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors, or proteins, which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes.

Exemplary gene viral vectors are described below. Vectors may be administered via any route including, but not limited to, intramuscular, buccal, rectal, intravenous or intracoronary administration, and transfer to cells may be enhanced using electroporation and/or iontophoresis. In one embodiment, vectors are locally administered.

In one embodiment, an isolated polynucleotide or vector having that polynucleotide comprises nucleic acid encoding a polypeptide or fusion protein that has substantial identity, e.g., at least 80% or more, e.g., 85%, 87%, 90%, 92%, 95%, 97%, 98%, 99% and up to 100%, amino acid sequence identity to one of SEQ ID NOs. 1-9, and may, when administered, promote cartilage growth or repair.

### Peptides, Polypeptides and Fusion Proteins

The peptide or fusion proteins of the invention can be synthesized in vitro, e.g., by the solid phase peptide synthetic method or by recombinant DNA approaches (see above). The solid phase peptide synthetic method is an established and widely used method. These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, chemically modified derivatives of a given peptide or fusion thereof, can be readily prepared. For example, amides of the peptide or fusion thereof of the present invention may also be prepared by techniques well known in the art for converting a carboxylic acid group or precursor, to an amide. One method for amide formation at the C-terminal carboxyl group is to cleave the peptide or fusion thereof from a solid support with an appropriate amine, or to cleave in the presence of an alcohol, yielding an ester, followed by aminolysis with the desired amine.

Salts of carboxyl groups of a peptide or fusion thereof may be prepared in the usual manner by contacting the peptide, polypeptide, or fusion thereof with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide or fusion thereof may be prepared by utilizing an N-acyl protected amino acid for the final condensation, or by acylating a protected or unprotected peptide, polypeptide, or fusion thereof. O-acyl derivatives may be prepared, for example, by acylation of a free hydroxy polypeptide or polypeptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the polypeptide. Other amino-terminal modifications include aminooxypentane modifications.

In one embodiment, a peptide or fusion protein has substantial identity, e.g., at least 80% or more, e.g., 85%, 87%, 90%, 92%, 95%, 97%, 98%, 99% and up to 100%, amino acid sequence identity to one of SEQ ID NOs. 1-9.

Substitutions may include substitutions which utilize the D rather than L form, as well as other well known amino acid analogs, e.g., unnatural amino acids such as α, α-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, w-N-methylarginine, and other similar amino acids and imino acids and tert-butylglycine.

Conservative amino acid substitutions may be employed—that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; trp, tyr, phe.

The invention also envisions a peptide, polypeptide or fusion polypeptide with non-conservative substitutions. Non-conservative substitutions entail exchanging a member of one of the classes described above for another.

Acid addition salts of the peptide, polypeptide or fusion polypeptide or of amino residues of the peptide, polypeptide or fusion polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

### Formulations and Dosages

The polypeptides or fusions thereof, or nucleic acid encoding the polypeptide or fusion, or modulators of Lef-1/Wnt signaling, can be formulated as pharmaceutical compositions and administered to a mammalian host, such as a human patient in a variety of forms adapted to the chosen route of administration, e.g., orally or parenterally, by intravenous, intramuscular, topical or subcutaneous routes. In one embodiment, the polypeptide or nucleic acid encoding the polypeptide is administered prophylactically.

In one embodiment, the polypeptides or fusions thereof, or nucleic acid encoding the polypeptide or fusion, modulators of Lef-1/Wnt signaling, may be administered by infusion or injection. Solutions of the polypeptides or fusions thereof, or nucleic acid encoding the polypeptide or fusion, modulators of Lef-1/Wnt signaling, or salts thereof, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders forthe preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the polypeptides or fusions thereof, or nucleic acid encoding the polypeptide or fusion, or modulators of Lef-1/Wnt signaling, can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the polypeptides or fusions thereof, or nucleic acid encoding the polypeptide or fusion, or modulators of Lef-1/Wnt signaling, in a liquid composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the polypeptides or fusions thereof, or nucleic acid encoding the polypeptide or fusion, or modulators of Lef-1/Wnt signaling, required for use alone or with other agents will vary with the route of administration, the nature of the condition being treated and the age and condition of the patient and will be ultimately at the discretion of the attendant physician or clinician.

The polypeptides or fusions thereof, or nucleic acid encoding the polypeptide or fusion, or modulators of Lef-1/Wnt signaling, may be conveniently administered in unit dosage form; for example, containing 5 to 1000 mg, conveniently 10 to 750 mg, or conveniently 50 to 500 mg of active ingredient per unit dosage form.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

The invention will be described by the following non-limiting examples.

### Example 1

### Summary

The mouse trachea is thought to contain two distinct stem cell compartments that contribute to airway repair-basal cells in the surface airway epithelium (SAE) and an unknown submucosal gland (SMG) cell type. Whether a lineage relationship exists between these two stem cell compartments remains unclear. Using lineage tracing of glandular myoepithelial cells (MECs), we demonstrate that MECs can give rise to seven cell types of the SAE and SMGs following severe airway injury. MECs progressively adopted a basal cell phenotype on the SAE and established lasting progenitors capable of further regeneration following reinjury. MECs activate Wnt-regulated transcription factors (Lef-1/TCF7) following injury and Lef-1 induction in cultured MECs promoted transition to a basal phenotype. Surprisingly, dose-dependent MEC conditional activation of Lef-1 in vivo promoted self-limited airway regeneration in the absence of injury. Thus, modulating the Lef-1 transcriptional program in MEC-derived progenitors may have regenerative medicine applications for lung diseases.

### INTRODUCTION

Tissue-specific stem cells (SCs) remain one of the greatest frontiers in biomedical science and regenerative medicine. However, processes that regulate SC self-renewal, survival, and differentiation are not uniformly understood in different organs. Epithelial tissues that are exposed to the external environment, such as those of the lung, intestine, and skin, often demonstrate an incredible capacity to regenerate following injury (Hogan et al., 2014; Rajagopal and Stanger, 2016; Tetteh et al., 2015). However, limitation persist in our understanding of how epithelial SCs respond to injury and how repair after injury may differ from cellular renewal at steady state homeostasis.

Lineage-tracing studies in the mouse lung suggest that multiple region-specific progenitors contribute to regenerative plasticity of the airway epithelia (Hogan et al., 2014). For example, extensive evidence has demonstrated that basal cells are the primary homeostatic SC for the tracheal pseudostratified columnar epithelium (Ghosh et al., 2011; Hogan et al., 2014; Rock et al., 2011). However, following selective ablation of airway basal cells, alternative regenerative mechanisms induce fully committed surface airway epithelial (SAE) club cells to dedifferentiate into functional SAE basal SCs (Tata et al., 2013). The severity of injury can also influence when SC niches are mobilized in distal airways (Giangreco et al., 2009). Such findings emphasize the flexibility of SCs and their niches in responding to diverse environmental insults. In addition, the mouse trachea also contains epithelial submucosal glands (SMGs), which can also act as a regenerative SC niche for the SAE (Hegab et al., 2011; Lynch et al., 2016; Lynch and Engelhardt, 2014; Xie et al., 2011).

SMGs are grape-like tubuloacinar structures embedded within the mesenchyme beneath the SAE of all cartilaginous airways in humans and the proximal trachea of mice. Four major anatomical domains specified by their morphologydefine SMGs: ciliated ducts, collecting ducts, mucous tubules and serous acini (Liu et al., 2004). Ciliated ducts are generally considered to be an extension of the SAE and contain similar cell types: basal, ciliated, and secretory cells. Collecting ducts, which are more extensive in larger mammals than in mice, are composed of a poorly defined simple columnar epithelium. Mucous tubules and serous acini comprise the most distal components of the glands. Finally, contractile myoepithelial cells line the collecting ducts, mucous tubules, and serous acini, but are absent in ciliated ducts. Together, these cellular compartments control the secretion of proteins and mucus important in airway innate immunity.

Progenitors have been shown to reside within gland ducts (Hegab et al., 2011). However, slowly cycling glandular progenitors that retain multiple nucleotide labels following repeated injury also reside deeper within the tubular network of SMGs (Lynch et al., 2016; Lynch and Engelhardt, 2014; Xie et al., 2011). Focal regions of high tonic Wnt-signaling appear to be an integral component of the SMG SC niche, as label-retaining cells exist in these niches (Lynch et al., 2016). Wnt-signaling also plays an important role in establishing the glandular SC niche during post-natal development of the mouse trachea (Lynch and Engelhardt, 2014). During SMG morphogenesis, myoepithelial cells (MECs) are born early during the elongation phase as tubules invade the lamina propria and these progenitors have the capacity to differentiate into other glandular cell types but do not contribute to the SAE (Anderson et al., 2017). Thus, glandular MECs may be a resident SC for adult SMG regeneration.

By way of analogy to SC niches within intestinal and pyloric crypts (Aloia et al., 2016; Gehart and Clevers, 2015), it stands to reason that tracheal SMGs might serve as a protected SC niche, sequestering epithelial SCs from the more exposed environment of the SAE (Lynch and Engelhardt, 2014). It was hypothesized that, following severe injury, reserve SCs located deep within the SMGs are able to regenerate the SAE. In this context, “reserve SCs” means multipotent cells capable of imparting a regenerative response in the setting of a specific type or severity of injury and giving rise to professional SCs. “Professional SCs” are multipotent progenitors that are the primary source of cellular regeneration for a tissue under most conditions. Using lineage tracing, it was demonstrated that glandular MECs are multipotent progenitors of both SAE and SMG cell types following severe injury. Furthermore, it was demonstrated that the Wnt-signaling transcription factor, Lef-1, is sufficient to activate lineage commitment of MECs and their regenerative responses. “MEC lineage commitment” is a process whereby MECs exit their endogenous niche and assume an altered progenitor cell phenotype capable of multipotent differentiation. Given that humans possess SMGs throughout the cartilaginous airways, this SC niche may play a significant role in lung regeneration and disease.

### Materials and Methods

### Animal Studies

Experiments involving mice were performed according to protocols approved by the Institutional Animal Care and Use Committee of the University of Iowa. The C57BL/6 mice (stock number 000664), B6.129(Cg)-Gt(ROSA)26Sor^{tm4(ACTB-tdTomato,-EGFP)Luo}/J (ROSA-TG) Cre-reporter mice (stock number 007676), and MYH11-Cre^{ERT2} mice (stock number 019079) were purchased from The Jackson Laboratory. The ACTA2-Cre^{ERT2} mice (C57BL/6 background) were generously provided by Dr. B. Paul Herring's lab and were described previously (Wendling et al., 2009). The previously described ROSA26-CAG-^{LoxP}EGFP^{StopLoxP}Lef-1 knock-in mouse model (Sun et al., 2016) on a C57BL/6 background was used over express the human LEF1 transgene in response to Cre. For the purposes of this manuscript, this line is called Lef-1KI. In all studies both male and female mice were utilized with the exception that only male MYH11-Cre^{ERT2}:ROSA-TG mice were evaluated, since the MYH11-Cre^{ERT2} transgene is on the Y-chromosome. Mice were maintained in house under SPF conditions. For lineage tracing experiment experiments Cre-mediated recombination was induced in mice by i.p. injection of 75 μg tamoxifen per gram bodyweight every 24 hrs for a total of 5 consecutive days. Mice were allowed to recover for 5 or 21 days between tamoxifen treatment and injury. Mice were typically induced with tamoxifen between 6-8 weeks of age unless otherwise stated. For naphthalene injury experiments mice were injured with a single i.p. injection of either 200 mg/kg or 300 mg/kg naphthalene per gram body weight. Double injury experiments were performed as specified in the figure legends and text and typically separated by a 21 day recovery period. For SO₂ injury experiments, mice were exposed to 600 ppm SO₂ under atmospheric pressure for 4 hours. The following summarizes the conditions used for various mouse experiments.

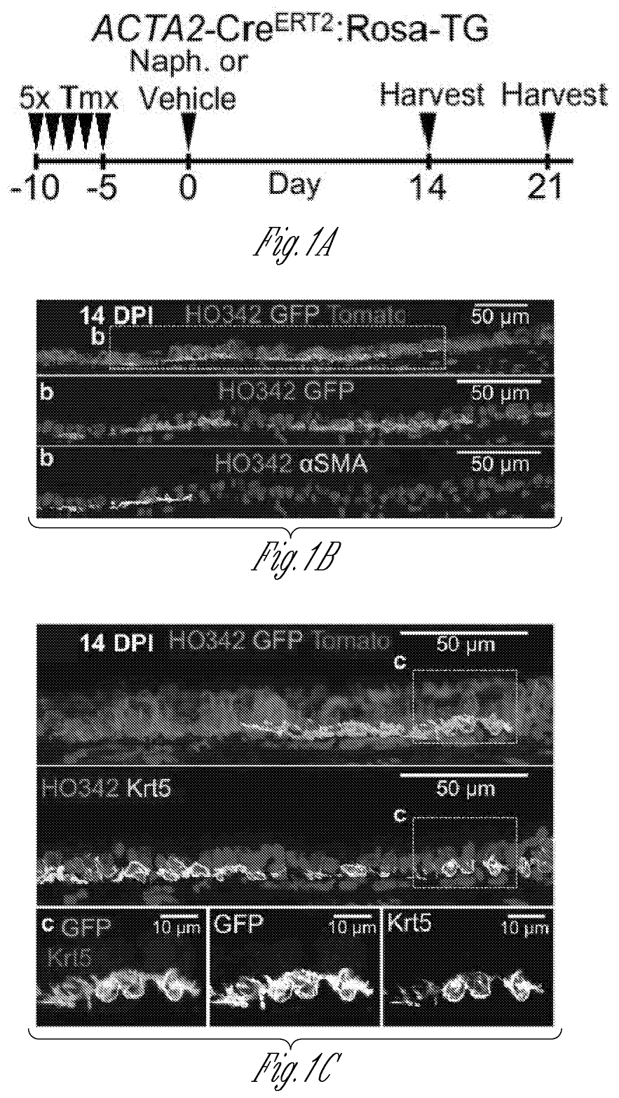

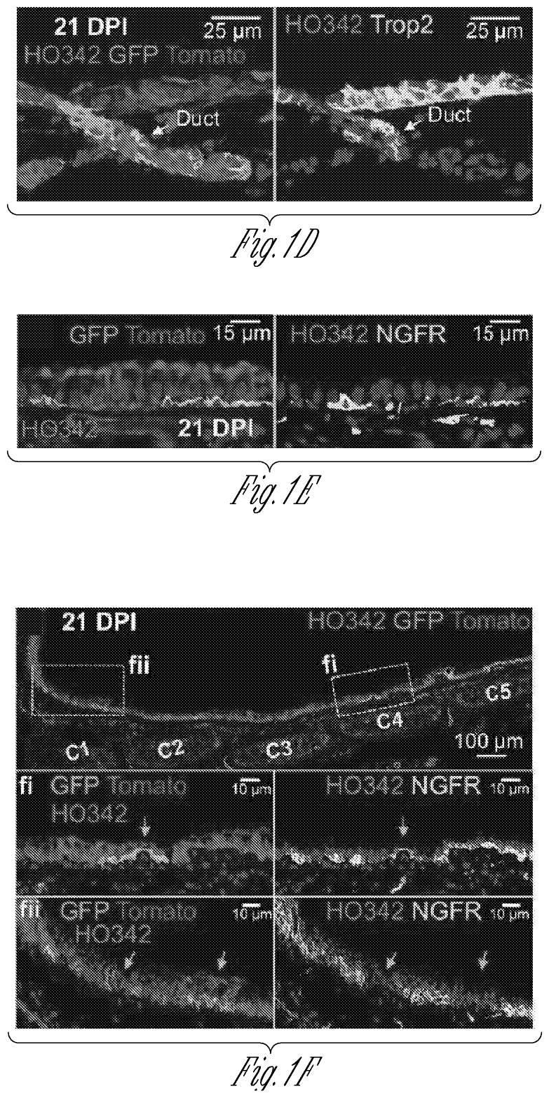

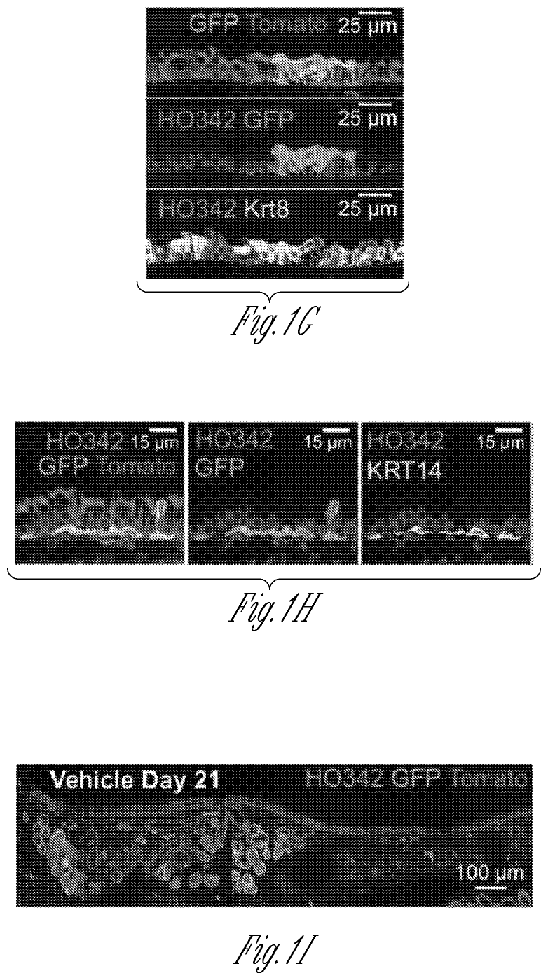

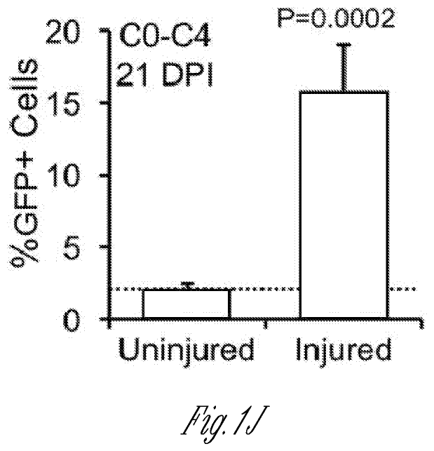

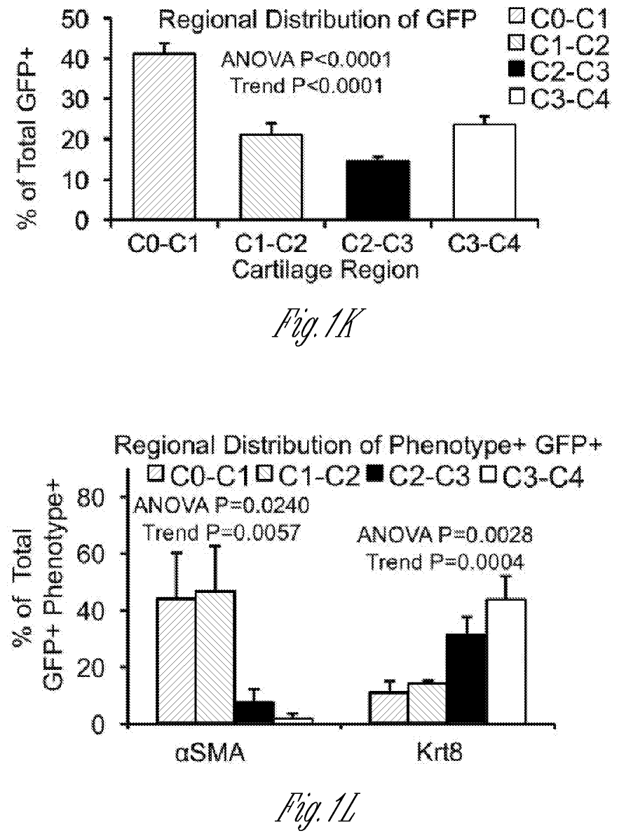

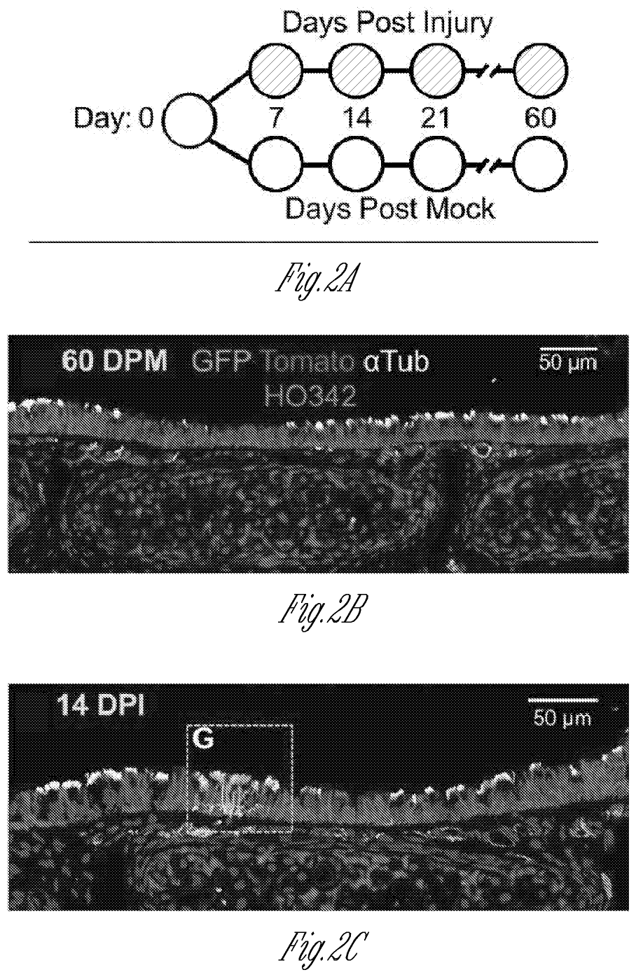

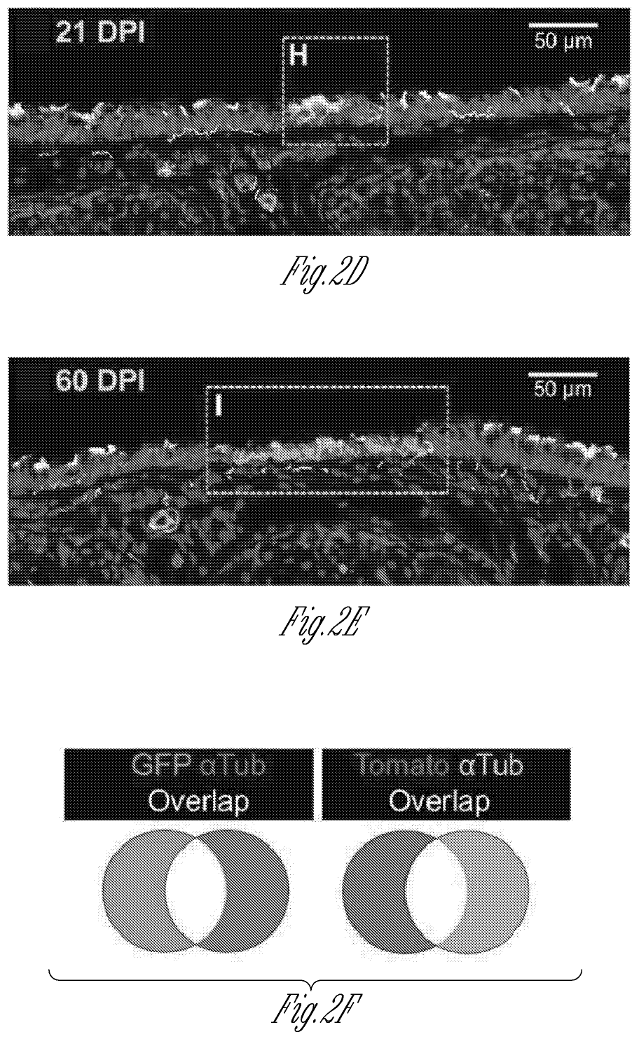

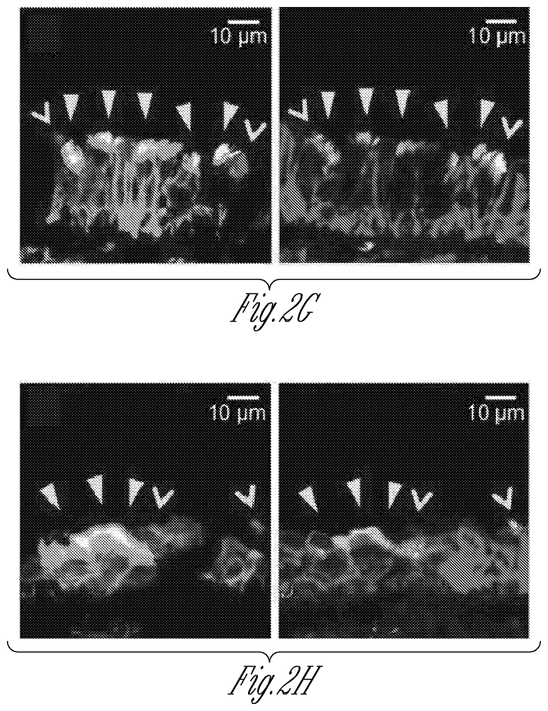

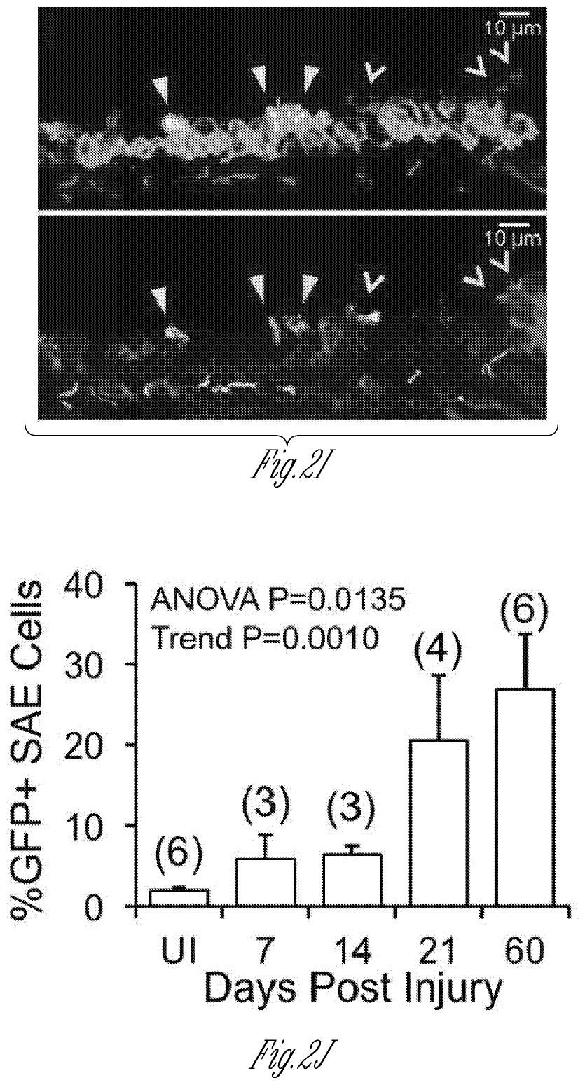

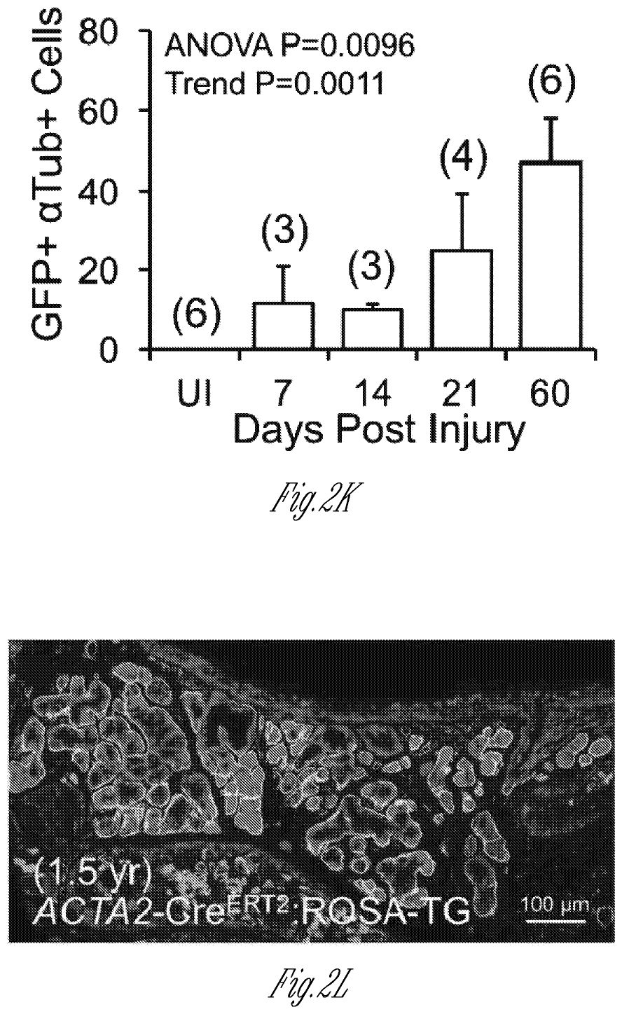

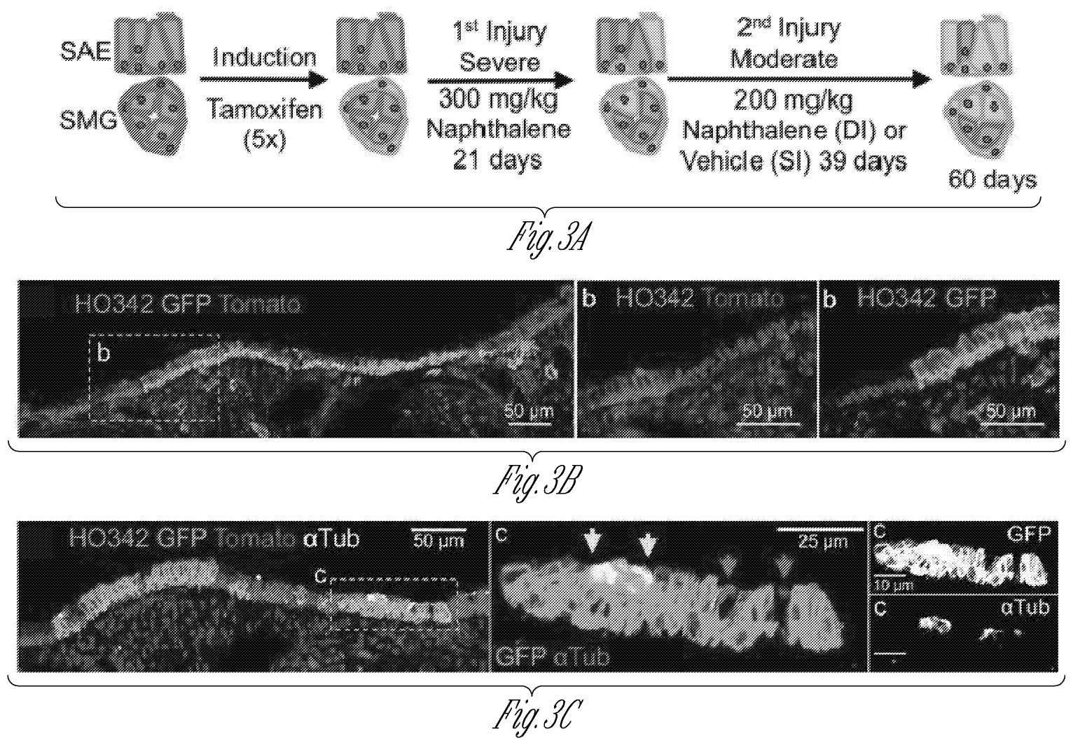

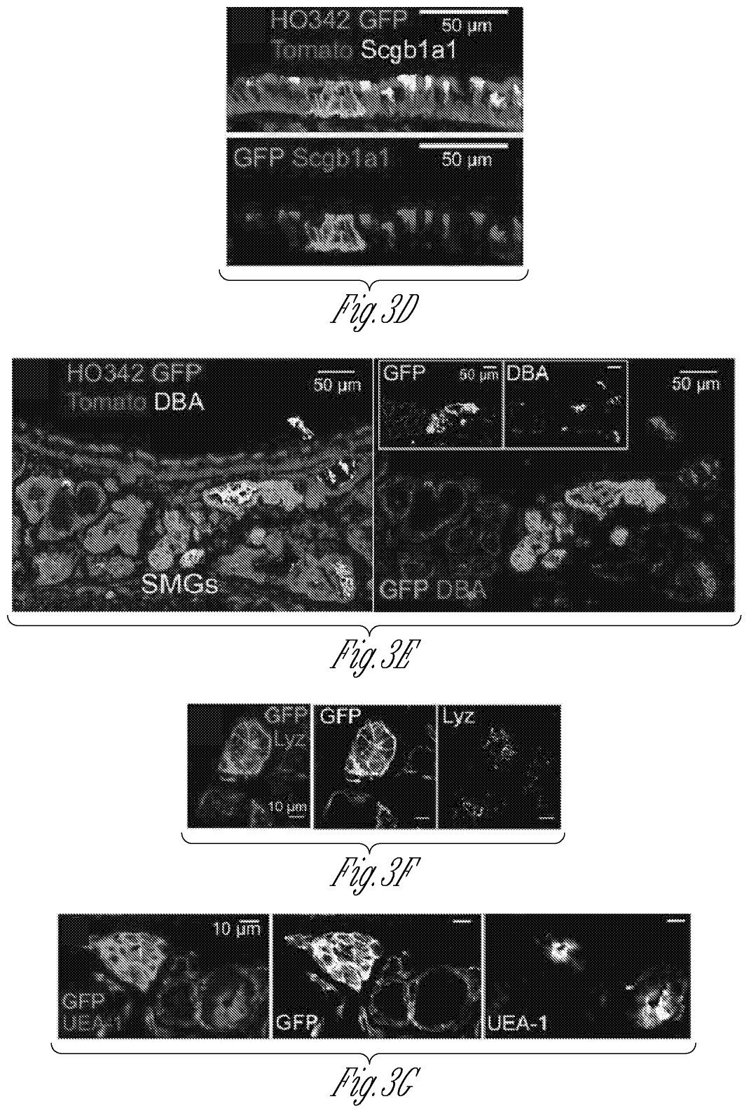

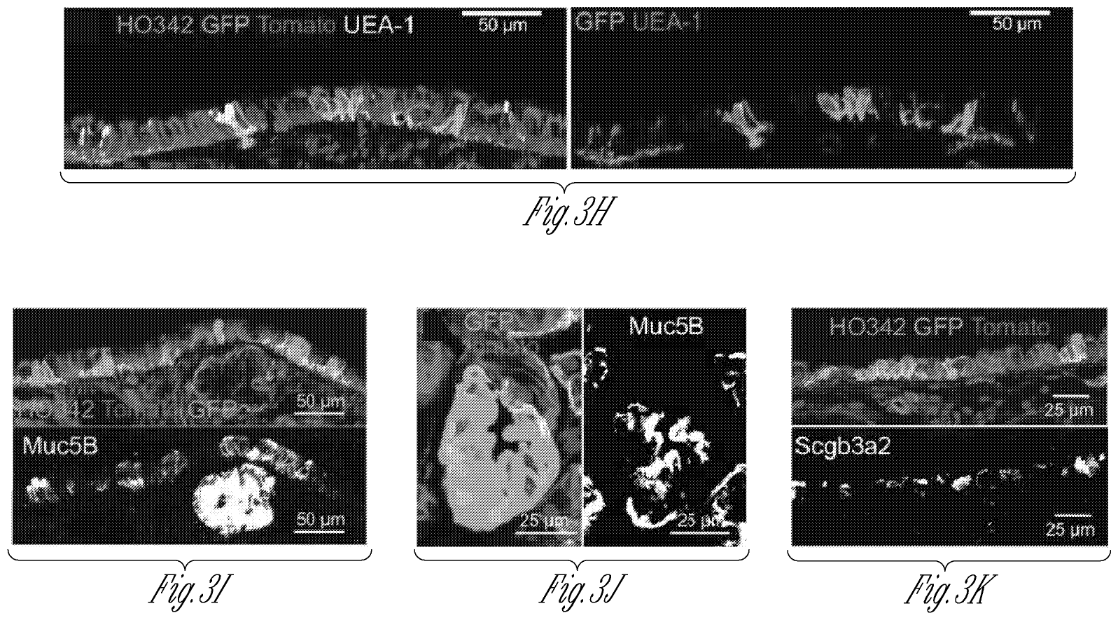

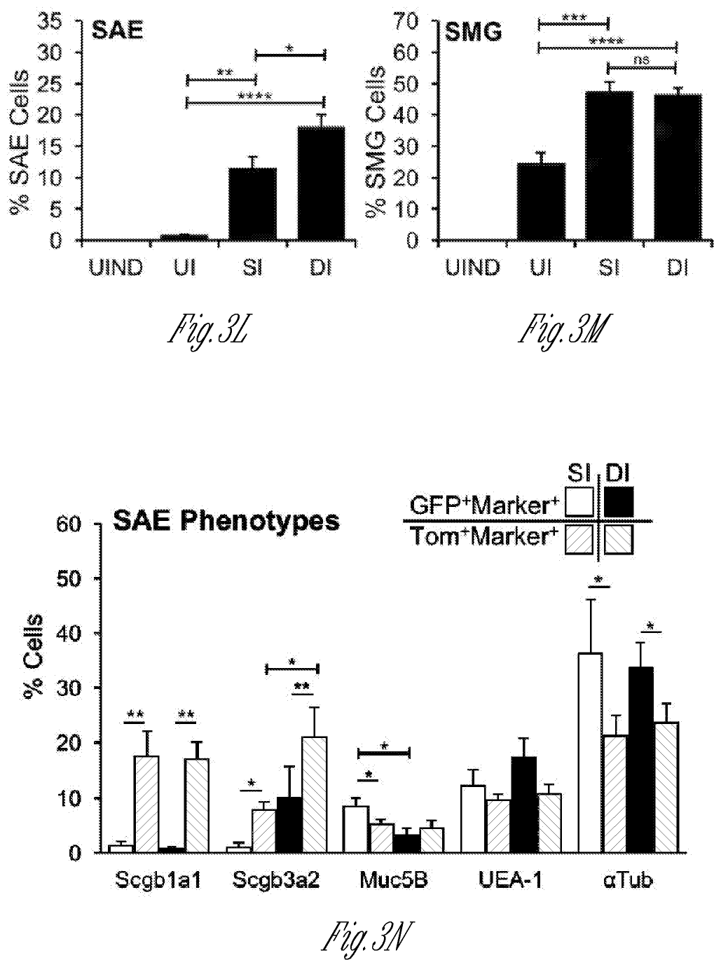

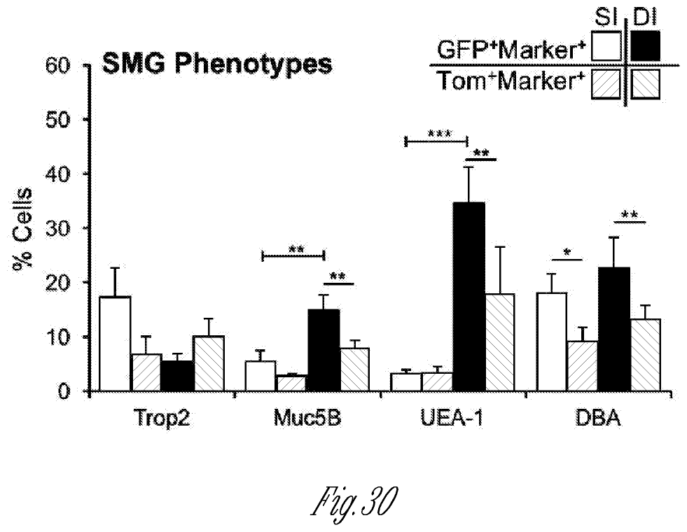

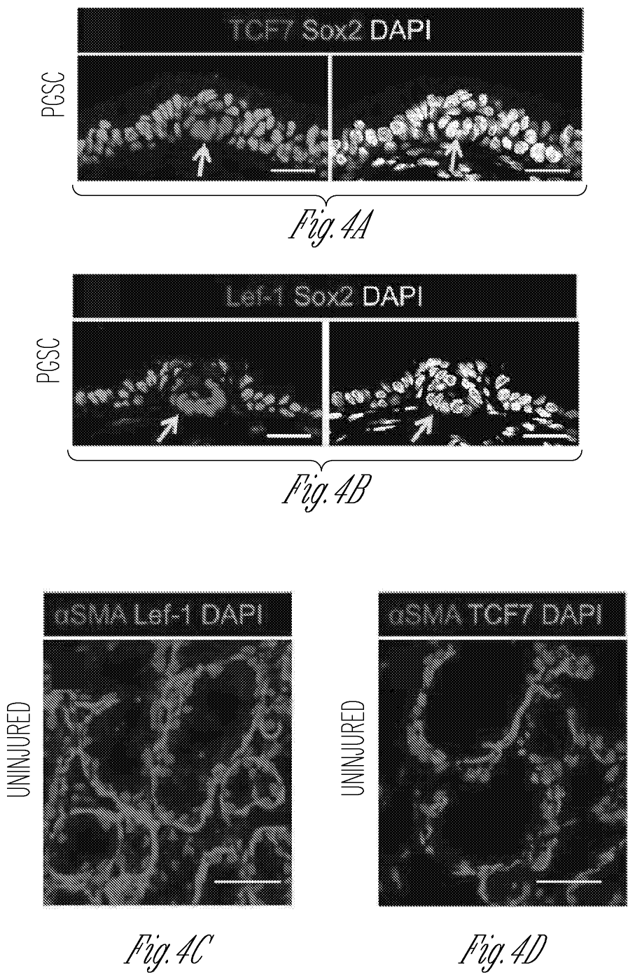

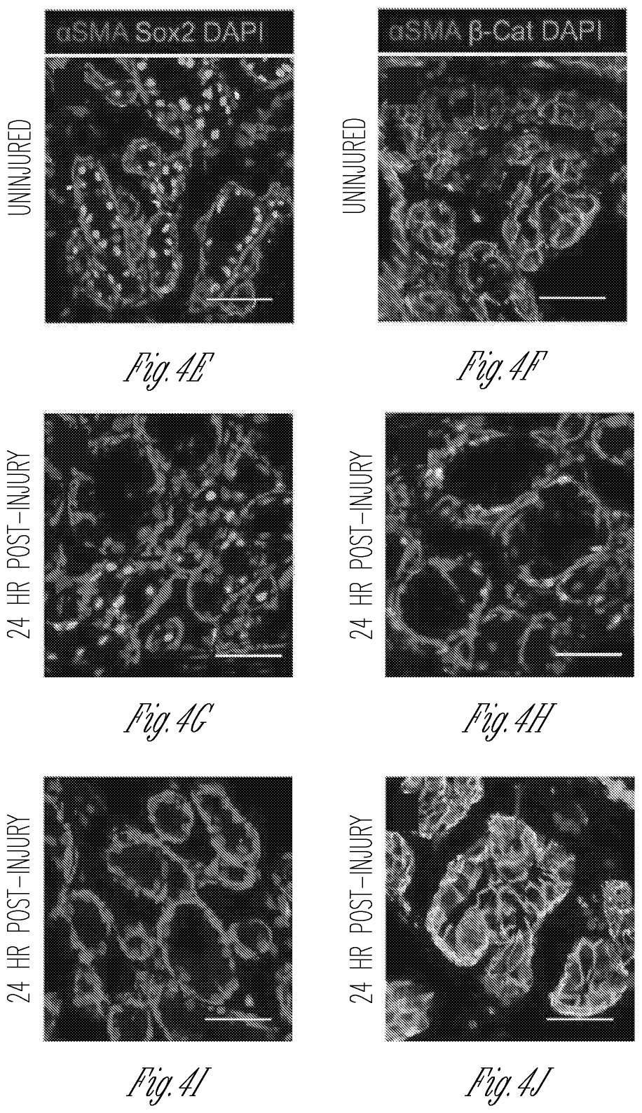

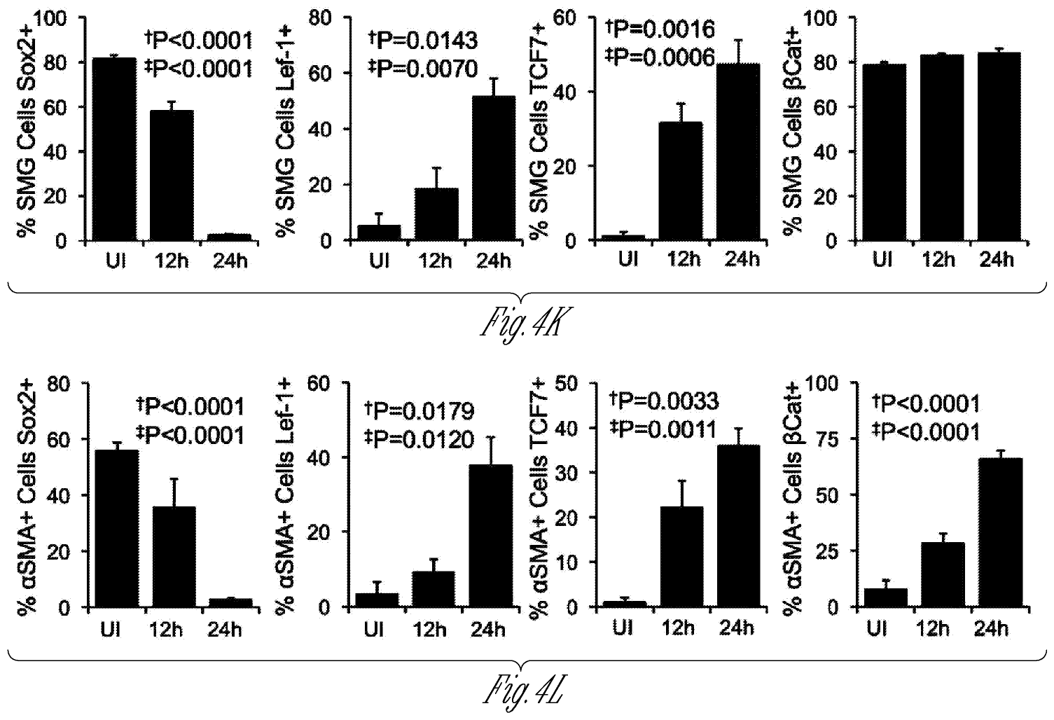

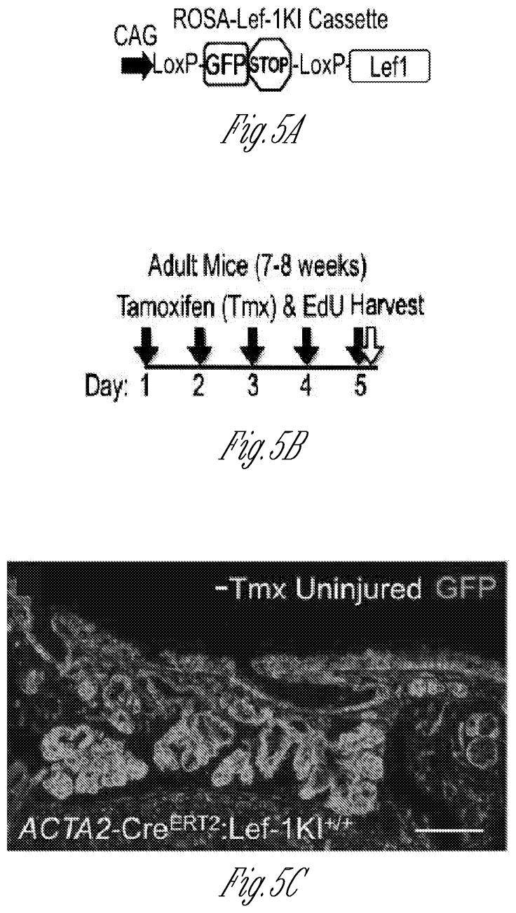

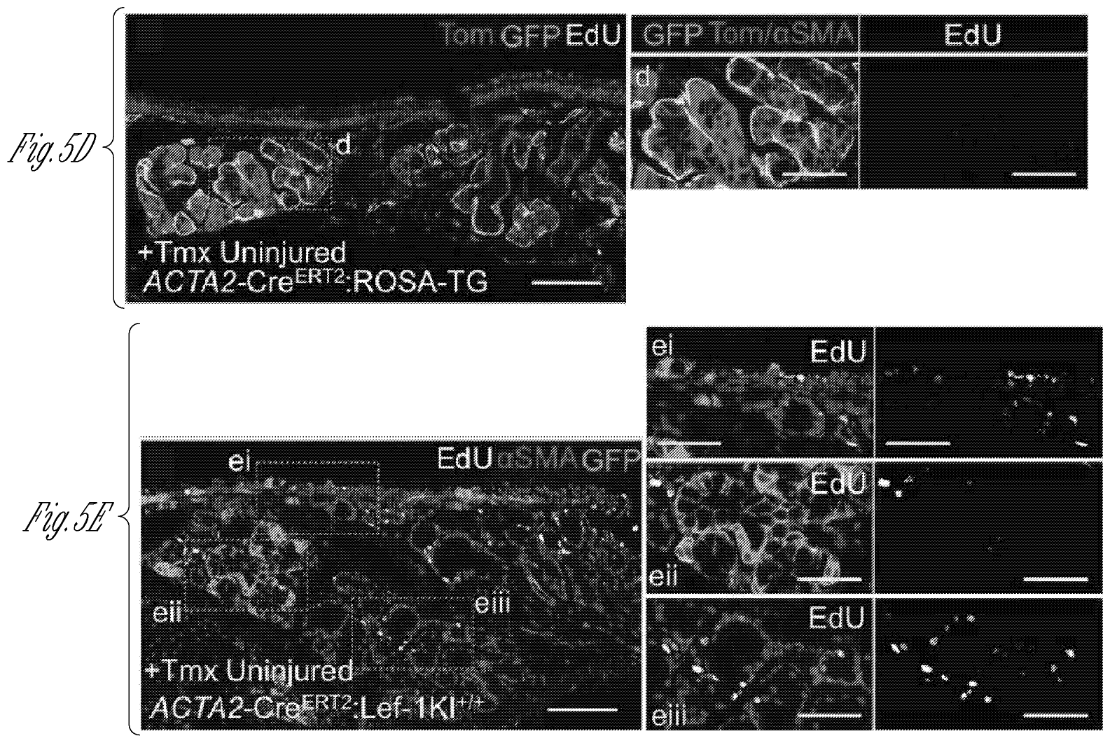

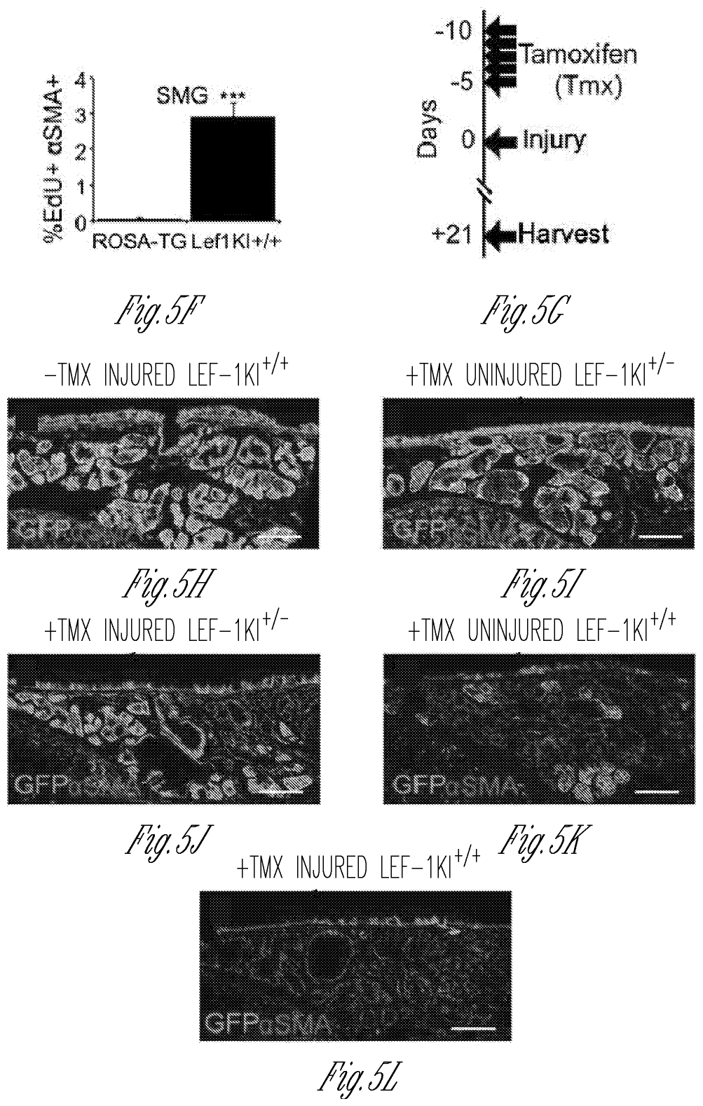

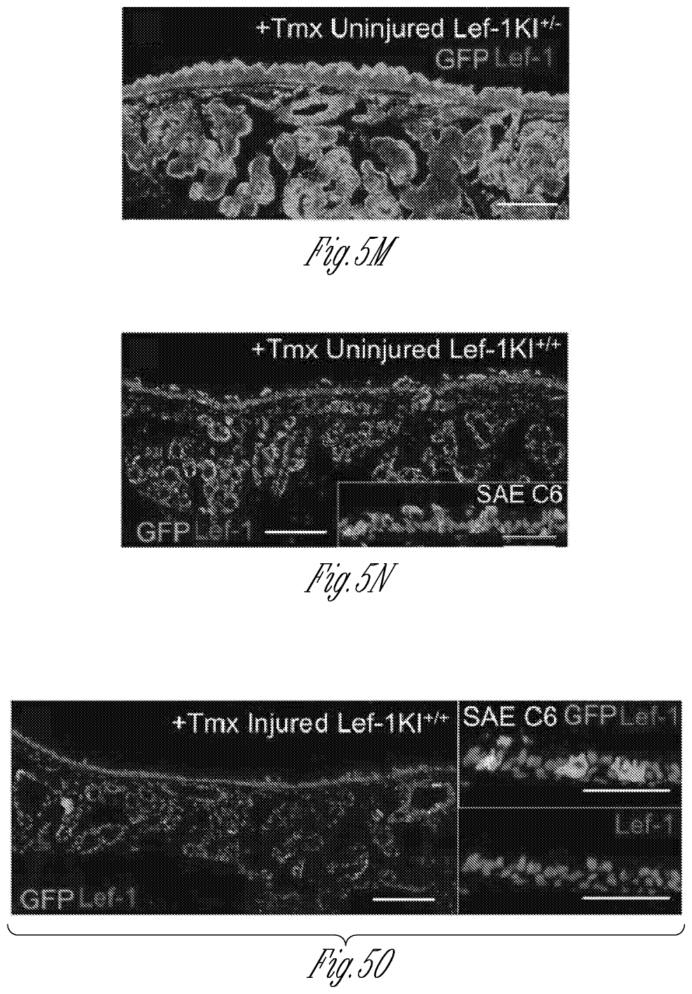

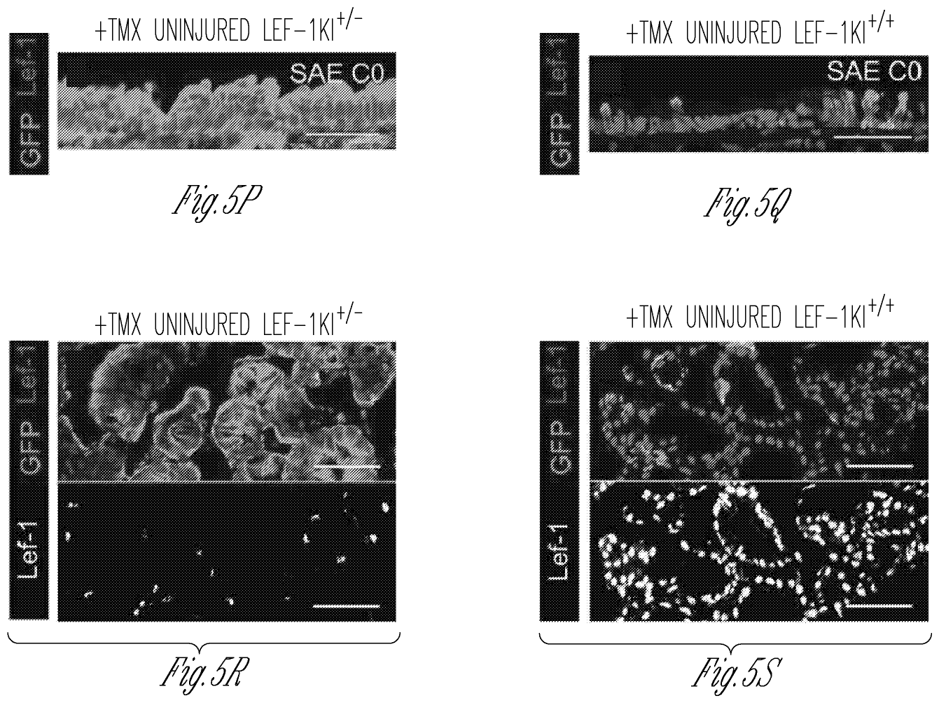

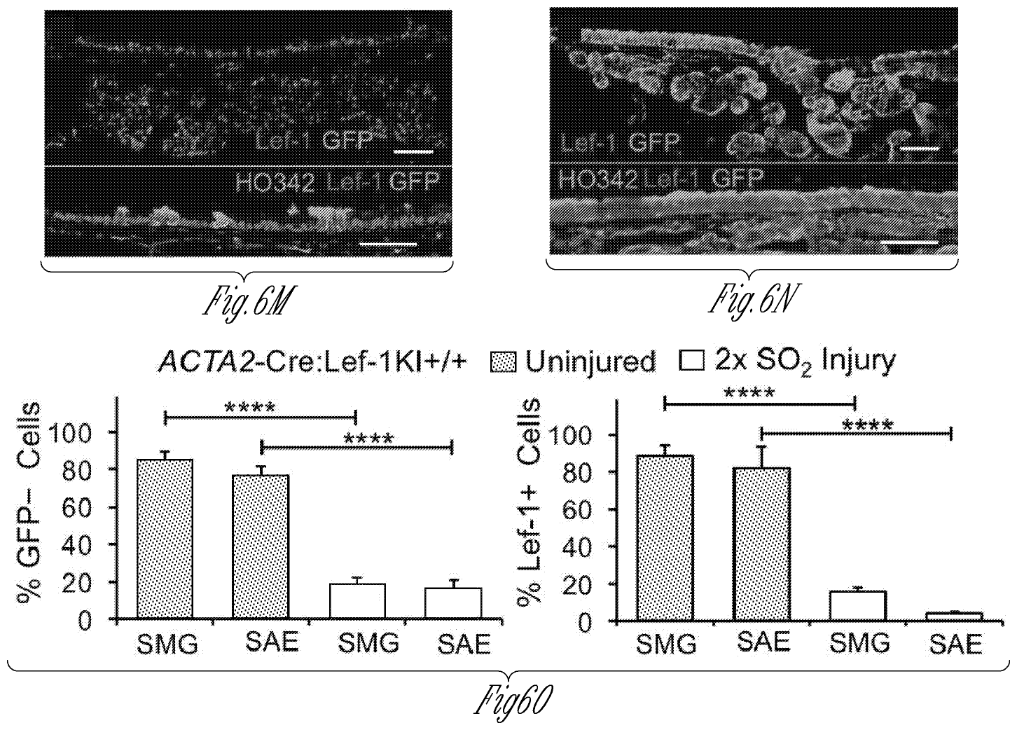

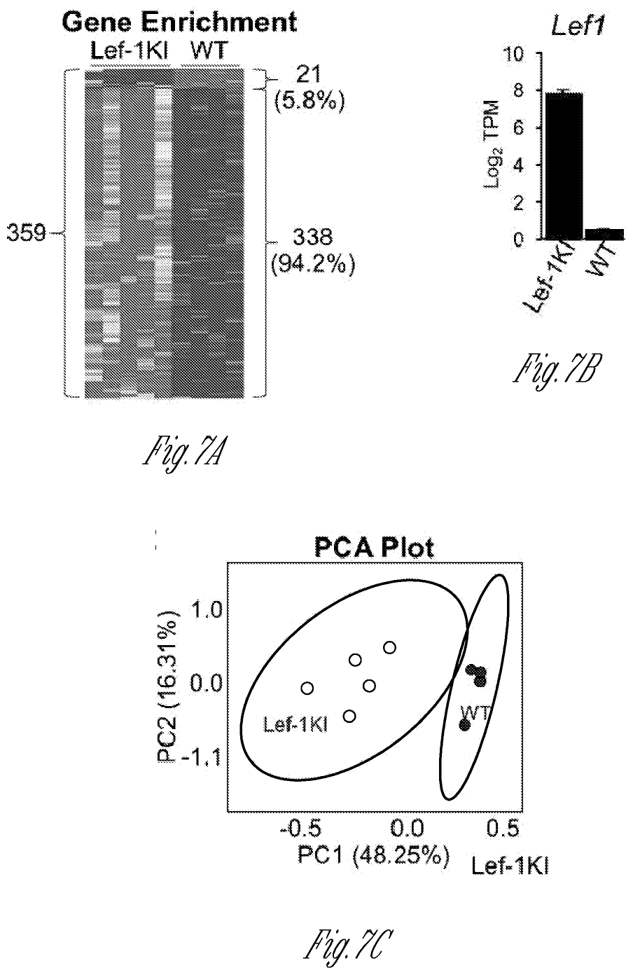

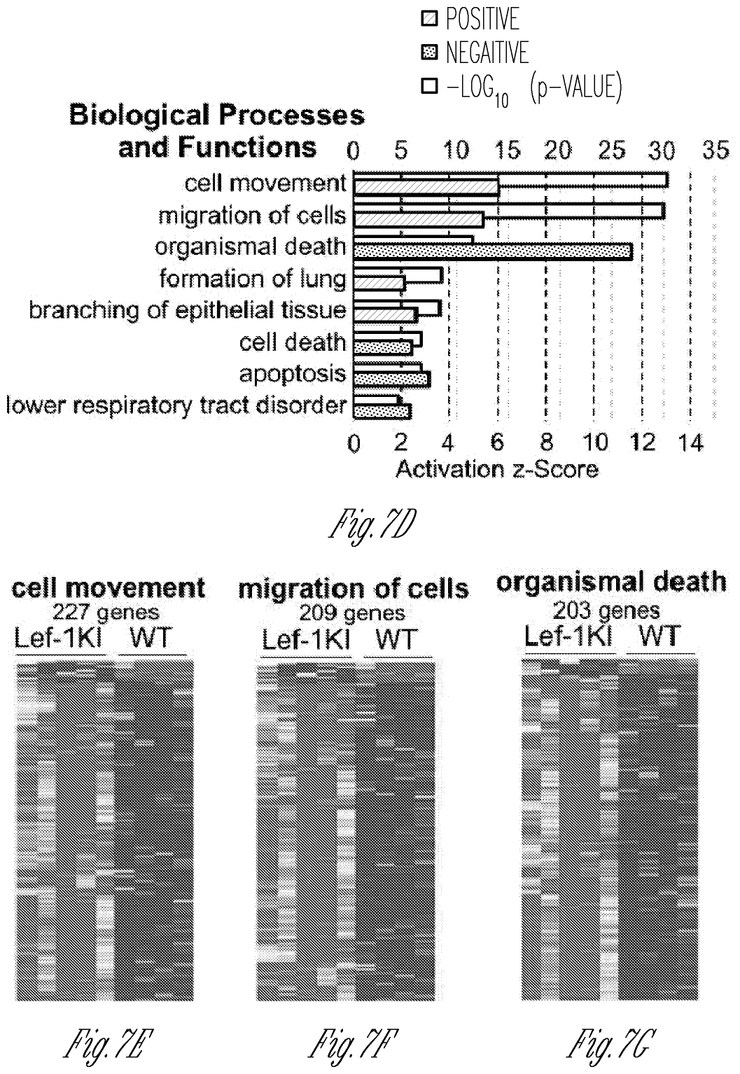

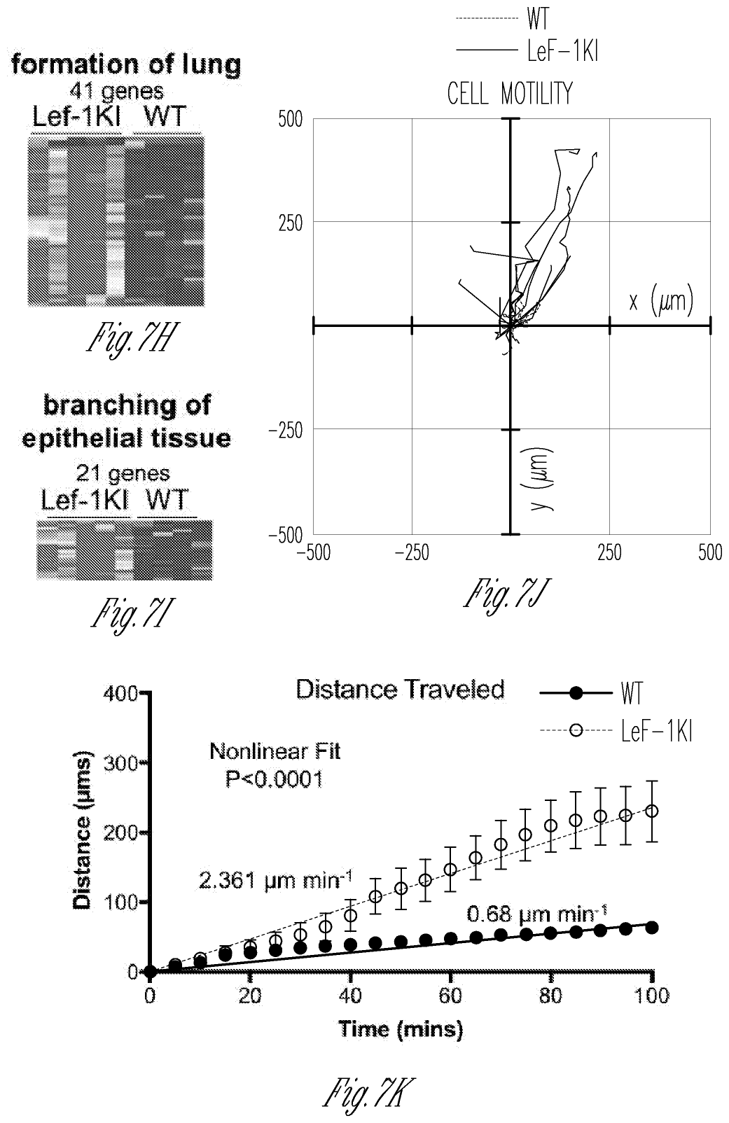

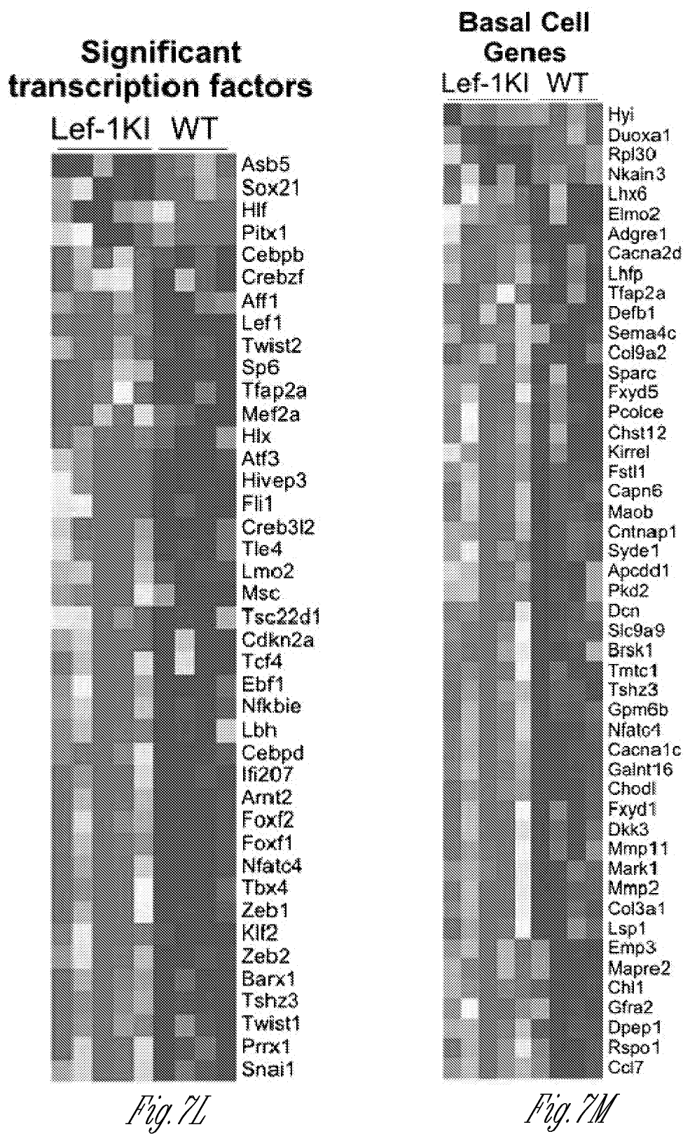

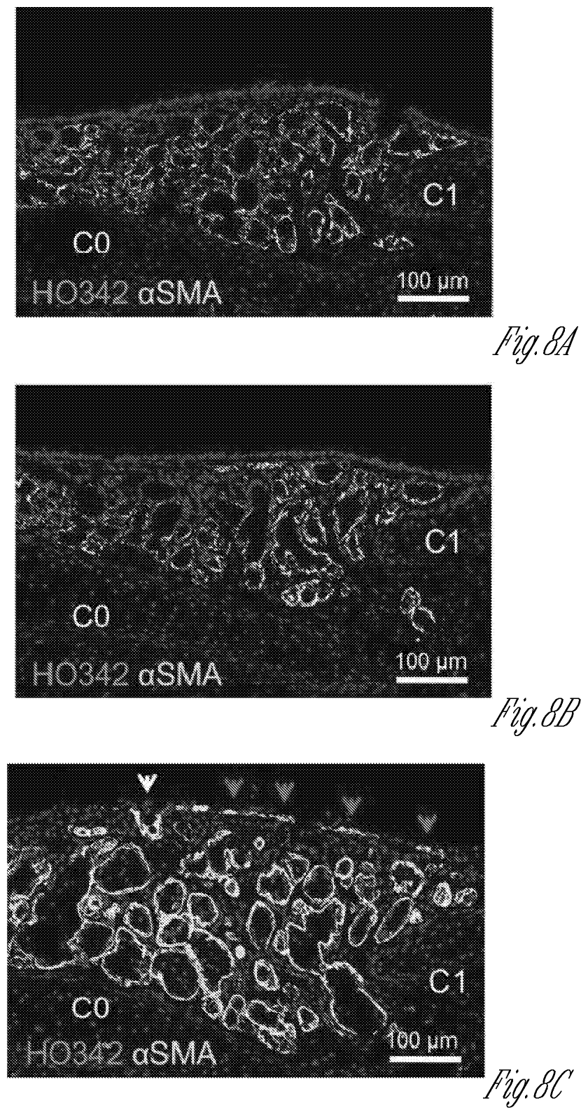

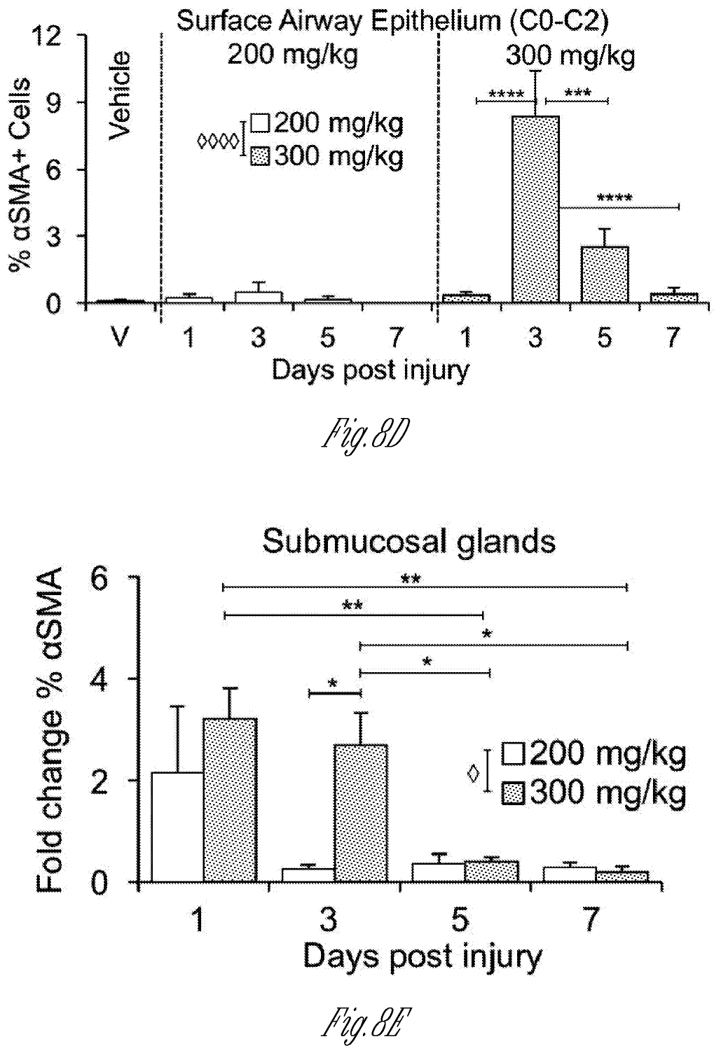

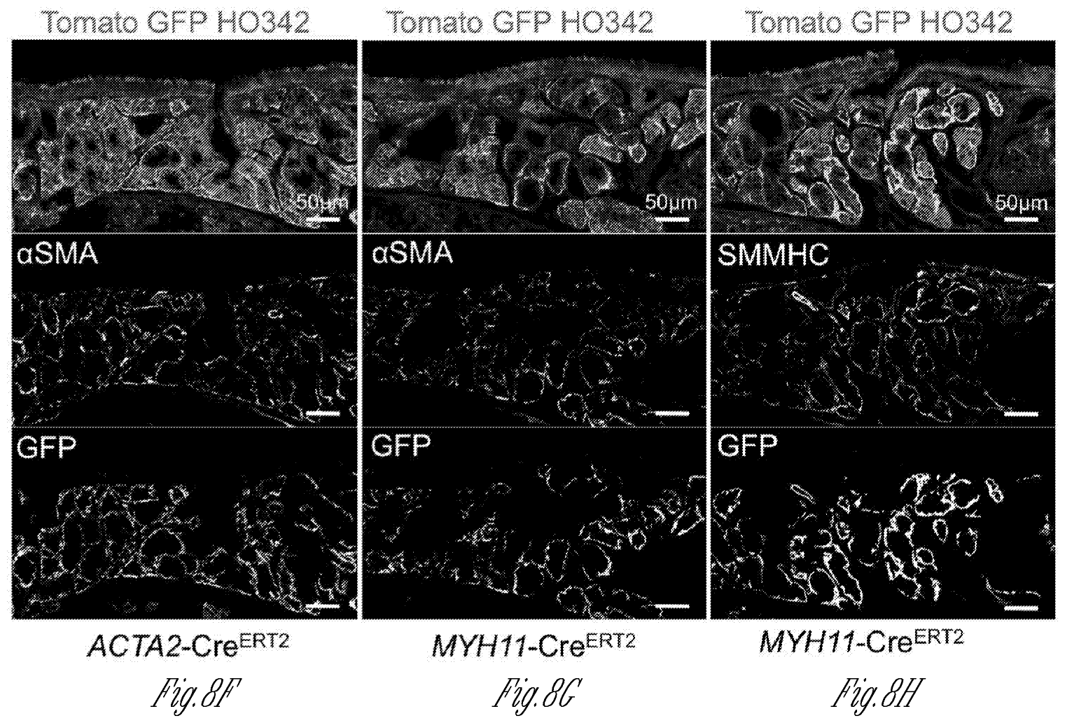

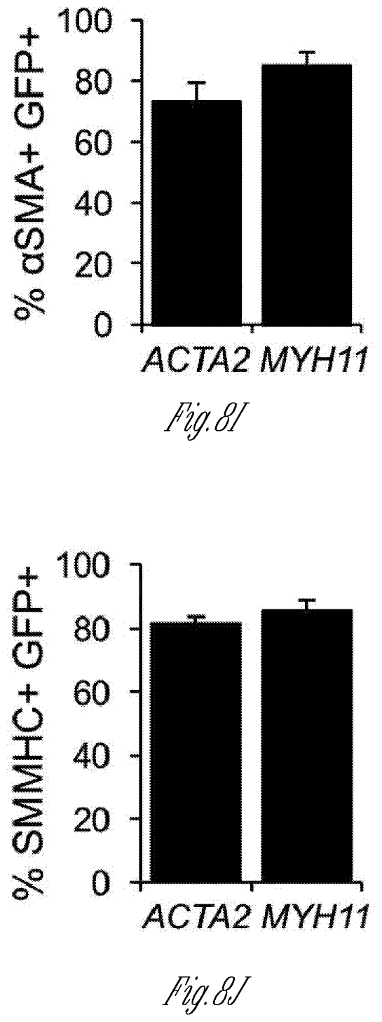

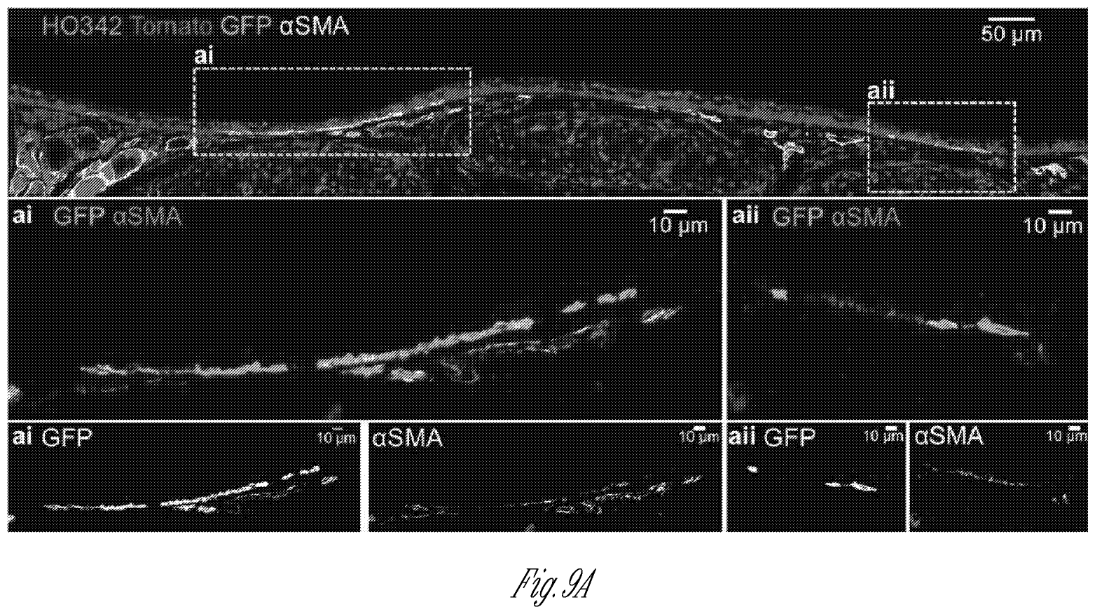

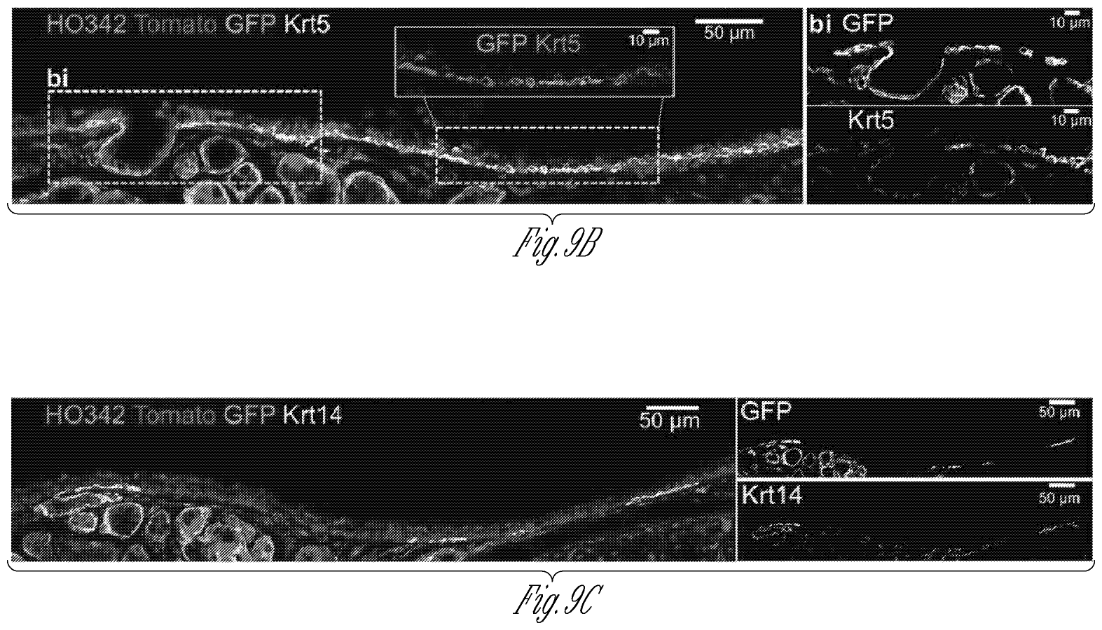

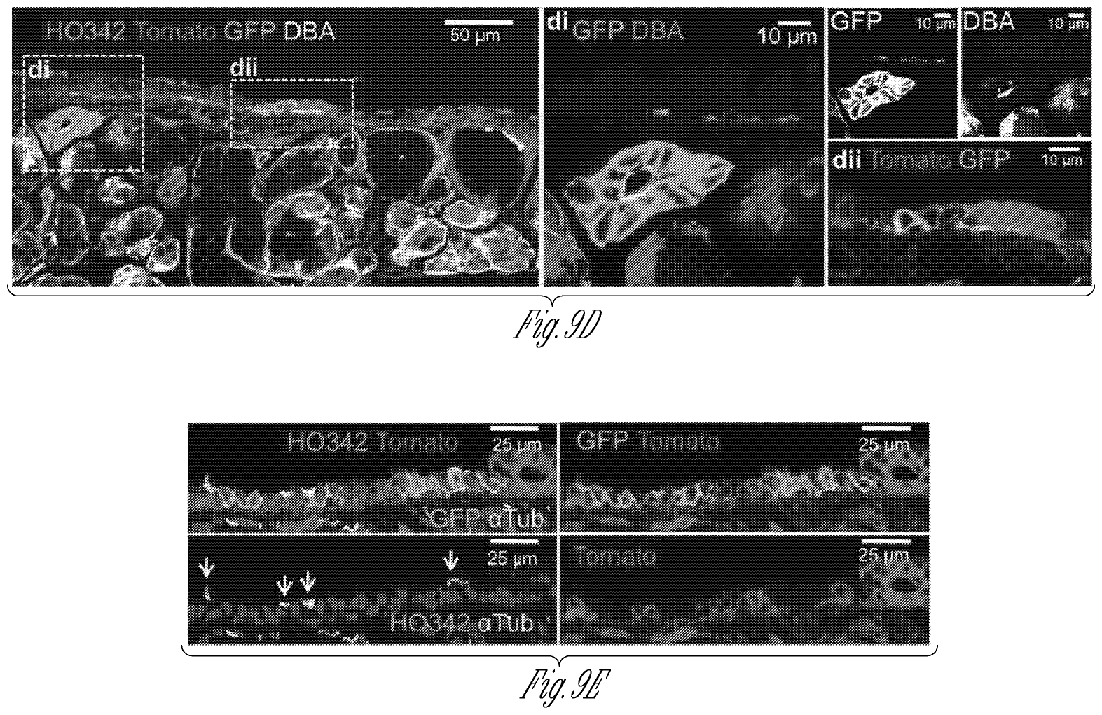

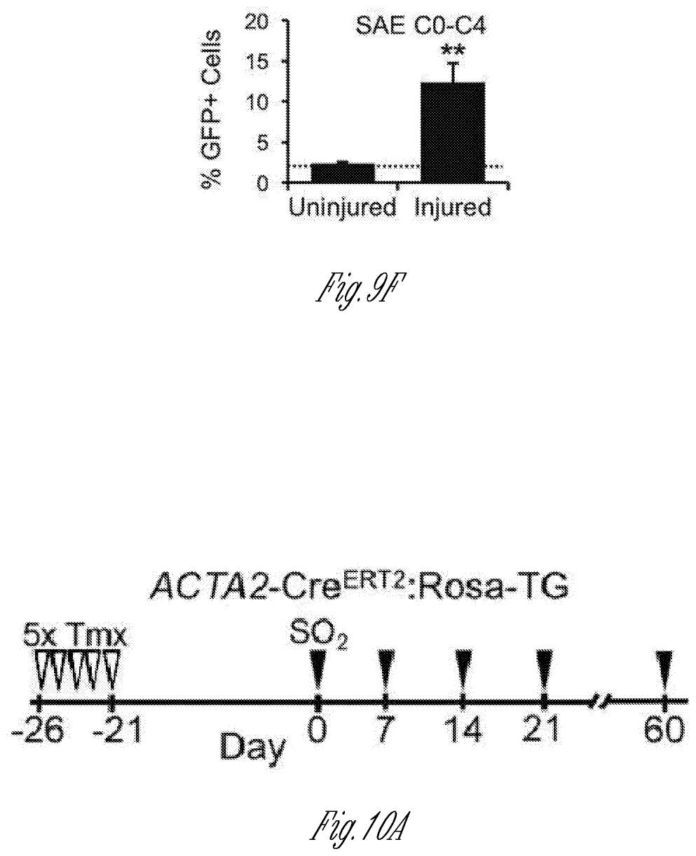

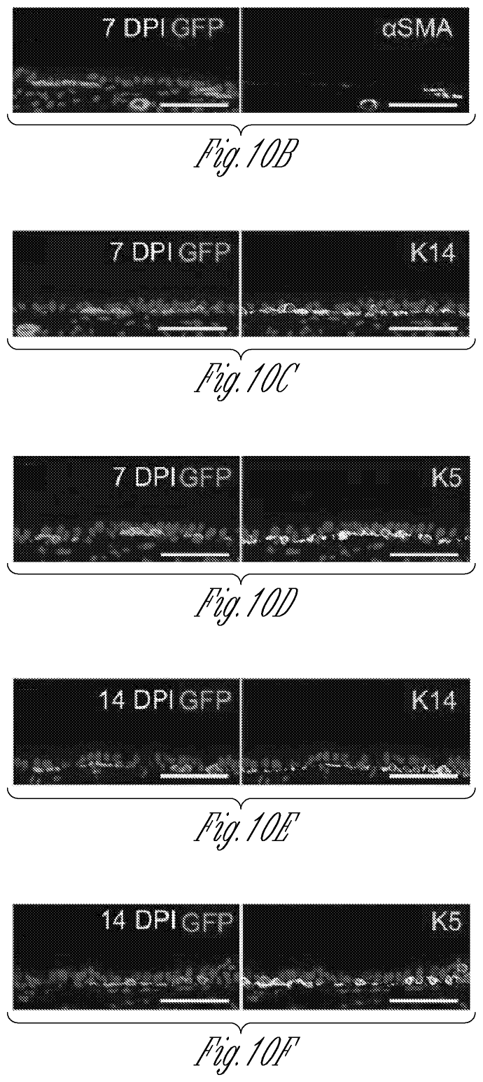

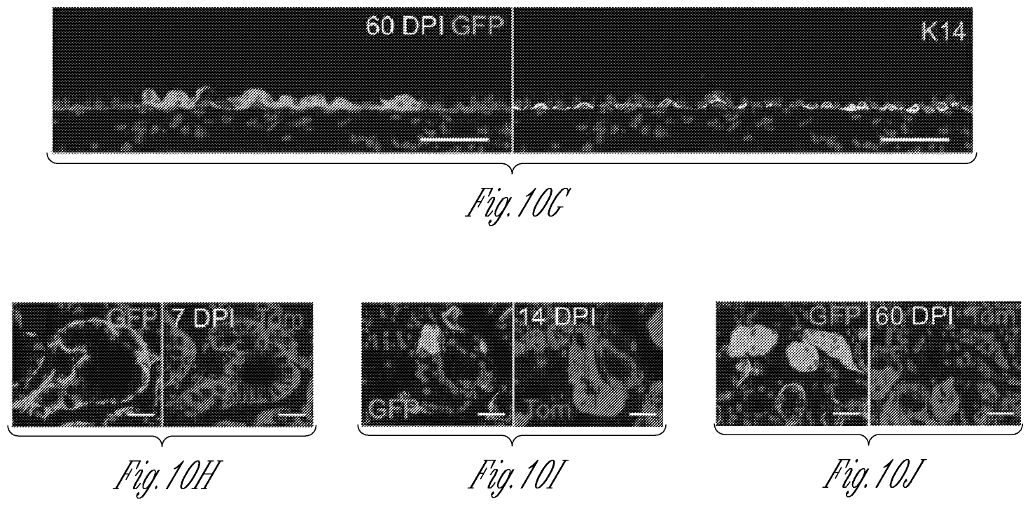

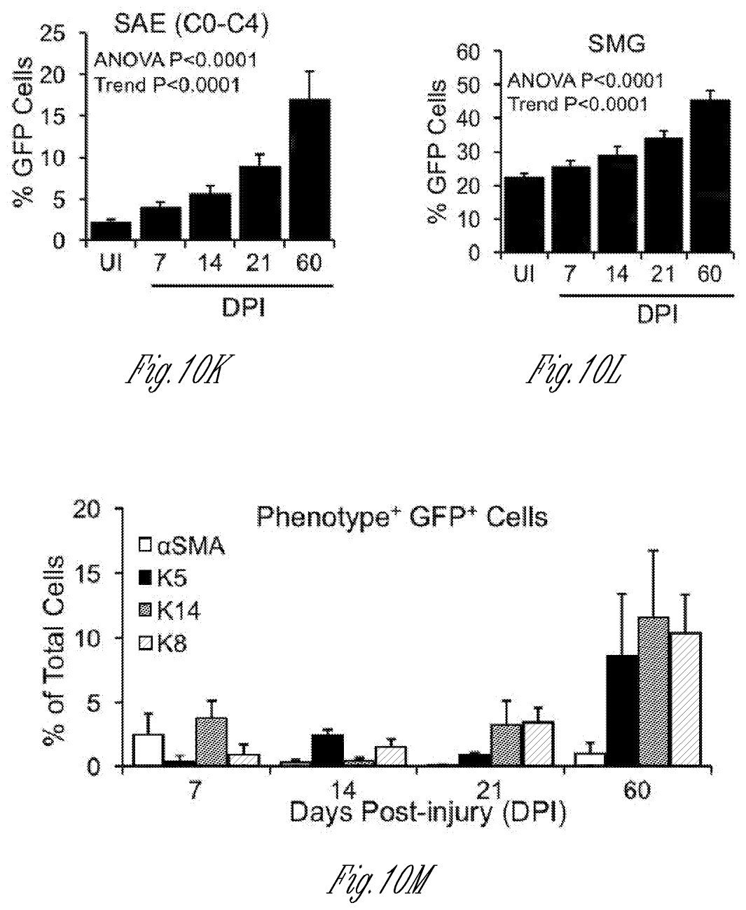

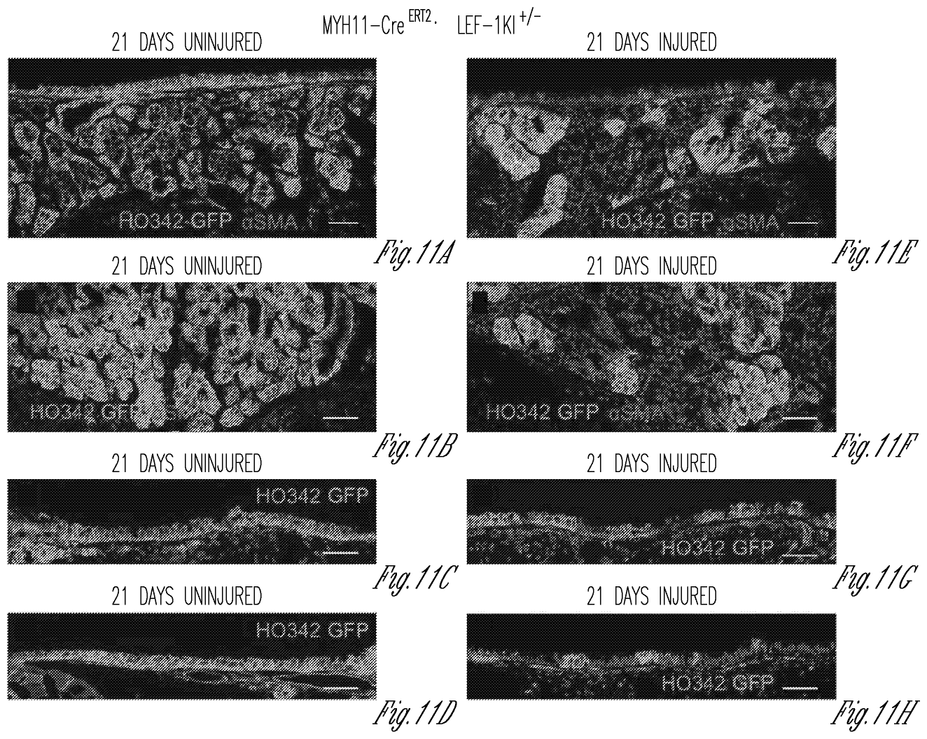

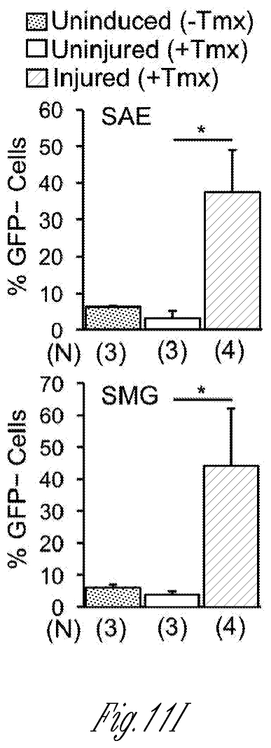

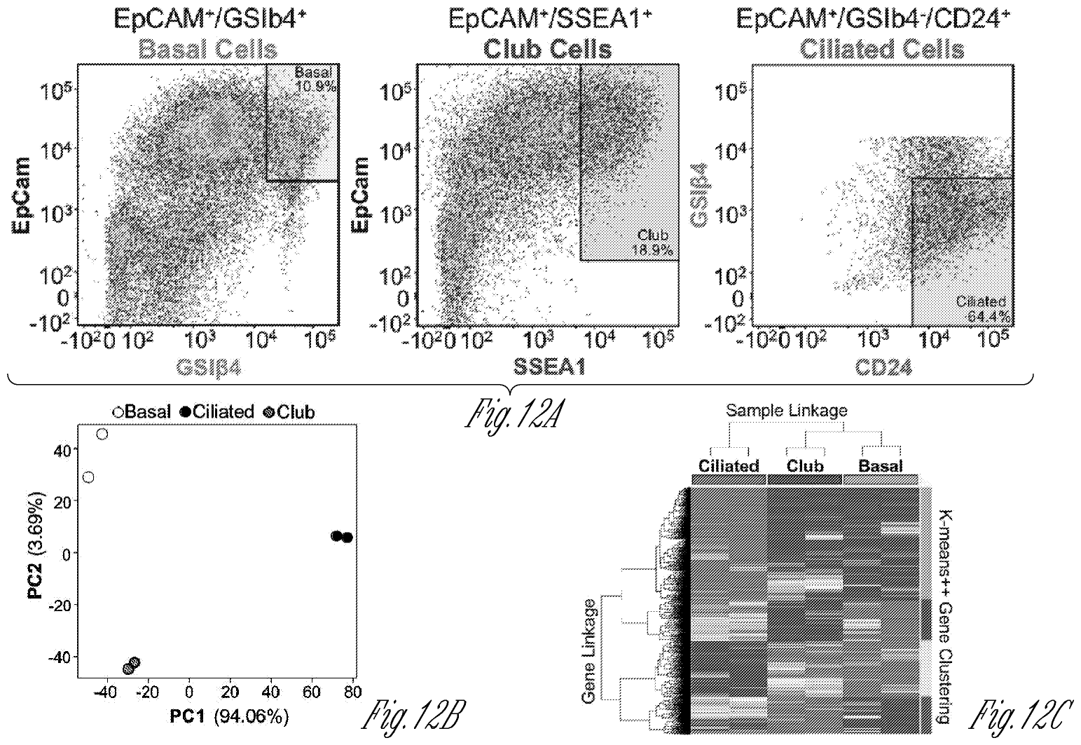

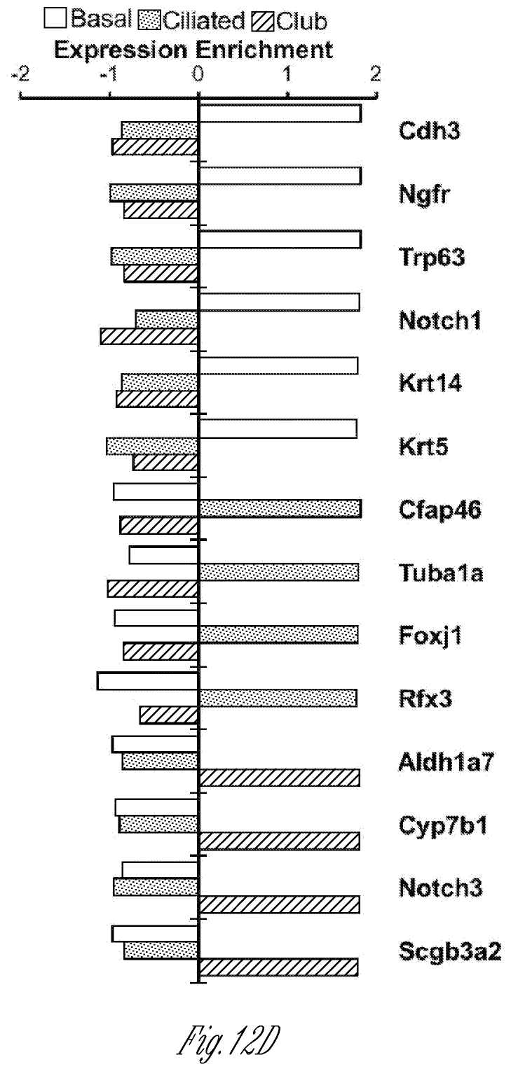

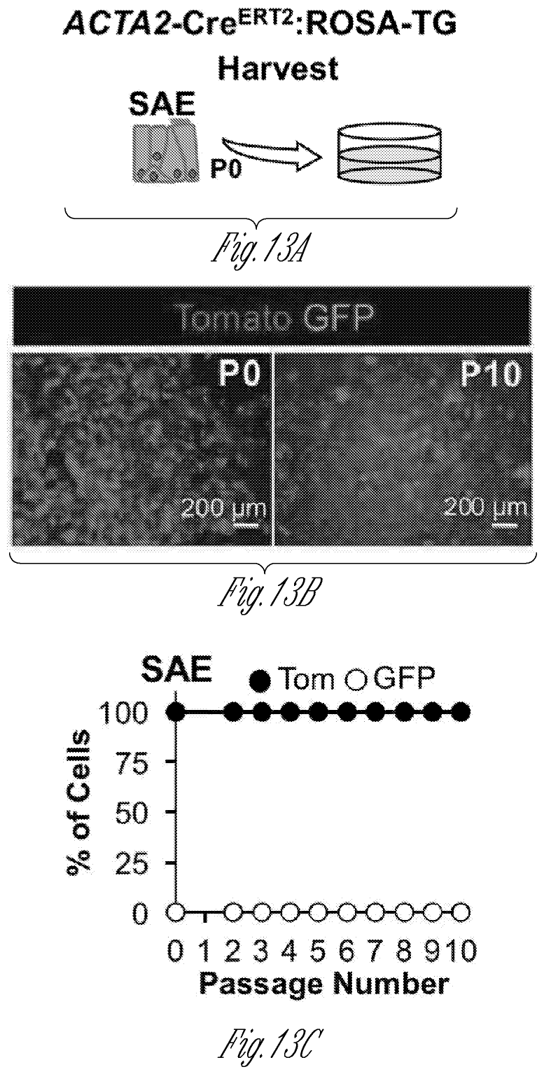

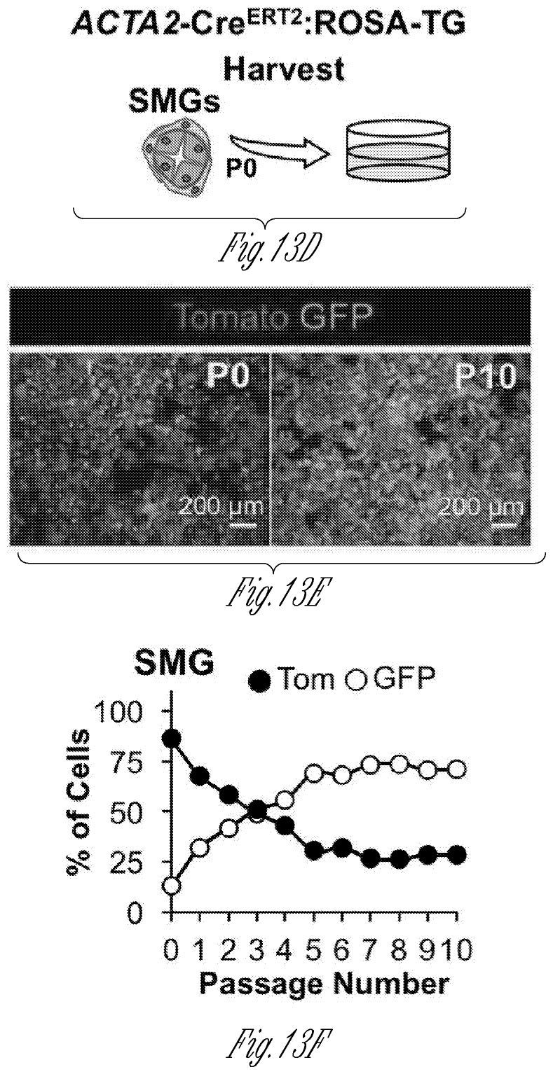

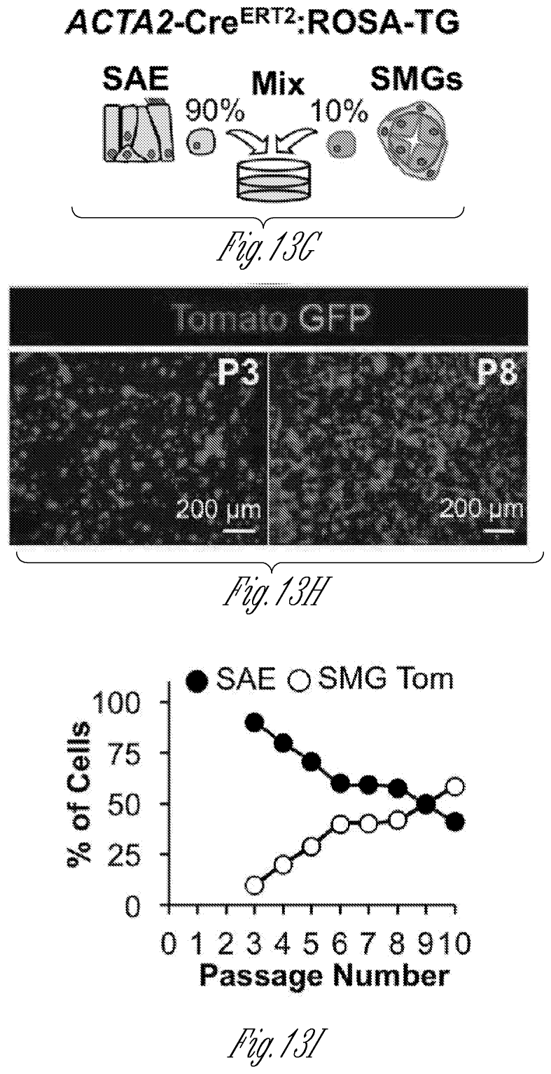

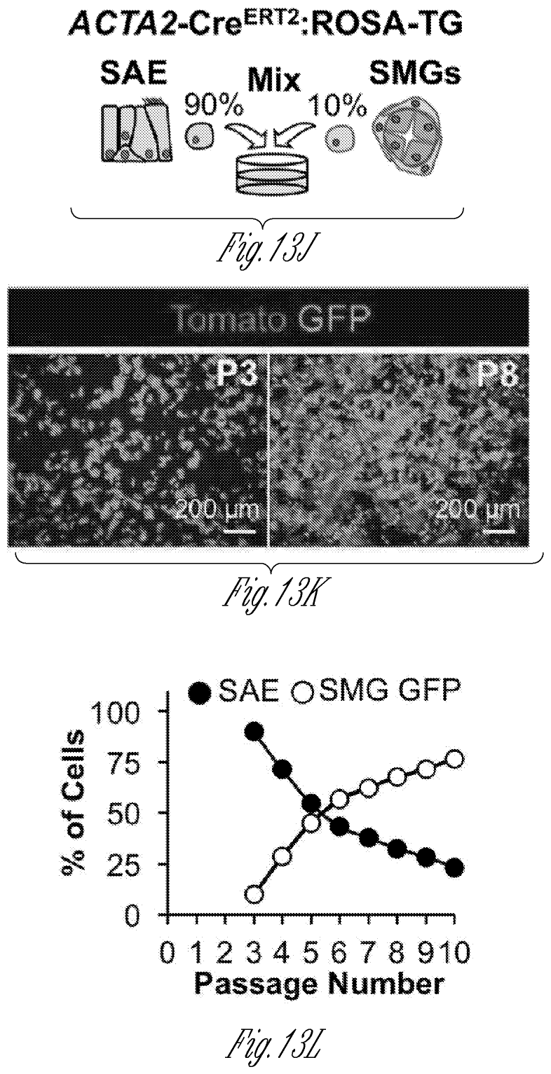

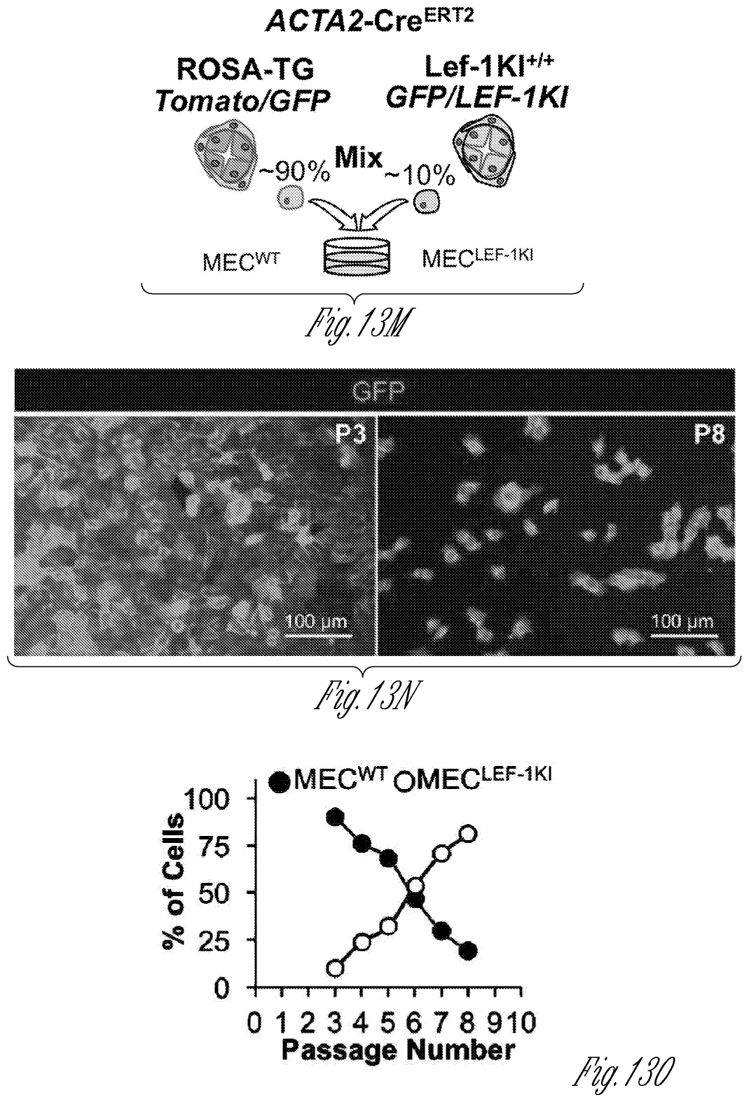

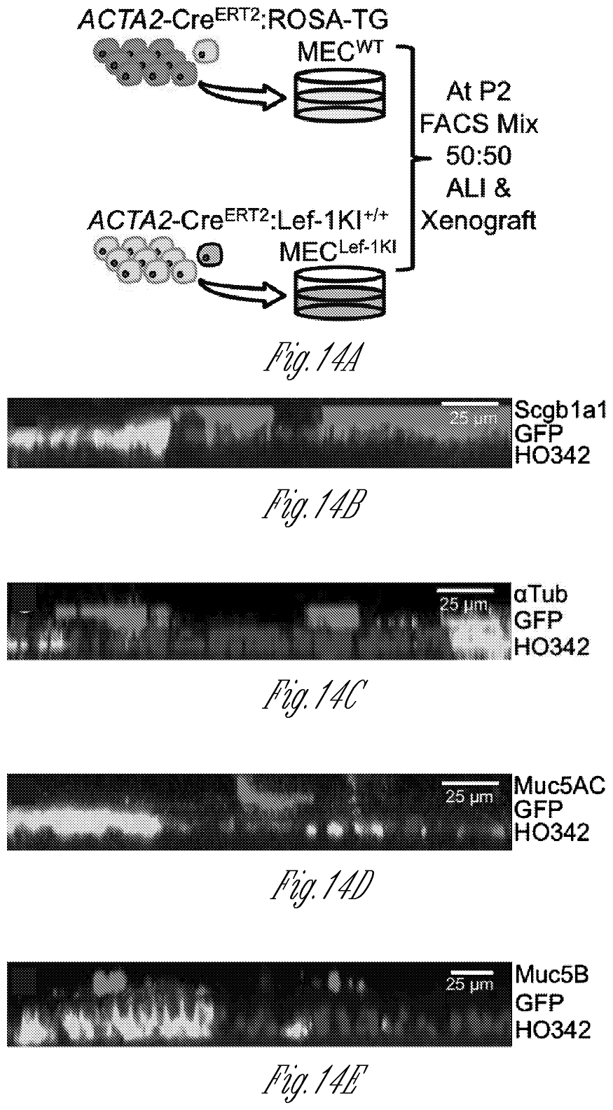

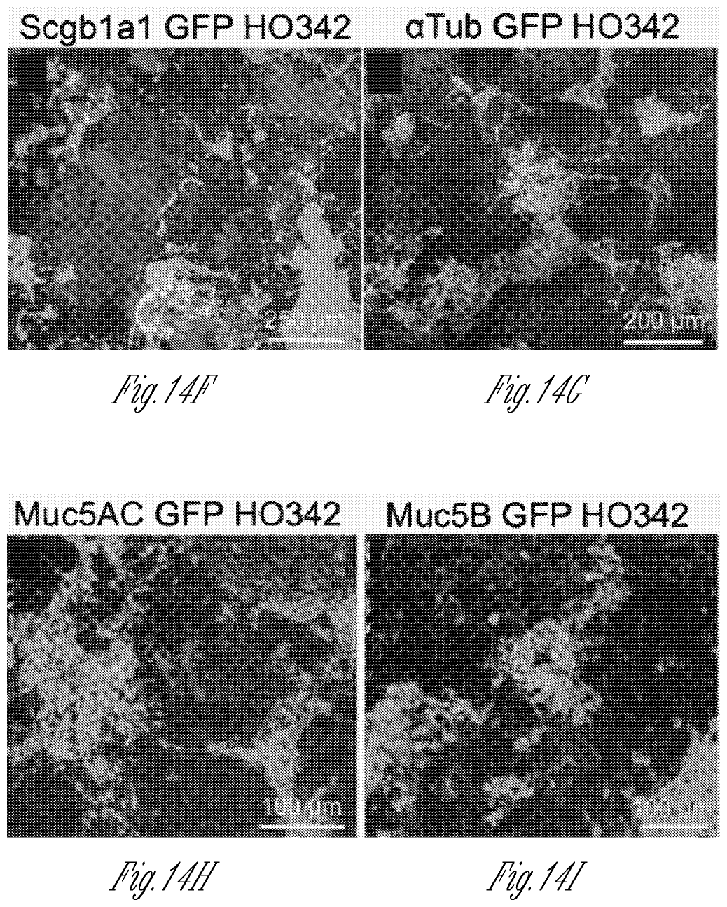

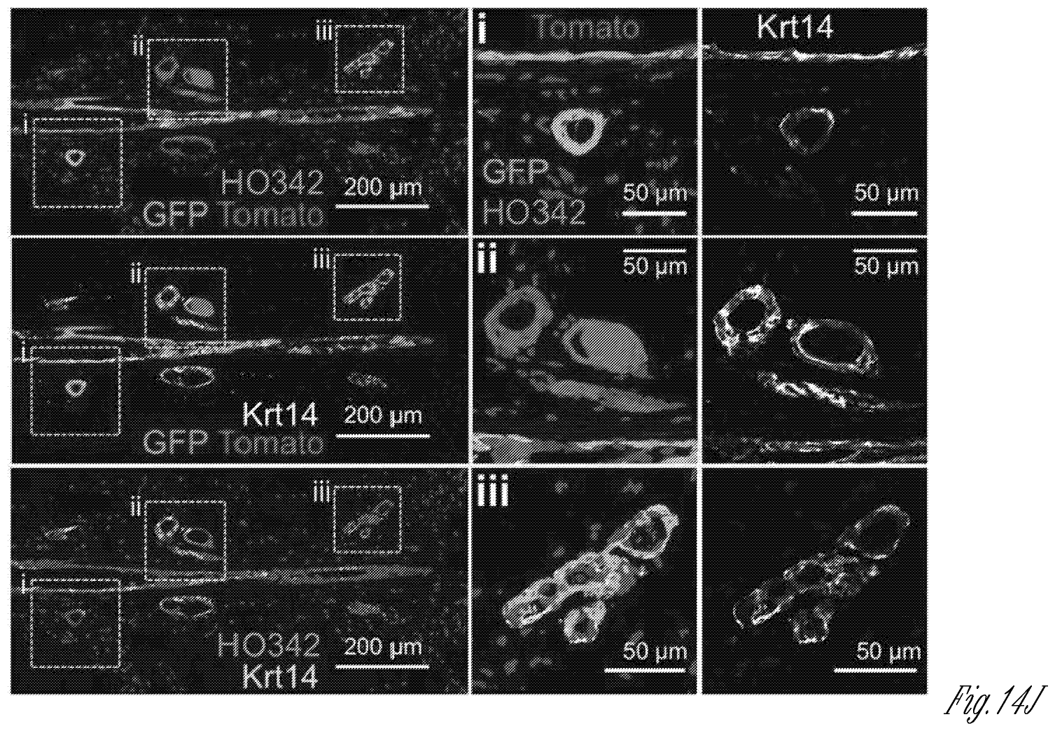

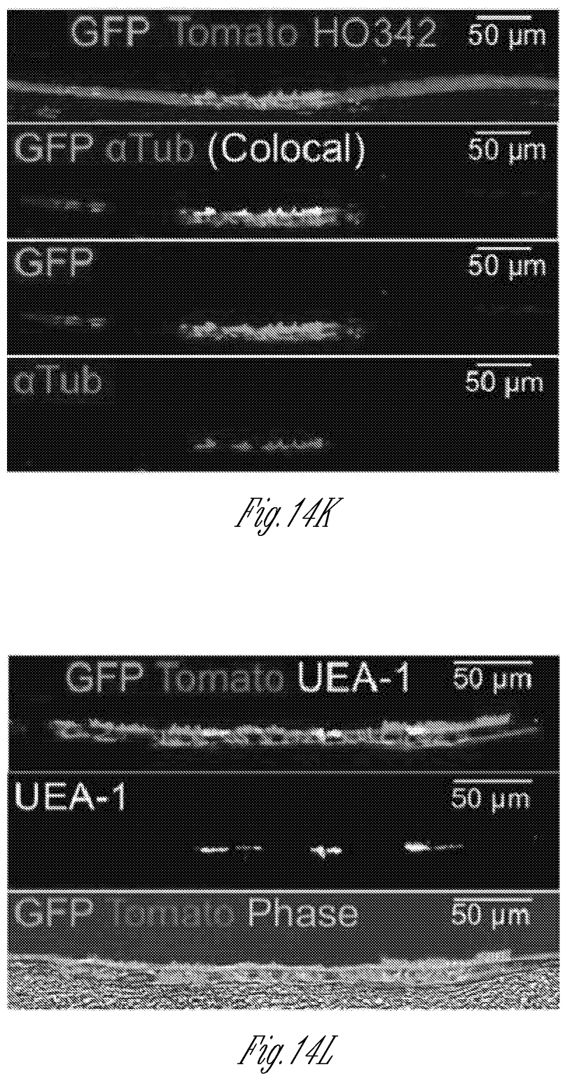

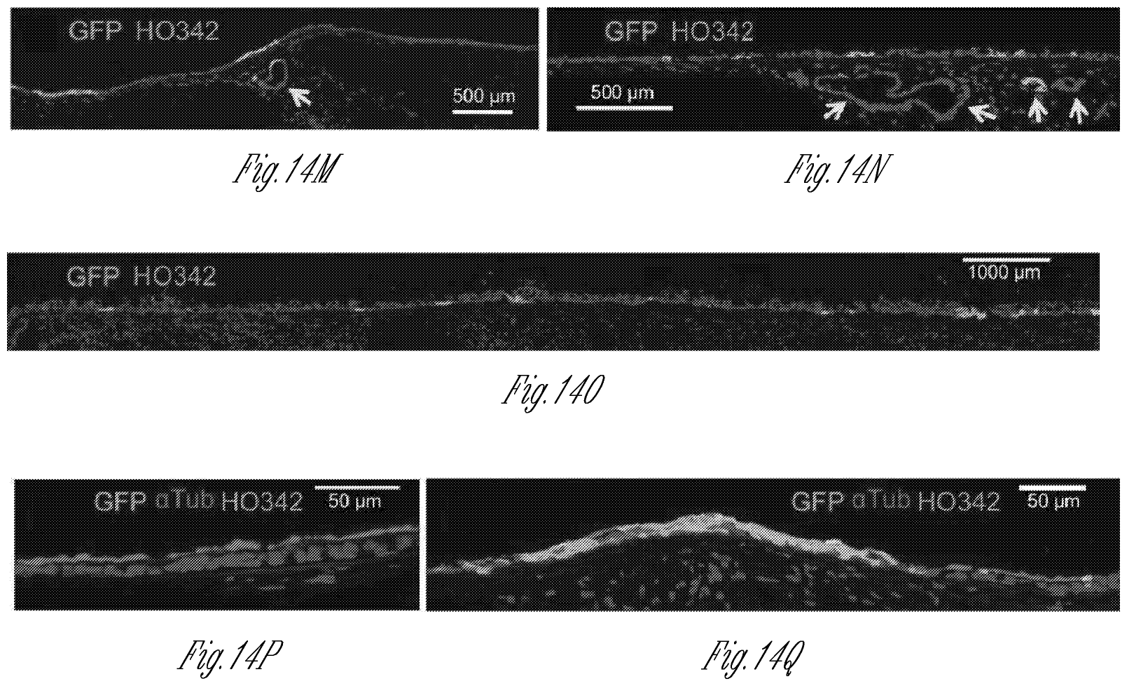

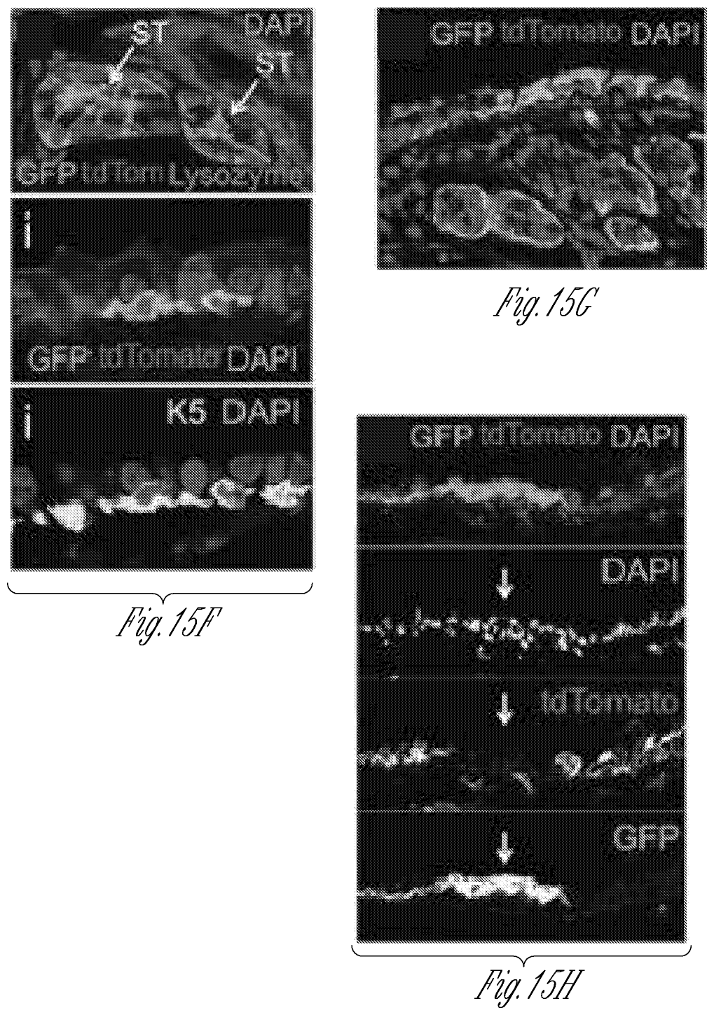

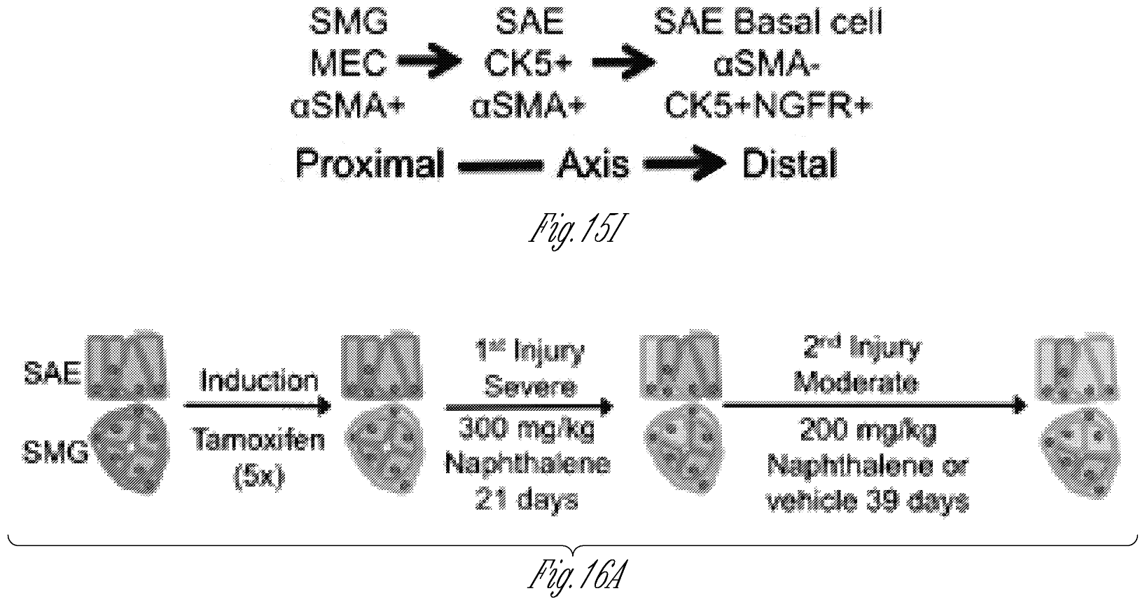

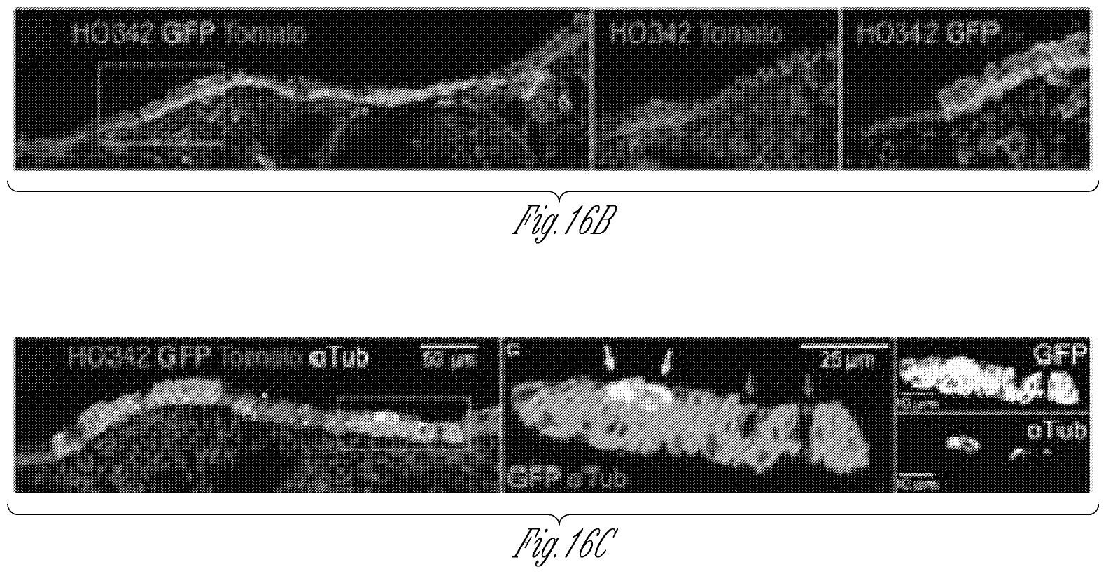

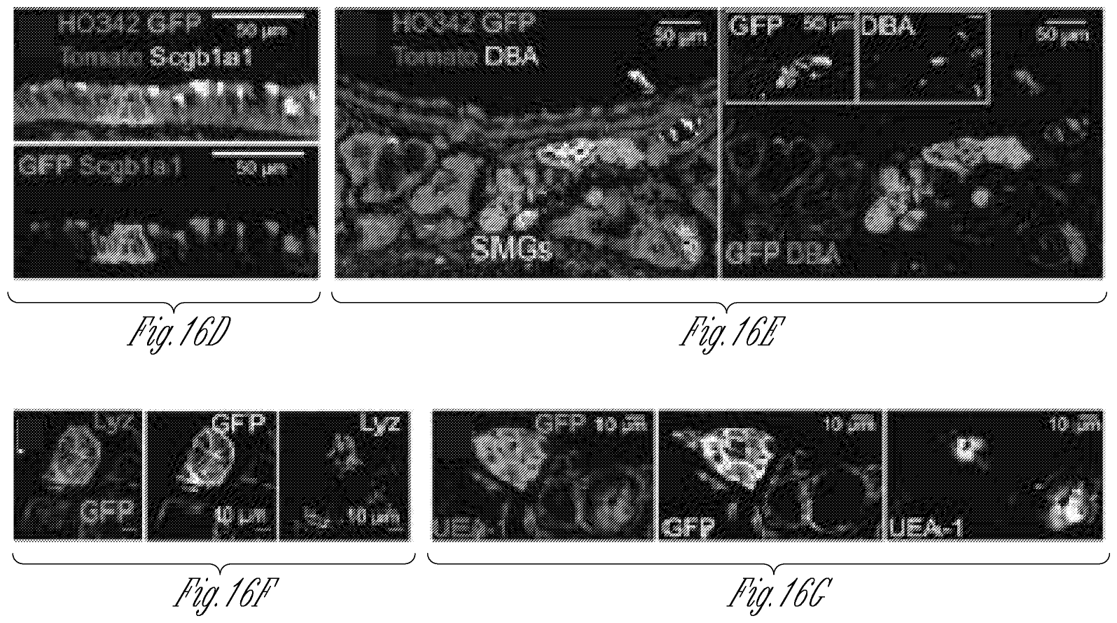

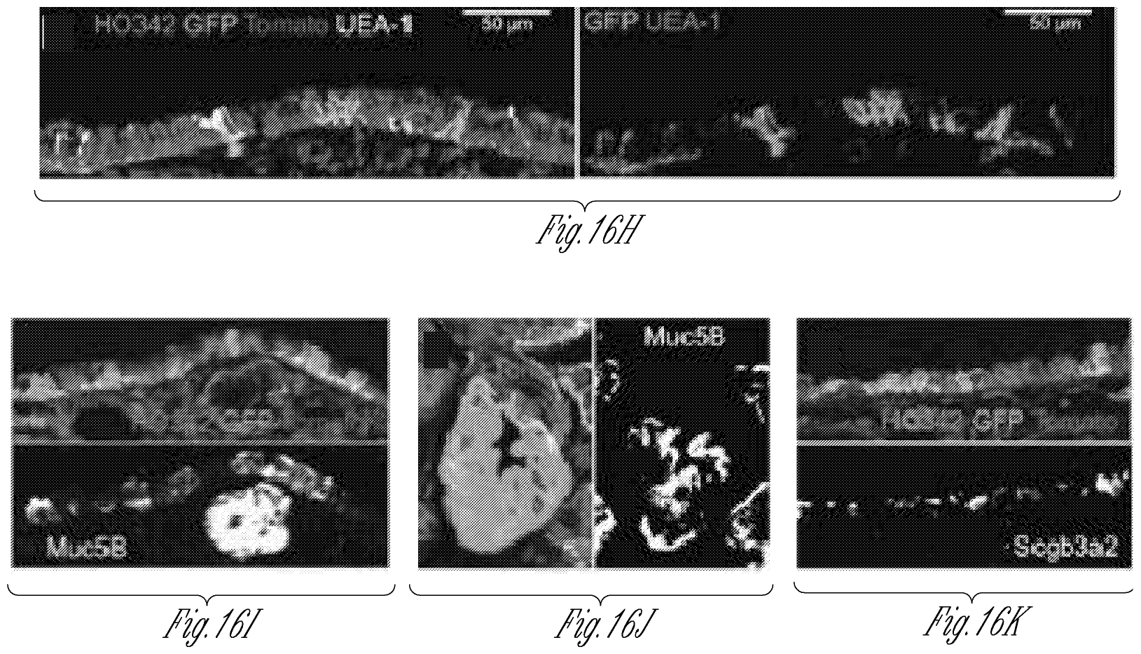

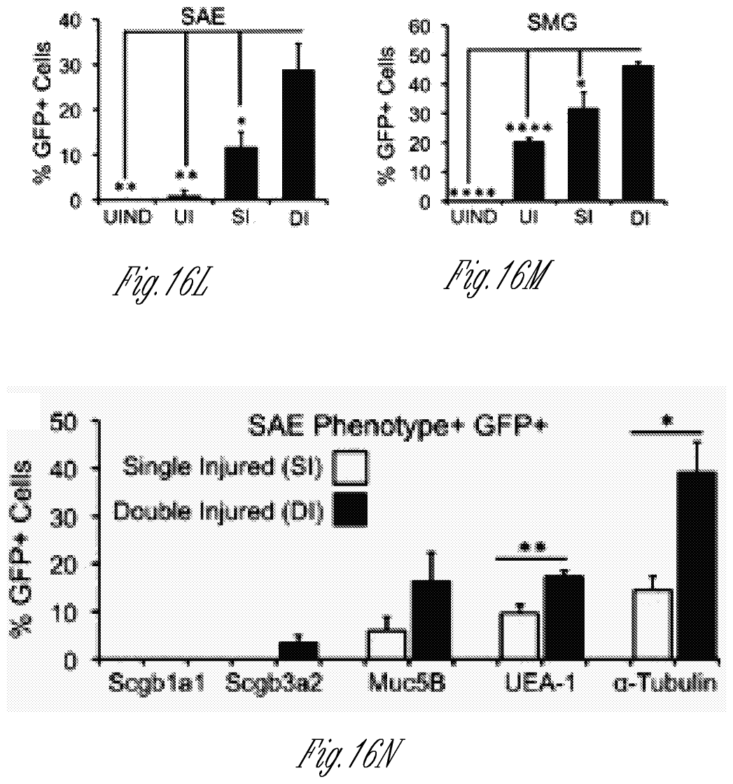

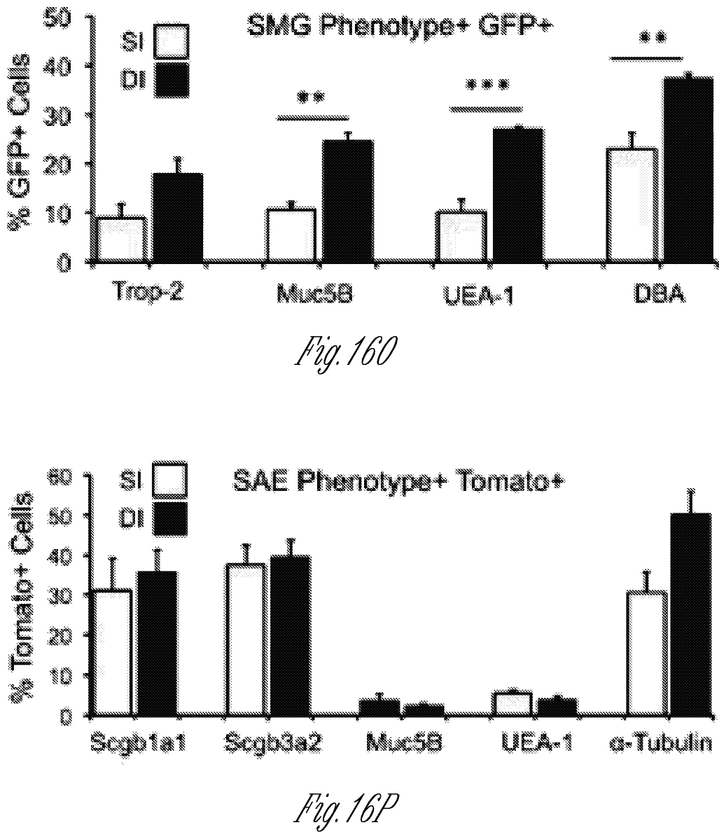

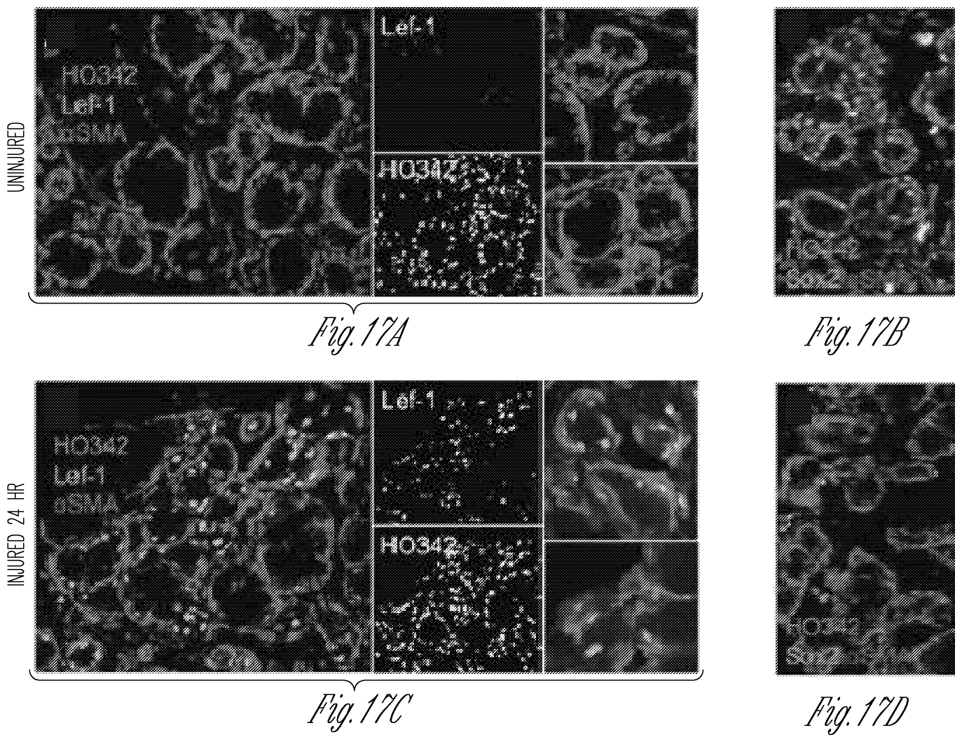

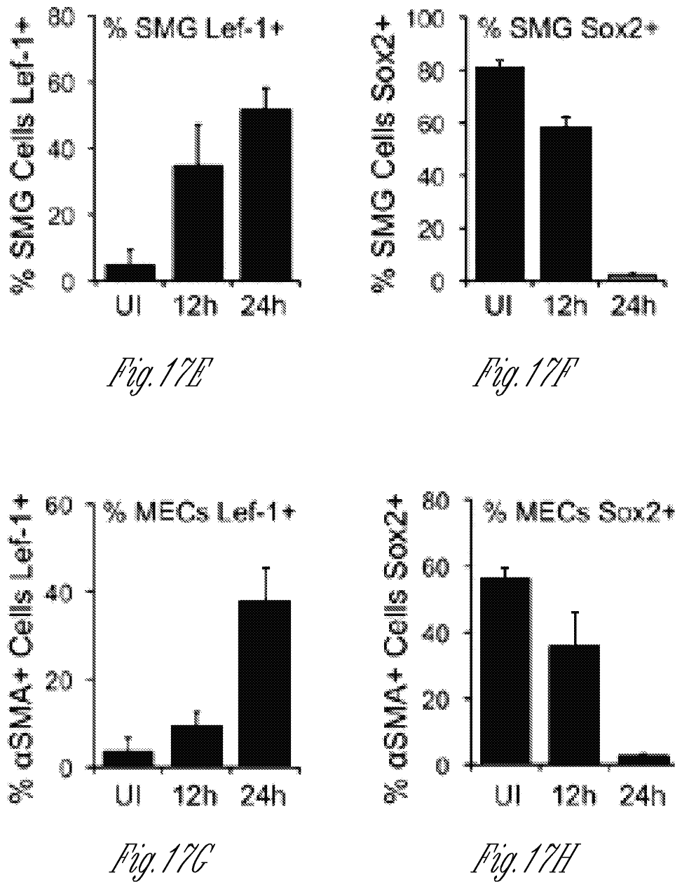

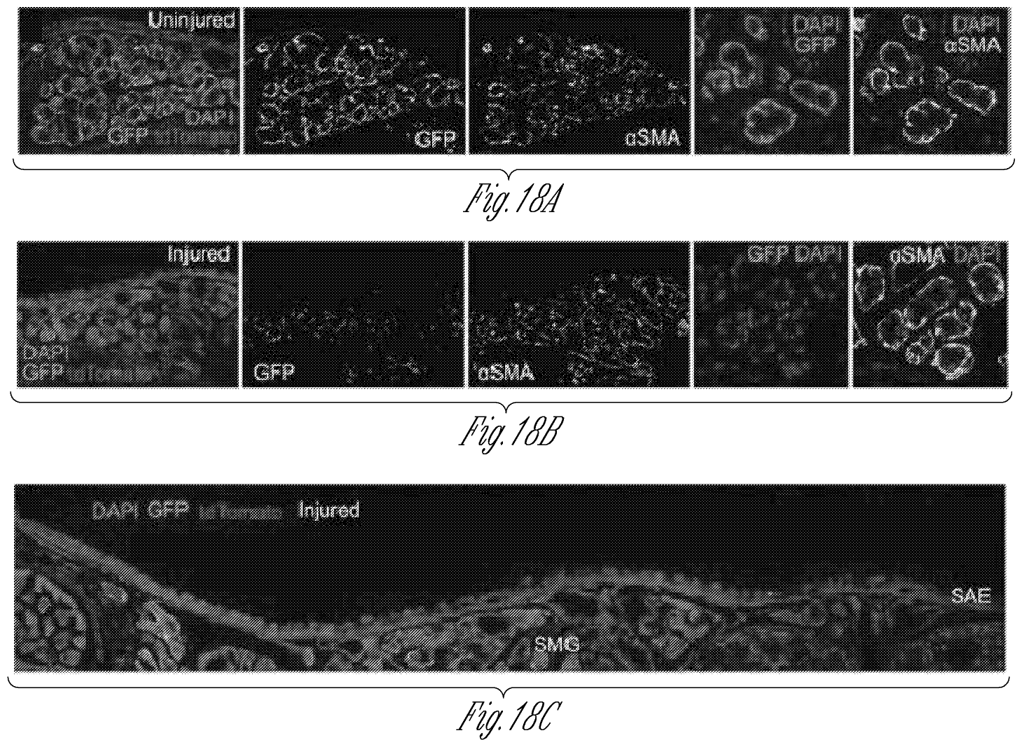

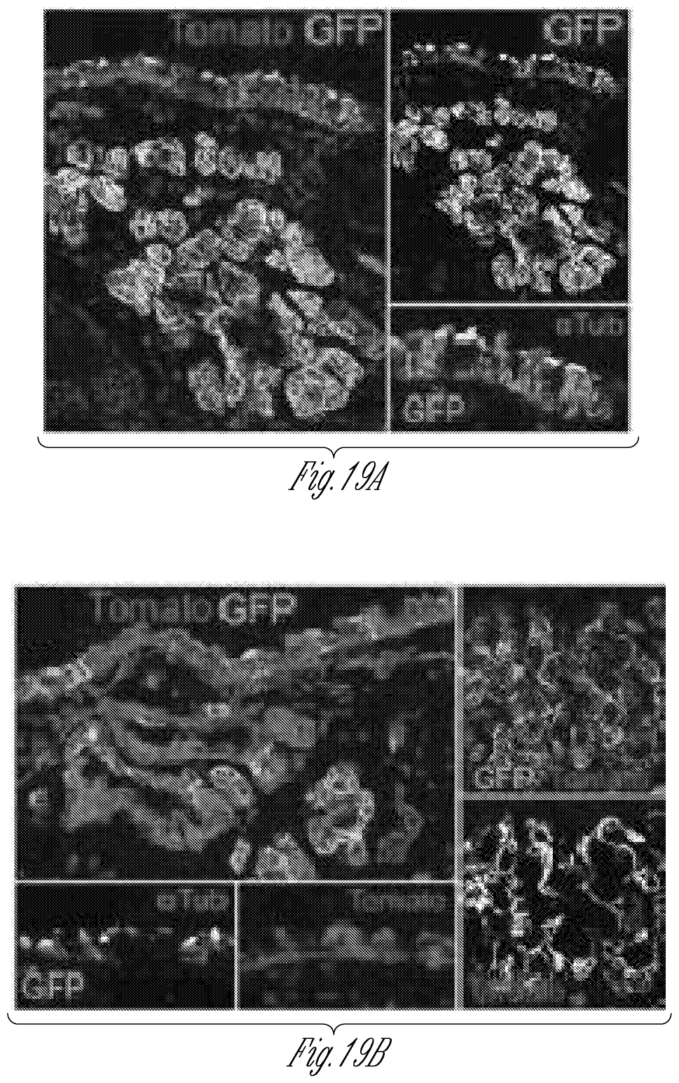

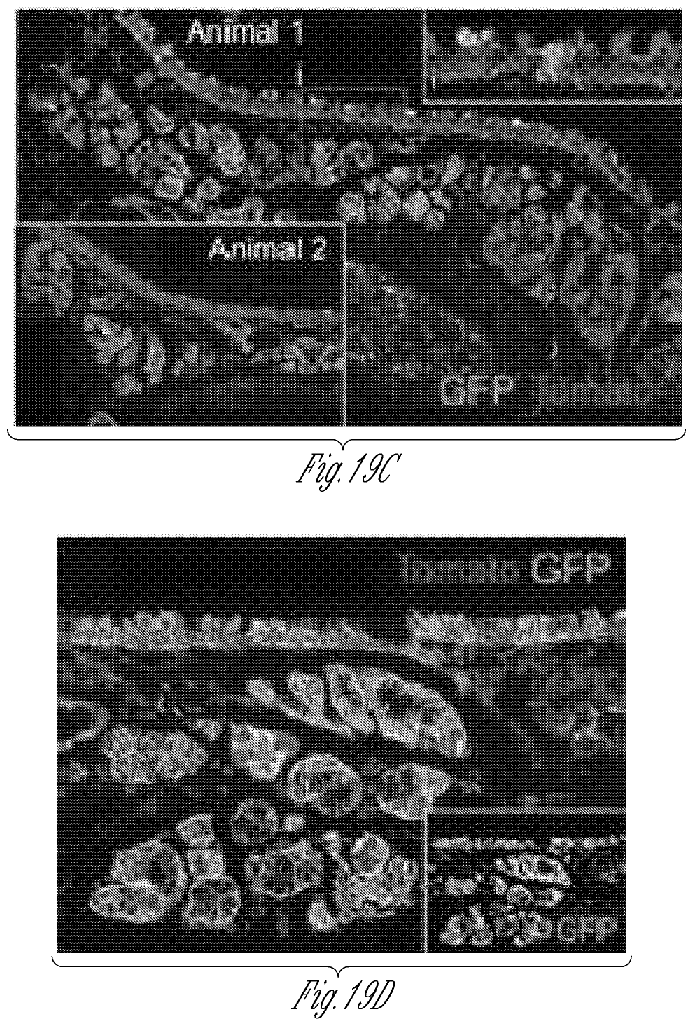

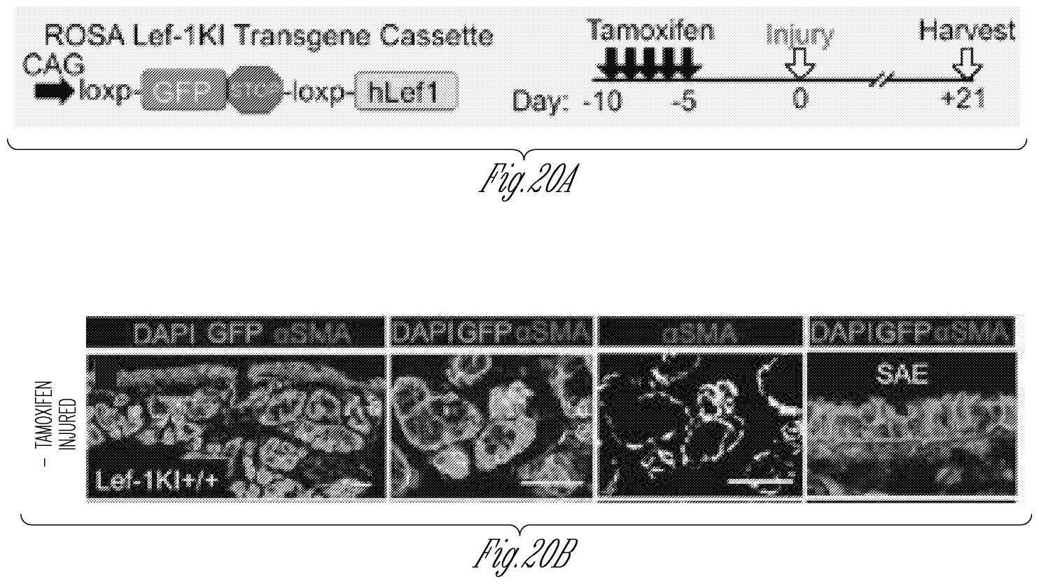

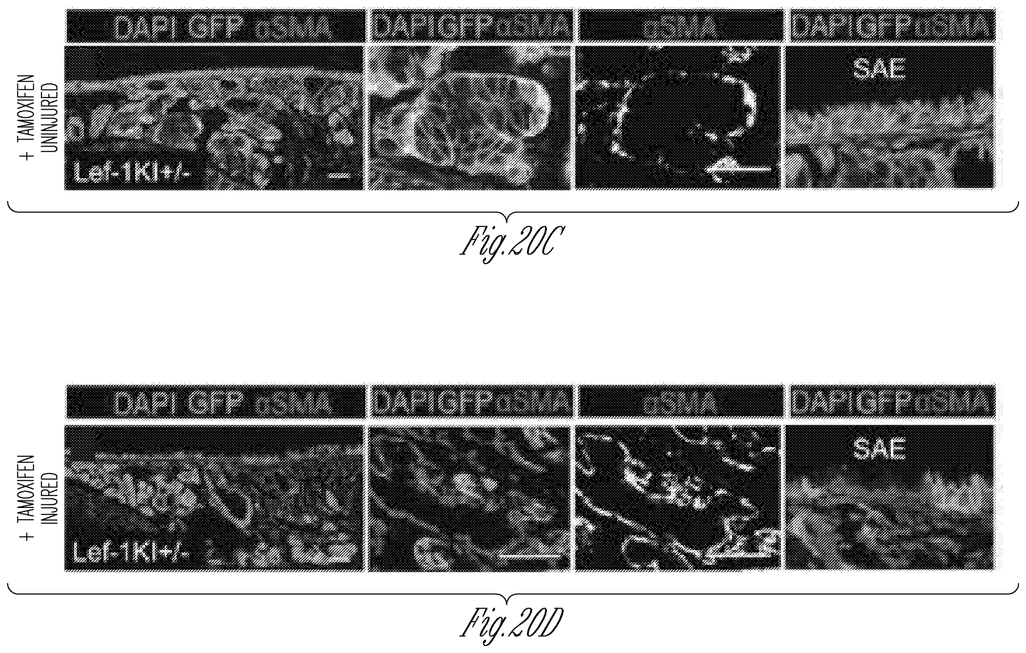

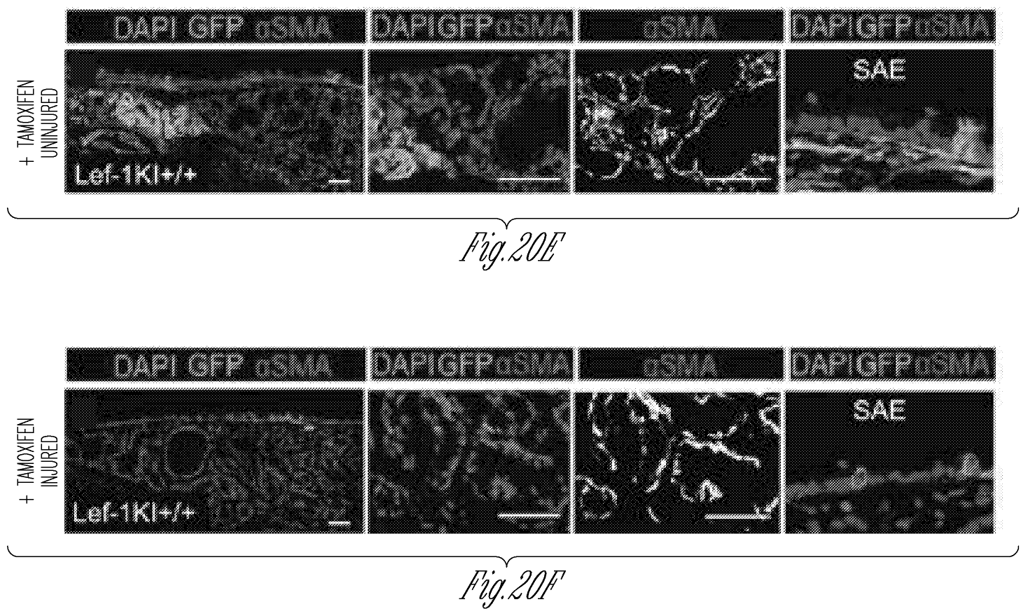

### Summary of Mouse Experiments.

| | | | | | | Time |
|---|---|---|---|---|---|---|
| | | Induction | Chase | | Injury | Post-injury |
| FIG. | Mouse Line | Time | Time | Injury Type | Quantity | to Harvest |
| FIG. 1 | ACTA2-Cre^{ERT2}: ROSA-TG | 5 days | 5 days | Naphthalene | Single | 21 days |
| FIG. 2 | ACTA2-Cre^{ERT2}: ROSA-TG | 5 days | 5 days | Naphthalene | Single | 7, 14, 21, |
| | | | | | | 60 days |
| FIG. 3 | ACTA2-Cre^{ERT2}: ROSA-TG | 5 days | 5 days | Naphthalene | Single and | 60 days |
| | | | | | Double | |
| FIG. 4 | C57BL/6 | N/A | N/A | Naphthalene | Single | 12 and |
| | | | | | | 24 hours |
| FIG. 5 | ACTA2-Cre^{ERT2}: ROSA-TG and | 5 days | 0 days | N/A | N/A | N/A |
| (Panels A-F) | ACTA2-Cre^{ERT2}: Lef-1K1^{+/+} | | | | | |
| FIG. 5 | ACTA2-Cre^{ERT2}: Lef-1K1^{+/-} and | 5 days | 5 days | Naphthalene | Single | 21 days |
| (Panels G-U) | ACTA2-Cre^{ERT2}: Lef-1K1^{+/+} | | | | | |
| FIG. 6 | ACTA2-Cre^{ERT2}: Lef-1K1^{+/-} and | 5 days | 21 days | SO₂ | Single and | 21 and |
| | ACTA2-Cre^{ERT2}: Lef-1K1^{+/+} | | | | Double | 42 days |
| FIG. 8 | C57BL/6 | N/A | N/A | Naphthalene | Single | 1, 3, 5, |
| (Panels A-E) | | | | | | 7 days |
| FIG. 8 | ACTA2-Cre^{ERT2}: ROSA-TG and | 5 days | 5 days | N/A | N/A | N/A |
| (Panels F-J) | MYH1/-Cre^{ERT2}: ROSA-TG | | | | | |
| FIG. 9 | MYH1/-Cre^{ERT2}: ROSA-TG | 5 days | 5 days | Naphthalene | Single | 21 days |
| FIG. 10 | ACTA2-Cre^{ERT2}: ROSA-TG | 5 days | 21 days | SO₂ | Single | 7, 14, 21, |
| | | | | | | 60 days |
| FIG. 17 | MYH1/-Cre^{ERT2}: Lef-1K1^{+/-} | 5 days | 5 days | Naphthalene | Single | 21 days |

### Tissue Processing and Cell Isolation

Epithelia from resected mouse tracheae were isolated using a sequential enzymatic digestion strategy as previously described with slight modifications (Lynch et al., 2016). Tracheae were opened longitudinally to expose the SAE and then digested in 1.5 mg/ml Pronase (Roche) in DMEM:F12 at 37° C. for 60 minutes with gentle nutation. Tissues were gently agitated to remove SAE and then passed through a 100 μm cell strainer. The flow through containing SAE cells was changed into DMEM:F12 and then into modified SAGM (Lonza) (Mou et al., 2016) prior to plating for culture. The remaining tracheal tissue was then dissected with fine tip surgical scissors into tissue pieces 3 mm³ and further digested to isolate SMG cells after washing tissue fragments to remove lightly adherent cells (5 changes of DMEM:F12 by pipetting up and down using a 5 ml plastic pipette). Tissue fragments were then incubated in 2× Collagenase/Hyaluronidase buffer (Stemcell Technologies) diluted in DMEM:F12 at 37° C. for 45 minutes with gentle nutation. Pre-warmed 0.25% Trypsin-EDTA (Life Technologies) was then added to the cell mixture to a final concentration of 0.05% Trypsin-EDTA and incubated for an additional 30 minutes at 37° C. with gentle nutation. After pipetting up and down using a P1000 pipette, a single cell suspension was obtained by passing the cell mixture through a 100 μm cell strainer. The flow through containing SMG cells was changed into DMEM:F12 and then into modified small airway growth media (SAGM, Lonza) prior to plating for culture. All centrifugations were performed at 250×g for 7 min. Primary cells were cultured in modified SAGM with the addition of 10 μM Y-27632, 1 μM DMH-1, 1 μM A83-01, and 1 μM CHIR 99021 (Tocris) on tissue culture plastic pre-treated with filter-sterilized laminin-enriched 804G-conditioned media as previously described (Mou et al., 2016).

Collection of SAE for downstream isolation of basal, club and ciliated cell populations was done as previously described (Zhao et al., 2014). Mouse tracheae were resected and separated from the proximal, SMG-containing portion of the airway and minced. Fragments were incubated in a dissociation solution containing Papain (20 U/mL), EDTA (1.1 mM), 2-Mercaptoethanol (0.067 mM), Cysteine-HCl (5.5 mM) and DNAse I (100 U/mL) for 1 hour and 30 minutes. The reaction was stopped with Ovomucoid protease inhibitor (Worthington biochemical Corporation) on a rocker at 4° C. for 20 minutes. Cells were then immunostained for FACS analysis as described below prior to resuspending in FACS buffer (2.0% FBS in PBS).

### Naphthalene and SO₂ Experiments

Adult mice (˜8-12 weeks of age) were injured with a single intraperitoneal injection of either 200 μg or 300 μg naphthalene per gram bodyweight. For severe SO₂ injury with 600 ppm was administered for 4 hours to adult mice. Mice were hydrated with subcutaneous injections of D5NS (5% dextrose in normal saline) during the first 48 hrs following naphthalene injury. Mock injury was performed with corn oil injection, and served as a baseline control. Mice were allowed to recover following injury (length of time is indicated in each figure legend) before being either re-injured or euthanized for study.

### Flow Cytometry

Flow cytometric analysis was performed on cultured primary SAE and SMG cells isolated from A CTA2-Cre^{ERT2}:ROSA-TG or ACTA2-Cre^{ERT2}:Lef-1KI mice. Cells were dissociated from plastic plates using Accutase (Stemcell Technologies), changed into HBSS containing 2% FBS, and passed through a 40 μm cell strainer. GFP⁺ and Tomato⁺ (ACTA2-Cre^{ERT2}:ROSA-TG mice) or GFP⁺ and GFP⁻ (ACTA2-Cre^{ERT2}:Lef-1KI mice) cell populations were identified after gating for viability using Hoechst 33258 (Molecular Probes) at a final concentration of 4 μg/ml. Cells were analyzed and sorted on a FACS Aria II (BD Biosciences). For fractionating SAE into basal, club, and ciliated populations, cells were stained with EpCAM-PECy7 (eBiosciences), GSI84-FITC (Sigma), SSEA1-Alexa Fluor® 647 (BioLegend), and CD24-PE (BD Pharmingen) for 30 minutes on ice as previously described (Zhao et al., 2014), prior to FACS. Basal cells were considered EpCAM+ and GSIβ4+. Secretory cells were considered EpCAM+ and SSEA1+. Ciliated cells were considered EpCAM+, GSIβ4- and CD24+. Cell populations were sorted directly into TRIzol (ThermoFisher Scientific) for mRNA isolation.

### Competitive Cell Growth Assay

Primary cells from ACTA2-Cre^{ERT2}:ROSA-TG, ACTA2-Cre^{ERT2}:Lef-1K1^{+/+}, and C57BL/6 mice were recovered in modified SAGM (Lonza) as described above. At the time of passage total cells were counted using a Countess Automated Cell Counter (Invitrogen), and 1×10⁵ cells were seeded into one well of a freshly prepared 6-well dish. The remaining cells were analyzed with a BD LSR II flow cytometer (BD Biosciences) to determine the percentage of Tomato⁺, GFP⁺, and/or non-fluorescent cells. For reproportioned population mixing experiments, passage 1 (P1) cells were analyzed and sorted on a FACS Aria II (BD Biosciences) into Tomato⁺ and GFP⁺ (ACTA2-Cre^{ERT2}:ROSA-TG mice) or GFP⁺ and GFP⁻ (ACTA2-Cre^{ERT2}:Lef-1K1^{+/+} mice) populations. Each population was expanded separately to 80% confluence of a 6-well dish as P2 cultures. Competitive cell growth assays were established with 1×10⁵ total cells at P3 by mixing Tomato⁺ or GFP⁺ glandular progenitors with non-fluorescent SAE progenitors at a % ratio 10:90 (SMG^{Tomato+ or GFP+}:SAE). To compare wild type MECs (MEC^{WT}) with Lef-1-overexpressing MECs (MEC^{Lef-1KI}), competitive cell growth assays were established by mixing GFP⁺ cells isolated from ACTA2-Cre^{ERT2}:ROSA-TG mice (MEC^{WT}) with GFP⁻ cells isolated from ACTA2-Cre^{ERT2}:Lef-1K1^{+/+} mice (MEC^{LEF1KI}) at a % ratio of 10:90. All cultures were expanded to near confluency before passaging and quantification of populations.

### Migration Assay

Primary MEC^{WT} (GFP⁺ cells isolated from ACTA2-Cre^{ERT2}:ROSA-TG mice) and MEC^{Lef-1KI}(GFP⁻ cells isolated from ACTA2-Cre^{ERT2}:Lef-1KI^{+/+} FE mice) were plated separately at 1×10⁵ cells per well on a 6-well dish. Cells were grown as described above. Living cell nuclei were labeled with NucRed Live 647 ReadyProbes Reagent (Invitrogen) by incubating the cells with two drops of reagent per milliliter of media for 30 minutes, and prior to imaging the media was replaced with fresh modified SAGM. Starting eight hours after seeding, live-cell mobility was recorded using a Leica spinning disk confocal microscope fitted with a CO₂ incubation chamber and 37° C. heated stage. Images were collected using differential interference contrast (DIC) and a 630 nm wavelength far red laser every five minutes for three hours. For each genotype, four separate regions from within three independent wells were imaged every five minutes over three hours. Movies were analyzed using the Multidimensional Motion Analysis application in MetaMorph imaging software. To display cell motility paths on a subset of cells in an unbiased manner and to statistically test the difference between MEC^{WT} and MEC^{Lef-1KI} motility, single cells from each movie were selected using an online random number generator.

### Immunofluorescence

Mouse tracheae were fixed in 4% PFA in PBS for 48 hrs prior to washing in PBS and embedding in OCT frozen blocks. Frozen sections were cut at 10 μm. Frozen tissue sections were post-fixed in 4% PFA for 20 minutes and rinsed in three changes of PBS. Antigen retrieval using citrate boiling was performed on C57BL/6 mice and ACTA2-Cre^{ERT2}:Lef-1KI when staining for nuclear Lef-1, Sox-2, TCF7, and β-catenin antigens (note that this antigen retrieval leads to a more diffuse GFP staining pattern in Lef-1 KI mice, but is required to detect nuclear Lef-1 and TCF7). Slides were incubated in blocking buffer containing 20% normal donkey serum, 0.3% Triton X-100, and 1 mM CaCl₂) in PBS for 1 hr. The slides were incubated with primary antibody (or a mixture of primary antibodies) in diluent buffer containing 1% normal donkey serum, 0.3% Triton X-100, and 1 mM CaCl₂ in PBS overnight at 4° C. Slides were washed in three changes of PBS and incubated with secondary antibody (or a mixture of secondary antibodies) in diluent buffer overnight at 4° C. Fluorescent images were collected with a Zeiss LSM 700 line-scanning confocal microscope (Carl Zeiss, Germany). Nuclei were stained using Hoechst 33342 (Invitrogen) or DAPI (4′,6-diamidino-2-phenylindole) (Invitrogen). Slides were mounted with ProLong Gold (Invitrogen).

### Lectin Staining

To stain for mucous cell types of the SMGs and SAE, slides were stained with biotinylated lectins subsequent to immunostaining and prior to coverslipping. Slides were washed in three changes of PBS, and endogenous avidin and biotin were blocked using an Avidin/Biotin Blocking kit (Vector Laboratories) per the manufacturer instructions. Biotinylated lectins, Dolichos biflorus agglutinin (DBA) (Vector Laboratories) or Ulex europaeus agglutinin I (UEA-1) (Vector Laboratories), were used at a concentration of 10 μg/ml for 30 mins at room temperature. Slides were washed in three changes of PBS and incubated with Alexa Fluor 647-conjugated Streptavidin (Jackson ImmunoResearch 016-600-084) at a concentration of 2 μg/ml for 30 mins at room temperature.

### Image Analysis

For quantification of tile-scanned fluorescent images, multiple fluorescent channels were quantified using MetaMorph Software's Multi Wavelength Cell Scoring Application Module per manufacturer's instructions. Typically three sections separated by at least 60 μms were analyzed for each animal, and the average values for each animal were used to calculate the mean±SEM for each group. Unless otherwise stated, quantification was performed in the C0-C4 region of the trachea from tiled scanned longitudinal images that included both sides of the tracheal epithelium.

### Air-Liquid Interface Cultures

Expanded primary cells were grown at an air-liquid interface (ALI) on 0.33 cm² polyester transwell membranes (Corning) that were pre-treated with 804G-conditioned media. Each well was seeded with 2×10⁵ cells suspended in modified SAGM expansion media (see above). At 16-24 hrs post-seeding, cultures were moved to an air-liquid interface and maintained with Pneumacult ALI media (Stemcell Technologies) for at least 21 days. Mixed-cell ALI cultures were established using FACS purified MEC^{WT} (GFP⁺ cells isolated from ACTA2-Cre^{ERT2}:ROSA-TG mice) and MEC^{Lef-1KI}(GFP⁻ cells isolated from ACTA2-Cre^{ERT2}:Lef-1K1^{+/+} mice) P2 populations seeded at a 1:1 ratio.

### Tracheal Xenografts

The proliferative capacity and multipotency of SMG-derived MECs progenitors and SAE-derived progenitors were evaluated in an ex vivo tracheal xenograft model as previously described with slight modifications (Engelhardt et al., 1995). Primary cells were isolated from tracheal SAE of wild type mice and SMG of tamoxifen-induced ACTA2-Cre^{ERT2}:ROSA-TG mice and expanded in vitro as described above to P2. SMG-derived cells including GFP-expressing cells (lineage-traced MECs) and tdTomato-expressing cells (untraced gland cells) were mixed at a ratio of 1:9 (SMG cells:SAE cells) with wild type SAE-derived cells. Denuded tracheal xenografts were also reconstituted with FACS purified SMG cells isolated from tamoxifen-induced ACTA2-Cre^{ERT2}:ROSA-TG (GFP⁺) and ACTA2-Cre^{ERT2}:Lef-KI^{+/+} (GFP⁻) mice and seeded at a ratio of 1:1 (GFP⁺WT MECs:GFP⁻ Lef-1KI MECs). Two-to-three week old ferret tracheal xenograft scaffolds were freeze-thawed three times and the lumen was rinsed in MEM to remove dead cells. Tracheae were then seeded with 2×10⁶ cells total, ligated to flexible tubing, and transplanted subcutaneously into athymic mice. Xenografts were irrigated 1-2 times a week with F12 media and harvested at 5-6 weeks post-transplant.

### RNAseq of Culture-Expanded MECs

Primary glandular cells were isolated from ACTA2-Cre^{ERT2}: ROSA-TG and ACTA2-Cre^{ERT2}:Lef-1K1^{+/+} mice after five sequential injections of tamoxifen. Primary cells from three to five mice were pooled for each sample. P1 cells were analyzed and sorted on a FACS Aria II (BD Biosciences) collecting lineage-tagged MECs-GFP+ cells (MEC^{WT}) were isolated from ACTA2-Cre^{ERT2}:ROSA-TG cultures, and GFP⁻ cells (MEC^{Lef-1K}) were isolated from ACTA2-Cre^{ERT2}:Lef-1K1^{+/+} cultures. Cells were sorted directly into RNA lysis buffer and RNA was extracted using an RNeasy Plus Mini Kit (Qiagen). Samples were treated with DNase and RNA Integrity Numbers (RIN) were assessed using an Agilent BioAnalyzer 2100. All samples had RIN values ≥10. Indexed cDNA libraries were constructed using a TruSeq mRNA stranded preparation. Normalized libraries were sequenced using 75 bp paired-end reads on a HiSeq 4000 (Illumina). The number of transcripts per million was calculated for each RNAseq sample using RSEM (Li and Dewey, 2011) and aligned to the Ensembl's mm10 transcriptome. Genes with a mean expression value greater than three times the standard deviation of that gene within MEC^{WT} or MEC^{Lef-1KI} sample groups were selected from the dataset as stably expressed genes. Differential expression was determined in R using Benjamini-Hochberg corrected comparisons between MEC^{WT} and MEC^{Lef-1KI} sample groups. This gene set was used for all subsequent analysis. Principle components analysis was performed using the prcomp function in R (version 0.99.903). Pathway analysis was performed by Ingenuity Pathway Analysis (QIAGEN Bioinformatics).

### RNA Microarray Analysis on FACS Isolated Surface Airway Epithelial Cells

Freshly harvested SAE cells from mouse trachea were FACS sorted into basal, club, and ciliated populations and collected directly into TRIzol (Invitrogen). Total RNA was extracted following a TRIzol RNA isolation protocol and treated with DNase before being assayed in experimental duplicate on a GeneChip Mouse Gene 1.0 ST Array (Affymetrix). Raw array CEL files were pre-processed using the mogenel Ostprobeset.db function within the biocLite package in R and normalized using the Robust Multi-array Average (RMA) algorithm. Multiple probe values for the same gene were aggregated by max probe value. To obtain the gene list corresponding to genes which captured a large amount of the variance in the dataset, we first performed principal components analysis via the prcomp funciton in R (version 0.99.903). We then found the gene subset which correlated (Pearson 10.9) with either the first or second principal component vectors. Cell type specific genesets were determined through k-means++ via the kmeanspp function in the LICORS package with R, with k=4. A heatmap display of these genes and groups (FIG. 12C) was created using the heatmap.2 function in the gplots library within R.

### Results

αSMA⁺ Epithelial Cells Emerge on the Airway Surface after Severe but not Moderate Injury.

It was hypothesized that the extent of injury to the SAE is a determinant of whether reserve progenitors residing deep within the SMGs are mobilized for airway repair. To test this, the proliferative responses of cells in the SAE and SMGs following moderate (200 mg/kg naphthalene) and severe (300 mg/kg naphthalene) epithelial injury were evaluated. As hypothesized, severe injury increased EdU incorporation (2 hr pulse) within the SMG epithelium to a significantly greater extent than moderate injury (8.6-fold, P<0.0001, N=3-6 mice) at 3 days post-injury (DPI). Interestingly, αSMA⁺ epithelial cells emerged on the SAE only following severe injury, peaking at 3 DPI (FIG. 8A-D) and coinciding with a 2.7-fold increase in the number of αSMA⁺ MECs in the SMGs as compared to moderate injury (FIG. 8E). Given that the only αSMA-expressing epithelial cells in this region of the trachea are glandular MECs, these findings suggested that glandular MECs might transiently expand following severe injury and migrate to the SAE to facilitate repair.

Glandular MECs have the Capacity to Repair the Tracheal SAE Following Severe Naphthalene Injury.

To determine if glandular MECs contribute to repair of the tracheal SAE following severe injury, the suitability of two Cre drivers for lineage-tracing, which were predicted to mark MECs based on expression of αSMA/ACTA2 (alpha smooth muscle actin-2) or SMMHC/MYH11 (smooth muscle myosin heavy chain or myosin heavy chain 11) were evaluated. These Cre drivers were crossed to a ROSA^{LoxP}tdTomato^{StopLox}EGFP Cre reporter (ROSA-TG), to obtain ACTA2-Cre^{ERT2}: ROSA-TG and MYH11-Cre^{ERT2}:ROSA-TG mice. Tamoxifen induction with either ACTA2-Cre^{ERT2} or MYH/1-Cre^{ERT2} resulted in a MEC labeling efficiency of 77% and 85%, respectively (FIG. 8F-J). Thus, both Cre drivers appeared suitable for lineage tracing MECs following injury.

To test the hypothesis that MECs contribute to airway repair following severe injury, lineage-traced ACTA2-Cre^{ERT2}:ROSA-TG mice were injured with vehicle or high dose naphthalene (300 mg/kg) and examined the distribution of lineage-traced cells at 14 or 21 days post-injury (DPI) (FIG. 1A). At 14 DPI, lineage-traced (GFP⁺) cells emerged on the SAE and assumed a basal cell-like morphology (FIG. 1B-C). Interestingly, lineage-traced cells in the proximal tracheal SAE retained αSMA expression, whereas more distal GFP⁺ cells lacked αSMA expression (FIG. 1B). Lineage-traced cells on the SAE adopted a basal cell phenotype, expressing cytokeratin 5 (Krt5) (FIG. 1C), Krt14 (FIG. 1H), and neural growth factor receptor (NGFR) (FIG. 1E,F). Notably, both NGFR⁺ and NGFR⁻ lineage-traced basal-like cells on the SAE were observed (FIG. 1E,F), suggesting that MECs adopt an NGFR⁺ phenotype on the airway surface. Lineage-traced cells expressing tumor-associated calcium signal transducer 2 (Trop2), which specifically marks SAE and SMG duct progenitors (Hegab et al., 2011), were also observed in gland ducts (FIG. 1D). By 21 DPI, infrequent lineage-traced Krt8⁺ columnar cells appeared in the SAE (FIG. 1G), an indication of basal cell differentiation. In the absence of injury, lineage-traced SAE cells were not observed in the SAE (FIG. 11).

These findings suggest that MEC-derived progenitors on the SAE progressively extinguish αSMA expression while adopting a basal cell phenotype (Krt5⁺Krt14⁺NGFR⁺) with the ability to differentiate into Krt8⁺ luminal columnar cells. Thus the percentage and phenotype of lineage-traced cells along the proximodistal axis of the tracheal SAE at 21 DPI (FIG. 1J-L) were quantified. Lineage-traced cells accounted for 14% of the SAE between the cricoid cartilage (C0) and cartilage ring 4 (C4) (FIG. 1J), with the highest percentage in the C0-C1 region and 2-3 fold lower levels further from the glands at C2-C4 (FIG. 1K). This later finding supports the notion that MEC-derived progenitors emerge from the most proximal and largest glands in the C0-C1 region and migrate distally. Interestingly, the percentage of GFP⁺αSMA⁺ lineage-traced cells declined along the C0-C4 proximodistal axis of the trachea, while the percentage of GFP⁺Krt8⁺ demonstrated the opposite trend (FIG. 1L). Despite the observed heterogeneity of NGFR expression in lineage-traced SAE basal-like cells, there was no proximodistal axis pattern of expression following quantification. Cumulatively, these findings demonstrate several important features regarding MEC-derived progenitors on the airway surface: 1) MEC contribution to the SAE is maximal above the most proximal tracheal gland; 2) MEC-derived in the SAE adopt a basal cell phenotype as they move distally down the trachea by extinguishing αSMA expression and increasing their ability to differentiate into Krt8⁺ columnar cells; and 3) the vast majority of lineage-traced MECs remain in an undifferentiated basal-like state at 21 DPI.

The use of lineage-restricted Cre-drivers for fate mapping comes with caveats that include both the specificity of the promoters used and lineage tracing efficiency (Kretzschmar and Watt, 2012; Rios et al., 2016). Thus, to validate these findings, MYH/1-Cre^{ERT2} lineage tracing, which also efficiently marks MECs in adult SMGs (FIG. 8G-J), was used. Similarly to ACTA2-Cre^{ERT2}:ROSA-TG mice, naphthalene injury of MYH11-Cre^{ERT2}ROSA-TG mice also led to the appearance of Krt5 and Krt14 lineage-traced basal cells in the SAE (FIG. 9A-C,F). Furthermore, extending the chase period following tamoxifen induction from 5 to 21 days did not alter MEC contribution to SAE repair following naphthalene injury of ACTA2-Cre^{ERT2}:ROSA-TG mice (data not shown).

Glandular MEC-Derived Basal Cells in the SAE have the Capacity to Differentiate into Ciliated Cells.

Next the potential of MEC-derived progenitors to expand over time in the SAE and to generate ciliated cells following severe naphthalene injury of ACTA2-Cre^{ERT2}:ROSA-TG mice (FIG. 2A) was examined. The percentage of lineage-traced cells in the SAE rose 4.5-fold between 7 and 60 DPI (FIG. 2J), suggesting that MEC progenitors expand within the SAE overtime. Similarly, lineage-traced acetylated αtubulin⁺ ciliated cells also increased 4-fold during this time frame (FIG. 2C-I,K), confirming that MEC-derived basal cells take time to mature and differentiate. Naphthalene injury of MYH11-Cre^{ERT2}:ROSA-TG mice also produced infrequent ciliated cells at 21 DPI (FIG. 9E). In the absence of injury, lineage-traced MECs failed to migrate to the SAE even after a 1.5 year chase (FIG. 2L), demonstrating that MEC-derived progenitors do not participate in maintaining homeostatic turnover of the SAE. Taken together, these data demonstrate that MEC-derived progenitors can contribute to repair of the SAE following severe injury by adopting a basal cell phenotype and differentiating with time.

### MEC-Derived Basal Cells Establish Long Lasting Residence in the SAE Capable of Further Expansion Following Reinjury.

These findings suggest that MEC-derived progenitors on the SAE remain relatively undifferentiated at 21 DPI, but with increasing time after injury can differentiate into ciliated cells. However, it remained unclear if lineage-traced basal cells in the SAE indeed reestablished multipotent SC niches capable of responding to a second injury. To address this question, repeated epithelial injury was performed on tamoxifen-induced ACTA2-Cre^{ERT2}:ROSA-TG mice using first, a severe injury that largely ablates SAE basal cells, followed by a moderate injury that applies regenerative pressure to primarily SAE basal cells (FIG. 3A). Following this sequential injury, large lineage-traced clone-like patches on the airway surface contained multiple cell types typical of the SAE (FIG. 3B), including αtubulin⁺ ciliated cells, non-ciliated columnar cells (FIG. 3C), and goblet cells marked by Ulex europaeus agglutinin I (UEA-1) [lectin with specific affinity for Muc5AC (Pardo-Saganta et al., 2013)] (FIG. 3H), and Muc5B (FIG. 3I). Notably, while lineage-traced Scgb1a1⁺ club cells were rarely observed, a second injury led to the appearance of Scgb3a2⁺ lineage-traced club cells (FIG. 3D,K).

The abundance of lineage-traced cells in the SAE of ACTA2-Cre^{ERT2}:ROSA-TG mice was quantified after a single injury (SI) or double injury (DI) protocol as compared to uninduced (UNIND) and uninjured (UI) control mice. At 60 DPI, there was a 1.6-fold increase in the percentage of lineage-traced cells following double injury as compared to a single injury (FIG. 3L). This demonstrates that MEC-derived progenitors in the SAE are capable of further expansion following reinjury. By quantifying the distribution of ciliated, club, and goblet cells in the native (untraced/Tomato⁺) and MEC-derived (lineage-traced/GFP⁺) SAE of the same samples, it was asked whether MEC-derived progenitors adopt a similar multipotency as resident SAE progenitors (FIG. 3N). Following a single injury, MEC-derived progenitors retained a bias toward differentiating into Muc5B⁺ goblet cells and ciliated cells, while in the native untraced epithelium Scgb3a2⁺ and Scgb1a1⁺ club cells and ciliated cells were the predominant secretory cell types (FIG. 3N). Interestingly, a second injury partially reversed this secretory cell bias leading to fewer lineage-traced Muc5B⁺ goblet cells (2.6-fold) and greater numbers of Scgb3a2⁺ club cells (10-fold). Taken together, these findings suggest that the differentiation of potential MEC-derived basal cells is not equivalent that of SAE basal cells. However, with time and pressure to expand, MEC-derived basal cells appear to converge on a more native basal cell phenotype.

Glandular MECs have the Capacity to Differentiate into Other Glandular Cell Types Following Airway Injury.

ACTA2-Cre^{ERT2}:ROSA-TG labeled MECs were also capable of generating mucus secreting glandular tubules marked by UEA-1 (FIG. 3G) and Muc5B (FIG. 3I,J), as well as serous cells marked by lysozyme (FIG. 3F) and DBA (FIG. 3E). Following a single injury, lineage-traced cells in the SMGs doubled (FIG. 3M). As expected, the percentage of lineage-traced cells in the SMGs did not increase following a second mild injury, since this level of injury does not lead to MEC expansion. However, a second injury led to a decline in lineage-traced Trop2⁺ duct cells (˜3-fold) and a rise in Muc5B⁺ (˜3-fold) and UEA-1⁺ (˜10-fold) glandular cells (FIG. 3O). The decline in lineage-traced Trop2⁺ duct cells is consistent with gland ducts serving as a reservoir for SAE basal cells following moderate injury without selective pressure for repopulating this niche from glandular MECs. MEC lineage contribution to SMG tubules and ducts was also observed in MYH11-Cre^{ERT2}:ROSA-TG mice following a single severe naphthalene injury (FIG. 9B,D). These data demonstrate that MECs also can differentiate into other glandular cell types.

### Glandular MEC Progenitors Participate in Airway Repair Following SO₂ Injury.

In multiple organs, the type of insult and extent of injury can influence the type of stem cells that participate in epithelial regeneration (Hogan et al., 2014; Tata et al., 2013). This feature of stem cell plasticity is vital to the homeostatic maintenance and repair of organs in the face of diverse environmental insults. To determine if MEC-mediated repair of tracheal SAE and SMGs was specific to naphthalene injury, lineage-tracing experiments were performed in ACTA2-Cre^{ERT2}:ROSA-TG mice injured with SO₂ three weeks following tamoxifen induction (FIG. 10A). SO₂ injury has been used to rapidly ablate luminal cells in the trachea leading to a basal cell regenerative response (Tadokoro et al., 2016). Similar to naphthalene injury, mice exposed to SO₂ rapidly mobilized lineage-traced MECs to the SAE where they adopted a basal cell phenotype (FIG. 10B-G,K). Furthermore, lineage-traced glandular tubules emerged with time post-SO₂ injury (FIG. 10H-J,L). MEC-derived cells in the SAE extinguished αSMA expression with time post-injury as Krt5⁺ and Krt14⁺ basal cells expanded and differentiated into Krt8⁺ luminal cells (FIG. 10M). These studies demonstrate that MECs can also function as progenitors of SAE basal cells following SO₂ airway injury.

### The Wnt-Regulated Program of Primordial Glandular Stem Cells is Adopted by MECs Following Airway Injury.

Developmental programs that regulate stem cells during organ morphogenesis are often repurposed for regulating stem cell regenerative responses in adult tissues (Clevers et al., 2014; Lynch and Engelhardt, 2014; Tata and Rajagopal, 2017). During the earliest stages of SMG morphogenesis, Wnt-mediated transcriptional activation of lymphoid enhancer factor 1 (Lef-1) is required for primordial glandular stems cells (PGSCs) to initiate gland development from placodes in the SAE (Duan et al., 1999). Repression of SRY-Box 2 (Sox2) within PGSCs acts in concert with Wnt/β-Catenin signals to activate transcription at the Lef-1 promoter (Driskell et al., 2004; Filali et al., 2002; Liu et al., 2010; Lynch et al., 2016; Xie et al., 2014). In the absence of Lef-1, PGSCs fail to proliferate and gland development is aborted at an early stage of elongation (Driskell et al., 2007). Transcription Factor 7 (TCF7) is also activated within PGSCs in a similar fashion to Lef-1 (FIG. 4A,B). We hypothesized that these transcription factors may be similarly regulated during lineage commitment of adult MECs following airway injury.

To this end, the nuclear expression profiles of Lef-1, TCF7, Sox2, and pi-Catenin in SMGs were evaluated at 12 and 24 hrs following naphthalene injury (FIG. 4C-L). In the uninjured state, Sox2 was expressed in the majority of SMG cells and αSMA⁺ MECs, but Lef-1 and TCF7 expression was largely absent (FIG. 4C-E,K,L); nuclear G3-Catenin expression was largely confined to glandular cells that did not express the MEC marker αSMA (FIG. 4F,K,L). By contrast, airway injury with naphthalene induced nuclear Lef-1, TCF7, and 13-Catenin in a large proportion of MECs by 24 hrs, while Sox2 expression was largely extinguished in MECs and other glandular cell types (FIG. 4G-L). 5 DPI Lef-1 expression declined toward basal levels and Sox2 expression increased back to uninjured levels. Thus, injury induced changes in the expression of these transcription factors and nuclear β-Catenin within MECs appear conserved with the pattern seen in PGSCs during early stages of gland development.

Lef-1 Expression within Glandular MECs Activates Lineage Commitment and a Regenerative Response.

Given that Lef-1 is required for lineage commitment of PGSCs during gland development and is also activated in MECs shortly after airway injury, it was hypothesized that this transcription factor may also control lineage commitment of MECs following injury. To test this hypothesis, a ROSA^{LoxP}EGFP^{StopLoxP}Lef-1 (Lef-1KI) knock-in transgene capable of lineage-tracing cells that activate Lef-1 expression in response to Cre (FIG. 5A) was used. It was first asked if induction of Lef-1 expression in MECs over a 5 day time course leads to replication of MECs as detected by EdU incorporation (FIG. 5B). Surprisingly, tamoxifen induction of ACTA2-Cre^{ERT2}:Lef-1K1^{+/+} mice (homozygous for the Lef-1KI transgene) led to significant expansion of lineage-traced cells (GFP⁻) in the SMG and SAE, as compared to uninduced controls (FIG. 5C,E). Furthermore, lineage-traced regions of Lef-1K1^{+/+} SMGs contained more replicating MECs (i.e., EdU⁺) compared to untraced regions (FIG. 5E,F) and tamoxifen-induced uninjured ACTA2-Cre^{ERT2}:ROSA-TG mice (FIG. 5D,F). These findings support the hypothesis that Lef-1 induction in MECs controls lineage commitment to SAE and SMG cell types. However, the lack of EdU in the majority of Lef-1KI lineage-traced cells remained somewhat puzzling and suggested that the lineage commitment process may not always require replication of MECs.

To better understand the process by which Lef-1 activation in MECs controls regenerative expansion, uninjured and naphthalene injured ACTA2-Cre^{ERT2} lineage-traced mice heterozygous (Lef-1K1^{+/-}) and homozygous (Lef-1K1^{+/+}) for the Lef-1KI transgene (FIG. 5G-U) were evaluated. Importantly, without tamoxifen induction, injured Lef-1KI^{+/+} mice retained GFP expression in both SMGs and the SAE (FIG. 5H). Interestingly, while induced/uninjured Lef-1K1^{+/+} increased lineage-traced cells (i.e., GFP⁻) in both the SAE and SMGs, this did not occur in induced/uninjured Lef-1K1^{+/-} animals (FIG. 5I,K,T,U). Following naphthalene injury, both Lef-1K1^{+/-} and Lef-1K1^{+/+} animals demonstrated enhanced lineage contribution to the SAE (˜4-5 fold) (FIG. 5J,L,U) when compared to single injury ACTA2-Cre^{ERT2}:ROSA-TG animals lacking the Lef-1KI transgene (FIG. 1J). As anticipated, the extent of nuclear Lef-1 expression was similar to the extent of lineage-trace (i.e., GFP⁻) in various treatment groups and Lef-1KI genotypes (FIG. 5M-S), with Lef-1 expressing SAE cells also observed in the distal trachea of Lef-1K1^{+/+} animals (FIG. 5N,O insets). Interestingly, both lineage-traced (GFP⁻) and untraced (GFP⁺) cells expressed Sox2, and this was true in both the SAE and SMGs, suggesting that Lef-1 overexpression does not directly repress Sox2 expression. Similar findings of enhanced regeneration of the SAE and SMGs were also observed in MYH11-Cre^{ERT2}:Lef-1K1^{+/-} mice following naphthalene injury (FIG. 11). Thus, induction of Lef-1 in MECs enhances the regenerative properties of this progenitor cell following airway injury and a high level of Lef-1 expression (i.e., Lef-1K1^{+/+}) is sufficient to drive lineage commitment of MECs in the absence of injury.

Next, it was evaluated whether induction of Lef-1 in MECs altered the ability of MEC-derived progenitors to differentiate into various cell types of the SAE and SMGs. Following naphthalene injury, ACTA2-Cre^{ERT2}:Lef-1K1^{+/-} MECs (here forward called MEC^{Lef-1KI}) were able to differentiate into SAE basal (Krt5⁺), club (Scgb1a1⁺), and ciliated (αtubulin⁺) cells, but UEA-1⁺ secretory cells were infrequently observed (FIG. 6A-F,K). Thus, the secretory cell bias for MEC^{WT} progenitors (i.e., goblet) differed from that of MEC^{Lef-1KI} progenitors (i.e., club). Similarly, MEC^{Lef-1KI}-derived cells in the SMGs differentiated into glandular duct (Trop2⁺), ciliated duct (αtubulin⁺), and serous (UEA-1⁺) cells following injury (FIG. 6G-J).

Sufficient Lef-1 Expression in MECs Induces a Self-Limiting Regenerative Response by Directional Commitment without Self-Renewal of its SC Phenotype.

During mouse postnatal tracheal development, Lef-1 is specifically expressed in highly proliferative glandular progenitor cells and is extinguished as glands mature. By analogy, it is interesting that biallelic induction of Lef-1KI in adult MECs gave rise to a surprisingly robust regenerative response in the absence of injury. However, this response was not accompanied by unlimited proliferative expansion, suggesting that Lef-1 functions may be limited to glandular SC niches. It was hypothesized that high levels of Lef-1 expression might induce lineage commitment of MECs in the absence of self-renewing its precursor SC state. To this end, SO₂-injured ACTA2-Cre^{ERT2}:Lef-1K1^{+/+} mice sequentially at 21 and 42 days post-tamoxifen induction (FIG. 6L) and it was asked whether untraced MECs (i.e., GFP⁺) repopulated the SMG and SAE following re-injury. Indeed, induced/uninjured Lef-1K1^{+/+} animals retained large numbers of lineage-traced cells (GFP⁻) in the SMG and SAE out to 62 days following tamoxifen induction (FIG. 6M), while untraced cells (GFP⁺) repopulated most of the SAE and SMGs in sequentially injured Lef-1K1^{+/+} mice (FIG. 6N-O). This finding suggests that untraced MECs, which fail to activate Lef-1, repopulate the SMGs and expand following re-injury. Thus, sufficient Lef-1 expression in multipotent MECS may activate a state of limited potential to self-renew in the SAE and SMGs.

Lef-1 Expression in MECs Activates Pathways Consistent with a Regenerative Response.

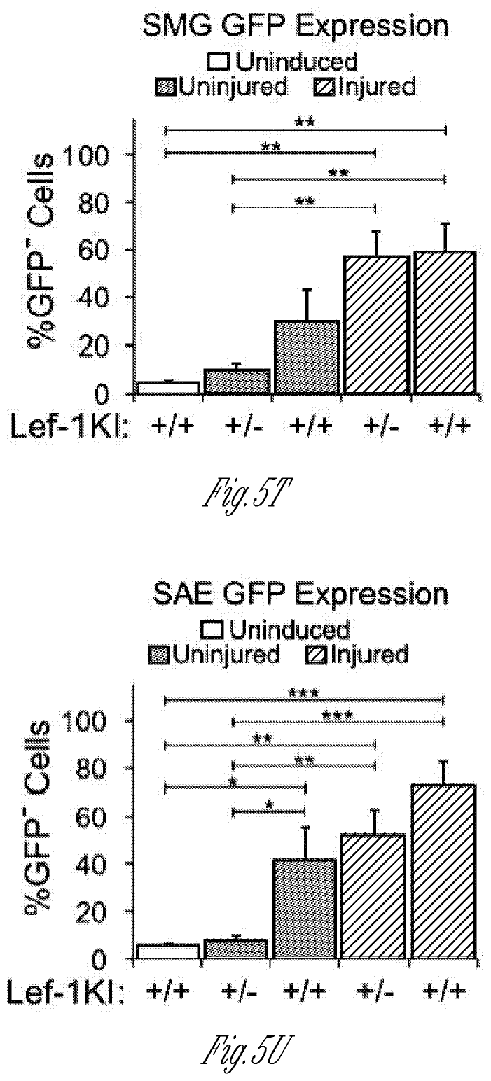

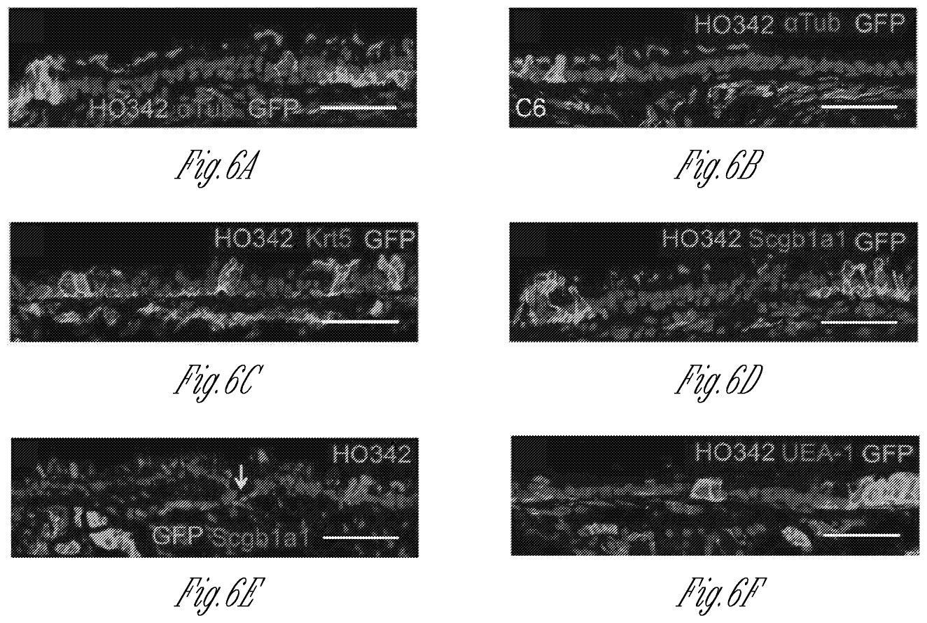

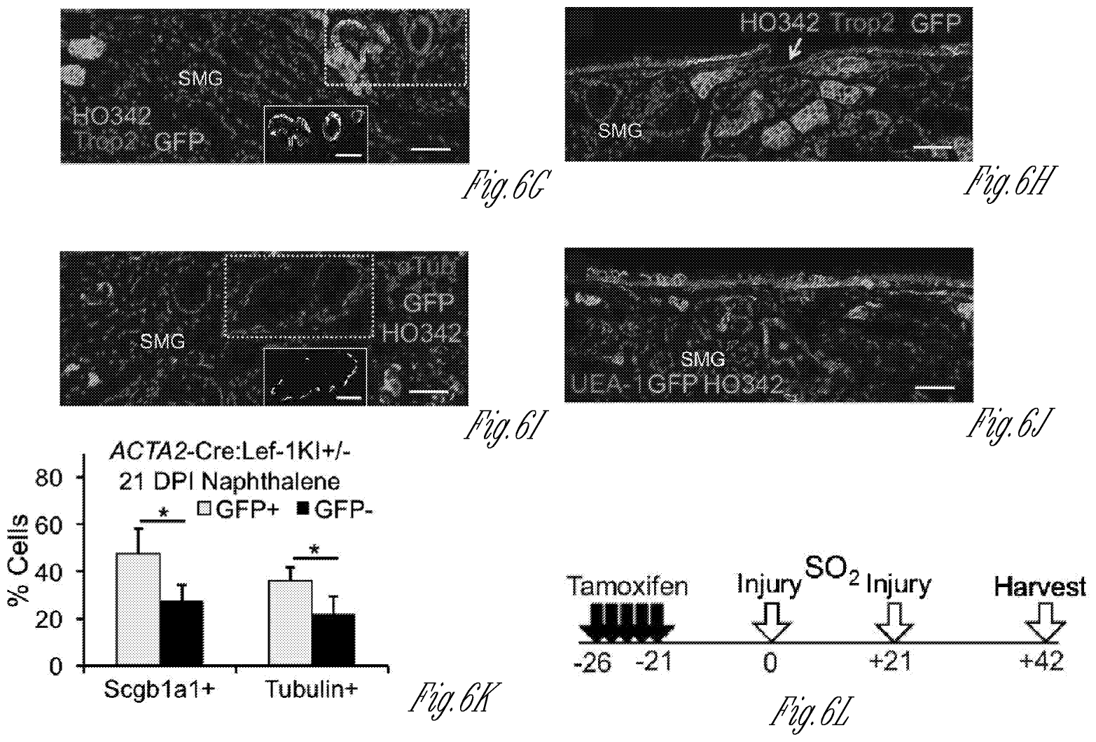

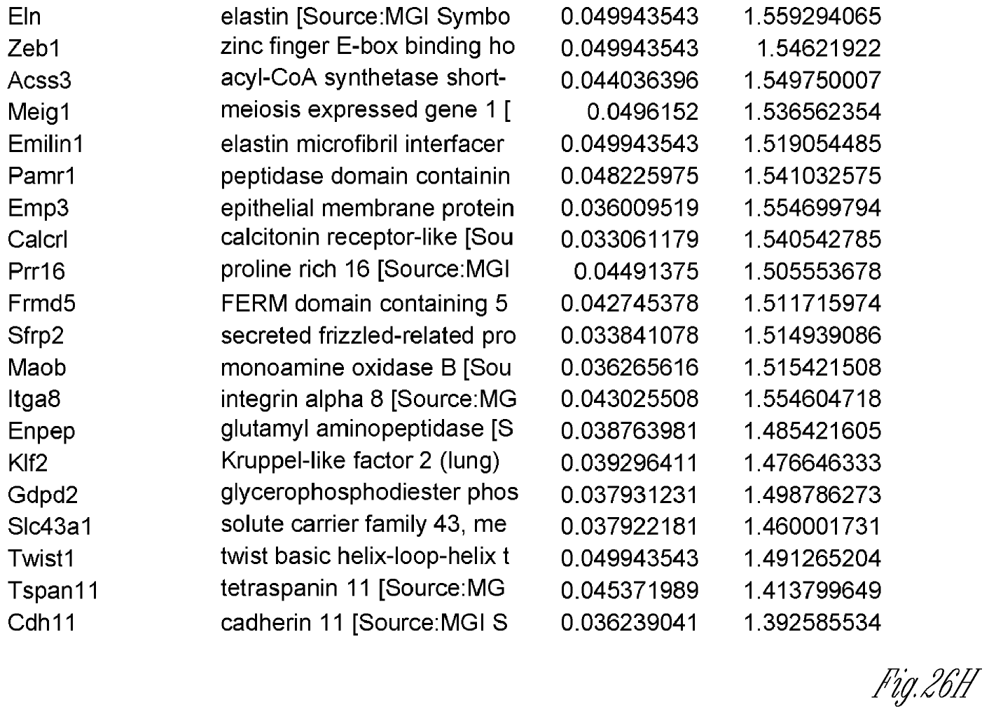

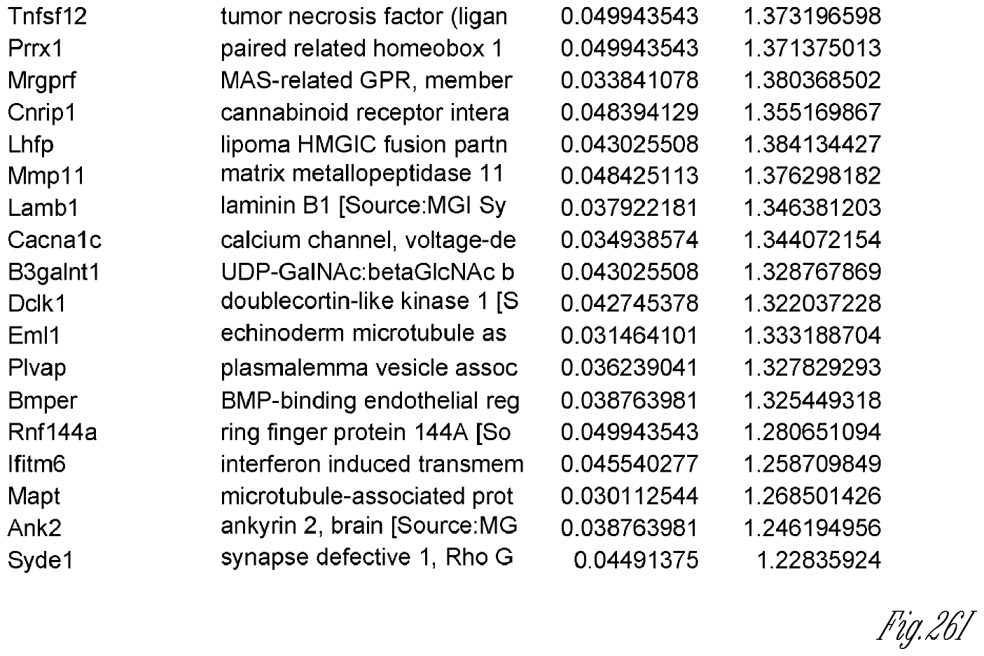

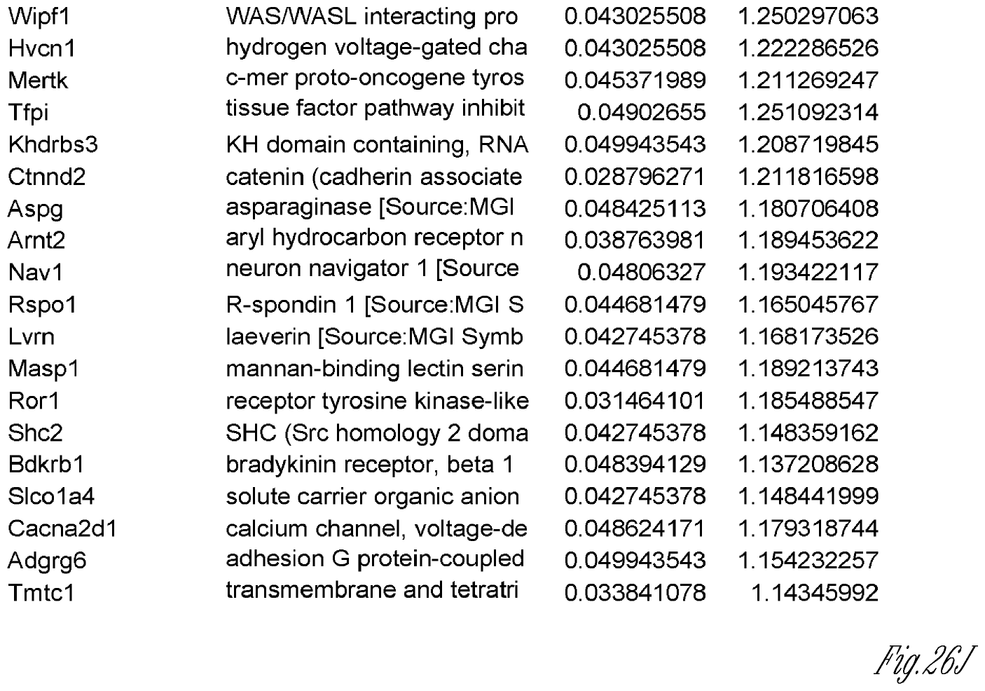

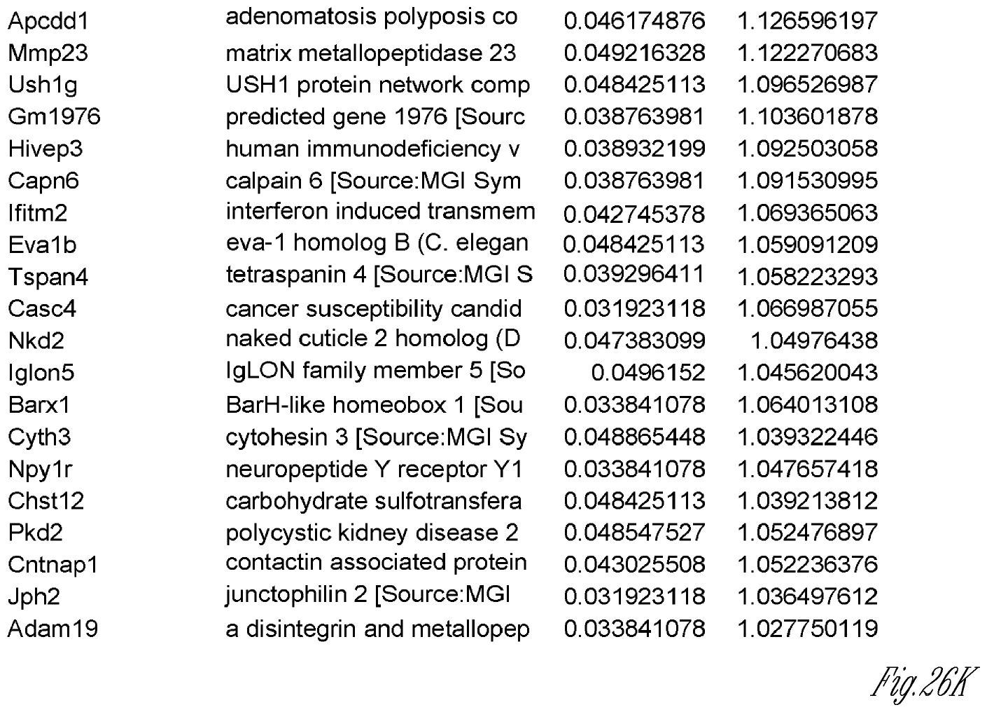

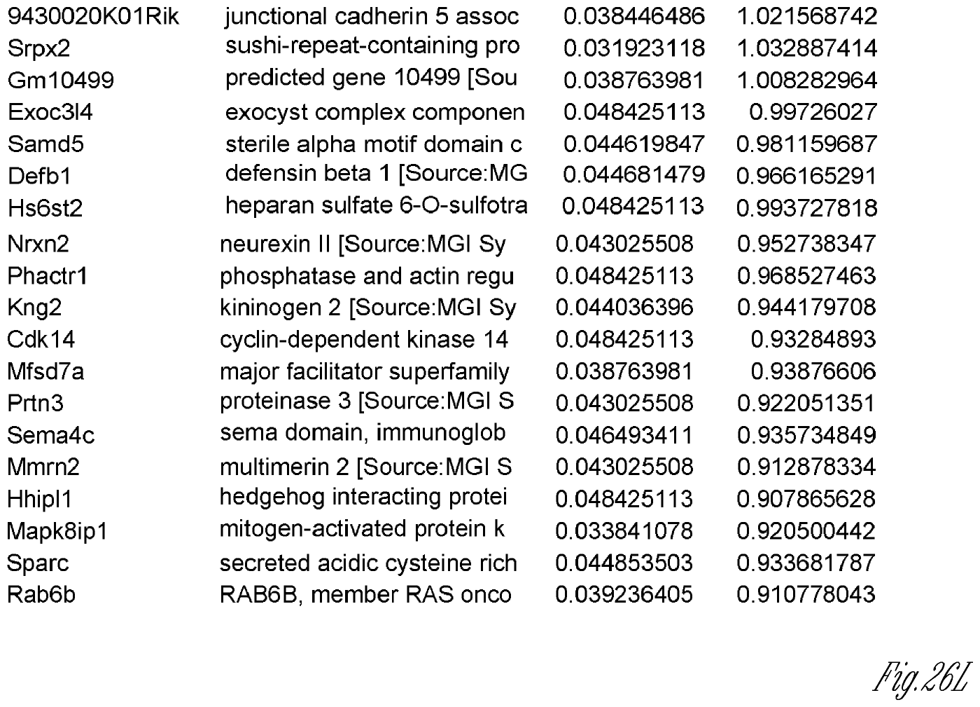

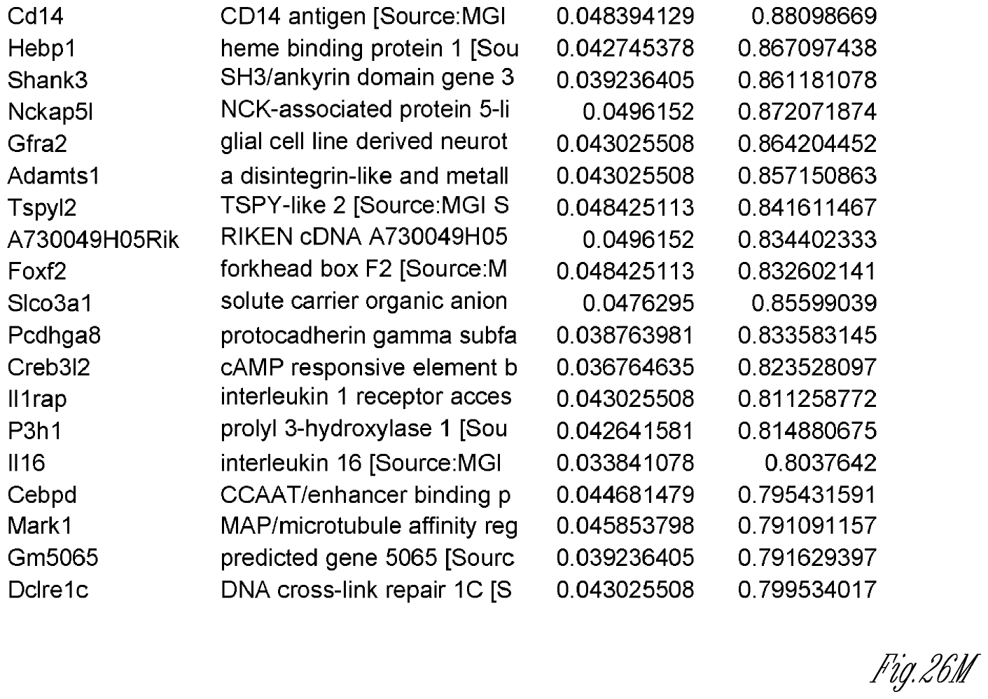

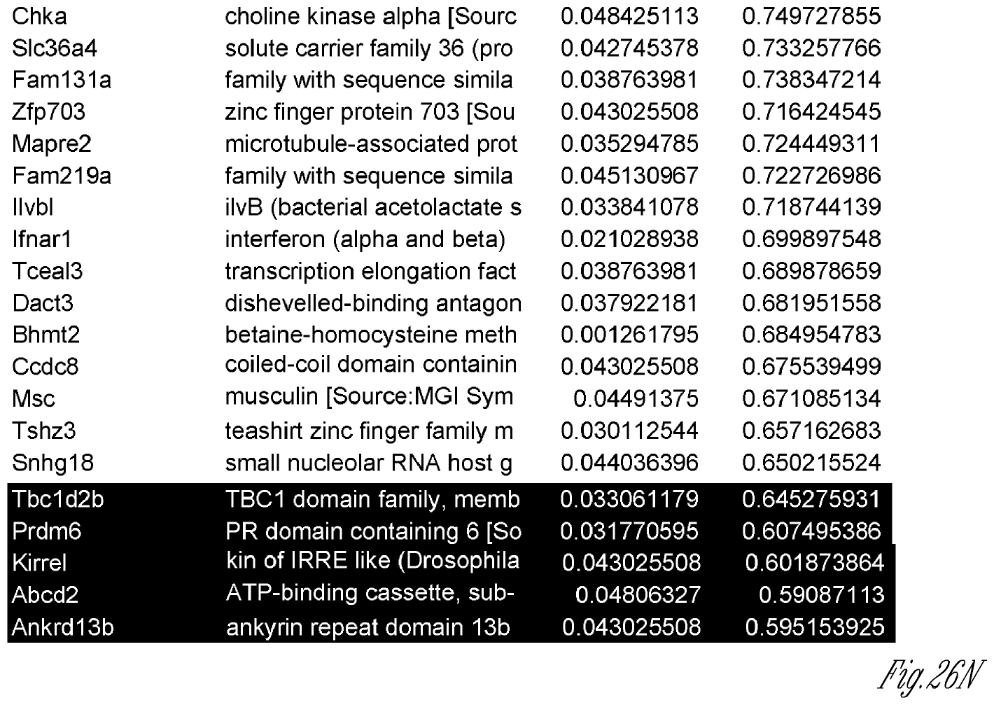

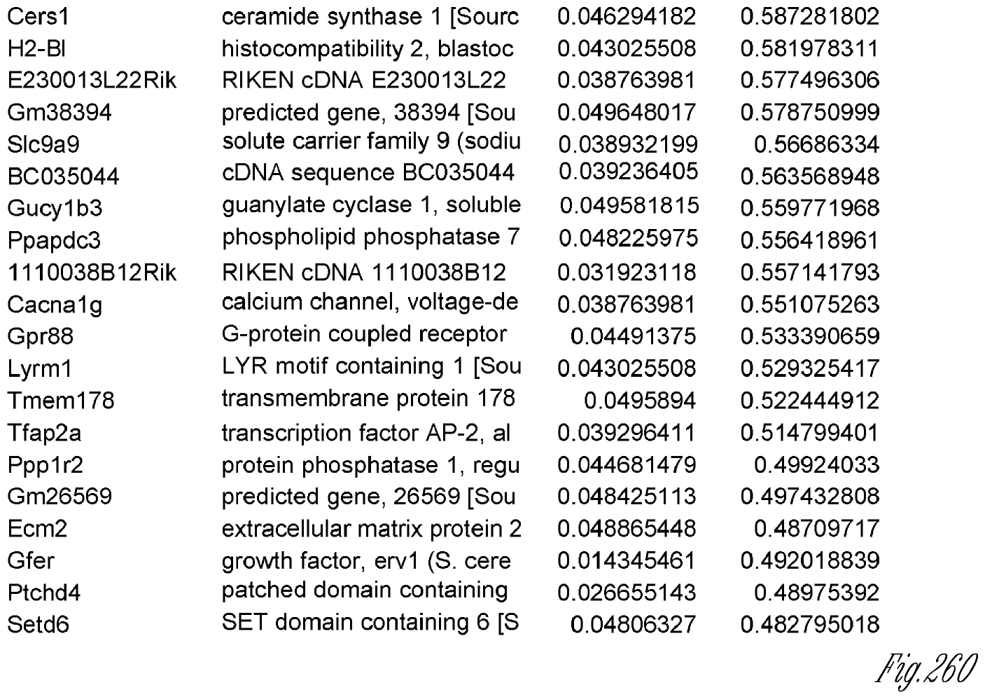

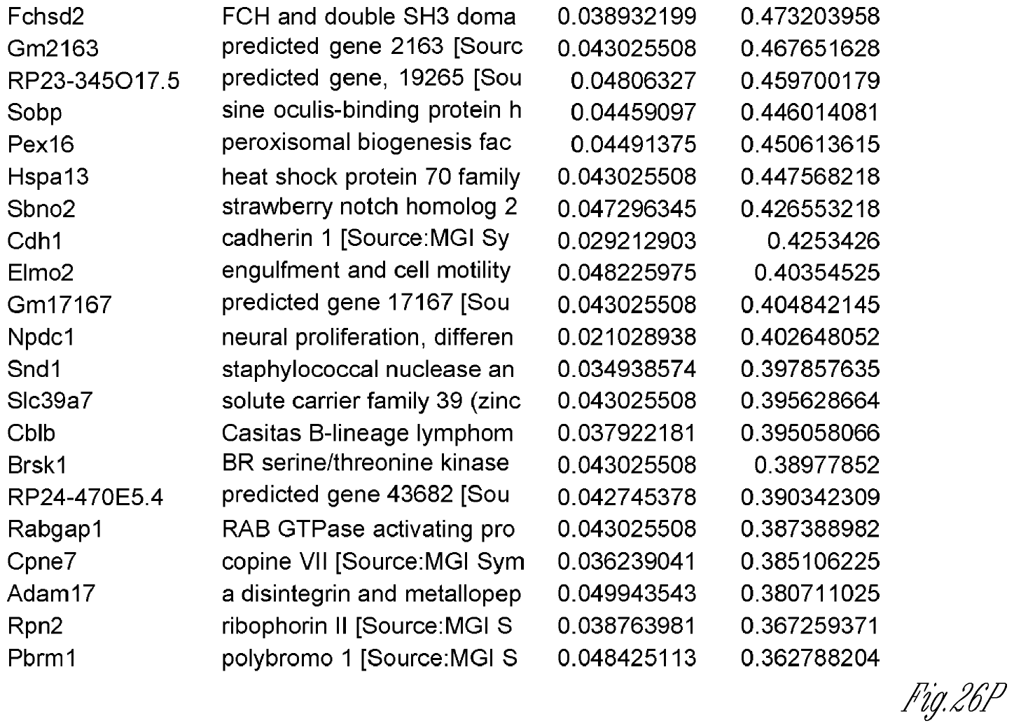

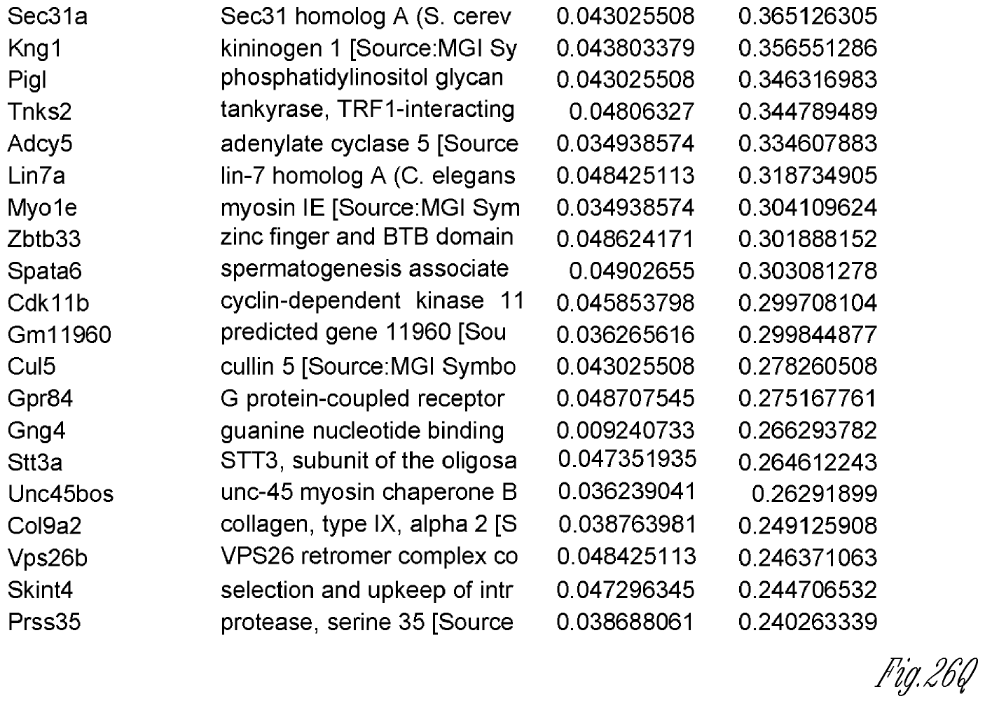

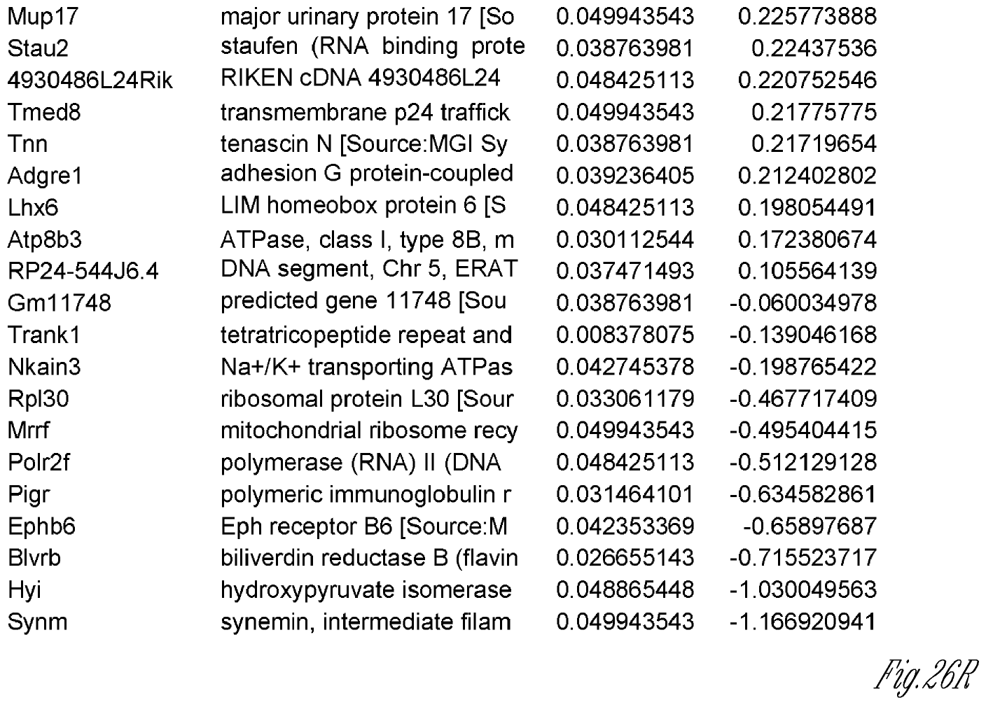

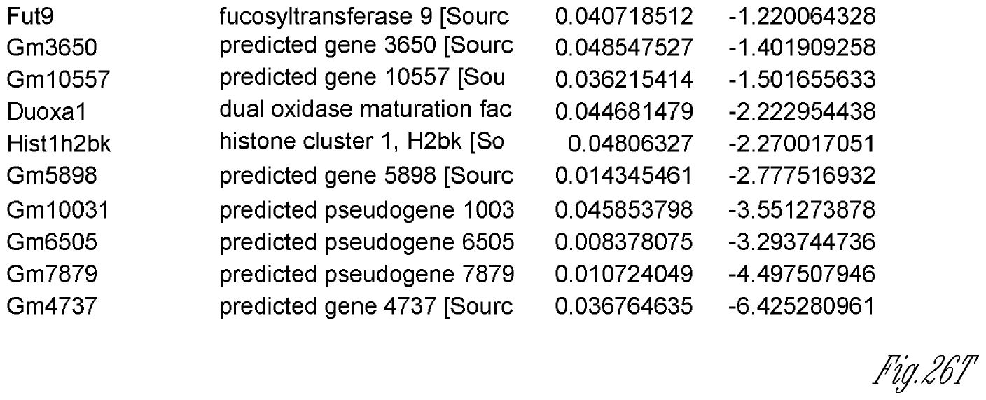

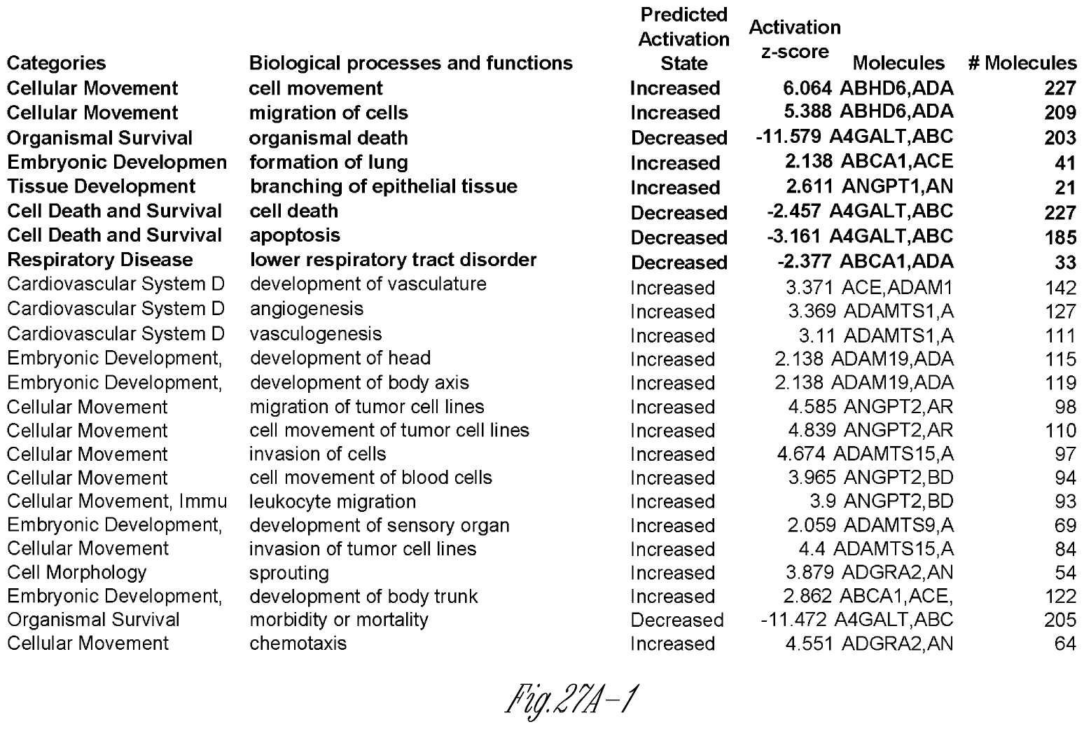

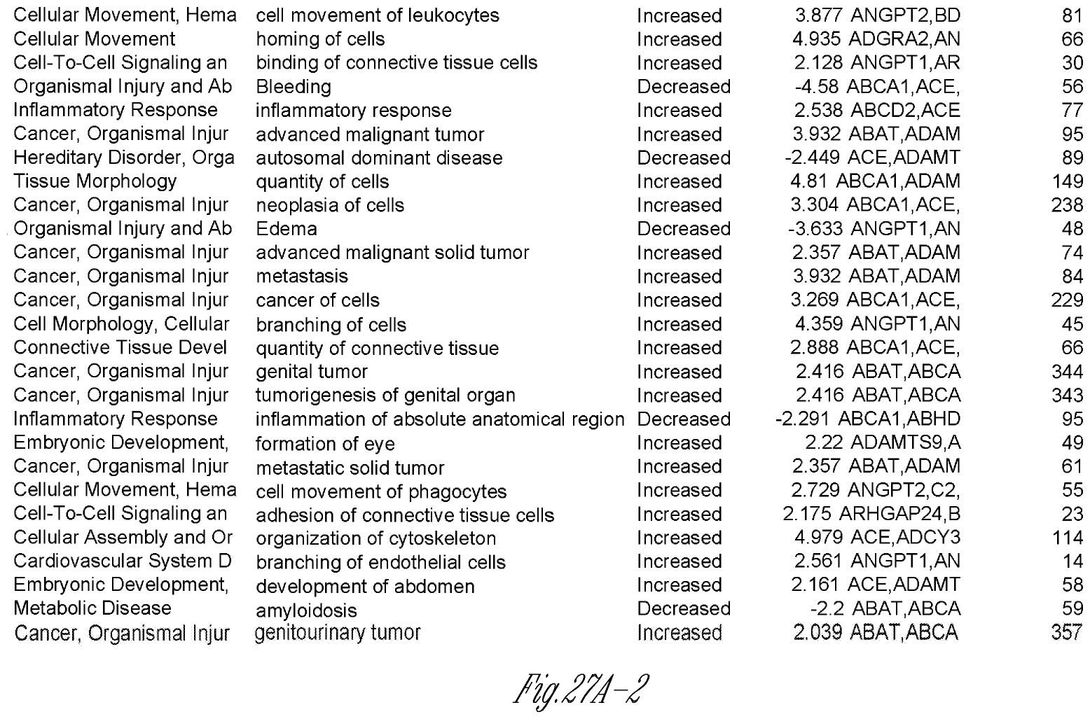

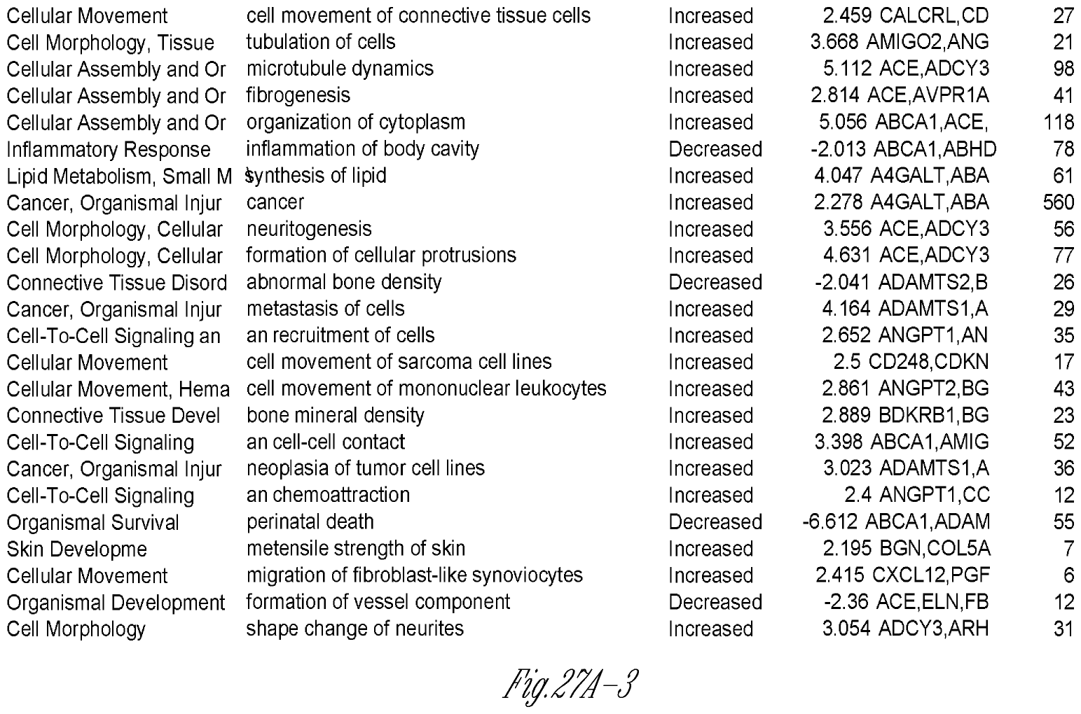

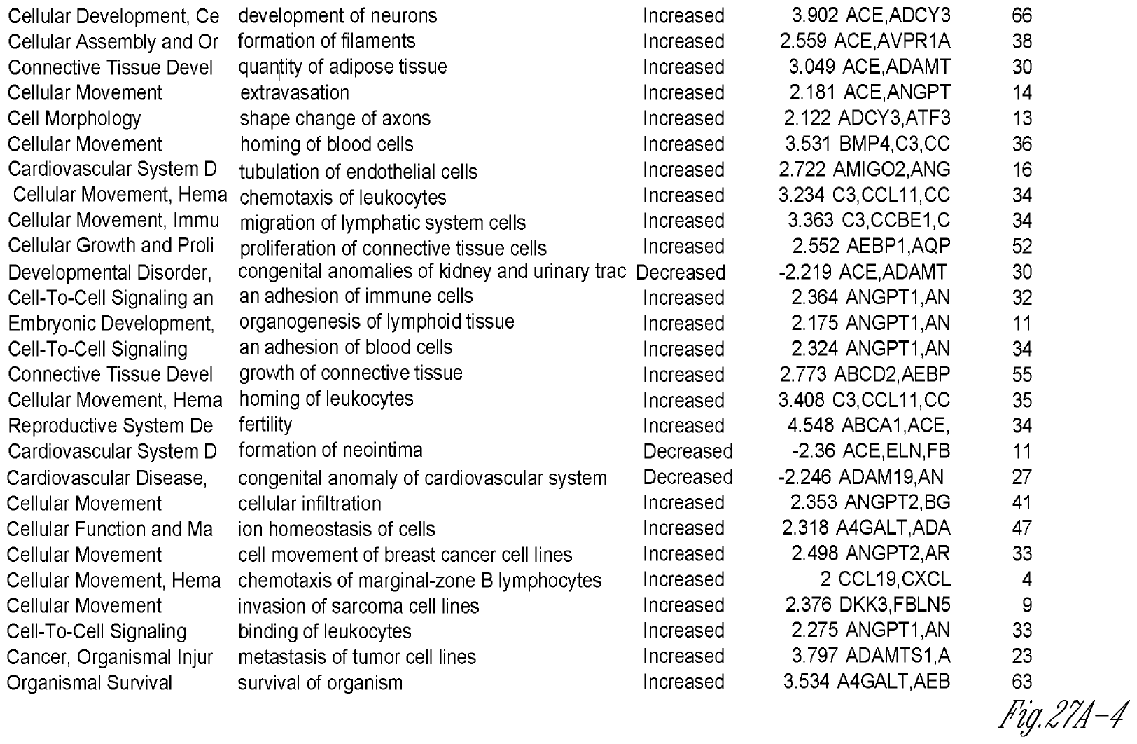

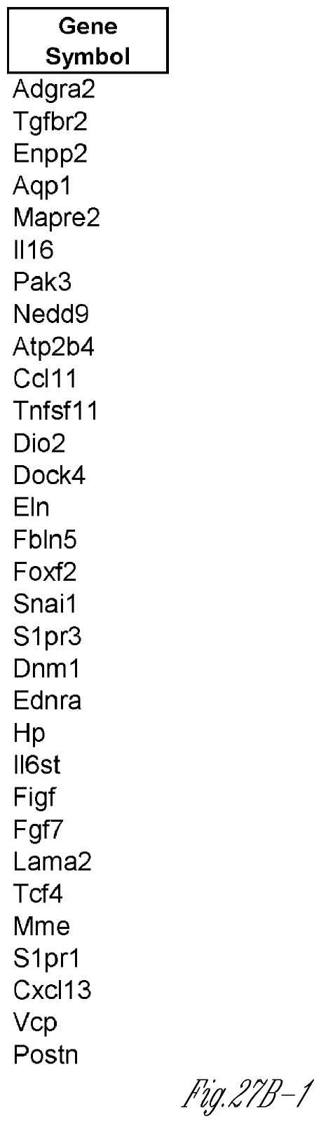

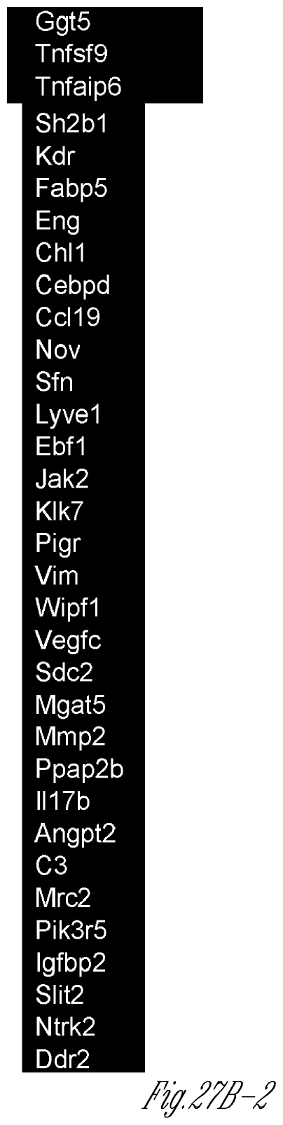

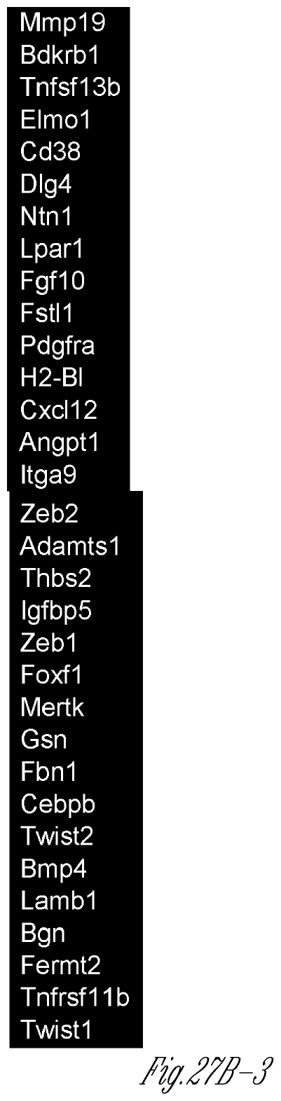

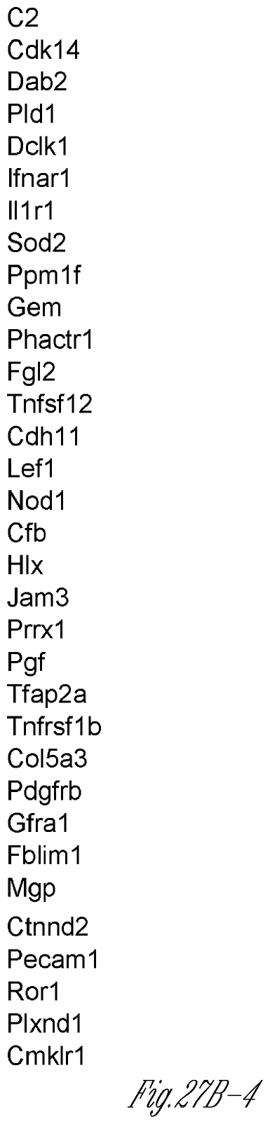

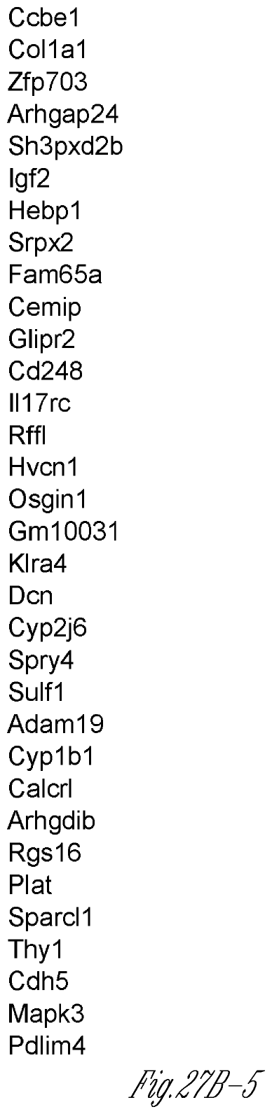

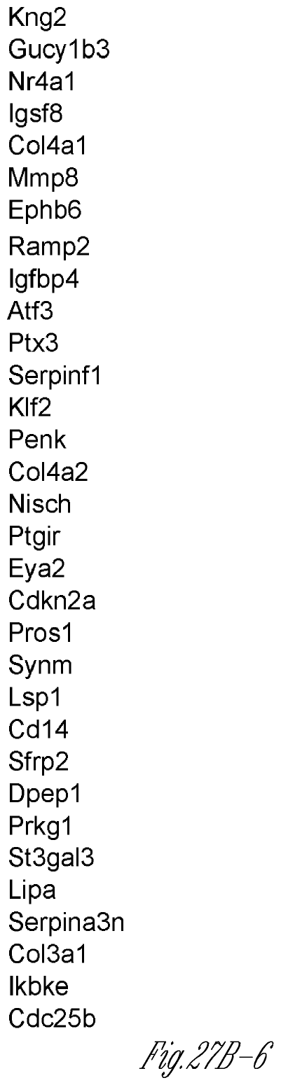

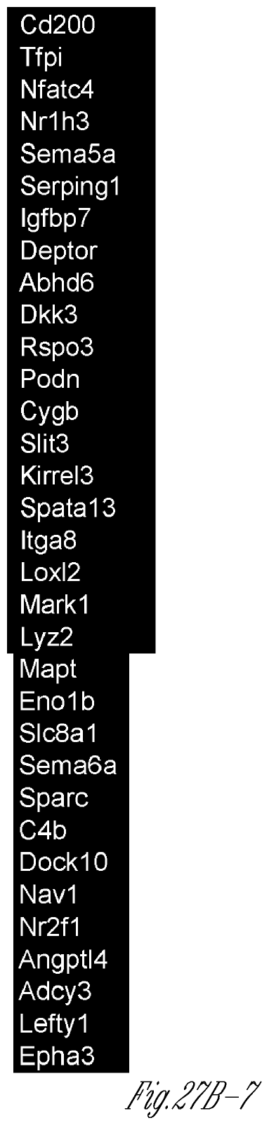

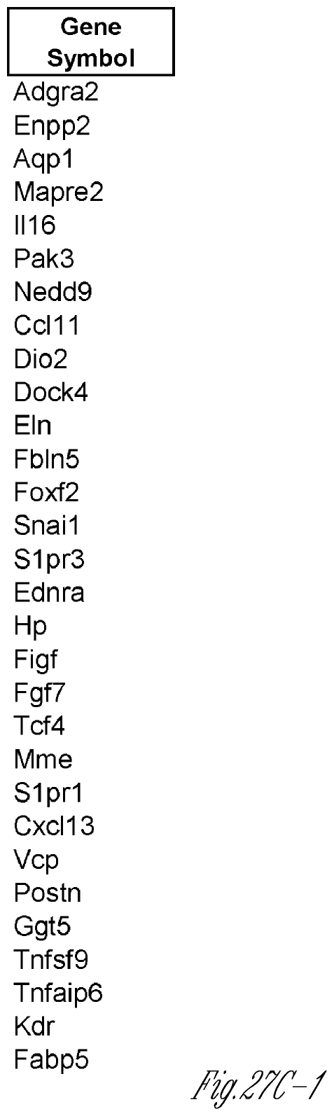

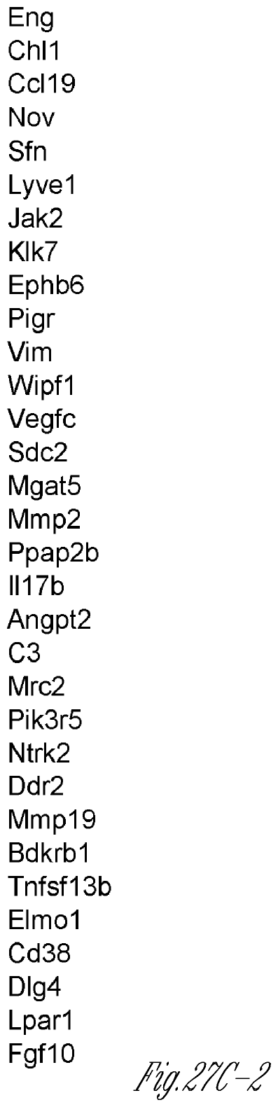

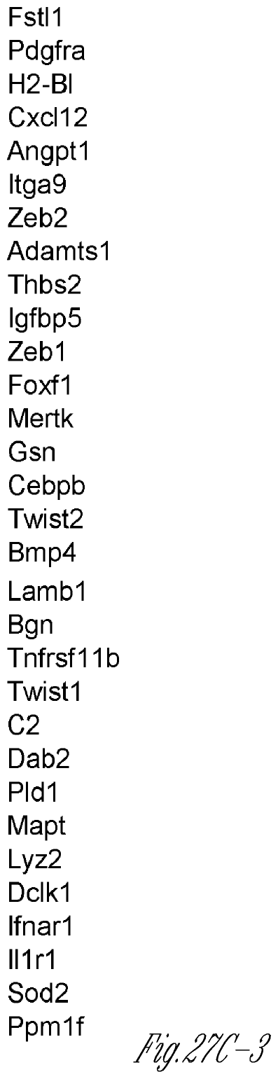

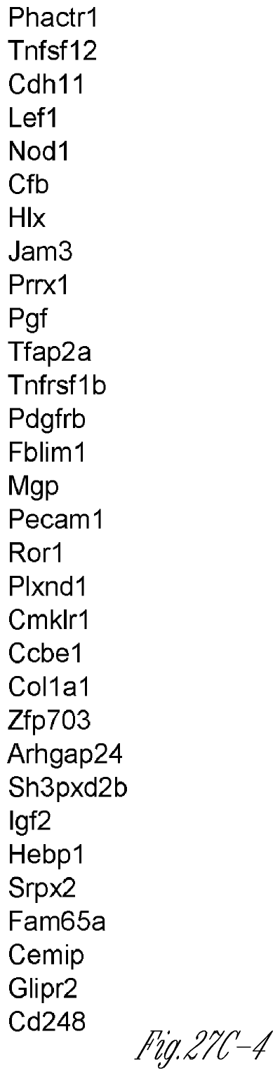

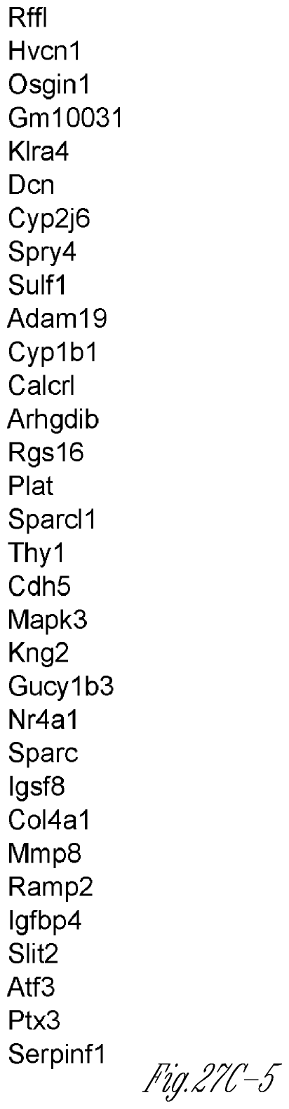

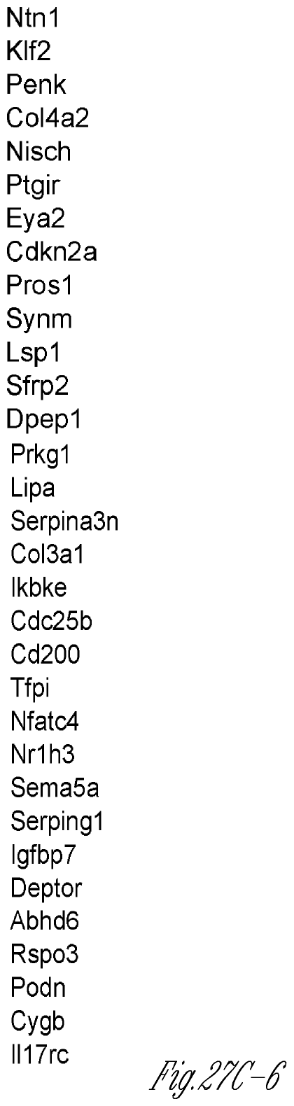

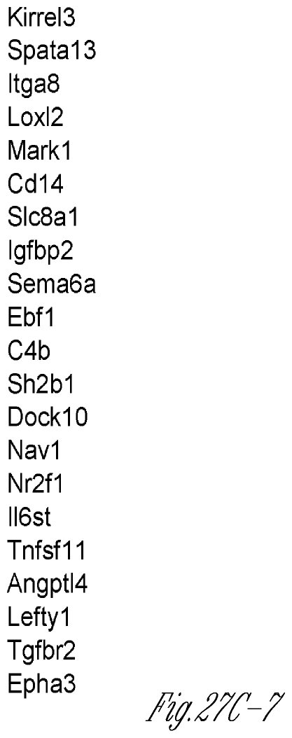

Wnt signals play important roles in regulating stem cells and their niches in many organs (Clevers et al., 2014). To evaluate how Lef-1, a component of canonical Wnt signaling, alters the phenotype and regenerative response of MECs, we performed RNAseq on passage-1 (P1) FACS isolated lineage-traced MECs harvested from SMGs of tamoxifen induced ACTA2-Cre^{ERT2}:Lef-1K1^{+/+} and ACTA2-Cre^{ERT2}:ROSA-TG mice. Of the 13,336 expressed genes identified, 699 genes had altered expression >2-fold between MEC^{Lef-1KI} and MEC^{WT} populations, the majority (537 genes) being induced in MEC^{Lef-1KI} (FIG. 26). There were 359 differentially expressed genes (Benjamini-Hochberg adjusted t-test; P<0.05) of which 94% were upregulated by Lef-1 (FIG. 7A). Lef-1 expression was induced 150-fold in MEC^{Lef-1KI} (FIG. 7B). Principle components analysis (PCA) of all 13,336 expressed genes demonstrated a clear separation of MEC^{WT} from MEC^{Lef-1KI} transcriptomes with the first two PCs accounting for 64.55% of the total variance (FIG. 7C). Ingenuity pathway analysis was used to discover biological pathways that were significantly differentially regulated in MEC^{WT} and MEC^{Lef-1KI} transcriptomes (FIG. 7D) and demonstrated positive z-scores for pathways involved in cell movement (FIG. 7E), migration of cells (FIG. 7F), formation of the lung (FIG. 7H), and branching of epithelial tissues (FIG. 7I) (FIG. 27). By contrast, significant negative z-scores included gene sets involved in organismal death (FIG. 7G), cell death, and apoptosis (FIG. 7D). These findings are consistent with Lef-1 activation of a transcriptional program that drives migration of MECs to the SAE and promotes a regenerative response. In this regard, 41 transcription factors were differentially regulated in MEC^{WT} and MEC^{Lef-1KI} populations (FIG. 7L). For example, MEC^{Lef-1KI} induced Tbx4 (3.3-fold), which has been implicated in regulating proliferation, migration, and invasion of lung myofibroblasts (Xie et al., 2016). Two other Lef-1-induced transcription factors, TWIST2 (3.9-fold) and Zeb1 (3.4-fold), regulate epithelial cell adhesion, motility and proliferation (Browne et al., 2010; Teng and Li, 2014; Vandewalle et al., 2009). Consistent with enhanced transcriptional pathways involved in migration, MEC^{Lef-1KI} cells in Wnt-stimulatory culture conditions demonstrated enhanced motility compared to MEC^{WT} cells in culture (FIG. 7J,K).

### Lef-1 Expression Facilitates Lineage Commitment of MECs Toward a SAE Basal Cell Phenotype.

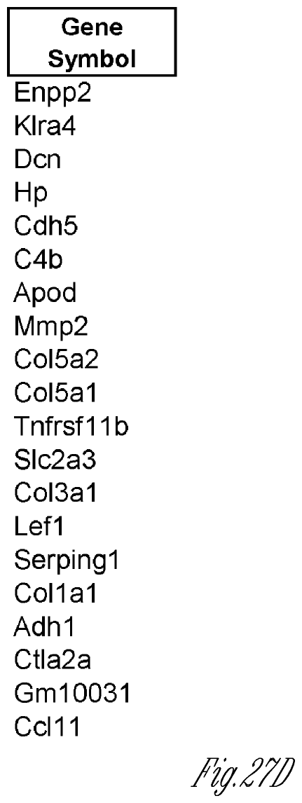

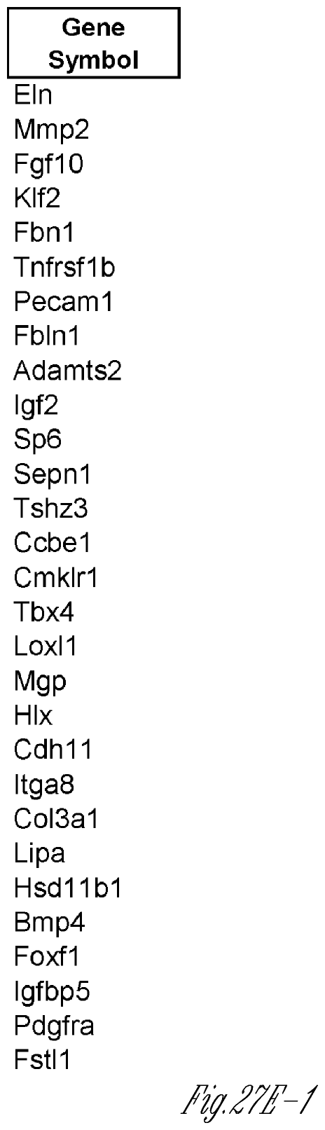

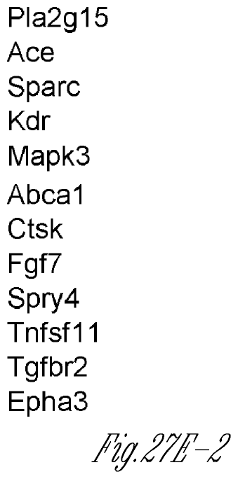

Given that Lef-1 expression in MECs led to more rapid regeneration of the SAE, it was hypothesized that Lef-1 may induce MEC lineage commitment toward a SAE basal cell phenotype. To address this hypothesis, we asked whether the transcnptome of primary cultures of MEC^{Lef-1KI} cells is more closely related to that of SAE basal cells than that of MEC^{WT} cells. To identify key genes that define SAE basal cells, basal, ciliated, and club cell populations were isolated (Zhao et al., 2014) and microarray analysis was performed on isolated mRNA (FIG. 12A). Principal component analysis (FIG. 12B) and hierarchical clustering (FIG. 12C) demonstrated robust differences in gene expression between basal, ciliated and club cell samples including enrichment of previously identified cell-type specific genes (e.g., basal cells: Cdh3, Ngfr, Trp63, Notch1, Krt14, Krt5; ciliated cells: Cfap46, Tuba1a, Foxj1, Rfx3; and club cells: Aldh1a7, Cyp7b1, Notch3, Scgb3a2) (FIG. 12D)(FIG. 28). Of the 1215 genes enriched in basal cells (z-score >1.5; FIG. 28), 50 genes were differentially regulated >2-fold between MEC^{WT} and MEC^{Lef-1KI} populations and 92% of these genes were upregulated in MEC^{Lef-1KI} (FIG. 7M). These findings demonstrate that Lef-1 induces a phenotypic shift in MECs toward SAE basal cells, supporting the notion that Lef-1 induction in MECs following airway injury controls lineage commitment and migration to the airway surface where MECs differentiate into basal cells.

### MECs are Highly Proliferative Self-Renewing Progenitors.

Important criteria for sternness include the ability to self-renew and maintain multipotency for differentiated cell types in a given biologic trophic unit (Lanza and Atala, 2014). The ability to demonstrate these criteria in vitro provides important support for stemness. To this end, the ability of SAE basal cells and MEC^{WT} populations were compared for their ability to self-renew in culture. Primary SAE and SMG cells were differentially isolated from tamoxifen-induced ACTA2-Cre^{ERT2}:ROSA-TG mice and expanded them in vitro (FIG. 13A-F). Lineage-traced MECs were not present among the isolated SAE cells (FIG. 13B,C), but were found in SMG epithelia (FIG. 13E,F). As expected from in vivo quantification, lineage-traced MECs (GFP⁺) represented a minority of glandular cells upon initial plating (˜15%), but with time expanded more extensively than did untraced glandular progenitors, stabilizing at ˜75% of cultures by P5-P7 (FIG. 13F). Assuming 23% of MECs are untraced (FIG. 8I,J), this stabilized ratio likely represents the outgrowth of traced and untraced MECs. However, an alternative explanation for the persistence of untraced cells could be the contamination of glandular preparations with SAE basal cells with an equal capacity for self-renewal.

To better distinguish between these two possibilities, mixing experiments were performed with FACS isolated P3 cultures of ACTA2-Cre^{ERT2}:ROSA-TG SMG progenitors (untraced/Tomato⁺ or lineage-traced/GFP⁺) and transgene-negative SAE progenitors mixed at a ratio of 10% SMG:90% SAE (FIG. 13G-L). In both conditions lineage-traced (GFP⁺) and untraced (Tomato⁺) gland-derived cells expanded to a greater extent than SAE-derived cells in mixed cultures (FIG. 13I,L). Thus, it is unlikely that contaminating SAE basal cells are the untraced lineage that persists in glandular culture. Given the enhanced regenerative capacity of MEC^{Lef-1KI}-derived progenitors in vivo, it was hypothesized that Lef-1 expression may impart a greater capacity to proliferate in vitro. To this end, we compared the ability of MEC^{WT} and MEC^{Lef-1KI} populations for their ability to self-renew in mixed cultures containing 10%-MEC^{Lef-1KI}: 90%-MEC^{WT}. Results from this analysis demonstrated that indeed MEC^{Lef-1KI} outcompeted MEC^{WT} progenitors in culture (FIG. 13M-O). Thus, Lef-1 expression in MECs either enhances the extent of self-renewal or reduces cell cycle time under culture conditions that promote Wnt signaling and inhibit SMAD signaling (Mou et al., 2016). Limitations to the above comparisons include the fact that the specific conditions of the culture system could impact growth and self-renewal of diverse progenitor populations differently.

### MECs are Multipotent Progenitors for SAE Cell Types In Vitro and Rapidly Regenerate a Differentiated Airway Epithelium in Denuded Tracheal Xenografts.

To compare the capacity of MEC^{WT} and MEC^{Let-1KI} progenitors to differentiate in vitro, we mixed P1 populations (50:50) and seeded them into air-liquid interface (ALI) cultures and denuded tracheal xenografts (FIG. 14A). Phenotypic analysis of ALI cultures demonstrated that MEC^{Lef-1KI} more effectively generated Scgb1a1 expressing club cells (FIG. 14B,F) than MEC^{WT}, supporting in vivo findings. While both MEC^{WT} and MEC^{Lef-1KI} progenitors differentiated into α-tubulin expressing ciliated cells, MEC^{Lef-1KI} did this to a greater extent (FIG. 14C,G). Relatively few Muc5AC expressing secretory cells were observed and only in the MEC^{Lef-1KI} populations (FIG. 14D,H), while Muc5B expressing secretory cells were observed equally in both populations (FIG. 14E,I).

Reconstituted denuded tracheal xenografts were utilized in athymic nude mice to interrogate the capacity of SAE and SMG progenitors to both proliferate and differentiate (Lynch et al., 2016), by seeding mixed population of primary cells isolated from non-transgenic tracheal SAE and ACTA2-Cre^{ERT2}:ROSA-TG SMGs containing lineage-traced and untraced cells at a ratio of 1:9 (SMG:SAE). Notably, both SMG lineage-traced (GFP⁺) and untraced (Tomato⁺) cells generated gland-like clones, whereas unmarked SAE cells rarely contributed to glands despite the seeding of 9-fold more SAE cells (FIG. 14J). Lineage-traced MEC-derived progenitors also generated SAE clones containing ciliated cells (FIG. 14K) and luminal mucin secreting cells (FIG. 14L). Thus, in xenografts MEC-derived progenitors are capable of differentiating into both SMG and SAE cell types. Furthermore, SMG-derived cells contributed to a larger portion of the xenograft epithelium than did SAE-derived transgene negative cells, supporting the finding that MECs have enhanced growth properties in vitro relative to SAE cells.

To directly compare the capacity of MEC^{WT} and MEC^{Lef-1KI} progenitors to regenerate a denuded epithelium, xenografts were seeded with a 50:50 mixture of FACS isolated, lineage-traced, populations. Findings from these studies were similar to the in vitro expansion assays. The majority of the xenograft epithelium was reconstituted by the GFP⁻ MEC^{Lef-1KI} population (FIG. 14M-O). While both MEC^{WT} and MEC^{Lef-1KI} progenitors formed lineage-mixed gland-like structures, a greater number were observed with the MEC^{Lef-1KI} phenotype (GFP⁻) (FIG. 14M,N). Furthermore, both MEC^{WT} and MEC^{Lef-1KI} progenitors had the ability to differentiate into ciliated cells (FIG. 14P,Q). These ex vivo findings confirm that Lef-1 expression in MECs enhances the regenerative capacity of this stem cell, as observed both in vivo and in vitro.

### DISCUSSION

SC niches coordinate tissue maintenance and repair in adult organs and these processes often require regenerative plasticity capable of adapting to the extent and type of injury (Hogan et al., 2014; Rajagopal and Stanger, 2016). For example, reversal in SC hierarchies can occur when professional SCs are depleted and a differentiated cell type reacquires properties of its parent SC (i.e., facultative SCs). Alternatively, when multiple types of SCs exist within an organ, selective environmental pressure can lead to expansion of one SC population over the other (Visvader and Clevers, 2016). In proximal airways, two anatomically distinct SC niches are thought to exist in SAE and SMGs. While basal cells have been formally defined as SCs of the SAE using in vivo lineage-tracing and in vitro criteria, the identity of SMG SCs has remained undefined. The present findings demonstrate that glandular MECs are precursors of multipotent SAE basal SCs and other glandular cell types following severe airway injury. Given the anatomical separation of these two SC compartments and distinct biologic functions of each epithelium, we conclude that glandular MECs are reserve multipotent SCs of the SAE and professional SCs of SMGs.

Glandular MECs only contributed to SAE repair following severe airway injury. The lack of MEC involvement in the homeostatic maintenance of the SAE over 1.5 yrs is consistent with MECs serving as reserve SCs for the SAE. Interestingly, lineage-traced MECs in the SAE progressively extinguished αSMA expression in a proximodistal pattern along the trachea as they as they adopted a basal cell phenotype in the SAE. This maturation process coincided with increased differentiation into luminal cells. While MEC-derived basal cells in the SAE were multipotent, forming ciliated, secretory, and non-ciliated columnar cells by 60 days following a single injury, their differentiation potential was not equivalent to that of native SAE basal cells. For example, MEC-derived Scg3a2⁺ club cells only emerged following a second mild injury, and these cells lacked Scg1a1 expression typical of native club cells. Thus, while MEC-derived progenitors can establish lasting residence in the SAE and expand following a second injury, they take considerable time to mature into professional basal cells.

Mammary gland MECs have been extensively studied by fate mapping and may be analogous to airway gland MECs. Both mammary MECs and luminal cells are long-lived lineage-restricted progenitors during development, puberty, and pregnancy; yet, isolated mammary MECs, but not isolated luminal cells, can form whole mammary glands in transplantation assays (Prater et al., 2014; Van Keymeulen et al., 2011). Similar to these studies, in the absence of severe injury adult glandular MECs also appear lineage-restricted, but only airway gland MECs, not SAE basal cells, generate both a well-differentiated surface epithelium and gland-like structures in xenograft transplantation assays. Moreover, during development a subset of mammary MECs have the capacity to differentiate into luminal cells (Rios et al., 2014). In this regard, we have similarly shown that early-born MECs are able to differentiate into multiple SMG cell types during tracheal development (Anderson et al., 2017). We now show that adult tracheal MECs are multipotent SCs for serous, mucous, and duct cells of SMGs following airway injury.

Wnt/β-catenin signaling is integral to many developmental programs involved in organogenesis and these pathways are often repurposed by SC niches to regulate regenerative responses in adult tissues (Clevers et al., 2014; Nusse and Clevers, 2017). In this regard, we find striking similarities in the dynamic expression of several Wnt-regulated transcription factors (Lef-1^{Hi}, TCF7^{Hi}, and Sox2^{Low}) during lineage commitment of primordial glandular SCs (PGSCs) (Lynch and Engelhardt, 2014) and adult glandular MECs following airway injury. Interestingly, SCs at the tips of pseudoglandular stage embryonic human airways also retain a similar expression pattern (Nikolic et al., 2017). These three types of airway SCs also likely share invasive and migratory phenotypes during development and regeneration. Our findings in MECs suggest that Lef-1 expression may drive this phenotype. Consistent with this notion, biallelic Lef-1KI expression in MECs was sufficient to activate lineage commitment and migration to the SAE in the absence of injury, while also enhancing transcriptional pathways that control migration, invasiveness and proliferation in cultured MECs. Biallelic Lef-1KI expression in primary MECs also shifted their transcriptome toward a basal cell phenotype, which was consistent with an enhanced capacity of MEC^{Let-1KI} to differentiate into Scgb1a1⁺ club cells in vivo and in vitro.

Canonical Wnt/β-catenin signaling mediated by TCF/Lef-1 family members is thought to be primarily regulated through post-transcriptional processes that control the availability of nuclear β-catenin to engage DNA-bound TCF/Lef-1 transcription factors (Nusse and Clevers, 2017). In the absence of nuclear β-catenin, enhancer-bound TCF/Lef-1 complexes are thought to repress transcription. However, PGSCs during airway gland development appear to utilize a slightly altered mode of Wnt signaling, where Wnt3a induces both transcription of Lef-1 and levels of nuclear β-catenin (Driskell et al., 2004; Filali et al., 2002; Liu et al., 2010; Lynch et al., 2016; Xie et al., 2014). In this regard, MECs appear to behave similarly since they also induce Lef-1 expression and nuclear β-catenin following injury. Given that slowly cycling SMG SCs reside near these Wnt-active niches (Lynch et al., 2016), we hypothesize that the SMG niche responds to severe SAE injury by modulating Wnt signals that induce Lef-1 gene expression, which leads to self-renewal and asymmetric production of multipotent MEC-daughter cells. A requirement for an inductive injury signal (i.e., Wnt stimulated nuclear (3-catenin) to promote lineage commitment of MECs is consistent with the minimal lineage contribution to SMGs and the SAE in uninjured ACAT2-Cre^{ERT2}:Lef-1K1^{+/-} mice. However, the finding that ACAT2-Cre^{ERT2}:Lef-1K1^{+/+} mice (with two Lef-1KI alleles) spontaneously induce proliferation and lineage commitment of MECs suggests that this process may be activated by tonic levels of nuclear β-catenin when sufficient Lef-1 is present. Thus, the balance of occupied Lef-I/β-catenin binding sites in the genome, rather than the absolute amount of β-catenin, maybe be most important to lineage commitment of MECs (Nusse and Clevers, 2017).

While the finding that Lef-1 over-expression enhances the regenerative capacity of multipotent MECs for both SMG and SAE compartments is significant for the field of regenerative medicine, there remain unknown features of this mechanism. For example, it appears that not all MECs with biallelic Lef-1KI expression (MEC^{Lef-1KI+/+}) are actively replicating in vivo and while regenerative expansion occurs, it is not indefinite and appears self-limiting. One explanation for these findings is that available Wnt signals weaken as MEC-daughter cells exit the glandular SC niche. Such a process could limit activation of Lef-1 in MEC daughters as nuclear β-catenin and/or other Lef-1 co-factors decline with distance from the glandular SC niche. Thus, as MEC^{Lef-1KI+/+} SCs differentiate into other glandular cell types and basal cells on the SAE, Lef-1 may no longer have a functional impact. In support of this hypothesis, overexpressing Lef-1 under a club cell-specific promoter (Scgb1a1/CC10) in transgenic mice, or in human tracheal xenografts using viral vectors, had no impact on airway biology (Duan et al., 1999). By contrast, our in vitro culture studies were performed under conditions that promote Wnt signaling (e.g., Wnt agonists) and as such the enhanced proliferative and migratory capacity of MEC^{Lef-1KI+/+} would be retained.

A second explanation for limited proliferative expansion of MEC^{Lef-1KI+/+} daughter cells in vivo could be that Lef-1K1^{+/+} overexpression induces symmetric division of MECs to two differentiated daughter cells and/or directed differentiation in the absence of replication. In support of these hypotheses, repeated severe injury of induced ACAT2-Cre^{ERT2}:Lef-1K1^{+/+} mice clearly demonstrates repopulation of the SAE and SMGs with untraced (GFP⁺Lef-1⁻) cells. Thus, high-level unregulated Lef-1 expression in MECs reduces self-renewal of the SC state. Given that Lef-1K1^{+/-} MECs do not spontaneously engage a regenerative response in the absence of injury, the level of Lef-1 and/or its activated state is likely highly regulated in MECs during lineage commitment. For example, the partitioning of Lef-1-bound DNA to daughter cells could be critical for MEC self-renewing and maintenance of an undifferentiated SC state.

Taken together, our results demonstrate that glandular MECs are multipotent reserve SCs of both the SAE and SMGs. Induction of Lef-1-mediated Wnt/β-catenin signaling plays an integral role in lineage commitment of MECs and maturation toward SAE basal SCs. Further studies on the MEC SC population identified here will provide greater clarity on transcriptional and environmental signals that control fate decisions in the context of severe airway injury. Such studies are likely to yield important and broadly relevant information regarding epithelial tissue plasticity, in both normal and disease states. Whereas mice possess SMGs only in the proximal trachea, the glandular SC niche and MEC SCs may play a more significant role in lung regeneration and disease processes for other species that, like humans, possess SMGs throughout the cartilaginous airways.

### Example 2

Glandular myoepithelial cells (MECs) function as multipotent progenitors for 7 cell types within the surface airway epithelium (SAE) and SMGs. Furthermore, MECs have the ability to form SMGs de novo in denuded xenografts, and are the first airway stem cells known to have this functional attribute. Also central to this proposal is the finding that the Lef-1 transcription factor controls both the lineage commitment of MECs and their ability to migrate to the SAE, where they undergo directed dedifferentiation into multipotent basal cells (BCs). The proposed research will capitalize on this biology to facilitate the development of CF stem cell-based therapies. As disclosed herein below, Lef-1 expression in MECs altered the expression of genes that direct lineage commitment, proliferation, and rapid migration from glands to the airway surface. The central therapeutic hypothesis is that the unique cell-intrinsic properties of MECs can be harnessed to improve stem cell-based therapies to the lung through directed reprogramming.

Define the set of Lef-1-dependent factors that regulate MEC lineage-commitment, proliferation, and migration from SMGs to the SAE. The data from αSMA-Cre^{ERT2} lineage tracing experiments in mice demonstrate that glandular MECs contribute to regeneration of the tracheal SAE following naphthalene injury. Following airway injury, the induction of the Lef-1 transcription factor within MECs is required for lineage commitment and migration to the SAE. Furthermore, the conditional expression of Lef-1 in MECs using an αSMA-Cre^{ERT2}: RosA26-^{LoxP}EGFP^{StopLoxP}-hLef-1 knock-in (Lef-1KI) transgene enhances this regenerative capacity in a dose-dependent fashion. Based on RNAseq results comparing MEC^{Lef-1KI} to MEC^{WT}, the regulation of matrix remodeling proteins and cell surface receptors/adhesion molecules by Lef-1 is the primary reprogramming event that controls the exit of glandular MEC progenitor cells from their glandular niche and their migration to the airway surface. This aim will define Lef-1 target genes using RNAseq time courses and ChIPseq following Lef-1 induction, and in vivo localization of candidates following airway injury and/or induction of Lef-1. These Lef-1-dependent candidate genes will then be functionally interrogated for the ability to enhance proliferation, migration, or matrix invasion, using semi-high throughput in vitro assays utilizing Cas9-P2A-tdTomato-expressing primary MEC^{Lef-1KI} and MEC^{WT}.
Determine whether Lef-1 activation in MECs and basal cells (BCs) enhances their regenerative potential. This aim will test whether enhancing Lef-1 expression in surface airway BCs or in glandular MECs augments properties important for airway cell engraftment, such as cell attachment, proliferation, and reestablishment of stem cell niches on the airway surface. Rates of stem cell attachment and proliferation will be assessed in vitro, using denuded mouse tracheas, and ex vivo tracheal xenograft competition experiments will be used to directly compare BC^{Lef-1KI} VS. BC^{WT} and MEC^{Lef-1KI} vs. MEC^{WT} for their abilities to regenerate a differentiated epithelium and SMGs. Lef-1 expression may enhance the engraftment of BCs and MECs, as well as the reestablishment of stem cell niches on the airway surface. We will test this hypothesis using in vivo engraftment into naphthalene-injured immunocompromised mice with two complimentary approaches: 1) transgenic induction in Lef-1KI in lineage-traced cells and 2) gRNA-mediated induction of Lef-1 in dCas9-VP64/p65 expressing stem cells (which can transiently induce Lef-1 up to 6000-fold). As additional Lef-1 target genes are identified in Aim 1, similar approaches will be used to evaluate whether they play important roles in the adhesion and proliferation of engrafted MECs and BCs.
Create an αSMA-IRES-Cre^{ERT2} ferret in which glandular progenitor cells can be lineage-traced. Although mouse is the most genetically pliable model species for stem cell research in the lung, its application to studies of SMG stem cells is limited to the trachea. In contrast to mice, ferrets, have SMGs throughout the cartilaginous airways like humans, and maintain surface airway cell types similar to those in humans. We recently generated a knock-in ROSH-26-CAG-^{LoxP}tdTomato^{stopLoxP}-EGFP Cre reporter ferret. In this aim, we will generate an αSMA-IRES-Cre^{ERT2} knock-in ferret in which glandular MEC biology can be interrogated. Importantly, this model will facilitate the development of therapies targeting this glandular stem cell. The advent of CRISPR/Cas9 methods for gene manipulation in ferret zygotes has made this goal cost/time-feasible.

The results facilitate a deeper understanding of (a) factors that facilitate MEC-mediated cell repair in the airway and how they differ from their BC counterparts in the SAE, and (b) how the unique biology of MECs might be harnessed for CF cell therapy of the lung. In addition, Aim 1 will lay the foundation for future in vivo testing of Lef-1 dependent factors in conditional knock-out or knock-in mice under the control of αSMA-Cre^{ERT2}, and Aim 3 will move lung stem cell research from mouse to the ferret, whose airway system is more similar to that of humans and for which a CF model exists with lung disease.

Scientific premise and significance: Wnt-regulated mechanisms were defined that control Lef-1 and Sox2 transcription factor activity required for the lineage commitment. Recent research suggests that similar pathways control the adult SMG stem cell niche following airway injury. Preliminary data demonstrate that SMG stem cell niches contribute to regeneration of the surface airway epithelium (SAE) following injury, and that these glandular stem cells have a unique capacity to regenerate both SAE and SMG cell types by virtue of unique Wnt signals. Whereas others provide evidence that gland ducts, which are an extension of the SAE, contain airway stem cells, the existence of other stem cell populations residing deeper within SMG has remained unclear. Multipotent myoepithelial cells (MECs, glandular lineage) were isolated that are born very early during SMG development and contribute to approximately 50% of glandular cell mass. These findings led us to test whether glandular MECs in adult mice are capable of contributing lineages to both SAE and SMG following injury. Preliminary lineage tracing data suggest that the glandular MECs, which reside deep within SMGs, indeed contribute to differentiated cell types in both SMGs and the SAE following naphthalene airway injury of mice, and that Lef-1 is involved in this process. These findings are lineage tracing-based evidence that gland-derived cells can contribute to the SAE following injury. Spatially-restricted, Wnt-active niches were identified within SMGs that appear to regulate the first progenitors that re-enter the cell cycle following airway injury. Using conditional deletion and overexpression of Lef-1, it was demonstrated that a Wnt-mediated mechanism controls self-renewal and the lineage commitment of SMG stem cells. In fact, conditional activation of Lef-1 selectively within MECs leads to accelerated airway regeneration mediated by MECs, as well as rapid migration of MEC daughter cells to the SAE. The RNAseq-based studies of primary glandular MECs isolated following lineage labeling and/or induction of Lef-1 have revealed many pathways that may contribute to the migration and proliferation of this stem cell compartment. Thus, the manipulation of airway stem cells to adopt MEC properties, in particular their Lef-1 activity, can be used to enhance cell therapy for CF. The data allow for (a) understanding the molecular basis of Lef-1-based enhancement of the regenerative capacity of glandular MECs, (b) applying the findings on unique aspects of MEC biology to enable the development of better approaches for CF cell therapy and gene editing technologies using more accessible sources of stem cells (i.e., basal cells (BCs) and/or iPSCs), and (c) generating a transgenic ferret model that is more effective for translation of our findings to humans, since both ferrets and humans have SMGs throughout their cartilaginous airways.

### Summary

Mechanisms underlyinq airway stem cell properties—to reveal Wnt-mediated mechanisms that are key aspects of stem cell regeneration in the airway, as well as Lef-1-dependent signaling molecules that are universally useful in stem cell therapy applications for CF.
Features of a precursor of both SMGs and the SAE—demonstrating that glandular MECs contribute to the regeneration of both SMGs and SAE following airway injury. A deeper understanding cell-intrinsic properties of MECs that control regeneration has great potential for developing treatments for SMG defects in CF when combined with gene editing technologies.
MEC progenitors that can form new SMGs—In CF patients, SMGs are severely affected but no therapeutic options for correcting such defects exist. The ability to form new SMGs using a cell therapy approach directed at the airway surface could have tremendous value in treating CF airway disease.

The research thus relates to elucidating airway stem-cell biology, applying this knowledge to stem cell therapies, and creating new ferret models that are well suited for the study of stem cell biology and CF therapy. The application of novel CRISPR/Cas9-mediated approaches to primary stem cells will enable us to assess targets of Lef-1 that impact stem cell behavior and phenotypes important for cell therapy and airway regeneration in the setting of injury. The findings enhance efforts toward reprogramming iPSCs and/or SAE BCs to improve the outcomes of cell therapy. The work includes the generation of ferret models capable of lineage-tracing MEC progeny, making it possible to determine how MECs participate in airway repair in a CF model that contains SMGs throughout its cartilaginous airways. These enable us to address important hypotheses concerning SMG stem cells and their therapeutic applications.

The data characterize a previously unrecognized glandular progenitor (i.e., MECs) of SAE BCs, and its properties using nCRISPR/dCas9-mediated approaches that could be therapeutically applied with dCAS9 ribonuclear protein (RNP) complexes to transiently manipulate stem cell phenotypes and improve cell therapy applications.

### Results

Glandular MECs contribute to regeneration of SMGs and the SAE following severe airway injury. SMG ducts have historically been considered a stem cell niche, since slowly-cycling nucleotide label-retaining cells (LRCs) reside in this region and isolated duct progenitors can give rise to SAE cell types). However, we found that glandular LRCs, which are able to cycle following repeated injury and to retain multiple nucleotide labels, can reside deeper within SMG tubules. Recent work has also demonstrated that multipotent glandular myoepithelial cells (MECs) are born early during gland development with the capacity to form various SMG cell types. In this study, the αSMA/ACTA2-CRE^{ERT2} and SMMHC/MYH11-CRE^{ERT2} drivers were used to trace MEC lineages during gland development. It was hypothesized that MECs of adult SMGs may also serve as progenitors for the SAE following airway injury. To test this possibility, the progeny of glandular MECs in the mouse trachea following naphthalene injury were traced, using an αSMA^{CreERT2} driver⁽¹⁷⁾ on a Rosa-26^{LoxP}tdTomato^{StopLoxP}-EGFP⁽¹⁸⁾ (ROSA-TG) Cre-reporter background. After 5 days of tamoxifen induction, the majority of αSMA⁺ glandular MECs were also GFP⁺ (FIG. 8A). Naphthalene injury of tamoxifen-induced mice led to the emergence of marked MECs within gland ducts (FIG. 8B,D-inset arrow), and some of these expressed Trop2, a marker of basal cells (BCs) and gland ducts⁽. Notably, marked MECs on the SAE appeared to expand clonally, with more proximal clones remaining αSMA⁺ and more distal clones adopting the αSMA⁻K5⁺ phenotype (FIG. 8C,D). Similar changes occurred for the BC marker NGFR, with clones in the proximal trachea being NGFR⁻ and those in the distal trachea NGFR⁺ (FIG. 8E). MECs also differentiated into lysozyme-expressing glandular serous cells (FIG. 8F), and columnar cells of the SAE (FIG. 8G,H).

These findings provide support for the hypothesis that glandular MECs are multipotent progenitors of both SMG and SAE cell types. Such a hypothesis is not without precedent, since K5⁺K14⁺ myoepithelial cells of mammary glands are thought to be multipotent progenitors of lumenal cell types. These preliminary phenotyping studies further suggest that MECs adopt a basal-cell program in the SAE, as they migrate distally down the trachea and repair injury. We have also performed airway injury studies in adult animals using the SMMHC/MYH11-CRE^{ERT2} driver, and our results are similar to those with αSMA-CRE^{ERT2} in terms of the extent to which this lineage contributes to SAE repair (data not shown due to space limitations).

Glandular MECs can establish stem cell niches in the SAE that respond to subsequent reinjury. The above results demonstrated that glandular MECs can give rise to BCs on the SAE following injury. However, it remained unclear whether these glandular MECs could reestablish basal stem-cell niches within the SAE and also differentiate into various airway cell types. To address this question, sequential injury experiments were performed on induced αSMA-Cre^{ERT2}:ROSA-TG mice and tested the ability of MEC-derived daughter cells on the SAE to expand and differentiate following a second mild injury (FIG. 9A). Indeed, lineage-tagged MEC daughter cells expanded on the airway surface following second injury giving rise to large clones (FIG. 9B,C). Phenotyping of lineage-tagged cells in the SAE and SMGs demonstrated that MECs gave rise to 7 cell types including: 1) α-tubulin⁺ ciliated cells in the SAE (FIG. 9C), 2) Scgb3a2⁺ club cells in the SAE (FIG. 9K), 3) UEA-1⁺ and Muc5B⁺ mucus-secreting cells in the SAE (FIG. 9H,I), 4) DBA⁺ and Muc5B⁺ mucous tubules in SMGs (FIG. 9E,J), 5) lysozyme⁺ and UEA-1⁺ serous cells in SMGs (FIG. 9F,G), 6) Trop2⁺ ductal cells in SMGs (FIG. 8B), and 7) NGFR⁺ and K5⁺ BCs in the SAE (FIG. 8Ci,Eiii). Notably, MECs did not give rise to Scgb1a1⁺ club cells (FIG. 2D). The contribution of MEC lineages to the SAE following single injury (SI) and double injury (DI) was ˜12% and ˜29%, respectively (FIG. 9L). Given that mild injury does not lead to substantial mobilization of glandular MEC-derived progenitor cells to the SAE, these findings demonstrate that MEC-derived progenitors on the SAE can reestablish niches capable of expansion following reinjury. In the SMGs, lineage-tagged MECs marked ˜20% of glandular cells in the uninjured state, suggesting that this proportion of glandular cells are MECs. Following double injury, lineage-tagged cells in the SMGs accounted for roughly 45% of glandular cells.

Of note, the percentage of GFP⁺ cells expressing cellular markers of differentiation in both the SMGs and SAE doubled following second injury (FIG. 9N,O), suggesting that following single injury MEC daughter cells remain incompletely differentiated, and upon reinjury have a greater ability to differentiate into other cell types. Furthermore, comparison to the differentiation profile of untraced cells in the SAE (FIG. 9P) demonstrated that MEC progenitors have a bias toward goblet cell fate and are less likely to differentiate into club cells (FIG. 9N). In humans, SMGs are present throughout the cartilaginous goblet cell-containing proximal airways, which lack club cells, whereas in mice the cartilaginous airways that lack SMGs contain club cells. Thus, it is possible that in species that utilize the SMG niche more extensively throughout their cartilaginous airways, this niche may be programmed to supply only MEC-derived progenitors that are capable of giving rise to basal, goblet, non-ciliated columnar, and ciliated cells. Thus, generation of an αSMA-CRE^{ERT2} ferret is needed to understanding the extent to which glandular MECs contribute to airway repair in humans.

Following airway injury, the Lef-1 and Sox2 transcription factors are dynamically regulated in MECs. Pathways important for regulating stem cell niches within SMGs can likely be informed through an understanding of processes that establish this compartment during development. Toward this end, it was shown that canonical Wnt/β-catenin signaling is activated during early stages of SMG development, and this same process appears to be conserved in SMG stem cell niches where slowly cycling label-retaining stem cells reside. Specifically, stem cells in the gland placode activate transcription of Lef-1 at the earliest stage of placode formation, and that sustained Lef-1 expression at the tip of invading glandular tubules is required for proliferation and SMG development. Moreover, Sox2 expression is suppressed in the glandular placode, and that this coordinates transcriptional activation of the Lef-1 gene in the presence of Wnt signals. Given the importance of Writ signaling in coordinating primordial glandular stem cells during gland development, we hypothesized that this signaling pathway may also regulate adult glandular MECs following airway injury. Analysis of Lef-1 and Sox2 expression in SMGs at early time points following airway injury (FIG. 10) revealed that Lef-1 was induced (FIG. 10A,C), and Sox2 suppressed (FIG. 10B,D), in a large proportion of glandular cells including MECs (FIG. 10E-H). These findings support the notion that processes controlling neonatal and adult glandular stem cells are similar and controlled by Lef-1 and Wnt signals.
Lef-1 is required for the lineage commitment of MECs and their contribution to airway repair following injury. To assess whether Lef-1 is required for the stem cell functions of MECs, conditional Lef-1 knockout mice (Lef-1)^{vs} bred to αSMA-Cre^{ERT2}:ROSA-TG mice were use. Deletion of Lef-1 specifically in glandular MECs had no effect on the persistence of this cell type up to 21 days post-induction (FIG. 11A). However, following airway injury, lineage-traced GFP⁺ MECs appeared fragmented and untraced MECs repopulated the majority of the gland (FIG. 11B). Importantly, the Lef-1 knockout MECs did not contribute to the SAE following injury (FIG. 11B,C).

Whole-body knockout of Lef-1 was used as a second approach, generating Lef-1^{Ftx/Ftx}:ROSA-TG:ROSA-Cre^{ERT2} mice in which tamoxifen induction of Cre deletes Lef-1 in most cells (FIG. 12). As in the case of MEC-directed deletion of Lef-1, ROSA-Cre^{ERT2} Lef-1^{KO} cells in the SMG and SAE persisted in the absence of injury (FIG. 12A,D). However, following injury, GFP⁺ Lef-1^{KO} cells were progressively replaced by tdTomato⁺ Lef-1″T cells in both the SAE and SMGs (FIG. 12B,C). Importantly, GFP⁺ Lef-1^{KO} MECs were repopulated by tdTomato⁺ Lef-1^{WT} MECs following injury (FIG. 12B vs. 12D). Cumulatively, these findings implicate Lef-1 as a transcription factor required for stem cell self-renewal in SMGs and the SAE following injury.

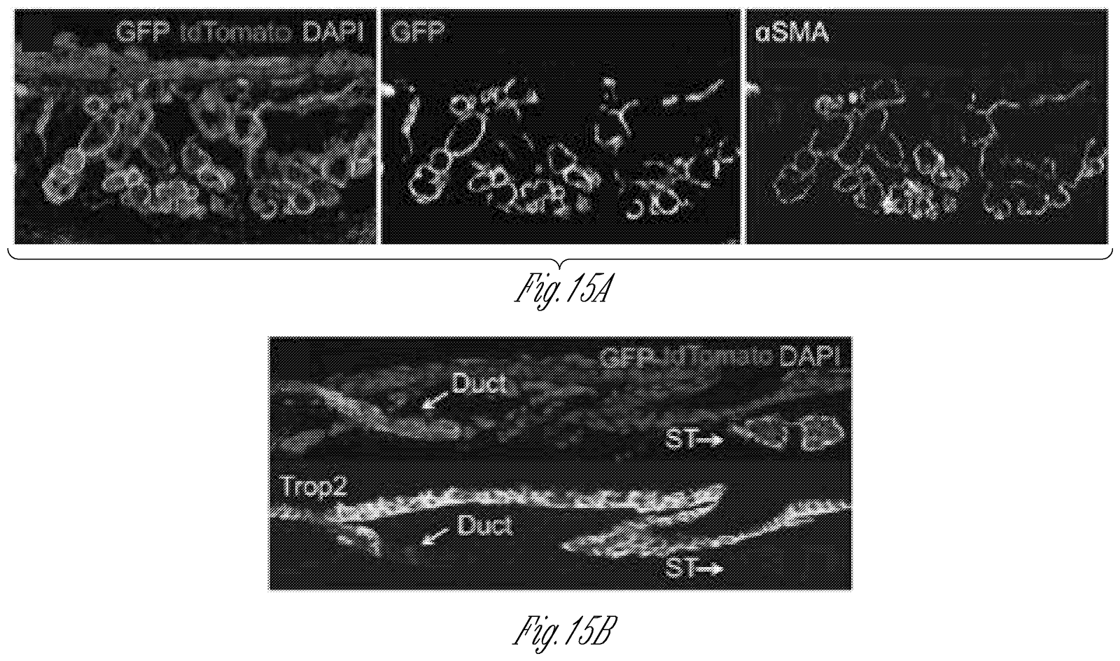

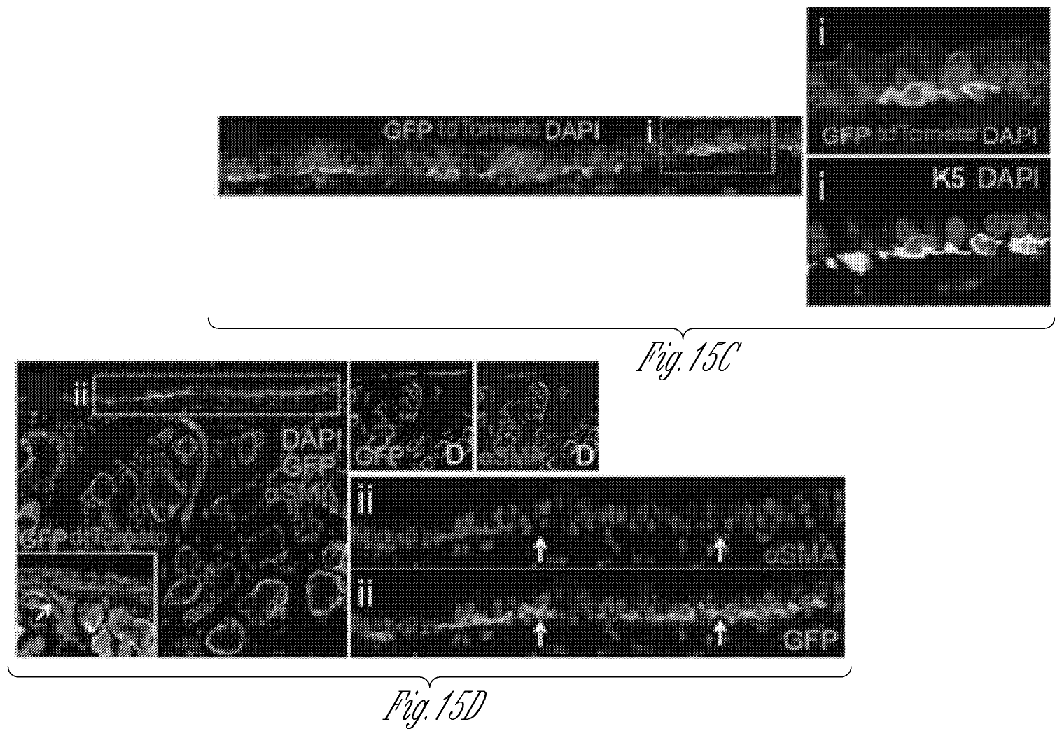

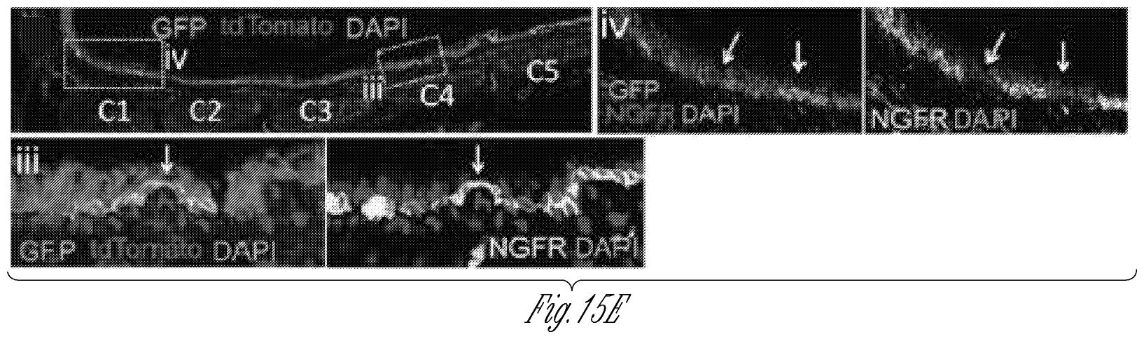

Induced expression of Lef-1 enhances the regenerative capacity of MECs in a dose-dependent fashion. Given that glandular MECs require Lef-1 to commit toward SAE cell fates following injury, it was hypothesized that inducing Lef-1 expression might enhance the regenerative capacity of MECs. To test this possibility, a ROSH-CAG-^{LoxP}EGFP^{stopLoxP}-hLef-1 transgenic mouse (Lef-1KI) (FIG. 13A) was generated, which can be used to conditionally overexpress Lef-1 in the presence of a ROSA-Cre^{ERT2} (FIG. 13I,J) or other Cre deriver. Induction of Lef-1 also leads to deletion of an EGFP reporter, enabling lineage tracing. Whereas uninduced mice retained GFP expression throughout the SAE and SMGs following injury (FIG. 13B), in those subjected to αSMA-Cre^{ERT2} tamoxifen induction, MECs contributed to SMGs and SAE even in the absence of injury when the transgene was at homozygosity (Lef-1K1^{+/+}) but not at heterozygosity (Lef-1K1^{+/-}) (FIG. 6C,E,G,H). Injury enhanced this level of MEC^{Lef-1KI} contribution, with a maximal contribution to the SAE and SMGs of 90% in Lef-1K1^{+/+} mice (FIG. 13F-H). These findings further implicate Lef-1 in the lineage commitment of MECs and their ability to rapidly migrate to the airway surface, even in the absence of injury (FIG. 13E). We have aged mice for greater than a year following αSMA-Cre^{ERT2} mediated activation of Lef-1K1^{+/+} in MECs, and find that lineage-traced cells move down the trachea to about cartilage ring 8-10 but then stop. Since proliferative expansion is not indefinite, we hypothesize that Lef-1 expression leads to lineage commitment of MECs to transient amplifying progenitors, and that MEC stem cells are
Define the set of Lef-1-dependent factors that regulate MEC lineage-commitment, proliferation, and migration from SMGs to the SAE. To identify the Lef-1-dependent factors that influence MEC behavior, RNAseq experiments were performed comparing passage 1 (P1) cultures of FACS-purified MECs isolated from tamoxifen-induced αSMA-Cre^{ERT2}:ROSA-TG mice (GFP⁺ cells) and αSMA-Cre^{ERT2}:Lef-1KI mice (GFP⁻ cells). This experiment identified 320 genes that are differentially expressed in MECs following genetic induction of Lef-1 expression (FIG. 14). Notably, Ingenuity Pathway Analysis (IPA) yielded significant positive Z-scores (i.e., activation) for Lef-1KI pathways involved in cell movement and invasion, but negative scores for cell-death pathways. Genes involved in proliferation and cell-cell contact were also upregulated. The goal of this aim is to identify the most proximal downstream targets of Lef-1, as well as effector genes and proteins that affect MEC progenitor cell behavior following airway injury.
Gene targets of Lef-1 in MECs control master regulators involved in migration and proliferation. Airway injury induces Lef-1 expression in glandular MECs (FIG. 10) and Lef-1 required for their migration to SAE and commitment to BC lineages (FIG. 11). Not all the differentially expressed genes shown in FIG. 14 will be relevant Lef-1 targets following in vivo airway injury in this subaim, direct targets of Lef-1 and the earliest genes activated or repressed following Lef-1 induction in MECs are identified. These may be direct Lef-1 targets, and potentially master regulators of the migration and proliferation pathway gene subsets in FIG. 14.
Approach: A time course RNAseq experiment is performed following conditional induction of Lef-1KI in cultures consisting predominantly of MECs, and compare the results to those for WT equivalents. Because in vivo induction of Lef-1KI leads to nearly immediate commitment of MECs to other cell types, we will perform this analysis using P7 cultures of glandular cells (at which time 90% are MECs) (FIG. 15). SMG cells are isolated from ROSA-Cre^{ERT2}:Lef-1K1^{+/-} mice, induced with hydroxy-tamoxifen at P7, and harvested for RNAseq at 0, 6, 12, 24, 36, and 48 hrs post-induction. SMG cells are isolated and plated into fibroblast-free conditionally reprogrammed culture (CRC) as used for the experiments in FIG. 15A-C. Second, ChIPseq is performed for Lef-1 binding sites in Lef1K1^{+/-} and WT SMG cells at 48 hrs post-induction. Cross referencing of genes whose expression is altered following Lef-1 induction against those in which Lef-1 binding sites are present within 10 kb of either side of the transcriptional start site (TSS) will reveal the downstream master regulators of MEC functions. The lead Lef-1 dependent candidates will be verified by immunolocalization, in SMGs at 0, 12, 24, 36 hrs post: (a) naphthalene injury in αSMA-Cre^{ERT2}:ROSA-TG mice, or (b) tamoxifen induction of αSMA-Cre^{ERT2}:Lef-1K1±/±mice.
Results: Our laboratory has previously performed ChIP, and although we have yet to perform ChIPseq we do not anticipate any procedural problems. The time course RNAseq experiments identify a handful of genes that are upregulated early following Lef-1 induction, that the cascade of gene networks activated in FIG. 14 will follow at later time points post-induction, and that ChIPseq defines the gene sets that are direct targets of Lef-1. scRNAseq of lineage-traced MECs at early time points following airway injury (i.e., 24 hrs) may be the most direct approach to studying injury-associated transcriptional signatures that correlate with Lef-1 induction.
Lef-1 expression in MECs enhances their migratory and invasive properties. RNAseq comparing primary cultures of MEC^{Lef-1KI+/+} and MEC^{WT} demonstrate that pathways controlling cellular adhesion, movement, invasion, and proliferation are significantly upregulated by Lef-1 (FIG. 14). Identifying the intrinsic properties of MECs that are influenced by Lef-1 will shed light on how MECs can leave their glandular niche and rapidly migrate to the airway surface to proliferate and expand as BCs. Functionally defining both the master regulators and downstream effectors of these processes will facilitate the development of targeted approaches to enhance these positive regenerative characteristics for stem cell based therapies in the CF airway. In this subaim, we will develop in vitro models effective for evaluating these properties.
Approach: Three aspects of MEC function are evaluated to determine how Lef-1 expression influences: 1) proliferation (using a fibroblast-free CRC method), 2) migration (using a monolayer culture scratch assay), and 3) matrix invasion and morphogenesis (using an organoid culture assay). Each assay is used with temporal induction of Lef-1 expression, viable imaging, and/or lineage tracing of MECs to define the intrinsic Lef-1 dependent mechanisms.
Proliferation assays: Two mouse models are used to differentially lineage-tag MEC^{Lef-1KI} and MEC in vivo with tamoxifen prior to isolation: αSMA-Cre^{ERT2}:ROSA-TG (Red→Green) for MEC^{WT}, and αSMA-Cre^{ERT2}: Lef-1KI:ROSA-LsL-tdTomato (Green→Red) for MEC^{Lef-1KI+/-}. Mice are induced with tamoxifen 2× at 12 hr intervals, and crude SMG cells are isolated at 24 hrs post-induction. These cells are then FACS purified to obtain lineage-tagged populations, and placed into fibroblast-free CRC at various ratios (MEC^{Lef-1KI}: MEC^{WT}, 10:90, 50:50, and 90:10). Cultures are FACS sorted as they are passaged to compare the rates of proliferation for each population (as shown in FIG. 15C).
Migration assays: Cells isolated as described for proliferation assays are placed in mixed cultures of 50:50 MEC^{Lef-1KI}:MEC^{WT} at near confluence density. The next day cultures are scratched, and viable imaging performed using a Leica DMR spinning disk confocal microscope in a temperature- and CO₂-regulated chamber. Migrating cells are imaged over a 16 hr period, and rates of migration calculated using Metamorph tracking software. Glass bottom dishes are coated with conditioned medium from 804G cells (rich in laminins) or with collagen IV, and these substrates may be varied to maximize migratory rates.
Matrix invasion/morphogenesis assays: Isolated SMG cells form unique tubular structures in organoid culture, whereas BCs from the SAE form spherical organoids (FIG. 15D,E). It was hypothesized that this difference is due to the greater invasive properties of MECs in the glandular epithelial cultures, and that Lef-1 expression will enhance this phenotype and lead to larger organoids with more tubular features. Mixed cultures of 50:50 MEC^{Lef-1KI}:MEC^{WT} differentially labeled with a tdTomato or GFP transgene, respectively, are assessed. The size (2D area) will be calculated using Metamorph software, and the extent of tubulogenesis will be determined by calculating the circularity index.
Results: Lef-1 induction enhances the proliferative capacity of MECs, eventually leading to the overgrowth of MEC^{Lef-1KI} in MEC^{Lef-1KI}: MEC^{WT} mixed cultures. Scratch assays in confluent MEC^{Lef-1KI}: MEC^{WT} cultures demonstrate that MEC^{Lef-1KI} migrate in to the wounded area more rapidly. Given that MMPs are activated in MEC^{Lef-1KI} cultures, greater invasive characteristics of this population in organoid culture are observed. Both clonal organoids and mixed organoids composed of both MEC^{Let-1KI} and MEC^{WT} cells will be present. However, given that each population is differentially labeled, mixed organoids serve as good controls for cell-intrinsic properties that mediate matrix invasion (e.g., the protrusions of tdTomato⁺ MEC^{Lef-1KI} tubules are longer than those of GFP⁺ MEC^{WT} tubules). Thus, the morphologies of non-clonal and clonal organoids are analyzed separately. It is possible that local non-cell autonomous paracrine effects induced by Lef-1KI might influence matrix invasion. In this case, however, the tubulogenesis of mixed non-clonal organoids is expected to differ less than that of clonal organoids. In this scenario, each cell population separately without mixing may be evaluated.
Disruption of key Lef-1 target genes and downstream effectors will impair the migratory and proliferative properties of MECs. Key genes that are identified are deleted, in combination and ultimately individually, to identify those that are responsible for Lef-1-dependent enhancement of migratory and proliferative capacities of MECs. A semi-high throughput gene editing approach to interrogate Lef-1-dependent genes in primary airway stem cells, using highly efficient biallelic gene disruption by Cas9, is employed. In this approach, primary airway cells are grown under CRC conditions, transduced with Cas9-lentivirus, and polyclonal pools selected for antibiotic resistance. These cells are then subjected to highly efficient liposome-mediated transfection with sgRNAs (FIG. 16A-F). The key to this system is an sgRNA spike that targets a reporter in transfected cells. LoxP, tdTomato, and EGFP sgRNAs can be used for tracing transfected cells while deleting other gene target(s). Using homozygous ROSA-TG Cas9-expressing BCs transfected with a single LoxP sgRNA, ˜99% of transfected cells undergo biallelic cleavage of the target site, converting tdTomato⁺ to either an tdTomato⁻EGFP⁺ (˜50%) or tdTomato⁻EGFP⁻ (˜25%) phenotype (FIG. 16A-E). Through sequencing of indels we have found that the tdTomato⁻EGFP⁻ phenotype results from incomplete excision of tdTomato that fails to activate EGFP expression. Importantly, in this assay only ˜1% of cells are tdTomato⁺EGFP⁺ (yellow), demonstrating that in transfected cells both alleles are targeted at high efficiency. Using an sgRNA targeting tdTomato (FIG. 16D,F) or EGFP (data not shown), we found similar levels of biallelic gene targeted disruption in 70-75% of cells in the sgRNA-transfected culture.
Approach: The experimental model system described above is used to interrogate Lef-1 gene targets responsible for the unique characteristics of MECs. sgRNAs are generated for various Lef-1 targets and screen them in vitro for efficiency of dsDNA target cleavage using purified Cas9 protein. Primary SMG cells isolated from ROSA-Cre^{ERT2}:Lef-1KI mice are used for this analysis. Primary glandular cells are isolated, placed into CRC, and transduced with Cas9-P2A-tdTomato expressing lentivirus. Polyclonal pools of tdTomato⁺ cells are isolated by FACS and passaged to P5. At this point, 90% of the cells in this culture are MECs. The ROSH-CAG-^{LoxP}EGFP^{StopLoxP}-Lef-1KI cassette is induced with hydroxy-tamoxifen (EGFP⁺dTomato⁺ is converted to EGFP⁻tdTomato⁺) and passage-matched cultures without Lef-1 induction (EGFP⁺dTomato⁺) are retained. These populations are transfected with complexes of sgRNAs targeting tdTomato and Lef-1-dependent effector genes. Those cells that inactive tdTomato in each population ae transfected and, based on the studies in FIG. 16, should be enriched for biallelic indels within experimental gene targets. Prior to proceeding to phenotyping experiments the efficiency of target gene disruption in FACS isolated populations is evaluated, using TIDE analysis of Cas9/gRNA target sites. Based on experience (FIG. 16), ˜75% of cells undergo tdTomato inactivation (FIG. 16F), and of these lineage tagged cells ˜99% also undergo biallelic targeting of the experimental loci (FIG. 16D,E). These mixed populations of transfection-traced, gene-disrupted cells are used to perform proliferation, migration, and invasion assays.

TABLE 2
IPA Analysis Defining Current Lef-1-dependent Candidates for Interrogation.

| Canoncial and | p- | Z- | # | |
|---|---|---|---|---|
| Functional Annotations | value | score | Molecules | Canididates for Testing (Fold Change RNA LefKI/WT) |
| Adhesion and Diapedesis | 4.0E-10 | — | 38 | MMP2 (5.7-fold), MMP3 (3.0-fold), MMP19 (3.5-fold) |
| Cell Movement | 3.7E-65 | 5.18 | 406 | SPARCL1 (7.9-fold), APBB1IP (4.6-fold), |
| | | | | ADGRA2 (3.8-fold), ITGA8 (3.1-fold), FERMT2 (1.9-fold), |
| Invasion of Cells | 7.9E-27 | 4.67 | 113 | ENPP2 (6.1-FOLD), FXYD5 (3.5-fold), LAYN (3.2-fold), |
| Cell-Cell Contact | 8.4E-13 | — | 62 | CDH5 (5.4-fold), JAM2 (3.7-fold), SLIT2 (3.0-fold), |
| | | | | CEACAM1 (−5.6-fold), |
| Integrin Signaling | 3.0E-10 | 0.688 | 25 | RHOJ (4.1-fold), WIPF1 (2.6-fold), CAPN6 (2.3-fold), RHOF |
| | | | | (−3.9-fold) |
| Respiratory system | 3.3E-12 | — | 65 | ADAMTS2 (6.3-fold), CDH11 (2.8-fold), FGF7 (2.3-fold), |
| development | | | | TGFB3 (2.1-fold) |
| Proliferation of Muscle | 8.3E-14 | — | 35 | IGF1 (6.7-fold), SCARA5 (6.3-fold), S1PR1 (3.5-fold) |
| Cells | | | | AGER (−6.8-fold) |
| Proliferation of Epithelial | 2.7E-13 | 0.91 | 82 | IGFBP4 (5.4-fold), ENG (3.9-fold) |
| Cells | | | | |
| Transcription | 2.1E-10 | 2.93 | 70 (27 | NR1h3 (4.3-fold), NR2F1 (4.0-fold), TWIST2 (3.9-fold), |
| | | | TF)* | Zeb1 (3.4-fold), Tbx4 (3.3-fold) |

*Of the genes in these pathways, 27 were transcription factors (TF), and among these 20 were significantly differentially regulated by Lef-1 expression.

Results: Studies using tamoxifen-induced MECs^{Lef-1KI} reveal targets important for Lef-1-dependent MEC phenotypes, while those using uninduced MECs^{Lef-1KI} will uncover MEC functions not augmented by Lef-1 expression. All assays have the advantage that untargeted cells (i.e., tdTomato⁺) are present, allowing for changes in phenotype that are dependent on Cas9-mediated gene deletion. For example, the Lef-1 induced (3.3-fold) transcription factor (TF) Tbx4 has been implicated in regulating proliferation, migration, and invasion of lung myofibroblasts. Two other Lef-1 induced TFs, TWIST2 (3.9-fold) and Zeb1 (3.4-fold), regulate epithelial cell adhesion, motility and proliferation. NR2F1 and NR1 h3 TFs are also induced by Lef-1 expression in MECs. Simultaneous targeting of all five of these TFs in tamoxifen-induced MECs^{Lef-1KI} cultures might reveal that tdTomato⁺ (untransfected) cells outgrow tdTomato⁻ (gene targeted) cells, and/or that tdTomato⁺ cells migrate more rapidly into wounded areas in a scratch assay, or exhibit enhanced invasiveness or altered morphogenetic behavior in organoid cultures. In the case of such an outcome, we would target each TF individually to determine which is responsible. Experiments provide important clues about three fundamental processes (migration, polarity, proliferation) concerning mechanisms by which Lef-1 controls glandular MEC migration to, and expansion on, the SAE. For example, functional interrogation of the Lef1-regulated genes could demonstrate that disrupting Lef-1 gene targets that regulate either cell-cell (FXYD5, LAYN or ADGRA2) or cell-ECM (SPARCL1, CEACAM1 or FERMT2) adhesion, invasive migration (MMPs, ENPP2, APBBI1P or ITGA8), collective cell migration (Cdh5, JAM2 or SLIT2), or cytoskeletal dynamics (RhoF, RhoJ, WIPF1 or CAPN6) impact the behavior of MECs in migration, proliferation, and invasion assays in vitro. Fundamental processes regarding collective migration and proliferation may also be linked and can be functionally interrogated using nucleotide labeling in organoid cultures to determine if invading cells are enriched or depleted for active cycling cells with a Cdh5⁺ or SLIT2⁺ phenotype. Ultimately, those genes indentified in vitro to facilitate Lef-1-mediated MEC behaviors are localized on lineage-traced MECs (MEC^{Lef-1KI} and MEC^{WT}) during the process of airway repair in vivo. For example, CEACAM1 and SPARCL1 Cas9-mediated disruption may prevent migration and invasion with in vitro assays, then these proteins are co-localized to lineage-traced MECs at various time points post-injury and along their path to the SAE. Beyond the scope of this work would be functional evaluation of top candidates using αSMA-Cre^{ERT2}:ROSA-TG conditional knockout mice, with the goal of identifying those Lef-1 effector genes responsible for MEC migration to the SAE and expansion as BCs.
Determine whether Lef-1 activation in MECs and basal cells (BCs) enhances their regenerative potential. This aim will seek to determine if the unique properties of MECs afford improved engraftment and regeneration over that of BCs following transplantation into airways using in vitro, ex vivo, and in vivo models. Furthermore, it will be determined whether Lef-1 activation in MECs and BCs enhances their engraftment and regenerative capacities following transplantation. Studies utilize two approaches: 1) permanent transgenic induction of Lef-1 gene expression and 2) transient induction of the Lef-1 gene expression via dCas9-VP64 in primary airway stem cells (FIG. 16G).
Activation of Lef-1 expression will enhance the ability of MECs to attach to a denuded airway basal lamina and proliferate. Activation of Lef-1 in MECs following airway injury likely controls multiple processes involved in directed migration, the remodeling of cell-cell contacts, the formation of lamellipodia, and actin rearrangements that allow cells to move fluidly along the basement membrane to the airway surface. It was hypothesized that the properties that are altered by Lef-1 expression enhance interactions with the basement membrane while reducing cell-cell contacts such as desmosomes, adherens and tight junctions. The rates of attachment to and expansion on denuded mouse tracheas in which the basal lamina is exposed, for both SAE BCs and glandular MECs, with and without forced expression of Lef-1, are compared.

### Approach:

In vitro adhesion and proliferative expansion assays: The rates of attachment to denuded mouse tracheas are generated by two methods: (A) three freeze-thaw cycles, each followed by flushing of cellular debris, and (B) naphthalene injury at 48 hrs (when denudation is maximal). Four groups of cells are compared: BC^{Lef-1KI}, MEC^{WT}, and MEC^{Lef-1KI} isolated from tamoxifen-induced mice in Table 1. Prior to testing, WT cells are transduced with a Firefly luciferase expressing lentivirus, whereas Lef-1KI cells are transduced with a Renilla luciferase expressing lentivirus, and polyclonal pools of each are antibiotic selected for transduced cells. The extent of attachment is monitored after the luminal surfaces of agarose anchored open tracheal cassettes are seeded with mixed populations (50:50) of BC^{WT}/BC^{Lef-1KI} or MEC^{WT}/MEC^{Lef-1KI}. After adhesion for various lengths of time, cassettes are washed and placed into Bronchial Epithelial Cell Growth Medium (BEGM). The extent of initial adhesion and growth expansion is monitored by biophotonic imaging (IVIS) over three days, using substrates specific for Firefly or Renilla luciferase. The ratio of the two measurements are used to calculate differential adhesion and growth rates. Since the Renilla and Firefly luciferase reporters have differing sensitivities, an equal fraction of the seeded cell mixture on the IVIS in the presence of each luciferase substrate is measured. This Renilla luciferase/Firefly luciferase baseline ratio is used to normalize readings from the tracheal measurements.
Tracheal xenograft competition assays and de novo gland formation: Total cells isolated from SMGs can generate glandular and surface epithelium in denuded tracheal xenografts. Moreover, when lineage marked SAE BCs were combined with SMG-derived cells, the SMG-derived cells outcompeted the BCs in generating surface epithelium 10-fold, and also contributed to newly formed SMGs whereas BCs did not⁽⁹⁾. It was hypothesized MECs have the greatest regenerative capacity in denuded tracheal xenografts, and that the expression of Lef-1 enhances both regeneration of the epithelium and de novo formation of SMGs. This hypothesis is tested by seeding xenografts with 50:50 mixtures of BC^{WT}:BC^{Lef-1KI} or MEC^{WT}/MEC^{Lef-1KI} isolated from induced mice in Table 1. The differential GFP or tdTomato reporters (Table 1) are used to quantify lineage contribution to regeneration of the SAE and formation of SMGs.

TABLE 1
Transgenic lines for the isolation of primary cells.

| | | Airway | Lineage |
|---|---|---|---|
| Cell Type | Lineage Marking Transgene | Source | Color |
| BC^{WT} | K5-Cre^{ERT2}:ROSA-L-tdTomato-sL-GFP | SAE | Green |
| BC^{Lef-1K1} | K5-CreERT2:ROSA-LsL-tdTomato:ROSA-L-GFP-sL- | SAE | Red |
| | Lef-1 | | |
| MEC^{WT} | αSMA-CreERT2:ROSA-L-tdTomato-sL-GFP | SMGs | Green |
| MEC^{Lef-1KI} | αSMA-CreERT2:ROSA-LsL-tdTomato:ROSA-L-GFP- | SMGs | Red |
| | sL-Lef-1 | | |

LsL: loxp-stop-loxp

Anticipated Results and Problems: Utilizing two different tracheal substrates for the in vitro adhesion assays (frozen/thawed or naphthalene injured), separately it is assessed whether cellular components of the trachea (fibroblasts, cartilage, etc.) influence adhesion and/or the proliferative behaviors of seeded stem cells. MECs^{Lef-1KI} likely adhere most rapidly to denuded tracheas, and this is indexed by a higher Renilla luciferase/Firefly luciferase ratio. BC^{Lef-1KI} may or may not adhere faster than BC^{WT}, but this will be an important test of the cellular specificity of Lef-1 function. The adhesive properties of BCs and MECs may be compared, which could easily be done by altering lentiviral reporters. Xenograft reconstitution experiments allow for the determination of regenerative capacities of the four comparative groups. A mixed SMG population has a higher regenerative capacity in this system than SAE BCs. Regenerative potential will likely follow the order of MEC^{Lef-1KI}>BC^{Lef-1KI}>MEC^{WT}>BC^{WT}. However, whether Lef-1 expression induces BCs to form glands is unclear. Mechanisms driving adhesion and regeneration can potentially also be elucidated by combining approaches with the deletion of selective Lef-1 target genes. For example, MEC^{Lef-1KI} display a 3.2-fold enhancement of the LAYN (layilin) mRNA as compared to MEC^{WT}. Layilin is a transmembrane hyaluronan receptor that associates with the cytoskeleton through the actin binding protein talin. The ITGA8 (integrin alpha 8) mRNA is also induced 3-fold by Lef-1 expression and plays an important role in wound-healing and organogenesis when in complex with beta-1 integrin⁽, and alpha-8/beta-1 integrin dimers bind to a variety of RGD motifs in ECM. Two other mRNAs induced by Lef-1 expression encode proteins that activate the adhesive functions of integrins. APBB1 IP (RIAM) targets talin to the plasma membrane to activate integrins, whereas FERMT2 (kindlin-2) links integrins to the actin cytoskeleton at focal adhesions. Thus, Lef-1 expression, which is activated in MECs following airway injury, likely controls cell intrinsic properties that dictate cell adhesion and migration, and these targets could be tested for their involvement in the regenerative process by combining approaches.
Activation of Lef-1 expression will enhance the ability of MECs to engraft into injured airways of immunocompromised mice. Cell engraftment rates and regenerative capacities of BC^{WT}, BC^{Lef-1KI}, MEC^{WT}, and MEC^{Lef-1KI} progenitors into naphthalene-injured SCID mice are compared. Mixtures of cell populations that are differentially lineage-labeled with GFP or tdTomato are directly compared. The extent of engraftment and types of differentiated cell progeny is quantified and compared to the native distribution of cell types in the non-transgenic airway epithelium.
Approach: Primary BCs and MECs are harvested from tamoxifen-treated mice bearing the transgenes. SAE and SMG cells are differentially isolated and cultured for two passages in CRC to obtain sufficient cells for transplantation. SCID mice are injured using 300 mg/kg naphthalene (sufficient to denude the airways) and 1×10⁶ cells are delivered at 36-48 hrs post-injury. The effects of Lef-1 expression on cellular engraftment in the injured lung by each progenitor cell type (i.e., BC^{WT}/BC^{Lef-1KI} and MEC^{Lef-1KI}) is evaluated. Mixed populations (50:50) of green and red cells (e.g. MEC^{WT}/MEC^{Lef-1KI}) are delivered to enable differential engraftment of both types of cells. Mice are euthanized at 21 days post-transplantation and evaluated for cellular engraftment in the trachea and conducting airways using Metamorph software. Also, tracheal sections are immunostained for the following cellular markers: αSMA (MEC); K5, p63, K14, NGFR (BC); Muc5AC (goblet cell), tubulin IV (ciliated cell), and Scgb1A1 and Scgb3A2 (club cell). The percentage of cells expressing each cell-type marker is evaluated for three populations (native cells with no transgene, GFP⁺, and Tomato⁺). The use of two differentially tagged populations of cells allow for clonal analysis, comparing the number and size of green and red clones on the airway surface. The number of clones may be proportional to the number of stem cells that engrafted into the airways. Furthermore, mixed population comparisons are an internal control for variable distribution of cells following delivery to the lung.
Results: Lef-1 expression enhances the level of engraftment of both MECs and BCs, and the contribution of MEC^{WT} to repair of epithelium is equal or greater than that of BC^{WT}. It is possible that permanent Lef-1 activation results in reduced engraftment, since under physiological conditions it is induced only transiently in MECs after injury and migration to the SAE. The ability of transient Lef-1 activation to improve the abilities of MECs and BCs to engraft is investigated. If sustained Lef-1 expression leads to improved long-term engraftment of MECs and/or BCs, stem cell niches that are reestablished within the airway are determined. To this end, a second mild injury is performed on transplanted mice to induce further expansion of cells (200 mg/kg naphthalene). It is hypothesized that the number of clones retained at various levels of the airway are proportional to the number of stem cells that are capable of reestablishing a niche that is sufficiently responsive to reinjury. Furthermore, properly adapted stem cells on the airway surface expand and give rise to larger clones following reinjury.
Transient activation of Lef-1 expression using dCas9-VP64 will enhance the ability of MECs and BCs to engraft into injured airways of mice. The engraftment rates of MECs and BCs into naphthalene injured SCID mice following transient activation of Lef-1 expression using dCas9-VP64 (FIG. 9G) are compared. This approach more accurately model the activation state of MECs transiently expressing Lef-1 following injury, thus providing more native reprogramming of MECs and BCs, as desirable in cell therapy.
Approach: BCs and MECs are isolated from tamoxifen-induced K5-CreERT2:ROSA-LsL-tdTomato and αSMA-CreERT2:ROSA-LsL-tdTomato mice, respectively. FACS-purified cells are sequentially transduced with dCas9-VP64 and Cpf1-P2A-GFP encoding lentiviruses (each with distinct selectable markers). Transient activation of Lef-1 is achieved by transfection with Lef-1 promoter dCas9 sgRNAs, while a co-transfected EGFP Cpf1 sgRNA inactivate the EGFP reporter. A switch from EGFP⁺ to EGFP⁻ marks transfected cells, which are enriched for activated Lef-1 expression. Lef-1KI transgene increases Lef-1 expression 150-fold over baseline in MEC^{WT} (FIG. 14). Thus, Lef-1 induction by dCas9-VP64, which provided 200- and 500-fold amplification of Lef-1 with sgRNA3 or sgRNA1-3, respectively, should suffice (FIG. 16G). Polyclonal pools of primary cells are transfected with Lef-1 promoter Cas9 sgRNAs sets and an EGFP Cpf1 sgRNA. Naphthalene injured transgenic SCID mice are seeded with these mixed populations of EGFP⁺ and EGFP⁻ cells, separately for BCs and MECs. Target transfection efficiencies are 50%, such that the extent of Tomato⁺GFP⁺ (control cells) to Tomato⁺GFP⁻ (Lef-1 activated cells) engraftment can be compared in each animal.
Results: Transient Lef-1 activation imparts a selective advantage for engraftment into the airways of injured mice, leading to greater engraftment of Tomato⁺GFP⁻ cells (enriched in gRNA transfection and Lef-1 activation) vs. Tomato⁺GFP⁺ cells (internal control; enriched for in non-transfected population).Tomato⁺GFP⁻ cells are enriched for Lef-1 expression, as shown using FACS and Q-PCR analysis, as in FIG. 16G, and c the levels of Lef-1 expression to those in Tomato⁺GFP⁺ cells are compared. MEC^{Lef-1} more efficiently engrafts than BC^{Lef-1}. However, both populations may be engrafted at higher rates than non-transfected Tomato⁺GFP⁺ MECs and BCs. If transient induction of Lef-1 facilitates engraftment, this approach can be adapted using ribonuclear complexes (RNPs) composed of dCas9-VP64 protein complexed to Lef-1 promoter sgRNAs for transfection.
Create an αSMA-IRES-Cre^{ERT2} ferret in which glandular progenitor cells can be lineage-traced. A transgenic ferret is created that can be used to translate findings from glandular stem cell studies in mice to CF ferrets. The long-term goal is to facilitate the creation of CF models in a species that, like humans, has SMGs throughout the cartilaginous airways, and to use these models to answer questions about glandular stem cell biology and to test novel approaches to cell therapy. Toward this aim, a ROSA-TG Cre reporter ferret (FIG. 17A-D) was created, using the transgene cassette that is present in the ROSA-TG mice. This model was generated using CRISPR/Cas9-directed insertion of the transgene cassette into intron 1 of the ROSA-26 locus. Four founder ferrets with this insertion were generated (FIG. 17B), and fibroblasts from these founders convert from tdTomato⁺ to EGFP⁺ following infection with an adenovirus vector encoding Cre (FIG. 10C,D). An αSMA-IRES-Cre^{ERT2} knock-in (KI) ferret is generated that can be used to trace and isolate glandular MECs when bred to the ROSA-TG transgenic background.
Glandular MECs are stem cells that are active throughout the ferret cartilaginous airways and contribute to airway repair in the CF ferret lung.
Approach: CRISPR/Cas9-mediated targeting in ferret zygotes has been used to generate four knock-in (KI) animal models (G551 D-CFTR, ΔF508-CFTR, ROSA-TG, and Z-allele alpha-1 antitrypsin). The principle for generating the αSMA-IRES-Cre^{ERT2} ferret is similar to the above in that it uses a single-stranded DNA (ssDNA) template for homology-directed repair (HDR) at gRNA cleavage site. The fragment in FIG. 10F is generated from gBlocks and cloned into a plasmid. A long ssDNA donor for HDR is prepared from the excised linear fragment that has been selectively dephosphorylated on one end. Use of Strandase (Clonetech), an exonuclease that selectively digests the phosphorylated strand, makes it possible to generate single strands from either the sense or antisense strand. The ssDNA template is purified for zygote injection by gel electrophoresis. Because the secondary structure at the target site can influence HDR efficiency, ssDNAs are created for both the sense and anti-sense strands, and tested separately. Single cell zygotes are generated by mating, and the pronucleus is injected with Cas9/sgRNA protein/RNA complex plus the ssDNA HDR template. Offspring are evaluated by Southern blotting of tail DNA, and integrity of the locus will be confirmed by PCR of the flanking sequences followed by sequencing.
Results: IRES2 (internal ribosome entry site) was chosen as the site of KI rather than a peptide cleavage fragment (e.g., P2A or T2A) since both ends of the αSMA protein associate with other proteins and such self-cleaving peptides leave a residual peptide on the target protein. While the efficiency of Cre^{ERT2} expression from the IRES is lower than that of a self-cleaving peptide, it has been successfully used in many KI mouse models, and Cre expression is sufficient for marking MECs. The αSMA-IRES-Cre^{ERT2} are bred to ROSA-TG ferrets, and it is confirmed that the labeling of glandular MECs following tamoxifen induction is cell-type specific. Triple transgenic ferrets on the G551D-CFTR background are also generated. This model can evaluate the contribution of glandular MECs to CF airway repair. These αSMA-IRES-Cre^{ERT2}:ROSA-TG ferrets are useful for a number of applications beyond the study of stem cell biology in a species with glands throughout the cartilaginous airways (unlike mice). For example, the ability to isolate glandular MECs by lineage tracing facilitates the evolution of rAAV vectors that specifically target this glandular progenitor. Given that glandular MECs contribute to both SMG and SAE lineages, we this stem cell population may be very valuable in directing the repair of CF SMGs.

### Example 3

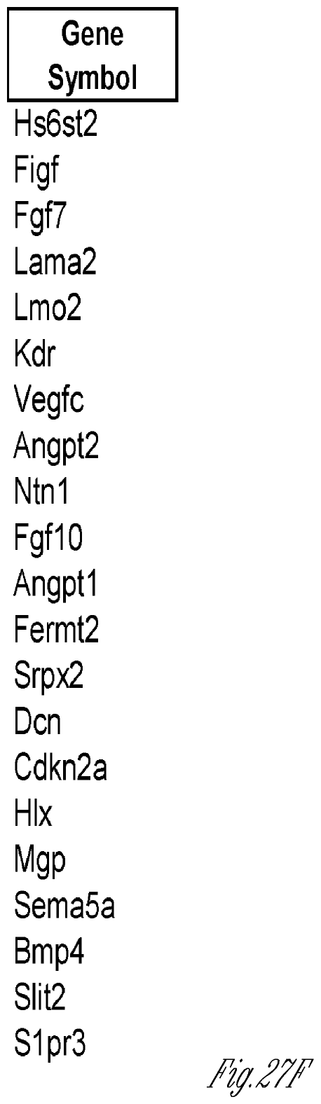

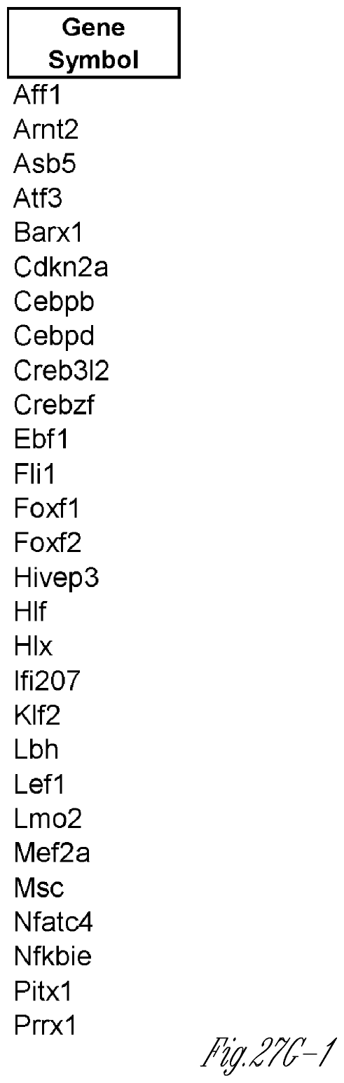

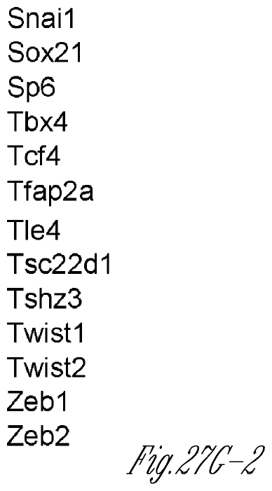

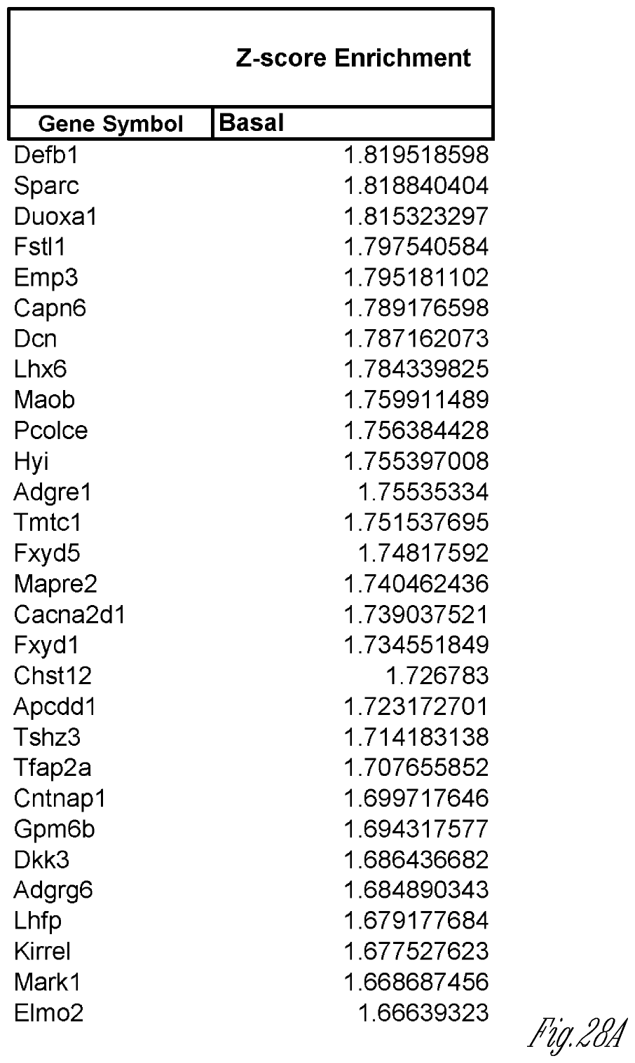

FIG. 29 shows expression of Lef1 in MECs induces ionocyte differentiation. Ionocytes are the top Cftr expressing cells in the airways (Montoro et al., 2018; Plasschaert et al., 2018). Since the ionocytes express Cftr at an extremely high levels, replenishing ionocyte will be highly beneficial to CF patients and for airway regeneration after airway injuries. The present method generates ionocytes which can be used as a cell based therapy, e.g., for CF patients.

### Example 4

The disclosure provides a high throughput screen for chemical and genetic activators/modulators of Lef1. In one embodiment the identified compounds or molecules may be found in miRNA screens that identify inhibitors of Lef1, or in a chemical library screen for compounds that activate Lef1 expression, in stem cells. In one embodiment, an IRES-GFP reporter could be knocked in to the 3′UTR of Lef1 and then employed in the screen.

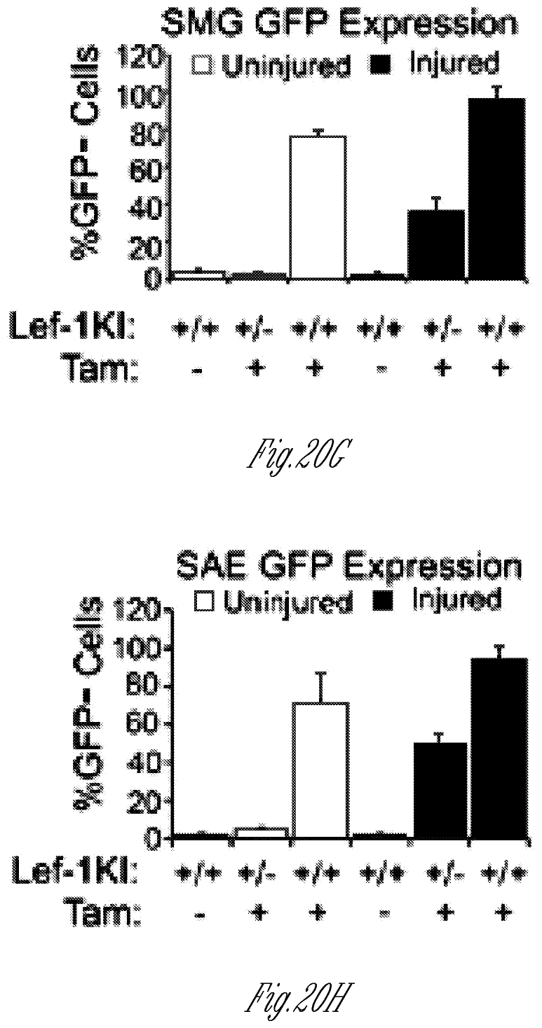

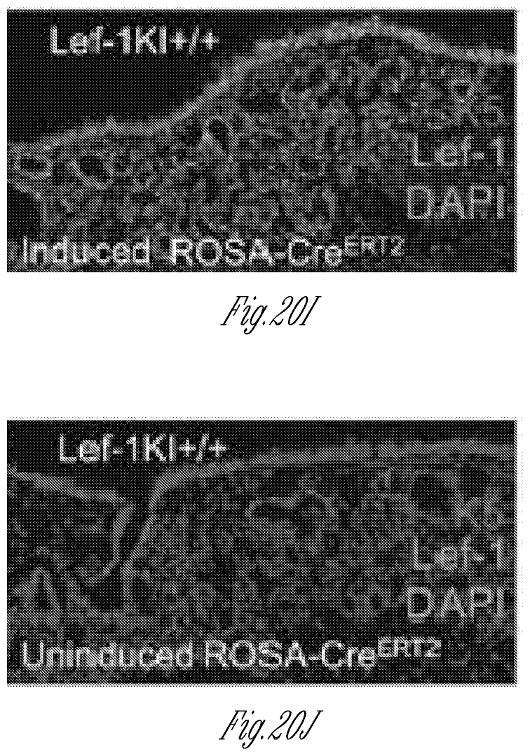

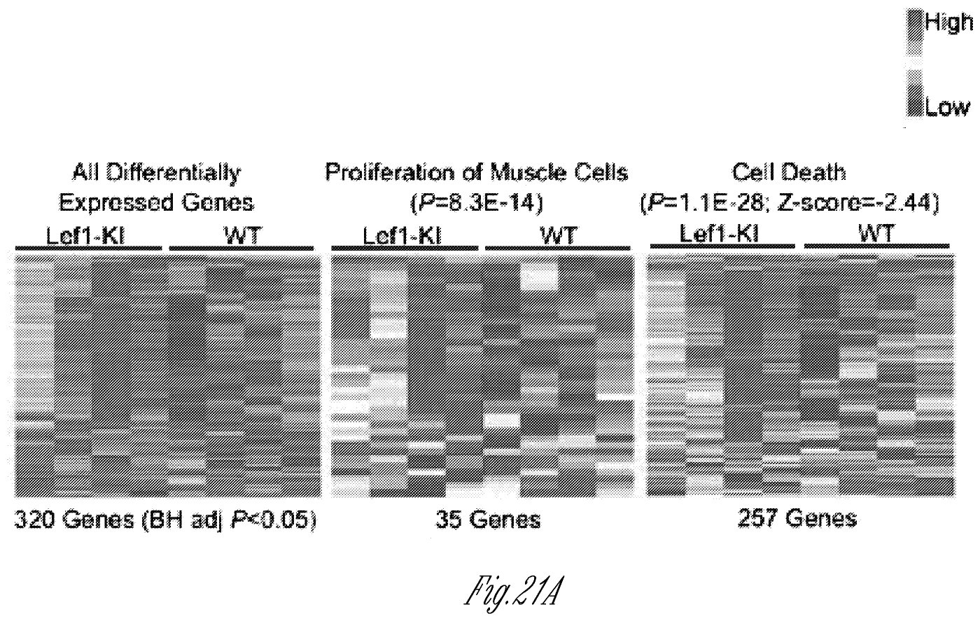

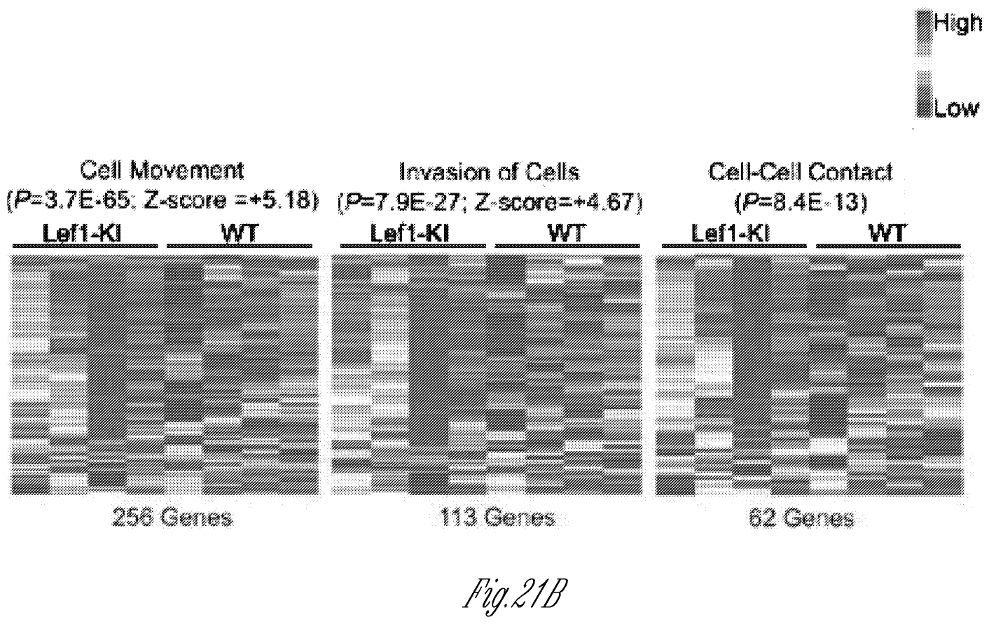

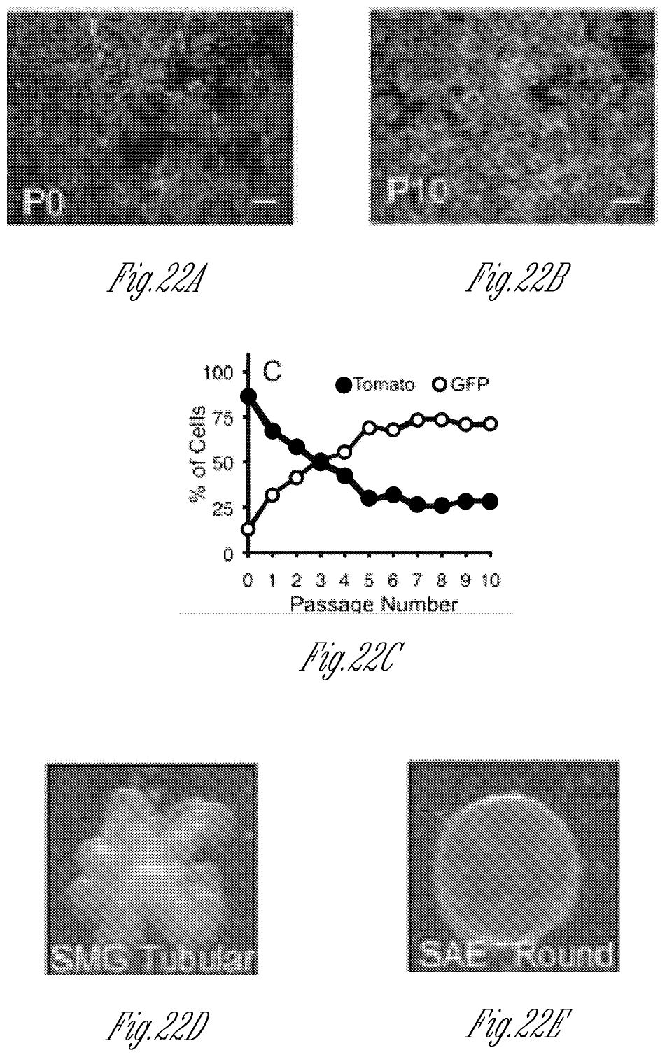

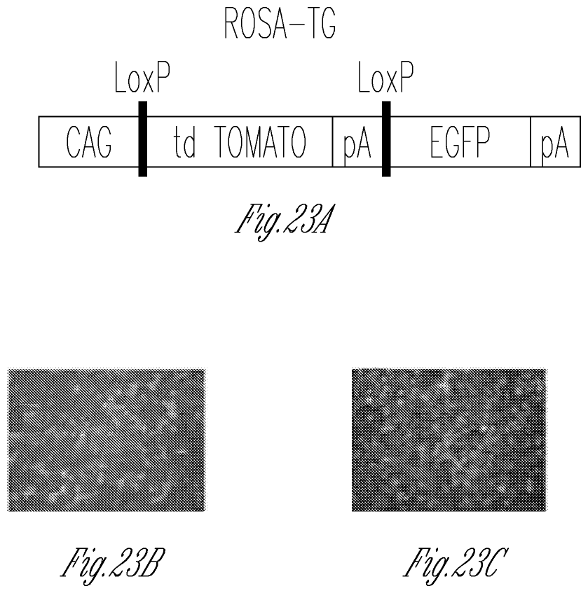

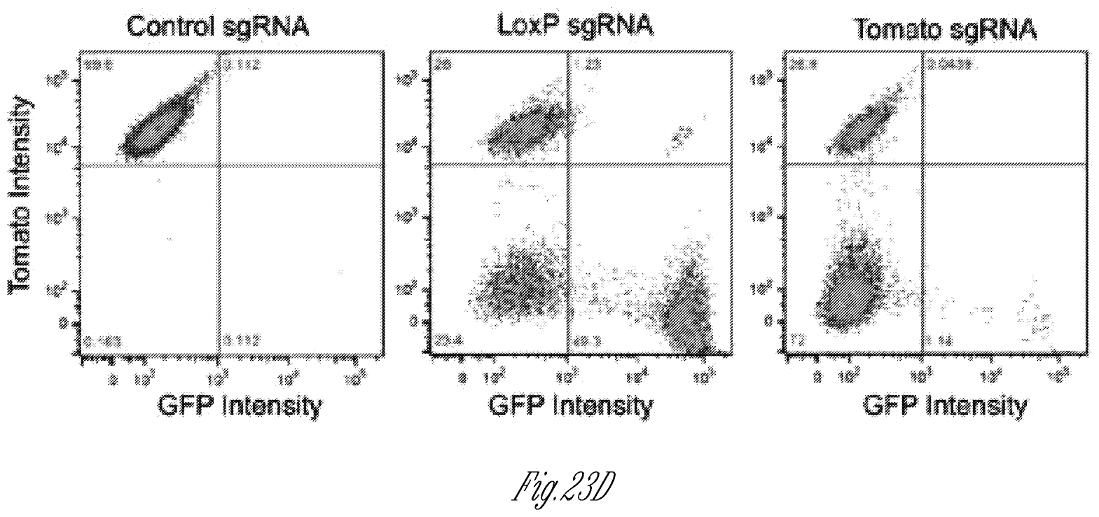

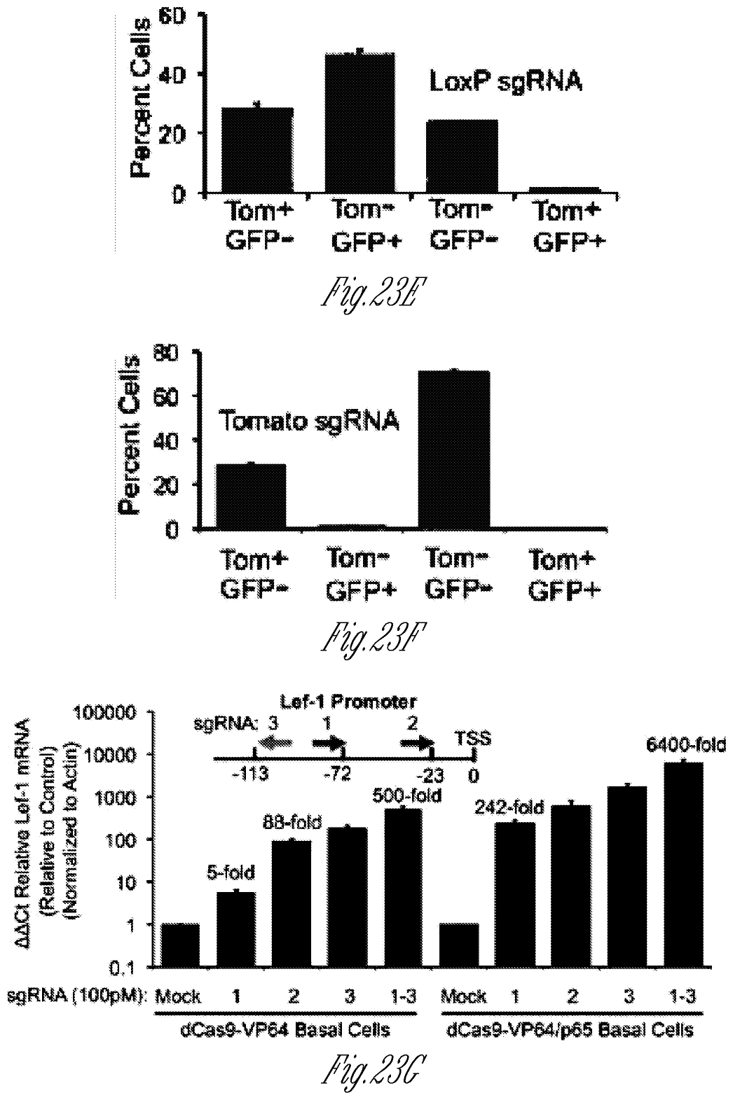

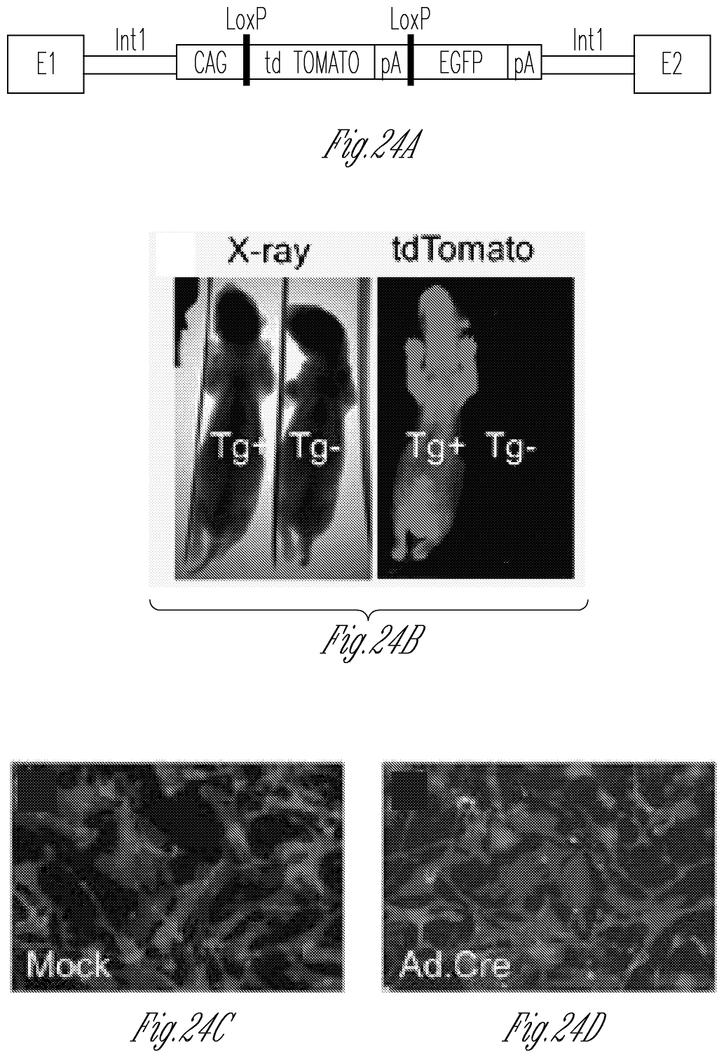

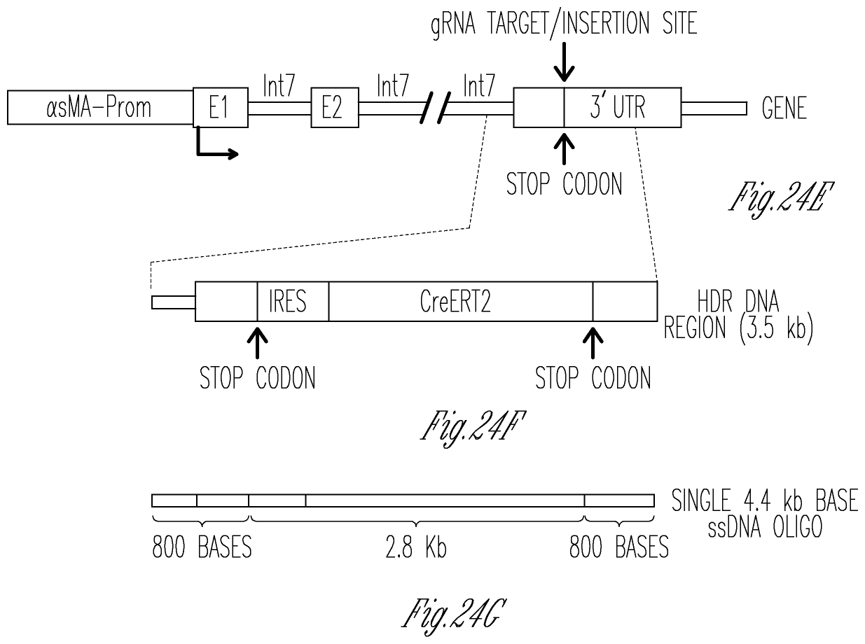

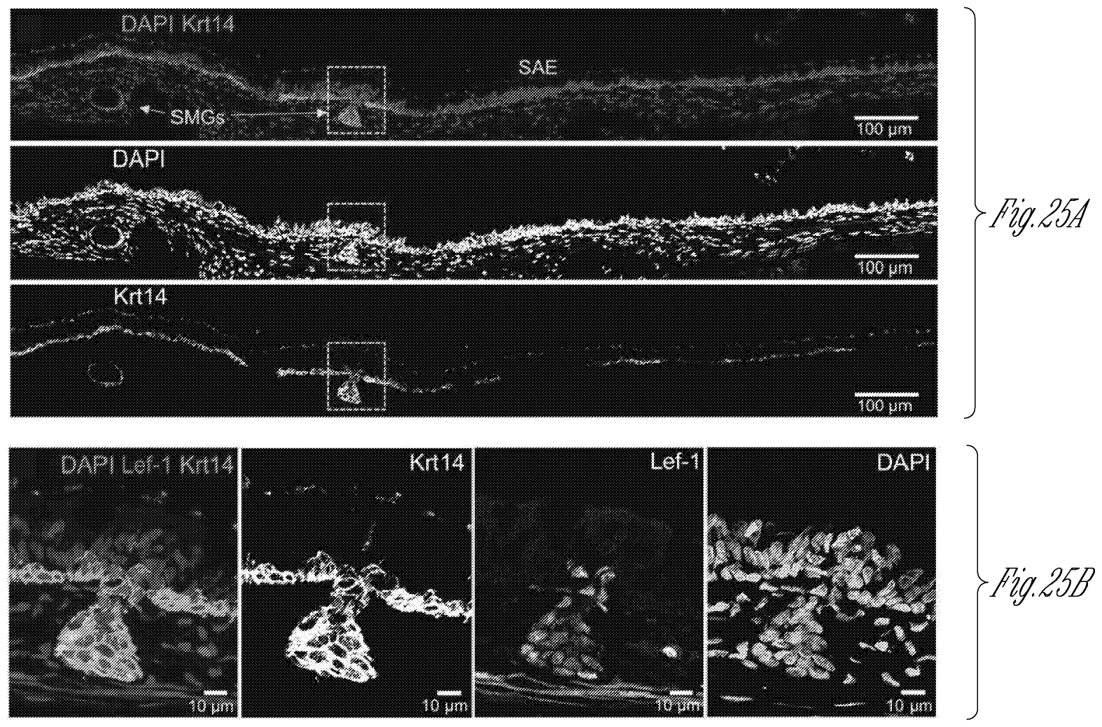

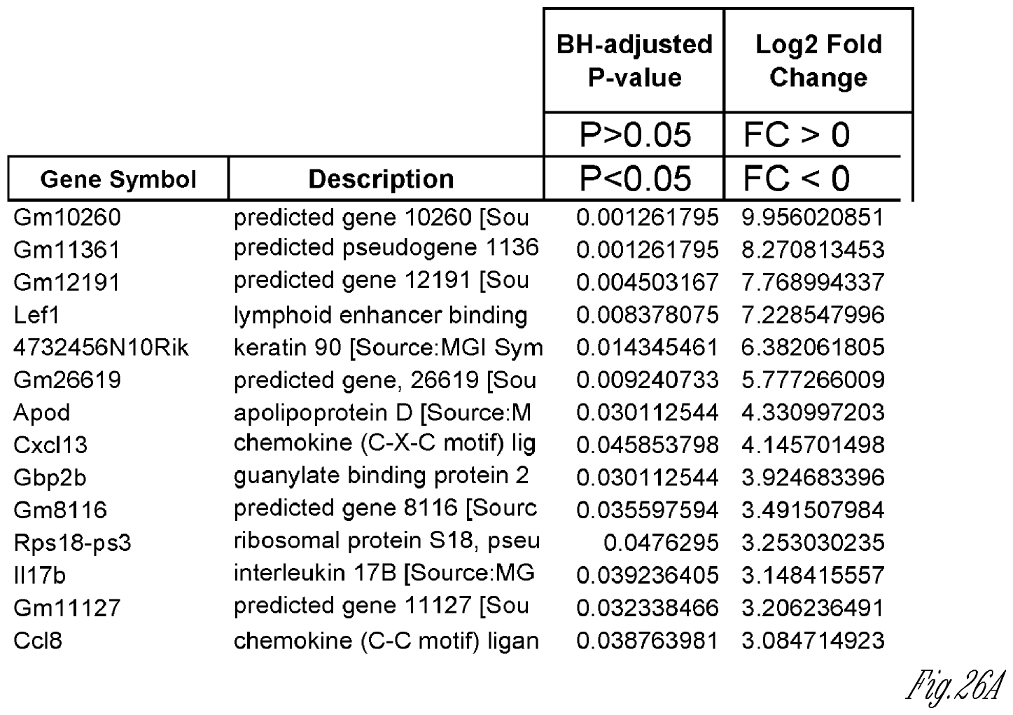

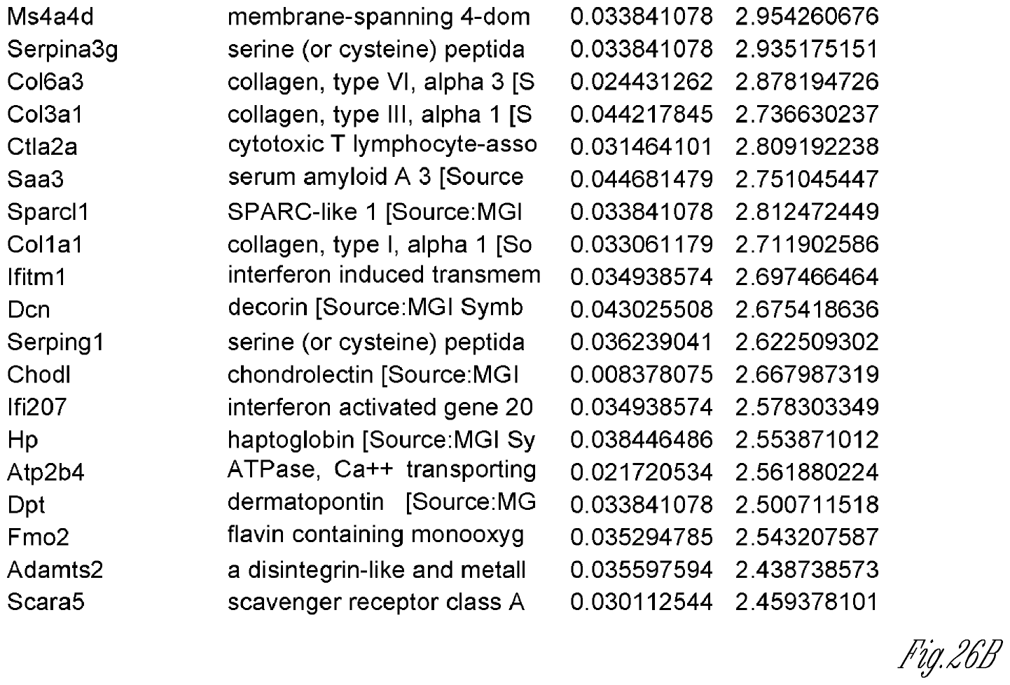

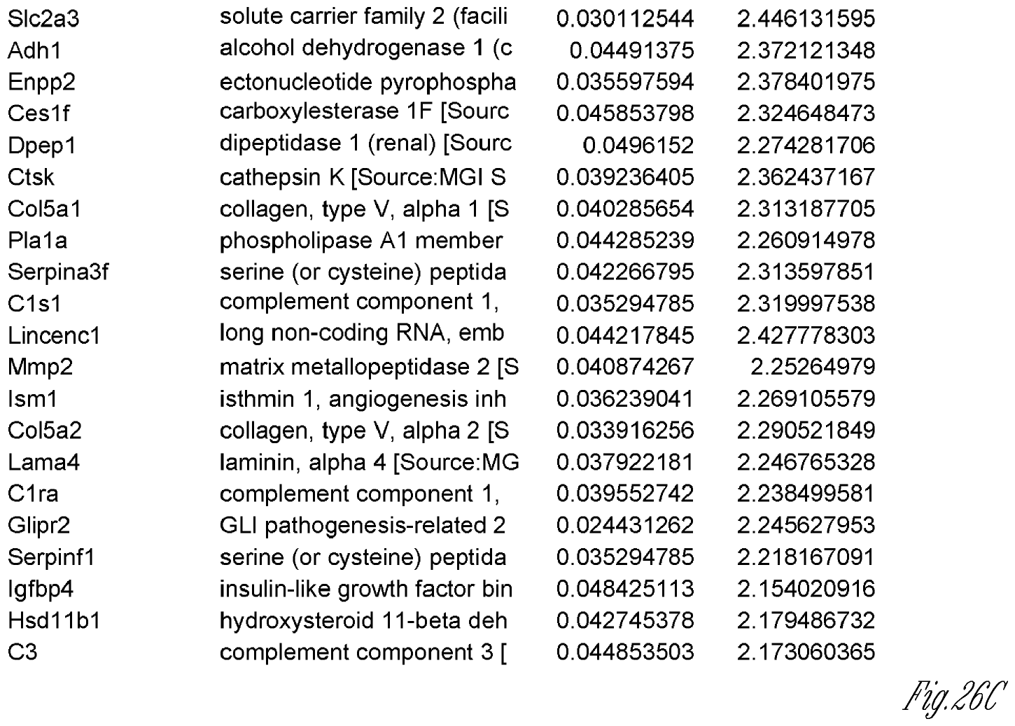

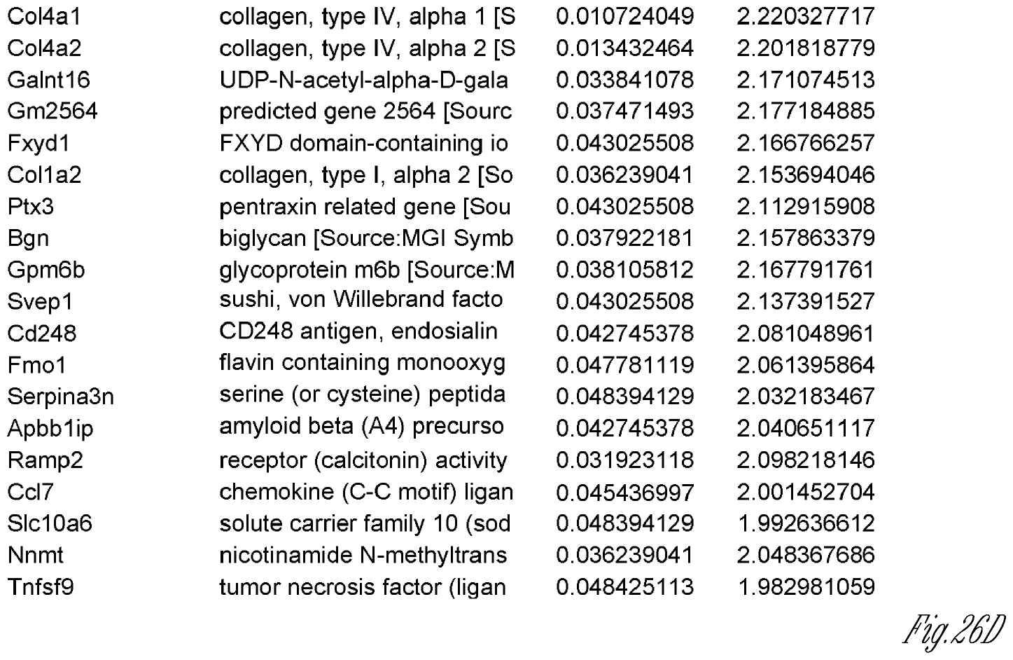

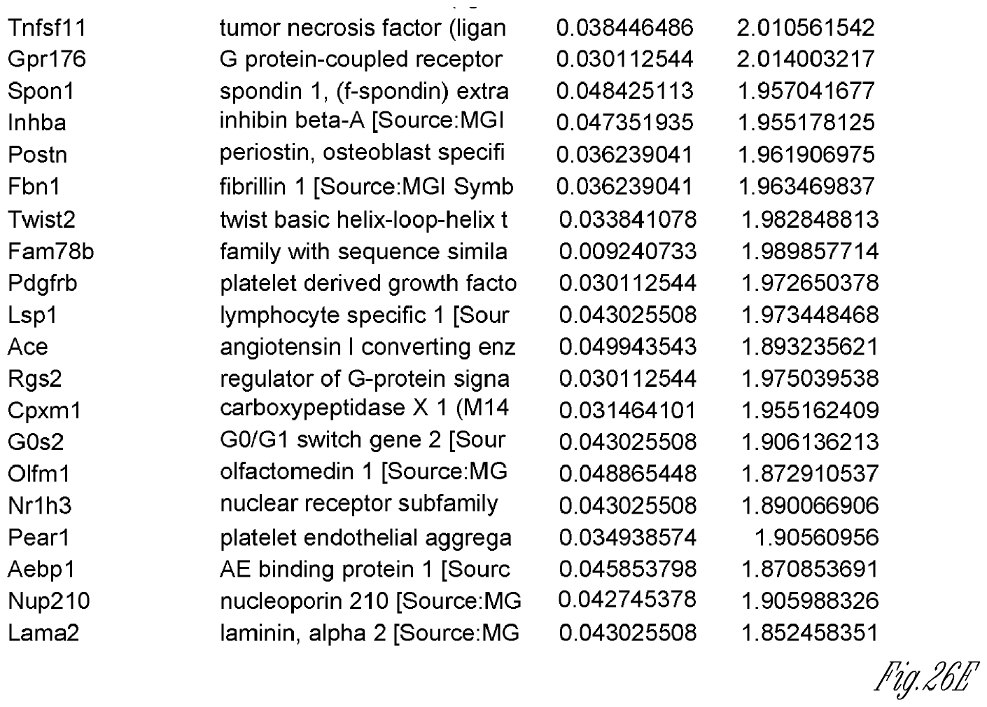

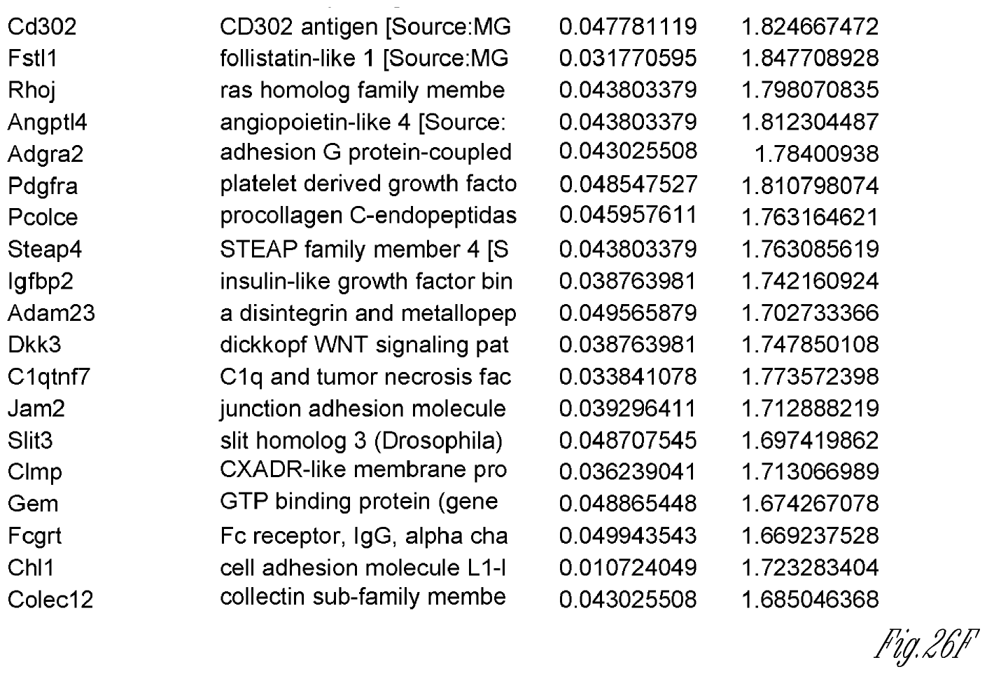

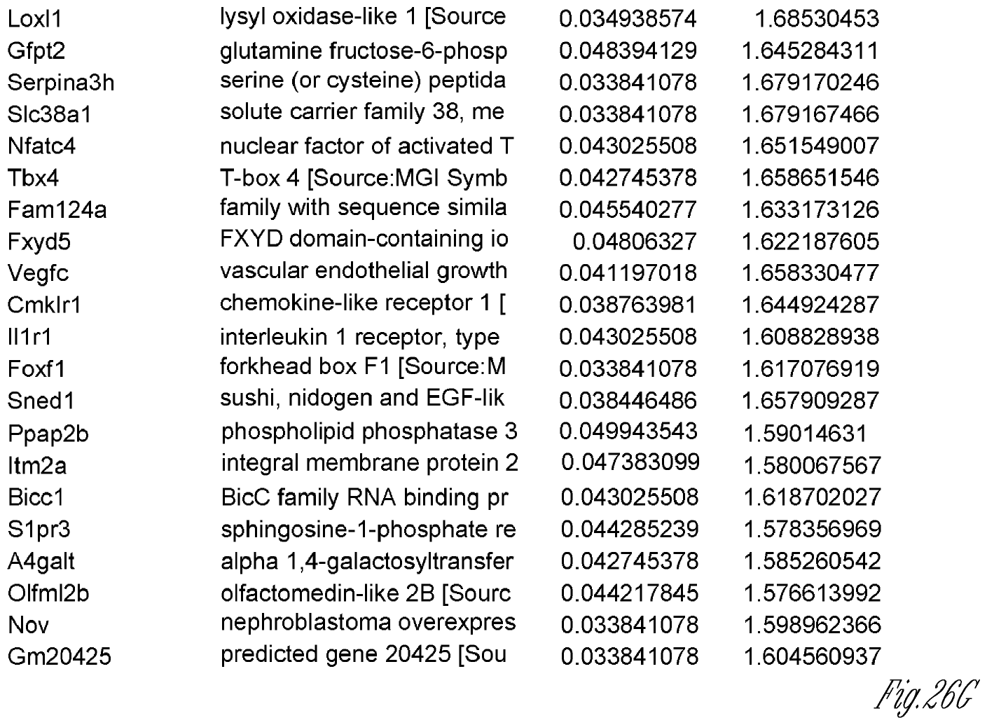

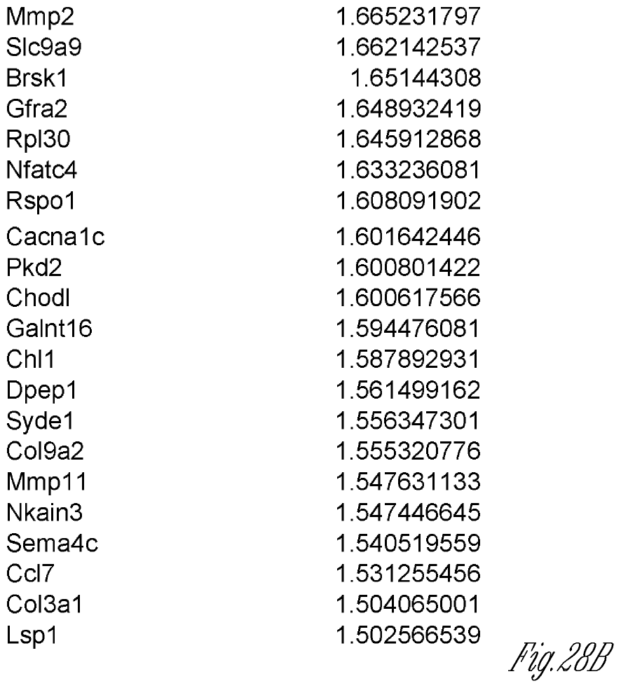

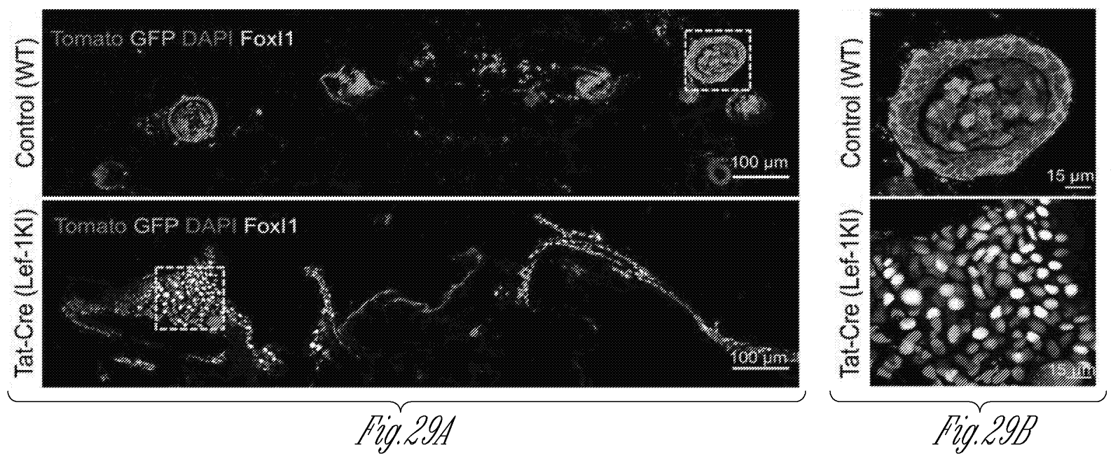

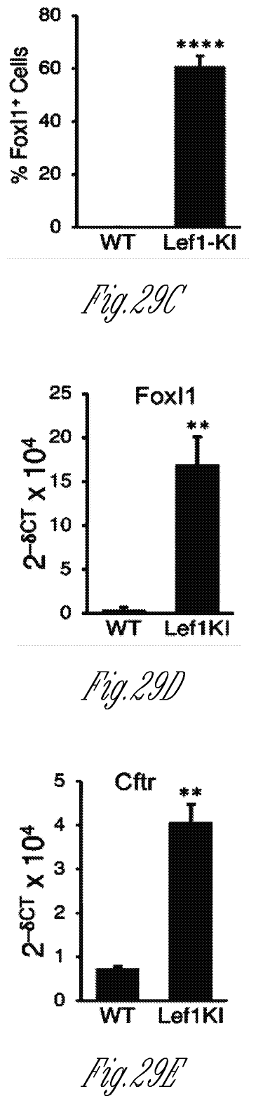

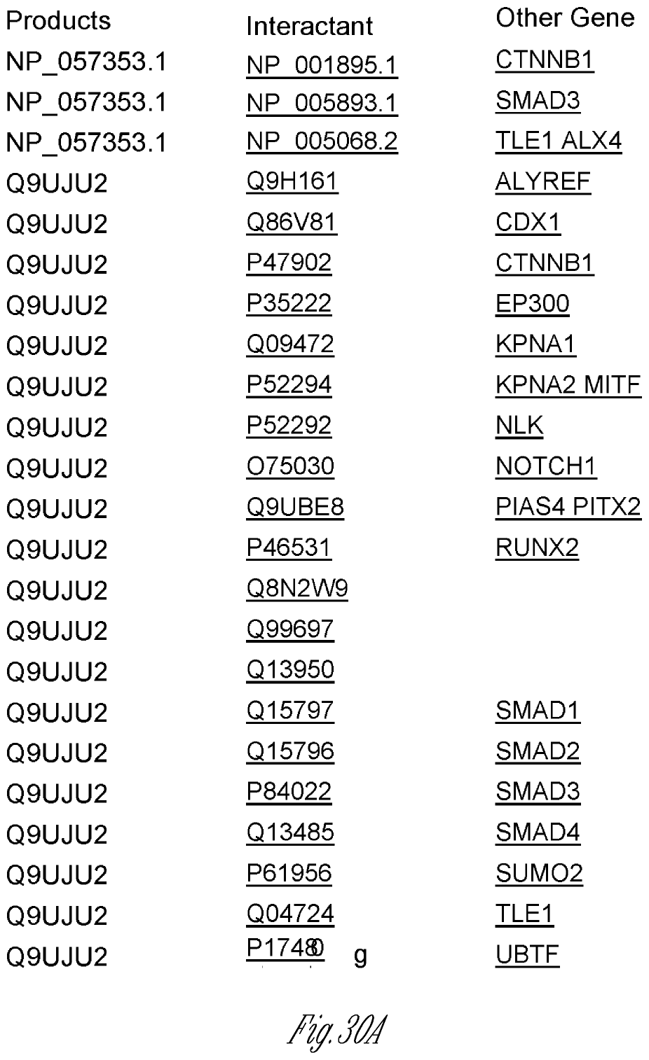

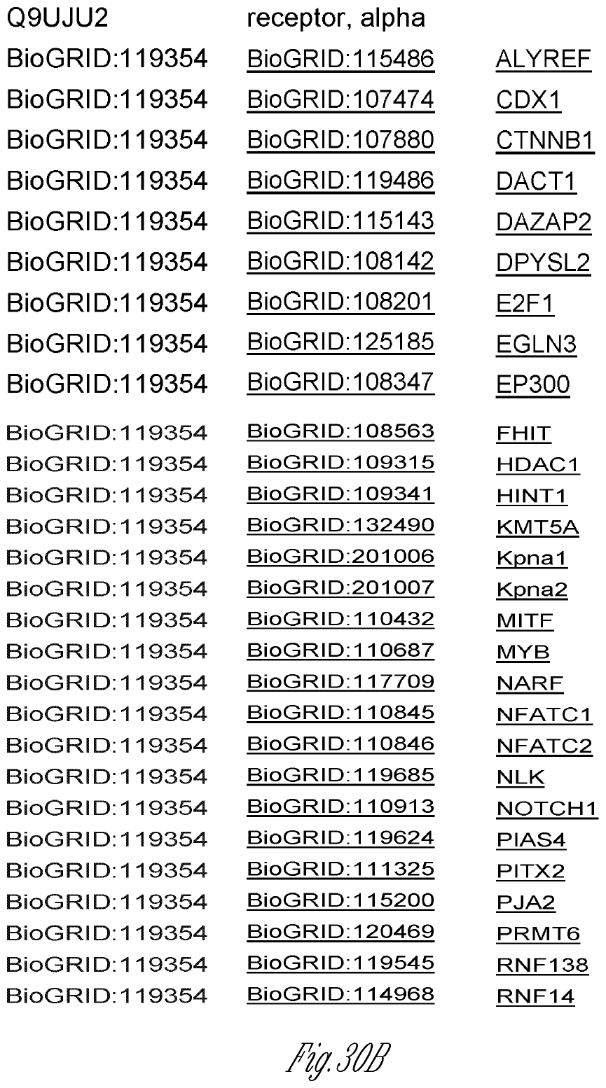

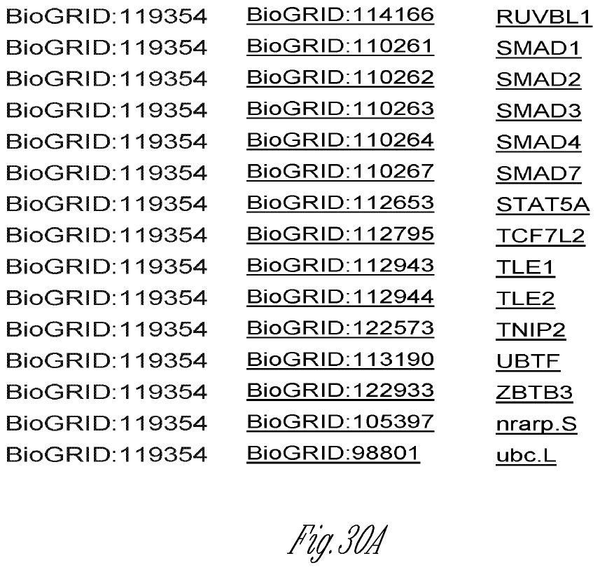

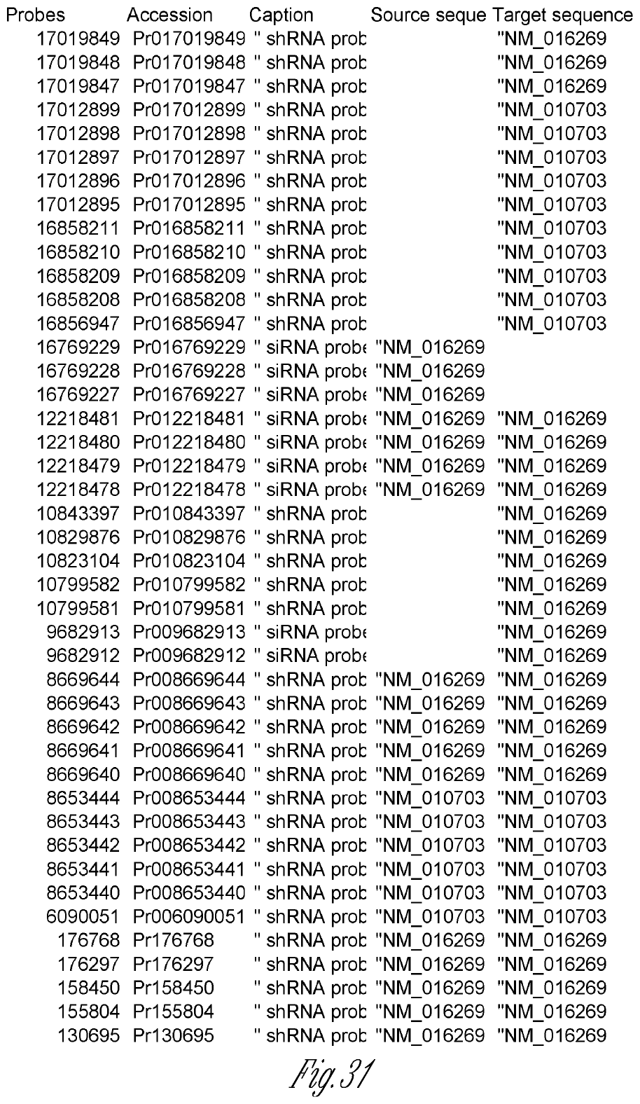

In one embodiment, compounds or molecules that modulate Lef-1 may be employed therapeutically, e.g., in airway stem cells. In one embodiment ethacrynic acid may be employed to inhibit the recruitment of LEF1 to DNA promoters and restore cylindromatosis (CYLD) expression in chronic lymphocytic leukemia (CLL) cells. Moreover, small molecule modulators of Glycogen synthase kinase 3 (GSK-3), which is a negative regulator of Wnt signaling and downstream Lef-1 activity, may be used to inactivate GSK-3, leading to increased Lef-1 activity. There are several genes that interact with Lef-1 (see FIG. 30). See also https://www.ncbi.nlm.nih.gov/gene/51176. There are also 43 known shRNA molecules used for Left expression silencing (FIG. 21).

### REFERENCES

Aloia et al., J. Physiol. 594:4815 (2016).
Anderson et al., Am. J. Respir. Cell. Mol. Biol., 56:716 (2017).
Arcangeli et al., Stem Cells 32:1043 (2014).
Benjamin et al., Dev. Biol. 335:407 (2009). pmcid:PMC2783258.
Benoit et al., Biol. Cell 101:695 (2009).
Bledzka et al., J. Cell Biol., 213:97 (2016).
Boecker et al., Mod. Pathol. 26:1086 (2013).
Bono et al., Mol. Biol. Cell 12:891 (2001). w
Borowsky et al., J. Cell Biol. 143:429 (1998).
Borthwick et al., Am. J. Respir. Cell Mol. Biol. 24:662 (2001).
Browne et al., Cell Cycle 9:886 (2010).
Cebrian et al., Cell Rep. 7:127 (2014).
Clevers et al., Science 346:1248012 (2014).
Deryugina et al., Cancer Metastasis Rev. 25:9 (2006).
Driskell et al., Am. J. Physiol. Lung Cell Mol. Physiol. 287:L752 (2004).
Driskell et al., Dev. Biol. 305:90 (2007).
Duan et al., Development, 126:4441 (1999).
Duan et al., Am. J. Respir. Cell Mol. Biol. 18:750 (1998).
Ebert et al., I. Cerebral cortex. Neuroscience 142:1145 (2006).
Engelhardt et al., Development 121:2031 (1995).
Engelhardt, Am. J. Respir. Cell Mol. Biol. 24:649 (2001).
Engelhardt et al., J. Clin. Investig. 90:2598 (1992).
Filali et al., J. Biol. Chem., 277:33398 (2002).
Forteza et al., J. Biol. Chem. 287:42288 (2012).
Friedl and Mayor Tuning Collective Cell Migration by Cell-Cell Junction Regulation. Cold Spring Harb Perspect Biol 9 (2017).
Fukuda et al., J. Biol. Chem. 289:28363 (2014).
Gagliardi et al., Crit. Rev. Oncol. Hematol. 109:63 (2017).
Gehart and Clevers, Trends Genet., 31:344 (2015).
Ghosh et al., Am. J. Respir. Cell. Mol. Biol. 45:459 (2011).
Ghosh et al., Am. J. Respir. Cell Mol. Biol. 56:1 (2017).
Giangreco et al., Proc. Natl. Acad. Sci. U.S.A. 106:9286 (2009).
Harris et al., Front Neural. Circuits 8:76 (2014).
Hayer et al., Nat. Cell Biol. 18:1311 (2016).
Hegab et al., J. Vis. Exp. :e3731 (2012).
Hegab et al., Respirology 17:1101 (2012).
Hegab et al., Stem Cells 29:1283 (2011).
Hernandez-Vasquez et al., J. Biol. Chem. 292:12178 (2017).
Hogan et al., Cell Stem Cell, 15:123 (2014).
Huang et al., J. Cell Sci. 112(Pt 23):4193 (1999).
Ino et al., Proc. Natl. Acad. Sci. U.S.A., 99:365 (2002).
Konermann et al., Nature 517:583 (2015).
Kretzschmar and Watt, Cell 148:33 (2012).
Lagarrigue et al., Nat. Commun. 6:8492 (2015).
Lanza and Atala, Essentials of stem cell biology, 3rd edn (Amsterdam; Boston: Academic Press) (2014).
Lee et al., J. Biol. Chem. 284:5119 (2009).
Li and Dewey, BMC Bioinformatics 12:323 (2011).
Liu et al., Am. J. Physiol. Lung Cell Mol. Physiol. 299:L694 (2010).
Liu et al., Curr. Top. Dev. Biol. 64:33 (2004).
Liu et al., Methods Enzmol., 419:285 (2006).
Liu et al., Proc. Am. Thorac. Soc. 5:682 (2008).
Lu et al., J. Cell. Sci. 115:4641 (2002).
Lynch and Engelhardt, J. Cell Biochem. 115:1637 (2014).
Lynch et al., Stem Cells, 34:2758 (2016).
Martignani et al., Exp. Cell. Res. 338:245 (2015).
Montoro et al., Nature 560:321 (2018).
Mou et al., Cell Stem Cell 19:217 (2016).
Muzumdar et al., Genesis 45:593 (2007).
Nikolic et al., Elife, 6 (2017).
Nusse and Clevers, Cell 169:985 (2017).
Pardo-Saganta et al., Am. J. Respir. Cell Mol. Biol. 48:364 (2013).
Plasschaert et al., Nature 560:377 (2018).
Prater et al., Nat. Cell Biol. 16:942 (2014).
Rajagopal and Stanger, Dev. Cell 36:133 (2016).
Ramesh et al., Mol. Cell Biol., 34:2600 (2014).
Rawlins and Perl, Am. J. Respir. Cell Mol. Biol. 46:269 (2012).
Regan and Smalley, Stem Cell Rev. 3:124 (2007).
Rios et al., Breast Cancer Res. 18:116 (2016).
Rios et al., Nature, 506:322 (2014).
Rock et al., Cell Stem Cell 8:639 (2011).
Schnapp et al., J. Biol. Chem. 270:23196 (1995).
Shibue et al., Cancer Cell 24:481 (2013).
Sun et al., Development 143:4115 (2016).
Tadokoro et al., Development 143:764 (2016).
Tata and Rajagopal, Development 144:755 (2017).
Tata et al., Nature 503:218 (2013).
Teng and Li, Clin. Exp. Metastasis 31:367 (2014).
Tetteh et al., Trends Cell Biol. 25:100 (2015).
Tonami et al., J. Cell Sci. 124:1214 (2011).
Umezu-Goto et al., J. Cell. Biol. 158:227 (2002).
Van Keymeulen et al., Nature 479:189 (2011).
Vandewalle et al., Cell Mol. Life Sci. 66:773 (2009).
Visvader and Clevers, Nat. Cell Biol. 18:349 (2016).
Wendling et al., Genesis 47:14 (2009).
Wilson et al., J. Cell Sci., 127:3039 (2014).
Xie et al., Am. J. Physiol. Lung Cell Mol. Physiol. 306:L645 (2014).
Xie et al., J. Clin. Invest. 121:3144 (2011).
Xie et al., J. Clin. Invest. 126:3063 (2016).
Xie et al., W., Am. J. Physiol. Lung Cell Mol. Physiol. 306:L645 (2014).
Yuan et al., Int. J. Oncol. 49:305 (2016).
Zhao et al., Dev. Cell 30:151 (2014).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. An in vitro method to identify modulators of LEF-1 or other TCF or Wnt signaling, comprising: contacting one or more test compounds with isolated mammalian myoepithelial stem cells (MECs) or basal cells derived therefrom, mammalian cells that exogenously express Lef-1 or TCF, or mammalian cells, the genome of which is altered with a reporter gene so as to detect LEF-1 or TCF expression or Wnt signaling; and detecting or determining whether the one or more compounds alter the expression of Lef-1 or TCF, or alter Wnt signaling.

2. The method of claim 1 wherein at least one of the compounds is a Lef-1, TCF or Wnt activator.

3. (canceled)

4. The method of claim 1 wherein the compound is miRNA, DNA or protein.

5. The method of claim 1 wherein the cells are human cells.

6. The method of claim 1 wherein the genome of the cells is genetically altered with a vector having a marker gene inserted into the 3′UTR of a Lef-1 or TCF-1 gene.

7. The method of claim 1 6 wherein the reporter gene is a fluorescent gene.

8-11. (canceled)

12. An in vitro method to culture and/or expand mammalian stem cells, mammalian glandular myoepithelial stem cells (MECs) and optionally differentiate the MECs, or to prepare or induce ionocytes, comprising:
culturing myoepithelial stem cells (MECs) or basal cells derived therefrom with a composition comprising an effective amount of one or more modulators of LEF-1 or other TCF, or Wnt signaling.

13. The method of claim 12 wherein the composition comprises LEF-1 or TCF having at least 80% amino acid sequence identity to SEQ ID Nos 1-2 or 5-9, or an agent that induces the expression of LEF-1 or TCF in a mammalian cell.

14. (canceled)

15. A method to expand mammalian glandular myoepithelial stem cells (MECs) and optionally differentiate the MECs, or to induce ionocytes, or prevent, inhibit or treat a degenerative lung disease or disorder, or enhance airway repair, in a mammal, comprising: administering to the mammal an effective amount of a composition comprising one or more modulators of LEF-1 or other TCF or Wnt signaling or comprising cells exposed ex vivo to one or more modulators of LEF-1 or other TCF or Wnt signaling.

16. The method of claim 15 wherein the composition comprises a LEF-1 having at least 80% amino acid sequence identity to SEQ ID NO:1 or SEQ ID NO:9, cells transduced with an expression cassette comprising a nucleic acid encoding the LEF-1, cells exposed ex vivo to isolated LEF-1 having at least 80% amino acid sequence identity to SEQ ID NO:1 or SEQ ID NO:9, or an agent that induces the expression of LEF-1 in a mammalian cell, or the composition comprises a TCF-1 having at least 80% amino acid sequence identity to SEQ ID NO:5, cells transduced with an expression cassette comprising a nucleic acid encoding the TCF-1, cells exposed ex vivo to isolated TCF-1 having at least 80% amino acid sequence identity to SEQ ID NO:5, or an agent that induces the expression of TCF-1 in a mammalian cell.

17. The method of claim 15 wherein the composition comprises cells transduced with an expression cassette comprising a nucleic acid encoding the LEF-1 or TCF, or cells exposed ex vivo to an activator of LEF-1 or other TCF or Wnt signaling.

18-19. (canceled)

20. The method of claim 15 wherein the disease is COPD, emphysema, cystic fibrosis, related to allograft rejection such as chronic lung allograft dysfunction (CLAD), including bronchiolitis obliterative syndrome and/or restrictive allograft syndrome, primary lung graft dysfunction or the result of graft versus host disease (GvHD).

21. The method of claim 15 wherein the mammal is a human.

22. The method of claim 15 wherein the amount is administered before or after, or both before and after, a lung transplant.

23. The method of claim 15 wherein the amount is administered during a lung transplant.

24. The method of claim 15 wherein the composition is intratracheally, systemically or intranasally administered.

25. The method of claim 15 wherein the composition is bronchoscopically administered.

26. The method of claim 21 wherein the mammal has cystic fibrosis.

27. The method of claim 15 wherein the amount increases the number of ionocytes and/or enhances airway regeneration.

28. The method of claim 15 wherein the composition comprises lithium, CHIR 99021, BIO, SB-216763, CAS 853220-52-7, WAY 262611, R-spondin, norri, ICG-001, PNU-74654 or windorphen or wherein the composition comprises an activator of GSK-3 including but not limited to valproic acid, iodotubercidin, naproxen, cromolyn, famotidine, curcurmin, olanzapine, or a pyrimidine derivative.

29-33. (canceled)
